(19) United States
(12) Patent Application Publication
(10) Pub. No.: US 2023/0054194 A1
(43) Pub. Date: Feb. 23, 2023

(54) Methods of Treating Cancer

(71) Applicant: ARIAGEN, INC., Menlo Park, CA (US)

(72) Inventor: Peter Colabuono, Half Moon Bay, CA (US)

(73) Assignee: ARIAGEN, INC., Menlo Park, CA (US)

(21) Appl. No.: 17/295,132

(22) PCT Filed: Nov. 19, 2019
(86) PCT No.: PCT/US2019/062166

§ 371 Date: May 19, 2021

Related U.S. Application Data
(60) Provisional application No. 62/769,201, filed on Nov. 19, 2018.

(51) Int. Cl. A61K 31/426; A61K 31/4245; A61P 35/00

(52) U.S. Cl. CPC A61K 31/426; A61P 35/00; A61K 31/4245

(57) ABSTRACT

The present disclosure relates to methods of treating cancer in a patient using a combination of an inhibitor of an immune checkpoint protein and an indole compound or its phosphate derivative.

### CROSS-REFERENCE TO RELATED APPLICATION

This application claims priority from U.S. Provisional Application 62/769,201, filed Nov. 19, 2018, the disclosure of which is incorporated herein by reference in its entirety.

### FIELD OF THE INVENTION

The present disclosure relates to methods of treating cancer in a patient using a combination of an immune checkpoint inhibitor and an indole compound.

### BACKGROUND OF THE INVENTION

The aryl hydrocarbon (Ah) receptor (AhR) is a ligand-inducible transcription factor and a member of the basic helix-loop-helix/Per-Arnt-Sim (bHLH/PAS) superfamily. Upon binding to its ligand, AhR mediates a series of biological processes, including cell division, apoptosis, cell differentiation, adipose differentiation, hypothalamus actions, angiogenesis, immune system modulation, teratogenicity, tumorigenicity, tumor progression, chloracne, wasting, actions of hormonal systems (e.g., estrogen and androgen), and expression of genes of the P450 family (Poland et al., Annu. Rev. Pharmacol. Toxicol. 22:517-554 (1982); Poellinger et al., Food Addit Contam. 17(4):261-6 (2000); Bock et al., Biochem. Pharmacol. 69(10):1403-1408 (2005); Stevens et al., Immunology 127(3):299-311 (2009); Puga et al., Biochem Pharmacol. 69(2):199-207 (2005); Safe et al., Int J Oncol. 20(6):1123-8 (2002); Dietrich et al., Carcinogenesis 31(8):1319-1328 (2010); U.S. Pat. No. 7,419,992). The liganded receptor participates in biological processes through translocation from cytoplasm into the nucleus, heterodimerization with another factor named Ah receptor nuclear translocator, and binding of the heterodimer to the Ah response element of AhR-regulated genes, resulting in enhancement or inhibition of transcription of those genes.

The AhR is able to bind, with different affinities, to several groups of exogenous chemicals, or artificial ligands, including polycyclic aromatic hydrocarbons, e.g., 3-methylchoranthrene (3-MC), and halogenated aromatic hydrocarbons, e.g., 2,3,7,8-tetrachlorodibenzo-p-dioxin (TCDD). Studies with those AhR artificial ligands have helped in advancing the understanding of the AhR system. An endogenous or physiological ligand for the AhR has been identified as 2-(1′H-indole-3′-carbonyl)-thiazole-4-carboxylic acid methyl ester (ITE), with the following structure:

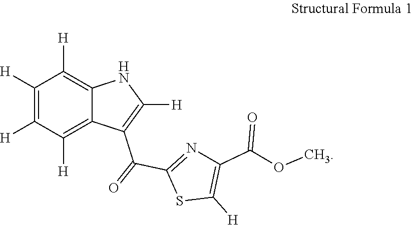 [SMILES: COC(=O)c1csc(C(=O)c2c[nH]c3ccccc23)n1]

See, e.g., Song et al., PNAS USA 99(23):14694-9 (2002); and U.S. Pat. No. 6,916,834.

### SUMMARY OF THE INVENTION

The present disclosure provides methods of treating cancer in a patient. In one embodiment, the method includes administering to the patient (1) a therapeutically effective amount of a compound of formula 2, or an enantiomer, diastereomer, or pharmaceutically acceptable salt thereof, and (2) a therapeutically effective amount of an inhibitor of an immune checkpoint protein,

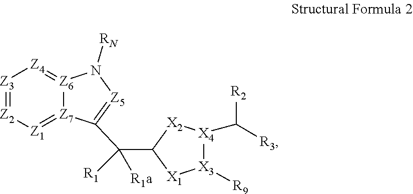

wherein:

X₁ is N (nitrogen), O (oxygen), S (sulfur), or C (carbon); X₂ is N (nitrogen), O (oxygen) S (sulfur), or C (carbon); X₃ is N (nitrogen), O (oxygen), S (sulfur) or C (carbon); and X₄ is N (nitrogen) O (oxygen), S (sulfur), or C (carbon), such that at least one of X₁, X₂, X₃ and X₄ is N, each of X₁, X₂, X₃ and X₄ is optionally selected to form a heteroaromatic, wherein the bond between X₁ and the adjacent carbon, between X₂ and the adjacent carbon, between X₁ and X₄, between X₂ and X₃, and between X₃ and X₄ can be a single bond or a double bond and the valence of X₁, X₂, X₃ and X₄ is completed with H or C₁-C₆ alkyl (i.e., the ring can be aromatic, partially saturated, or saturated);

Z₁ is N or CR₄, Z₂ is N or CR₅, Z₃ is N or CR₆, Z₄ is N or CR₇, Z₅ is N or CR₈, Z₆ is N or C, Z₇ is N or C, wherein no more than two of Z₁, Z₂, Z₃, Z₄, Z₅, Z₆, and Z₇ are N;

R₂ and R₃ are together selected from the group consisting of ═O, ═S, or ═NRₐ (Rₐ is H, C₁-C₆ alkyl, C₁-C₆ acyl, or —OR, R is H, C₁-C₆ alkyl, or C₁-C₆ acyl), or

R₂ and R₃ are each independently selected from the group consisting of —NRₐR_{b} (Rₐ and R_{b} are each independently H, C₁-C₆ alkyl, or C₁-C₆ acyl), hydrogen, deuterium, halo, amino, hydroxy, cyano, formyl, furyl, nitro, alkyl, haloalkyl, alkenyl, haloalkenyl, alkynyl, haloalkynyl, C₁-C₆ acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₀ (n=0 to 2, R₁₀ is directly connected to S), wherein R₁₀ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;

R₄, R₅, R₆, R₇, R₈, and R₉ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;

R_{N} is H, CN, C₁-C₆ alkyl, —OH, —(CO)—OR, or —OR, wherein R is H, C₁-C₆ alkyl, or C₁-C₆ acyl; and

R₁ and R₁ₐ are taken together to form ═NR_{b}, wherein R_{b} is H, C₁-C₆ alkyl, hydroxy, C₁-C₆ alkoxy (—O-alkyl), C₁-C₆ acyloxy, amino, or C₁-C₆ acyl, R₂ preferably can be ═O, R₃ preferably can be —OR, wherein R is H or C₁-C₆ alkyl; or

R₁ and R₁ₐ are taken together to form ═CR_{b}R_{c}, wherein R_{b} and R_{c} are each independently H, C₁-C₆ alkyl, alkoxy (—O-alkyl), thioalkoxy (—S-alkyl), cyano (—CN), or amino, R₂ preferably can be ═O, R₃ preferably can be —OR, wherein R is H or C₁-C₆ alkyl; or

R₁ and R₁ₐ are taken together to form ═O, ═NORₐ, or ═S, R₂ and R₃ preferably can be each independently —OR or —NRₐR_{b}, wherein R, Rₐ, and R_{b} are each independently H, C₁-C₆ alkyl, or C₁-C₆ acyl; or

R₁ and R₁ₐ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₂ (n=0 to 2, R₁₂ is directly connected to S), wherein R₁₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio; and optionally, adjacent R groups, together, can form a six- to twelve-membered ring.

In another embodiment, the method includes administering to the patient (1) a therapeutically effective amount of a compound of formula 2a, or an enantiomer, diastereomer, or pharmaceutically acceptable salt thereof, and (2) a therapeutically effective amount of an inhibitor of an immune checkpoint protein,

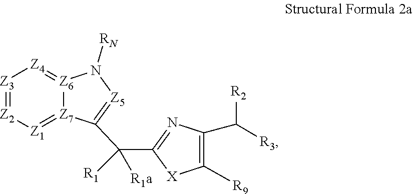

wherein:

X is either O (oxygen) or S (sulfur);

Z₁ is N or CR₄, Z₂ is N or CR₅, Z₃ is N or CR₆, Z₄ is N or CR₇, Z₅ is N or CR₈, Z₆ is N or C, Z₇ is N or C, wherein no more than two of Z₁, Z₂, Z₃, Z₄, Z₅, Z₆, and Z₇ are N;

R₂ and R₃ are together selected from the group consisting of ═O, ═S, or ═NRₐ (Rₐ is H, C₁-C₆ alkyl, C₁-C₆ acyl, or —OR, R is H, C₁-C₆ alkyl, or C₁-C₆ acyl), or

R₂ and R₃ are each independently selected from the group consisting of —NRₐR_{b} (Rₐ and R_{b} are each independently H, C₁-C₆ alkyl, or C₁-C₆ acyl), hydrogen, deuterium, halo, amino, hydroxy, cyano, formyl, furyl, nitro, alkyl, haloalkyl, alkenyl, haloalkenyl, alkynyl, haloalkynyl, C₁-C₆ acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₀ (n=0 to 2, R₁₀ is directly connected to S), wherein R₁₀ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;

R₄, R₅, R₆, R₇, R₈, and R₉ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;

R_{N} is H, CN, C₁-C₆ alkyl, —OH, —(CO)—OR, or —OR, wherein R is H, C₁-C₆ alkyl, or C₁-C₆ acyl; and

R₁ and R₁ₐ are taken together to form ═NR_{b}, wherein R_{b} is H, C₁-C₆ alkyl, hydroxy, C₁-C₆ alkoxy (—O-alkyl), C₁-C₆ acyloxy, amino, or C₁-C₆ acyl, R₂ preferably can be ═O, R₃ preferably can be —OR, wherein R is H or C₁-C₆ alkyl, or

R₁ and R₁ₐ are taken together to form ═CR_{b}R_{c}, wherein R_{b} and R_{c} are each independently H, C₁-C₆ alkyl, alkoxy (—O-alkyl), thioalkoxy (—S-alkyl), cyano (—CN), or amino, R₂ preferably can be αO, R₃ preferably can be —OR, wherein R is H or C₁-C₆ alkyl, or

R₁ and R₁ₐ are taken together to form ═O, ═NORₐ, or ═S, R₂ and R₃ preferably can be each independently —OR or —NRₐR_{b}, wherein R, Rₐ, and R_{b} are each independently H, C₁-C₆ alkyl, or C₁-C₆ acyl, or

R₁ and R₁ₐ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₂ (n=0 to 2, R₁₂ is directly connected to S), wherein R₁₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio; and

optionally, adjacent R groups, together, can form a six- to twelve-membered ring.

In another embodiment, the method includes administering to the patient (1) a therapeutically effective amount of a compound of formula 3, or an enantiomer, diastereomer, or pharmaceutically acceptable salt thereof, and (2) a therapeutically effective amount of an inhibitor of an immune checkpoint protein,

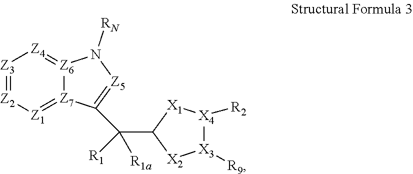

wherein:

X₁ is N (nitrogen), O (oxygen), S (sulfur), or C (carbon); X₂ is N (nitrogen), O (oxygen) S (sulfur), or C (carbon); X₃ is N (nitrogen), O (oxygen), S (sulfur) or C (carbon); and X₄ is N (nitrogen) O (oxygen), S (sulfur), or C (carbon), such that at least one of X₁, X₂, X₃ and X₄ is N, each of X₁, X₂, X₃ and X₄ is optionally selected to form a heteroaromatic, wherein the bond between X₁ and the adjacent carbon, between X₂ and the adjacent carbon, between X₁ and X₄, between X₂ and X₃, and between X₃ and X₄ can be a single bond or a double bond and the valence of X₁, X₂, X₃ and X₄ is completed with H or C₁-C₆ alkyl (i.e., the ring can be aromatic, partially saturated, or saturated);

Z₁ is N or CR₄, Z₂ is N or CR₅, Z₃ is N or CR₆, Z₄ is N or CR₇, Z₅ is N or CR₈, Z₆ is N or C, Z₇ is N or C, wherein no more than two of Z₁, Z₂, Z₃, Z₄, Z₅, Z₆, and Z₇ are N;

R₁ and R₁ₐ are taken together to form ═NR_{b}, wherein R_{b} is H, C₁-C₆ alkyl, hydroxy, C₁-C₆ alkoxy (—O-alkyl), C₁-C₆ acyloxy, amino, or C₁-C₆ acyl; or

R₁ and R₁ₐ are taken together to form ═CR_{b}R_{c}, wherein R_{b} and R_{c} are each independently H, C₁-C₆ alkyl, alkoxy (—O-alkyl), thioalkoxy (—S-alkyl), cyano (—CN), or amino; or

R₁ and R₁ₐ are taken together to form ═O, ═NORₐ, or ═S, wherein Rₐ is H, C₁-C₆ alkyl, or C₁-C₆ acyl, or

R₁ and R₁ₐ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₂ (n=0 to 2, R₁₂ is directly connected to S), wherein R₁₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio; and

R₂ and R₉ are each independently selected from the group consisting of hydrogen, aryl, heteroaryl, cycloalkyl, heterocycloalkyl, alkyl, —NR₂ₐC(O)OR_{2b}, —NR₂ₐC(O)R_{2b}, —(C₀-C₆ alkyl)-CONHSO₂R₂ₐ, —(C₀-C₆ alkyl)-CONHSO₂NR₂ₐR_{2b}, —(C₀-C₆ alkyl)-SO₂NHCOR₂ₐ, —(C₀-C₆ alkyl)-SO₂NHR₂ₐ, —(C₀-C₆ alkyl)-CONR₂ₐOR_{2b},

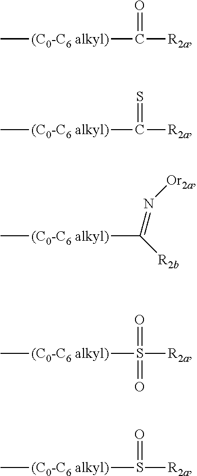

deuterium, halo, amino, hydroxy, cyano, formyl, nitro, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₀ (n=0 to 2, R₁₀ is directly connected to S), wherein R₁₀ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio, wherein R₂ₐ and R_{2b} are each independently H, C₁-C₆ alkyl, alkoxy (—O-alkyl), hydroxy, thioalkoxy (—S-alkyl), cyano (—CN), or amino;

R₄, R₅, R₆, R₇, and R₈ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;

R_{N} is H, CN, C₁-C₆ alkyl, —OH, —(CO)—OR, or —OR, wherein R is H, C₁-C₆ alkyl, or C₁-C₆ acyl; and

optionally, adjacent R groups, together, can form a six- to twelve-membered ring.

In another embodiment, the method includes administering to the patient (1) a therapeutically effective amount of a compound of formula 3c, or an enantiomer, diastereomer, or pharmaceutically acceptable salt thereof, and (2) a therapeutically effective amount of an inhibitor of an immune checkpoint protein,

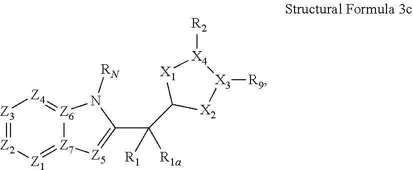

wherein:

X₁ is N (nitrogen), O (oxygen), S (sulfur), or C (carbon); X₂ is N (nitrogen), O (oxygen) S (sulfur), or C (carbon); X₃ is N (nitrogen), O (oxygen), S (sulfur) or C (carbon); and X₄ is N (nitrogen) O (oxygen), S (sulfur), or C (carbon), such that at least one of X₁, X₂, X₃ and X₄ is N, each of X₁, X₂, X₃ and X₄ is optionally selected to form a heteroaromatic, wherein the bond between X₁ and the adjacent carbon, between X₂ and the adjacent carbon, between X₁ and X₄, between X₂ and X₃, and between X₃ and X₄ can be a single bond or a double bond and the valence of X₁, X₂, X₃ and X₄ is completed with H or C₁-C₆ alkyl (i.e., the ring can be aromatic, partially saturated, or saturated);

Z₁ is N or CR₄, Z₂ is N or CR₅, Z₃ is N or CR₆, Z₄ is N or CR₇, Z₅ is N or CR₈, Z₆ is N or C, Z₇ is N or C, wherein no more than two of Z₁, Z₂, Z₃, Z₄, Z₅, Z₆, and Z₇ are N;

R₁ and R₁ₐ are taken together to form ═NR_{b}, wherein R_{b} is H, C₁-C₆ alkyl, hydroxy, C₁-C₆ alkoxy (—O-alkyl), C₁-C₆ acyloxy, amino, or C₁-C₆ acyl, or

R₁ and R₁ₐ are taken together to form ═CR_{b}R_{c}, wherein R_{b} and R_{c} are each independently H, C₁-C₆ alkyl, alkoxy (—O-alkyl), thioalkoxy (—S-alkyl), cyano (—CN), or amino, or

R₁ and R₁ₐ are taken together to form ═O, ═NORₐ, or ═S, wherein Rₐ is H, C₁-C₆ alkyl, or C₁-C₆ acyl, or

R₁ and R₁ₐ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₂ (n=0 to 2, R₁₂ is directly connected to S), wherein R₁₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio; and

R₂ and R₉ are each independently selected from the group consisting of hydrogen, aryl, heteroaryl, cycloalkyl, heterocycloalkyl, alkyl, —NR₂ₐC(O)OR_{2b}, —NR₂ₐC(O)R_{2b}, —(C₀-C₆ alkyl)-CONHSO₂R₂ₐ, —(C₀-C₆ alkyl)-CONHSO₂NR₂ₐR_{2b}, —(C₀-C₆ alkyl)-SO₂NHCOR₂ₐ, —(C₀-C₆ alkyl)-SO₂NHR₂ₐ, —(C₀-C₆ alkyl)-CONR₂ₐOR_{2b},

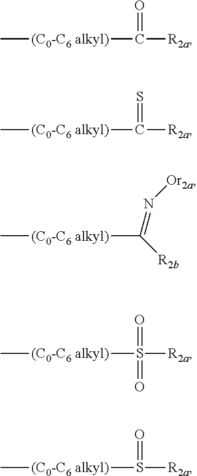

deuterium, halo, amino, hydroxy, cyano, formyl, nitro, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₀ (n=0 to 2, R₁₀ is directly connected to S), wherein R₁₀ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio, wherein R₂ₐ and R_{2b} are each independently H, C₁-C₆ alkyl, alkoxy (—O-alkyl), hydroxy, thioalkoxy (—S-alkyl), cyano (—CN), or amino;

R₄, R₅, R₆, R₇, and R₈ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;

R_{N} is H, CN, C₁-C₆ alkyl, —OH, —(CO)—OR, or —OR, wherein R is H, C₁-C₆ alkyl, or C₁-C₆ acyl; and

optionally, adjacent R groups, together, can form a six- to twelve-membered ring.

In another embodiment, the method includes administering to the patient (1) a therapeutically effective amount of a compound of formula 3a, or an enantiomer, diastereomer, or pharmaceutically acceptable salt thereof, and (2) a therapeutically effective amount of an inhibitor of an immune checkpoint protein,

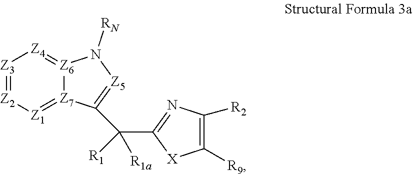

wherein:

X is either O (oxygen) or S (sulfur);

Z₁ is N or CR₄, Z₂ is N or CR₅, Z₃ is N or CR₆, Z₄ is N or CR₇, Z₅ is N or CR₈, Z₆ is N or C, Z₇ is N or C, wherein no more than two of Z₁, Z₂, Z₃, Z₄, Z₅, Z₆, and Z₇ are N;

R₁ and R₁ₐ are taken together to form ═NR_{b}, wherein R_{b} is H, C₁-C₆ alkyl, hydroxy, C₁-C₆ alkoxy (—O-alkyl), C₁-C₆ acyloxy, amino, or C₁-C₆ acyl, or

R₁ and R₁ₐ are taken together to form ═CR_{b}R_{c}, wherein R_{b} and R_{c} are each independently H, C₁-C₆ alkyl, alkoxy (—O-alkyl), thioalkoxy (—S-alkyl), cyano (—CN), or amino, or

R₁ and R₁ₐ are taken together to form ═O, ═NORₐ, or ═S, wherein Rₐ is H, C₁-C₆ alkyl, or C₁-C₆ acyl, or

R₁ and R₁ₐ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₂ (n=0 to 2, R₁₂ is directly connected to S), wherein R₁₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio; and

R₂ and R₉ are each independently selected from the group consisting of hydrogen, aryl, heteroaryl, cycloalkyl, heterocycloalkyl, alkyl, —NR₂ₐC(O)OR_{2b}, —NR₂ₐC(O)R_{2b}, —(C₀-C₆ alkyl)-CONHSO₂R₂ₐ, —(C₀-C₆ alkyl)-CONHSO₂NR₂ₐR_{2b}, —(C₀-C₆ alkyl)-SO₂NHCOR₂ₐ, —(C₀-C₆ alkyl)-SO₂NHR₂ₐ, —(C₀-C₆ alkyl)-CONR₂ₐOR_{2b},

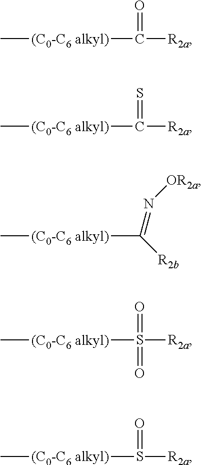

deuterium, halo, amino, hydroxy, cyano, formyl, nitro, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₀ (n=0 to 2, R₁₀ is directly connected to S), wherein R₁₀ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio, wherein R₂ₐ and R_{2b} are each independently H, C₁-C₆ alkyl, alkoxy (—O-alkyl), hydroxy, thioalkoxy (—S-alkyl), cyano (—CN), or amino;

R₄, R₅, R₆, R₇, and R₈ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;

R_{N} is H, CN, C₁-C₆ alkyl, —OH, —(CO)—OR, or —OR, wherein R is H, C₁-C₆ alkyl, or C₁-C₆ acyl; and

optionally, adjacent R groups, together, can form a six- to twelve-membered ring.

In another embodiment, the method includes administering to the patient (1) a therapeutically effective amount of a compound of formula 3b, or an enantiomer, diastereomer, or pharmaceutically acceptable salt thereof, and (2) a therapeutically effective amount of an inhibitor of an immune checkpoint protein,

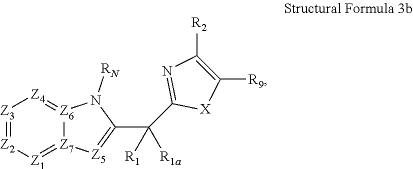

wherein:

X is either O (oxygen) or S (sulfur);

Z₁ is N or CR₄, Z₂ is N or CR₅, Z₃ is N or CR₆, Z₄ is N or CR₇, Z₅ is N or CR₈, Z₆ is N or C, Z₇ is N or C, wherein no more than two of Z₁, Z₂, Z₃, Z₄, Z₅, Z₆, and Z₇ are N;

R₁ and R₁ₐ are taken together to form ═NR_{b}, wherein R_{b} is H, C₁-C₆ alkyl, hydroxy, C₁-C₆ alkoxy (—O-alkyl), C₁-C₆ acyloxy, amino, or C₁-C₆ acyl; or

R₁ and R₁ₐ are taken together to form ═CR_{b}R_{c}, wherein R_{b} and R_{c} are each independently H, C₁-C₆ alkyl, alkoxy (—O-alkyl), thioalkoxy (—S-alkyl), cyano (—CN), or amino, or

R₁ and R₁ₐ are taken together to form ═O, ═NORₐ, or ═S, wherein Rₐ is H, C₁-C₆ alkyl, or C₁-C₆ acyl, or

R₁ and R₁ₐ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₂ (n=0 to 2, R₁₂ is directly connected to S), wherein R₁₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio; and

R₂ and R₉ are each independently selected from the group consisting of hydrogen, aryl, heteroaryl, cycloalkyl, heterocycloalkyl, alkyl, —NR₂ₐC(O)OR_{2b}, —NR₂ₐC(O)R_{2b}, —(C₀-C₆ alkyl)-CONHSO₂R₂ₐ, —(C₀-C₆ alkyl)-CONHSO₂NR₂ₐR_{2b}, —(C₀-C₆ alkyl)-SO₂NHCOR₂ₐ, —(C₀-C₆ alkyl)-SO₂NHR₂ₐ, —(C₀-C₆ alkyl)-CONR₂ₐOR_{2b},

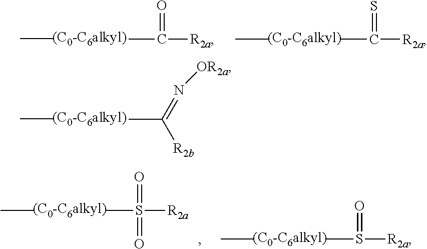

deuterium, halo, amino, hydroxy, cyano, formyl, nitro, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₀ (n=0 to 2, R₁₀ is directly connected to S), wherein R₁₀ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio, wherein R₂ₐ and R_{2b} are each independently H, C₁-C₆ alkyl, alkoxy (—O-alkyl), hydroxy, thioalkoxy (—S-alkyl), cyano (—CN), or amino;

R₄, R₅, R₆, R₇, and R₈ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;

R_{N} is H, CN, C₁-C₆ alkyl, —OH, —(CO)—OR, or —OR, wherein R is H, C₁-C₆ alkyl, or C₁-C₆ acyl; and

optionally, adjacent R groups, together, can form a six- to twelve-membered ring.

In another embodiment, the method includes administering to the patient (1) a therapeutically effective amount of a compound of formula 4, or an enantiomer, diastereomer, or pharmaceutically acceptable salt thereof, and (2) a therapeutically effective amount of an inhibitor of an immune checkpoint protein,

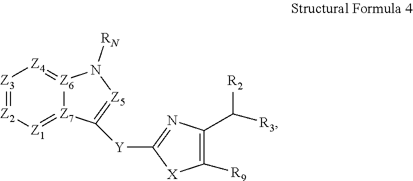

wherein:

X is O (oxygen) or S (sulfur);

Y is a bond, O (oxygen), S (sulfur), or

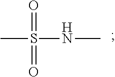

Z₁ is N or CR₄, Z₂ is N or CR₅, Z₃ is N or CR₆, Z₄ is N or CR₇, Z₅ is N or CR₈, Z₆ is N or C, Z₇ is N or C, wherein no more than two of Z₁, Z₂, Z₃, Z₄, Z₅, Z₆, and Z₇ are N;

R₄, R₅, R₆, R₇, R₈, and R₉ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₀ (n=0 to 2, R₁₀ is directly connected to S), wherein R₁₀ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;

R_{N} is H, CN, C₁-C₆ alkyl, —OH, —(CO)—OR, or —OR, wherein R is H, C₁-C₆ alkyl, or C₁-C₆ acyl;

R₂ and R₃ are together selected from the group consisting of ═O, ═S, or ═NRₐ (Rₐ is H, C₁-C₆ alkyl, C₁-C₆ acyl, or —OR, R is H, C₁-C₆ alkyl, or C₁-C₆ acyl), or

R₂ and R₃ are each independently selected from the group consisting of —NRₐR_{b} (Rₐ and R_{b} are each independently H, C₁-C₆ alkyl, or C₁-C₆ acyl), hydrogen, deuterium, halo, amino, hydroxy, cyano, formyl, furyl, nitro, alkyl, haloalkyl, alkenyl, haloalkenyl, alkynyl, haloalkynyl, C₁-C₆ acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio; and

optionally, adjacent R groups, together, can form a six- to twelve-membered ring.

In another embodiment, the method includes administering to the patient (1) a therapeutically effective amount of a compound of formula 5, or an enantiomer, diastereomer, or pharmaceutically acceptable salt thereof, and (2) a therapeutically effective amount of an inhibitor of an immune checkpoint protein,

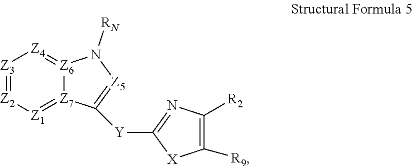

wherein:

X is O (oxygen) or S (sulfur);

Y is a bond, O (oxygen), S (sulfur), or

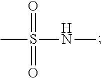

Z₁ is N or CR₄, Z₂ is N or CR₅, Z₃ is N or CR₆, Z₄ is N or CR₇, Z₅ is N or CR₈, Z₆ is N or C, Z₇ is N or C, wherein no more than two of Z₁, Z₂, Z₃, Z₄, Z₅, Z₆, and Z₇ are N;

R₂ and R₉ are each independently selected from the group consisting of hydrogen, aryl, heteroaryl, cycloalkyl, heterocycloalkyl, alkyl, —NR₂ₐC(O)OR_{2b}, —NR₂ₐC(O)R_{2b}, —(C₀-C₆ alkyl)-CONHSO₂R₂ₐ, —(C₀-C₆ alkyl)-CONHSO₂NR₂ₐR_{2b}, —(C₀-C₆ alkyl)-SO₂NHCOR₂ₐ, —(C₀-C₆ alkyl)-SO₂NHR₂ₐ, —(C₀-C₆ alkyl)-CONR₂ₐOR_{2b},

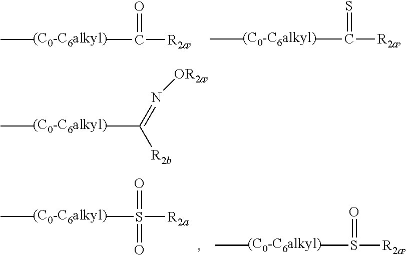

deuterium, halo, amino, hydroxy, cyano, formyl, nitro, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₀ (n=0 to 2, R₁₀ is directly connected to S), wherein R₁₀ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio, wherein R₂ₐ and R_{2b} are each independently H, C₁-C₆ alkyl, alkoxy (—O-alkyl), hydroxy, thioalkoxy (—S-alkyl), cyano (—CN), or amino;

R₄, R₅, R₆, R₇, and R₈ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;

R_{N} is H, CN, C₁-C₆ alkyl, —OH, —(CO)—OR, or —OR, wherein R is H, C₁-C₆ alkyl, or C₁-C₆ acyl; and

optionally, adjacent R groups, together, can form a six- to twelve-membered ring. In another embodiment, the method includes administering to the patient (1) a therapeutically effective amount of a compound of formula 6, or an enantiomer, diastereomer, or pharmaceutically acceptable salt thereof, and (2) a therapeutically effective amount of an inhibitor of an immune checkpoint protein,

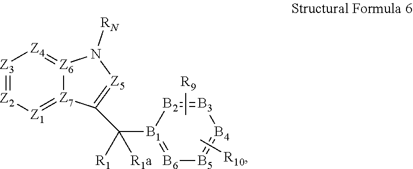

wherein:

R₁ and R₁ₐ are taken together to form ═NR_{b}, wherein R_{b} is H, C₁-C₆ alkyl, hydroxy, C₁-C₆ alkoxy (—O-alkyl), C₁-C₆ acyloxy, amino, or C₁-C₆ acyl, or

R₁ and R₁ₐ are taken together to form ═CR_{b}R_{c}, wherein R_{b} and R_{c} are each independently H, C₁-C₆ alkyl, alkoxy (—O-alkyl), thioalkoxy (—S-alkyl), cyano (—CN), or amino, or

R₁ and R₁ₐ are taken together to form ═O, ═NORₐ, or ═S, wherein Rₐ is H, C₁-C₆ alkyl, or C₁-C₆ acyl, or

R₁ and R₁ₐ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₄ (n=0 to 2, R₁₄ is directly connected to S), wherein R₁₄ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;

Z₁ is N or CR₄, Z₂ is N or CR₅, Z₃ is N or CR₆, Z₄ is N or CR₇, Z₅ is N or CR₈, Z₆ is N or C, Z₇ is N or C, wherein no more than two of Z₁, Z₂, Z₃, Z₄, Z₅, Z₆, and Z₇ are N;

R₄, R₅, R₆, R₇, and R₈ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;

R_{N} is H, CN, C₁-C₆ alkyl, —OH, —(CO)—OR, or —OR, wherein R is H, C₁-C₆ alkyl, or C₁-C₆ acyl;

B₁, B₂, B₃, B₄, B₅, and B₆ are each independently C or N;

R₉ and R₁₀, the number of which, together, complete the valence of each of B₁, B₂, B₃, B₄, B₅, and B₆, are each independently selected from the group consisting of hydrogen, aryl, heteroaryl, cycloalkyl, heterocycloalkyl, alkyl, —NR₂ₐC(O)OR_{2b}, —NR₂ₐC(O)R_{2b}, —(C₀-C₆ alkyl)-CONHSO₂R₂ₐ, —(C₀-C₆ alkyl)-CONHSO₂NR₂ₐR_{2b}, —(C₀-C₆ alkyl)-SO₂NHCOR₂ₐ, —(C₀-C₆ alkyl)-SO₂NHR₂ₐ, —(C₀-C₆ alkyl)-CONR₂ₐOR_{2b},

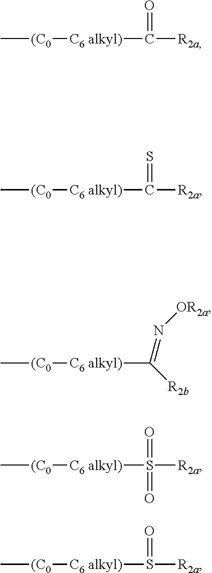

deuterium, halo, amino, hydroxy, cyano, formyl, nitro, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₂ (n=0 to 2, R₁₂ is directly connected to S), wherein R₁₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and

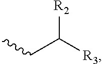

wherein R₂ₐ and R_{2b} are each independently H, C₁-C₆ alkyl, alkoxy (—O-alkyl), hydroxy, thioalkoxy (—S-alkyl), cyano (—CN), or amino;

wherein R₂ and R₃ are together selected from the group consisting of ═O, ═S, or ═NRₐ (Rₐ is H, C₁-C₆ alkyl, C₁-C₆ acyl, or —OR, R is H, C₁-C₆ alkyl, or C₁-C₆ acyl), or

R₂ and R₃ are each independently selected from the group consisting of —NRₐR_{b} (Rₐ and R_{b} are each independently H, C₁-C₆ alkyl, or C₁-C₆ acyl), hydrogen, deuterium, halo, amino, hydroxy, cyano, formyl, furyl, nitro, alkyl, haloalkyl, alkenyl, haloalkenyl, alkynyl, haloalkynyl, C₁-C₆ acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₃ (n=0 to 2, R₁₃ is directly connected to S), wherein R₁₃ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio; and

optionally, adjacent R groups, together, can form a six- to twelve-membered ring. In another embodiment, the method includes administering to the patient (1) a therapeutically effective amount of a compound of formula 7, or an enantiomer, diastereomer, or pharmaceutically acceptable salt thereof, and (2) a therapeutically effective amount of an inhibitor of an immune checkpoint protein,

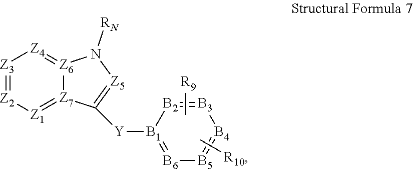

wherein:

Y is a bond, O (oxygen), S (sulfur), or

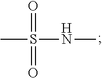

Z₁ is N or CR₄, Z₂ is N or CR₅, Z₃ is N or CR₆, Z₄ is N or CR₇, Z₅ is N or CR₈, Z₆ is N or C, Z₇ is N or C, wherein no more than two of Z₁, Z₂, Z₃, Z₄, Z₅, Z₆, and Z₇ are N;

R₄, R₅, R₆, R₇, and R₈ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;

R_{N} is H, CN, C₁-C₆ alkyl, —OH, —(CO)—OR, or —OR, wherein R is H, C₁-C₆ alkyl, or C₁-C₆ acyl;

B₁, B₂, B₃, B₄, B₅, and B₆ are each independently C or N;

R₉ and R₁₀, the number of which, together, complete the valence of each of B₁, B₂, B₃, B₄, B₅, and B₆, are each independently selected from the group consisting of hydrogen, aryl, heteroaryl, cycloalkyl, heterocycloalkyl, alkyl, —NR₂ₐC(O)OR_{2b}, —NR₂ₐC(O)R_{2b}, —(C₀-C₆ alkyl)-CONHSO₂R₂ₐ, —(C₀-C₆ alkyl)-CONHSO₂NR₂ₐR_{2b}, —(C₀-C₆ alkyl)-SO₂NHCOR₂ₐ, —(C₀-C₆ alkyl)-SO₂NHR₂ₐ, —(C₀-C₆ alkyl)-CONR₂ₐOR_{2b},

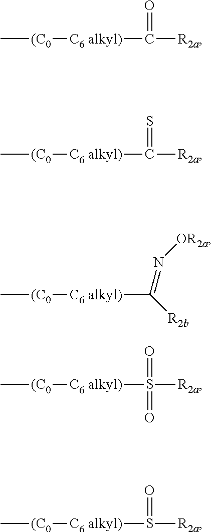

deuterium, halo, amino, hydroxy, cyano, formyl, nitro, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₂ (n=0 to 2, R₁₂ is directly connected to S), wherein R₁₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and

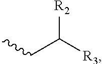

wherein R₂ₐ and R_{2b} are each independently H, C₁-C₆ alkyl, alkoxy (—O-alkyl), hydroxy, thioalkoxy (—S-alkyl), cyano (—CN), or amino;

wherein R₂ and R₃ are together selected from the group consisting of ═O, ═S, or ═NRₐ (Rₐ is H, C₁-C₆ alkyl, C₁-C₆ acyl, or —OR, R is H, C₁-C₆ alkyl, or C₁-C₆ acyl), or

R₂ and R₃ are each independently selected from the group consisting of —NRₐR_{b} (Rₐ and R_{b} are each independently H, C₁-C₆ alkyl, or C₁-C₆ acyl), hydrogen, deuterium, halo, amino, hydroxy, cyano, formyl, furyl, nitro, alkyl, haloalkyl, alkenyl, haloalkenyl, alkynyl, haloalkynyl, C₁-C₆ acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₃ (n=0 to 2, R₁₃ is directly connected to S), wherein R₁₃ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio; and

optionally, adjacent R groups, together, can form a six- to twelve-membered ring.

In each of formulae 2, 2a, 3, 3a, 3b, 3c, 4 and 5 of the methods, in some embodiments, each of R₄, R₅, R₆, and R₇ is hydrogen. In other embodiments, at least one of R₄, R₅, R₆, and R₇ can be F, Cl or Br and the others of R₄, R₅, R₆, and R₇ are hydrogen. In still other embodiments, at least two of R₄, R₅, R₆, and R₇, independently, can be F, Cl or Br and the others of R₄, R₅, R₆, and R₇ are hydrogen. The F, Cl or Br can be at the indole ring carbon 5, 6, or 7.

In each of formulae 3, 3a, 3b, 3c, and 5 of the methods, in certain embodiments, R₉ can be hydrogen. R₂ can be acyl, cyano, hydroxyl-substituted C1-C6 alkyl, amino-substituted C1-C6 alkyl, aryl, or heteroaryl. The aryl or heteroaryl can be substituted or unsubstituted. The substituted aryl or heteroaryl can be substituted with halo, amino, hydroxyl, or C1-C6 alkyl. The amino can be unsubstituted.

In each of formulae 2, 2a, and 4 of the methods, in certain embodiments, R₂ can be hydroxyl or amino and R₃ can be alkyl, aryl, nitro, or cyano. R₉ can be hydrogen. The amino can be substituted or unsubstituted.

In another embodiment, the method includes administering to the patient (1) a therapeutically effective amount of any one of the compounds in Table 1 and Table 2, or an enantiomer, diastereomer, or pharmaceutically acceptable salt thereof, and (2) a therapeutically effective amount of an inhibitor of an immune checkpoint protein. In some embodiments, the compound is selected from the group consisting of ARI-001, ARI-002, ARI-003, ARI-017, ARI-018, ARI-019, and ARI-020, and an enantiomer, diastereomer, or pharmaceutically acceptable salt thereof.

In another embodiment, the method includes administering to the patient (1) a therapeutically effective amount of a compound of formula 8, or an enantiomer, diastereomer, or pharmaceutically acceptable salt thereof, and (2) a therapeutically effective amount of an inhibitor of an immune checkpoint protein,

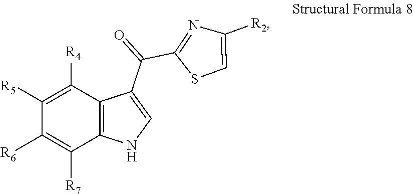

wherein R₂ is selected from the group consisting of substituted alkyl, heteroaryl, and

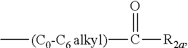

wherein R₂ₐ is H, C1-C6 alkyl, alkoxy (—O-alkyl), hydroxy, thioalkoxy (—S-alkyl), cyano (—CN), or amino; and

R₄, R₅, R₆, and R₇, are each, independently, selected from the group consisting of hydrogen and halo.

In some embodiments, R₂ is substituted alkyl, e.g., a C1-C6 alkyl substituted with one or more hydroxyl, amino, nitro, or cyano. In some embodiments, R₂ is heteroaryl, e.g., oxadiazolyl or thiadiazolyl, optionally substituted with one or more hydroxyl, amino, nitro, cyano, C1-C6 alkyl, or C1-C6 alkyl amino. In some embodiments, R₂ is —C(O)—R₂ₐ, and R₂ₐ is C1-C6 alkyl.

In one embodiment of the compound of structural formula 8, at least one of R₄, R₅, R₆, and R₇ is F, Cl or Br and the others of R₄, R₅, R₆, and R₇ are hydrogen. In another embodiment, at least two of R₄, R₅, R₆, and R₇ are F, Cl or Br, and the others of R₄, R₅, R₆, and R₇ are hydrogen.

In one embodiment, R₅ is F and R₄, R₆, and R₇ are hydrogen. In another embodiment, R₆ is F and R₄, R₅, and R₇ are hydrogen. In still another embodiment, R₇ is F, and R₄, R₅, and R₆ are hydrogen.

In one embodiment, R₅ is Cl and R₄, R₆, and R₇ are hydrogen. In another embodiment, R₆ is Cl and R₄, R₅, and R₇ are hydrogen. In still another embodiment, R₇ is Cl, and R₄, R₅, and R₆ are hydrogen.

In one embodiment, R₅ and R₆ are F and R₄ and R₇ are hydrogen. In another embodiment, R₅ and R₇ are F, and R₄ and R₆ are hydrogen. In still another embodiment, R₆ and R₇ are F, and R₄ and R₅ are hydrogen.

In one embodiment, R₅ and R₆ are Cl and R₄ and R₇ are hydrogen. In another embodiment, R₅ and R₇ are Cl, and R₄ and R₆ are hydrogen. In still another embodiment, R₆ and R₇ are Cl, and R₄ and R₅ are hydrogen.

In some embodiments, each of R₄, R₅, R₆ and R₇ is hydrogen.

In some embodiments, the compound of formula 8 is selected from Table 1 (e.g., ARI-017, ARI-018, ARI-019, ARI-020, ARI-031, ARI-060, ARI-083, ARI-087, ARI-090, ARI-118, ARI-120, ARI-140, ARI-143, ARI-145, ARI-146, ARI-148, ARI-149, or ARI-150), or an enantiomer, diastereomer, or pharmaceutically acceptable salt thereof. In some embodiments, the compound is selected from ARI-087, ARI-140, ARI-143, ARI-149, and ARI-150, or an enantiomer, diastereomer, or pharmaceutically acceptable salt thereof. In some embodiments, the compound is selected from ARI-031, ARI-060, ARI-083, ARI-090, ARI-118, ARI-120, ARI-145, ARI-146, and ARI-148, or an enantiomer, diastereomer, or pharmaceutically acceptable salt thereof.

In another embodiment, the method includes administering to the patient (1) a therapeutically effective amount of a compound of Formula I, and (2) a therapeutically effective amount of an inhibitor of an immune checkpoint protein,

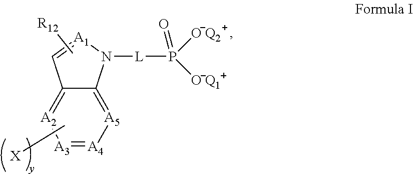

wherein:

R₁₂ is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio,

each of A₁, A₂, A₃, A₄, and A₅, independently, is CR₂ or N;

L is —(CR₂R₃—O)ₙ— or a bond;

R₂ is H or C1-C6 alkyl;

R₃ is H or C1-C6 alkyl;

or, together, R₂ and R₃ form a C₃-C₈ cycloalkyl;

n is 0, 1, 2, 3, 4, 5, or 6;

y is 0, 1, 2, 3, or 4;

each X, independently, is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio; and

Q₁⁺ and Q₂⁺ are each, independently, a monocation, or together are a dication or one of Q₁⁺ or Q₂⁺ is C1-C6 alkyl, benzyl, allyl or —(CR₂R₃—O)—R₂₃, and R₂₃ is H, or C1-C6 alkyl.

In some embodiments embodiment, the compound is of Formula II,

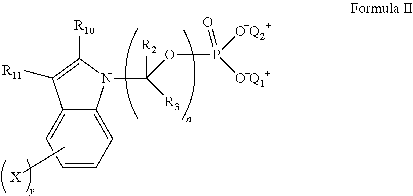

wherein:

R₁₀ is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio;

R₁₁ is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio, wherein one of R₁₀ and R₁₁ is H or C1-C6 alkyl;

R₂ is H or C1-C6 alkyl;

R₃ is H or C1-C6 alkyl;

or, together, R₂ and R₃ form a C3-C8 cycloalkyl;

y is 0, 1, 2, 3, or 4;

each X, independently, is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio;

Q₁⁺ and Q₂⁺ are each, independently, a monocation, or together are a dication or one of Q₁⁺ or Q₂⁺ is C1-C6 alkyl, benzyl, allyl or —(CR₂R₃—O)—R₂₃, and R₂₃ is H, or C1-C6 alkyl; and

n is 0, 1, 2, 3, 4, 5, or 6.

In some embodiments, the compound is of Formula III,

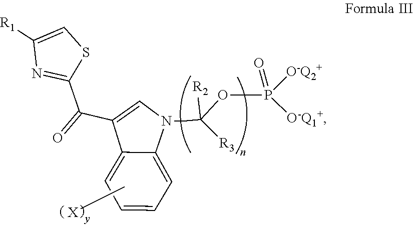

wherein:

R₁ is —C(═O)—R₄, cyano, an oxadiazole, or a thiadiazole;

R₂ and R₃ are each, independently, hydrogen, or C₁-C₆ alkyl; and

R₄ is selected from the group consisting of —NRₐR_{b} (Rₐ and R_{b} are each independently H, C₁-C₆ alkyl, or C₁-C₆ acyl), hydrogen, deuterium, halo, amino, hydroxy, cyano, formyl, furyl, nitro, alkyl, haloalkyl, alkenyl, haloalkenyl, alkynyl, haloalkynyl, unsubstituted or substituted C₁-C₆ acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₘR₂₂ (m=0 to 2, R₂₂ is directly connected to S), wherein R₂₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carbonyloxy, halocarbonyloxy, carbonylthio, halocarbonylthio, thiocarbonyloxy, halothiocarbonyloxy, thiocarbonylthio, and halothiocarbonylthio;

y is 0, 1, 2, 3, or 4;

each X, independently, is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio;

Q₁⁺ and Q₂⁺ is each, independently, a monocation, or together are a dication or one of Q₁⁺ or Q₂⁺ is C1-C6 alkyl, benzyl, allyl or —(CR₂R₃—O)—R₂₃, and R₂₃ is H or C1-C6 alkyl, and the other of Q₁⁺ or Q₂⁺ can be a monocation; and

n is 0, 1, 2, 3, 4, 5, or 6.

In some embodiments, the compound is of Formula IV,

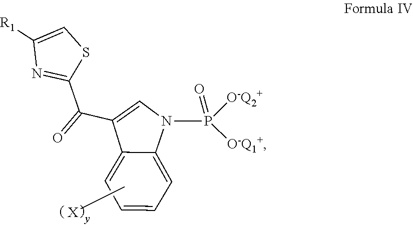

wherein:

R₁ is —C(═O)—R₄, cyano, an oxadiazole, or a thiadiazole; and

R₄ is selected from the group consisting of —NRₐR_{b} (Rₐ and R_{b} are each independently H, C₁-C₆ alkyl, or C₁-C₆ acyl), hydrogen, deuterium, halo, amino, hydroxy, cyano, formyl, furyl, nitro, alkyl, haloalkyl, alkenyl, haloalkenyl, alkynyl, haloalkynyl, unsubstituted or substituted C₁-C₆ acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₘR₂₂ (m=0 to 2, R₂₂ is directly connected to S), wherein R₂₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carbonyloxy, halocarbonyloxy, carbonylthio, halocarbonylthio, thiocarbonyloxy, halothiocarbonyloxy, thiocarbonylthio, and halothiocarbonylthio;

y is 0, 1, 2, 3, or 4;

each X, independently, is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio; and

Q₁⁺ and Q₂⁺ is each, independently, a monocation, or together are a dication or one of Q₁⁺ or Q₂⁺ is C1-C6 alkyl, benzyl, allyl or —(CR₂R₃—O)—R₂₃, and R₂₃ is H or C1-C6 alkyl, and the other of Q₁⁺ or Q₂⁺ can be a monocation.

In some embodiments, the compound is of Formula V,

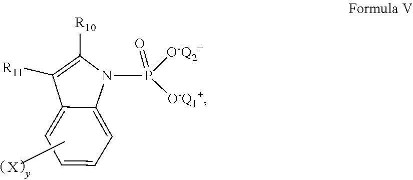

wherein:

R₁ is —C(═O)—R₄, cyano, an oxadiazole, or a thiadiazole;

R₂ and R₃ are each independently hydrogen, or C₁-C₆ alkyl; and

R₄ is selected from the group consisting of —NRₐR_{b} (Rₐ and R_{b} are each independently H, C₁-C₆ alkyl, or C₁-C₆ acyl), hydrogen, deuterium, halo, amino, hydroxy, cyano, formyl, furyl, nitro, alkyl, haloalkyl, alkenyl, haloalkenyl, alkynyl, haloalkynyl, unsubstituted or substituted C₁-C₆ acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₘR₂₂ (m=0 to 2, R₂₂ is directly connected to S), wherein R₂₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carbonyloxy, halocarbonyloxy, carbonylthio, halocarbonylthio, thiocarbonyloxy, halothiocarbonyloxy, thiocarbonylthio, and halothiocarbonylthio;

y is 0, 1, 2, 3, or 4;

each X, independently, is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio; and

Q₁⁺ and Q₂⁺ is each, independently, a monocation, or together are a dication or one of Q₁⁺ or Q₂⁺ is C1-C6 alkyl, benzyl, allyl or —(CR₂R₃—O)—R₂₃, and R₂₃ is H or C1-C6 alkyl, and the other of Q₁⁺ or Q₂⁺ can be a monocation.

In some embodiments, in any one of Formulae I, II, III, IV and V, Q₁⁺ and Q₂⁺ are each, independently, an alkali metal.

In some embodiments, in any one of Formulae I, II, III, IV and V, Q₁⁺ and Q₂⁺ are each, independently, selected from the group consisting of ammonium and alkyl ammonium.

In some embodiments, in any one of Formulae I, II, III, IV and V, Q₁⁺ and Q₂⁺ together are selected from the group consisting of an alkaline earth metal salt.

In some embodiments, in any one of Formulae I, II, III, IV and V, Q₁⁺ and Q₂⁺ are each independently selected from the group consisting of zinc, calcium and magnesium.

In some embodiments, in any one of Formulae I, II, III, IV and V, Q₁⁺ and Q₂⁺ are each independently lithium, sodium, or potassium, y is 0, 1 or 2, and X is F, Cl, or Br.

In some embodiments, in Formula III or IV, R₁ is —C(═O)—R₄, and R₄ is C₁-C₆ alkyl or C₁-C₆ alkoxy.

In some embodiments, in Formula III or IV, R₁ is an oxadiazole or a thiadiazole, and the oxadiazole, or the thiadiazole is optionally substituted by amino, alkyl amino, amino alkyl, alkoxy, alkyl or haloalkyl.

In some embodiments, in any one of Formulae I, II, and III, n is 0 or 1.

In some embodiments, the compound of Formula II is selected from the group consisting of:

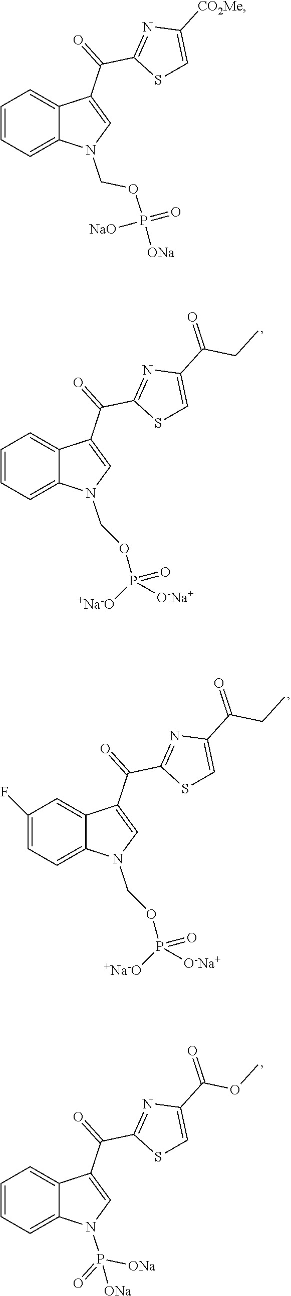

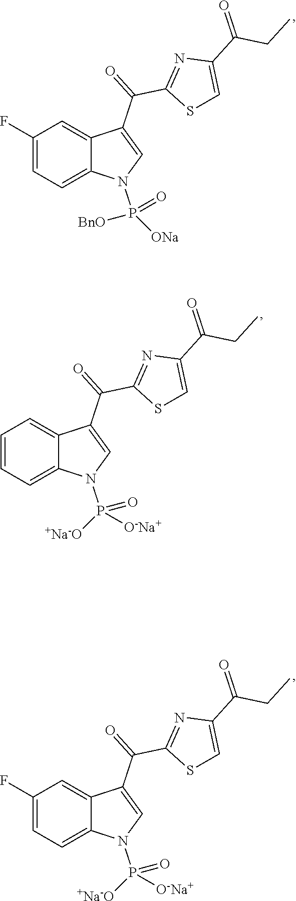

In some embodiments, in Formula III or IV, R1 is an unsubstituted or substituted oxadiazole. In some embodiments, the substituted oxadiazole is a C1-C6 alkyl oxadiazole, haloalkyl oxadiazole, halo oxadiazole, amino oxadiazole, alkyl amino oxadiazole, amino alkyl oxadiazole, or hydroxy oxadiazole. In some embodiments, n is 0. In some embodiments, Q₁⁺ and Q₂⁺ are each lithium, sodium, or potassium. In some embodiments, the indole is a fluorinated indole.

In some embodiments, the compound of Formula II is selected from the group consisting of:

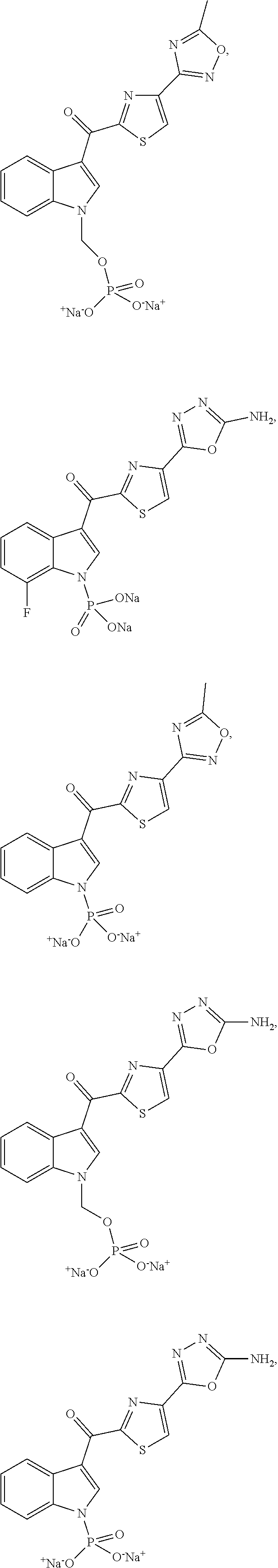

In another embodiment, the method includes administering to the patient (1) a therapeutically effective amount of a compound of Formula VI, and (2) a therapeutically effective amount of an inhibitor of an immune checkpoint protein,

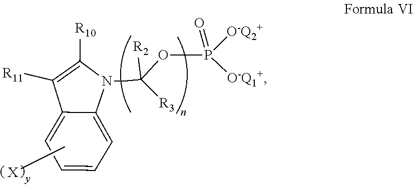

wherein:

R₁₀ is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio;

R₁₁ is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio, wherein one of R₁₀ and R₁₁ is H or C1-C6 alkyl;

R₂ is H or C1-C6 alkyl;

R₃ is H or C1-C6 alkyl;

or, together, R₂ and R₃ form a C3-C8 cycloalkyl;

y is 0, 1, 2, 3, or 4;

each X, independently, is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio;

R₂₀ and R₃₀ each, independently, is C1-C6 alkyl or benzyl, or one of R₂₀ or R₃₀ is H, C1-C6 alkyl, allyl, or benzyl and the other of R₂₀ or R₃₀ is a cation; and

n is 0, 1, 2, 3, 4, 5, or 6.

In some embodiments, the compound of Formula I or VI is any one of the compounds in Table 3.

In some embodiments of the methods, the immune checkpoint protein is PD-1, PD-L1, PD-L2, or CTLA-4.

In some embodiments of the methods, the inhibitor of the immune checkpoint protein is an anti-PD-1 antibody or an anti-CTLA-4 antibody.

In some embodiments of the methods, the cancer is refractory to anti-PD-1 antibody treatment.

In some embodiments of the methods, the cancer is a lymphoma or a solid tumor, e.g., diffuse large B-cell lymphoma, marginal zone lymphoma, chronic lymphocytic leukemia, small lymphocytic lymphoma, prolymphocytic leukemia, acute lymphocytic leukemia, Waldenstrom's Macroglobulinemia, follicular lymphoma, mantle cell lymphoma, Hodgkin lymphoma, non-Hodgkin lymphoma, multiple myeloma, prostate cancer, ovarian cancer, fallopian tube cancer, cervical cancer, breast cancer, lung cancer, skin cancer, colon cancer, colorectal cancer, stomach cancer, pancreatic cancer, liver cancer, kidney cancer, bladder cancer, soft tissue cancer, glioma, and head and neck cancer. In a particular embodiment, the cancer is colon cancer, breast cancer, pancreatic cancer, lung cancer, prostate cancer, kidney cancer, and melanoma.

The present disclosure also provides an indole compound for use in treating cancer in combination with an inhibitor of an immune checkpoint protein in a combination therapy method described herein.

The present disclosure further discloses the use of an indole compound for the manufacture of a medicament for treating cancer, and the use of an inhibitor of an immune checkpoint protein for the manufacture of a medicament for treating cancer, in a combination therapy method described herein.

The present disclosure provides also articles of manufacture, including kits, comprising an indole compound and an immune checkpoint inhibitor, for use in treating cancer in a combination therapy method described herein.

### BRIEF DESCRIPTION OF THE DRAWINGS

FIG. 1 shows a synthesis scheme for substituted indoles intermediates.

FIG. 2 shows a synthesis scheme for ester and amides.

FIG. 3 shows a synthesis scheme for nitriles.

FIG. 4 shows a synthesis scheme for ketones.

FIG. 5 shows a first synthesis scheme for heterocycle.

FIG. 6 shows a second synthesis scheme for heterocycle.

FIG. 7 shows a third synthesis scheme for heterocycle.

FIG. 8 shows a fourth synthesis scheme for heterocycle.

FIG. 9 shows a fifth synthesis scheme for heterocycle.

FIG. 10 shows a sixth synthesis scheme for heterocycle.

FIG. 11 shows a seventh synthesis scheme for heterocycle.

FIG. 12 shows an eighth synthesis scheme for heterocycle.

FIG. 13 shows a synthesis scheme for CF₃ ketone.

FIG. 14 shows a synthesis scheme for CF₃ amine.

FIG. 15 shows a synthesis scheme for α-aminonitrile.

FIG. 16 shows a scheme for preparing the key intermediates Int-A, Int-B and Int-C.

FIG. 17 shows a scheme for preparing the key intermediate Int-E.

FIG. 18 shows a synthesis scheme for ARI-064 according to Example 43.

FIG. 19 shows a synthesis scheme for ARI-075 according to Example 48.

FIG. 20 shows a synthesis scheme for ARI-121 according to Example 64.

FIG. 21 shows a synthesis scheme for ARI-041 (PTC17341-17) according to Example 65.

FIG. 22 shows a synthesis scheme for ARI-049 (PTC17341-06) according to Example 68.

FIG. 23 shows a synthesis scheme for ARI-058 (PTC17341-05) according to Example 71.

FIG. 24 shows a synthesis scheme for ARI-077 according to Example 75.

FIG. 25 shows a synthesis scheme for ARI-068 (PTC17341-16), ARI-092 (PTC17341-16A), and ARI-094 (PTC17341-16B) according to Example 77.

FIG. 26 shows a synthesis scheme for ARI-069 and ARI-070 (PTC17341-22-A and PTC17341-22-B) according to Example 78.

FIG. 27 shows a synthesis scheme for ARI-085 (PTC17341-46) according to Example 82.

FIG. 28 shows a synthesis scheme for ARI-086 (PTC17341-35) according to Example 83.

FIG. 29 shows a synthesis scheme for ARI-087 according to Example 84.

FIG. 30 shows a synthesis scheme for PTC17341-11A according to Example 87.

FIG. 31 shows a synthesis scheme for ARI-123 (PTC17341-95) according to Example 102.

FIG. 32 shows a synthesis scheme for ARI-127 (PTC17341-54) according to Example 106.

FIG. 33 shows a synthesis scheme for ARI-137 (PTC17341-108) according to Example 114.

FIG. 34 shows a synthesis scheme for ARI-138 (PTC17341-107) according to Example 115.

FIG. 35 shows a synthesis scheme for ARI-139 (PTC17341-109) according to Example 116.

FIG. 36 shows a synthesis scheme for ARI-141 (PTC17341-60) according to Example 118.

FIG. 37 shows a synthesis scheme for ARI-149 according to Example 125.

FIG. 38 shows a synthesis scheme for ARI-054 (PTC17341-21) according to Example 127.

FIG. 39 shows a synthesis scheme for ARI-150 according to Example 129.

FIG. 40 shows a synthesis scheme for 2-(1-(tert-butoxycarbonyl)-1H-indole-3-carbonyl) thiazole-4-carboxylic acid according to Example 130.

FIG. 41 shows a synthesis scheme for ARI-154 according to Example 131.

FIG. 42 shows a scheme of synthesizing dibromo compounds according to Example 135.

FIG. 43 shows exemplary compounds where thiazole and ester fragments are modified to potentially slow ester hydrolysis according to Example 136.

FIG. 44 describes a route of synthesis for ARI-1073 and ARI-024 according to Example 137.

FIG. 45 illustrates a synthesis route for ARI-068, ARI-092, and ARI-094 according to Example 138.

FIG. 46 illustrates a synthesis route for ARI-1029 and ARI-1030 according to Example 139.

FIG. 47 illustrates a synthesis route for amino amides and cyclic versions of compounds according to Example 140.

FIG. 48 illustrates a synthesis route for oxime compounds with hindered ketones according to Example 141.

FIG. 49 illustrates a synthesis route for pyrazine compounds according to Example 142.

FIG. 50 compares the properties of compounds with thiazole and indole replacements according to Example 143.

FIG. 51 shows a synthesis scheme for ARI-020 according to Example 144.

FIG. 52 shows a synthesis scheme for ARI-018 according to Example 145.

FIG. 53 shows a synthesis scheme for ARI-019 according to Example 146.

FIG. 54 shows a synthesis scheme for ARI-017 according to Example 147.

FIG. 55 shows a synthesis scheme for ARI-030 according to Example 148.

FIG. 56 shows a synthesis scheme for an aldehyde intermediate according to Example 149.

FIG. 57 shows a synthesis scheme for ARI-021 according to Example 150.

FIG. 58 shows a synthesis scheme for ARI-1057 according to Example 151.

FIG. 59 illustrates the synthesis of hindered ketones.

FIG. 60 is a graph showing the mean tumor volume on different study days in the study groups indicated for Study 1 in Example 152.

FIG. 61 is a graph showing the mean tumor volume on different study days in the study groups indicated for Study 2 in Example 152.

FIG. 62 is a graph showing the mean tumor volume on different study days in the study groups indicated for Study 3 in Example 152.

FIG. 63 is a graph showing the mean tumor volume on different study days in the study groups indicated for Study 4 in Example 152.

FIG. 64 is a graph showing the mean tumor volume on different study days in the study groups indicated for Study 5 in Example 152.

FIG. 65 is a graph showing the mean tumor volume on different study days in the study groups indicated for Study 6 in Example 152.

FIG. 66 is a graph showing the mean tumor volume on different study days in the study groups indicated for Study 7 in Example 152.

FIG. 67 is a graph showing the mean tumor volume on different study days in the study groups indicated for Study 8 in Example 152.

FIG. 68 is a graph showing the mean tumor volume on different study days in the study groups indicated for Study 9 in Example 152.

FIG. 69 is a graph showing the mean tumor volume on different study days in the study groups indicated for Study 10 in Example 152.

FIG. 70 is a graph showing the mean tumor volume on different study days in the study groups indicated for Study 11 in Example 152.

FIG. 71 is a graph showing the mean tumor volume on different days post tumor implant in four study groups according to Example 153.

FIG. 72 is a graph showing the median tumor volume on different days post tumor implant in four study groups according to Example 153.

FIG. 73 is a graph showing the mean tumor volume on different study days in the study groups indicated for Study 12 in Example 152.

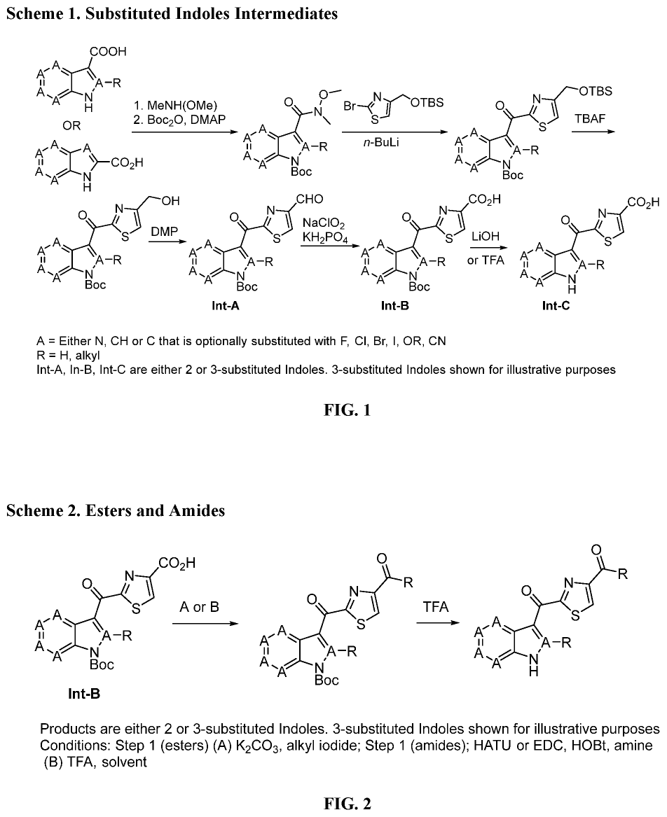

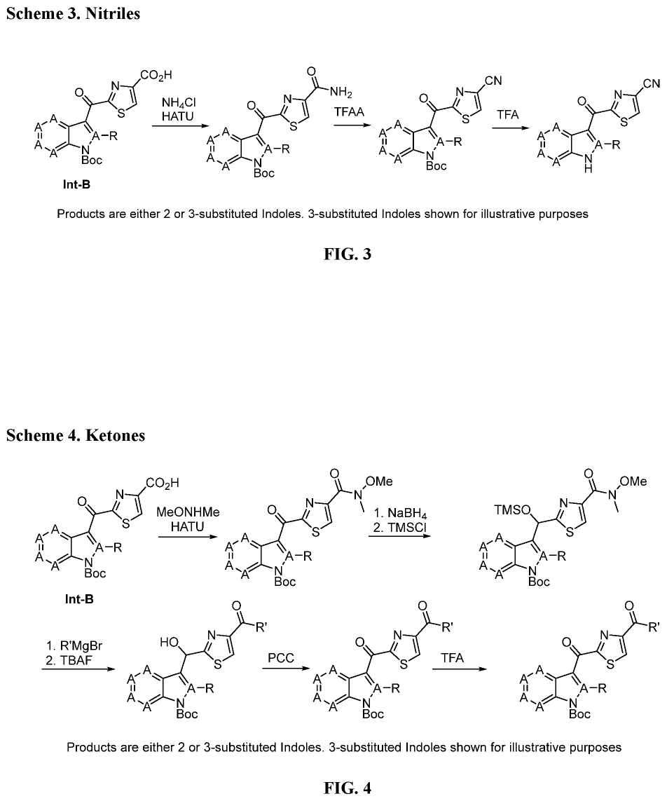

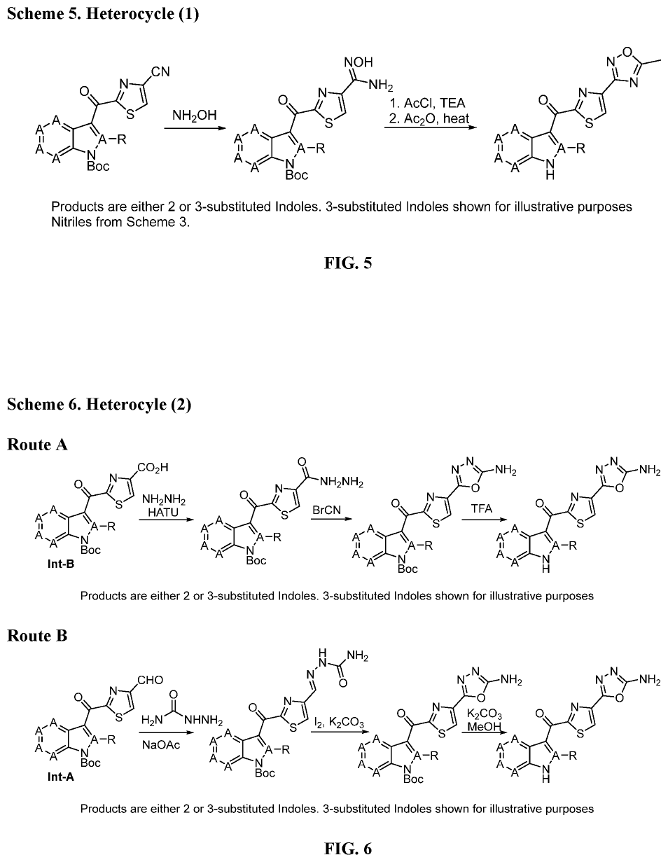

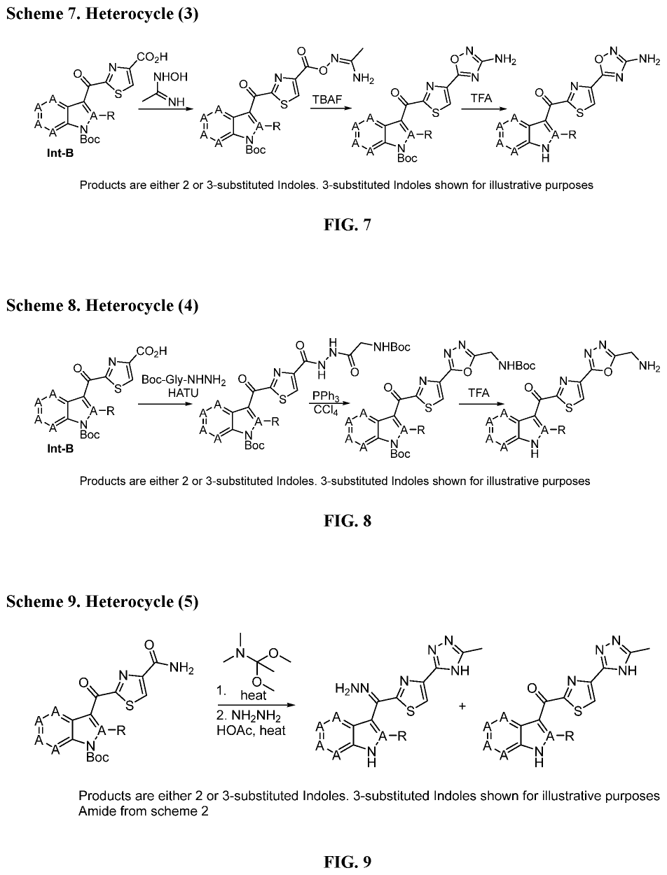

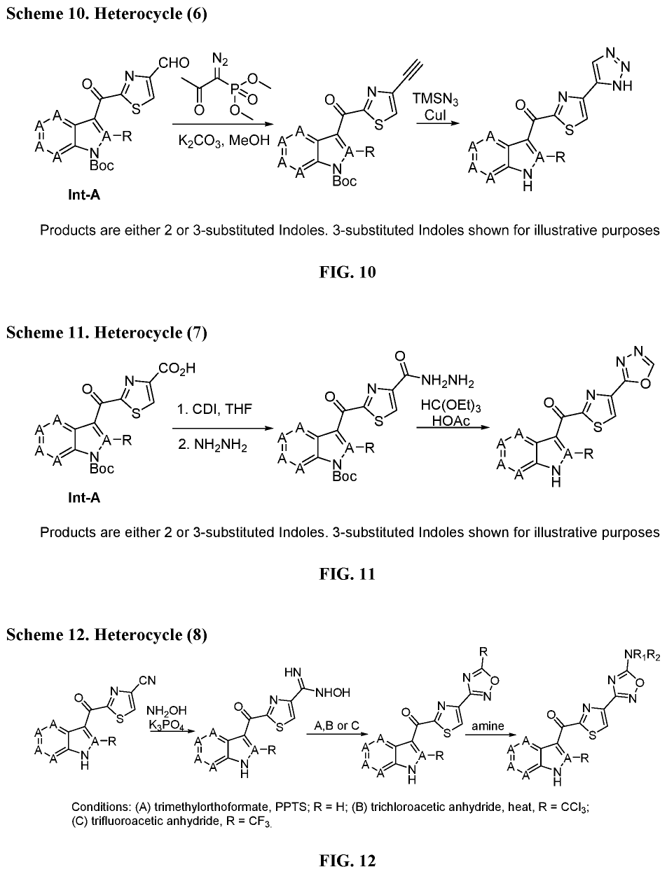

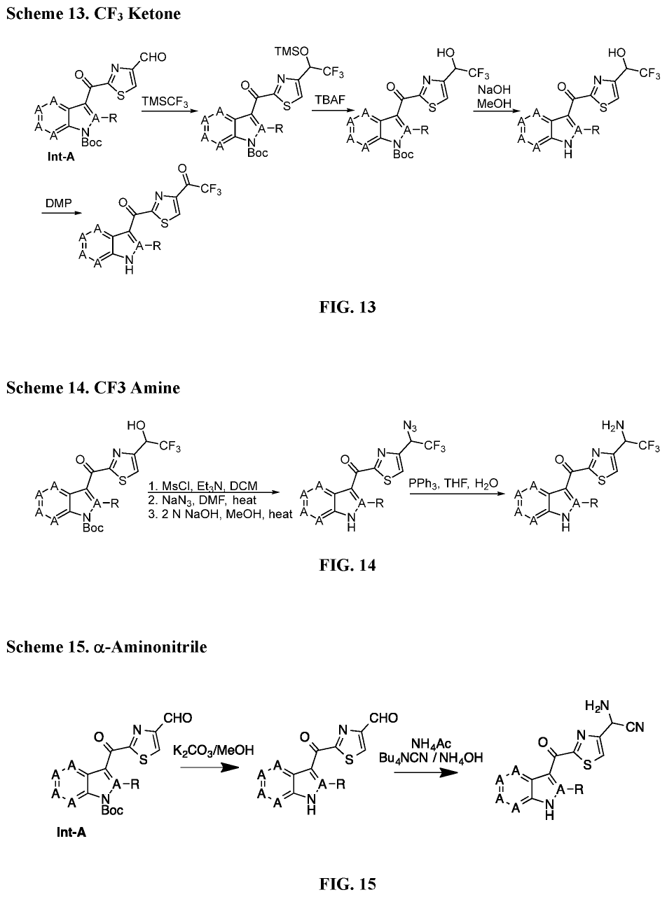

### DETAILED DESCRIPTION OF THE INVENTION

All technical and scientific terms used herein are the same as those commonly used by those ordinary skilled in the art to which the present invention pertains unless defined specifically otherwise.

The moieties described below can be substituted or unsubstituted. “Substituted” refers to replacement of a hydrogen atom of a molecule or an R-group with one or more additional R-groups such as halogen, alkyl, haloalkyl, alkenyl, alkoxy, alkoxyalkyl, alkylthio, trifluoromethyl, acyloxy, hydroxy, hydroxyalkyl, mercapto, carboxy, cyano, acyl, aryloxy, aryl, arylalkyl, heteroaryl, amino, aminoalkyl, alkylamino, dialkylamino, morpholino, piperidino, pyrrolidin-1-yl, piperazin-1-yl, nitro, phosphine, phosphinate, phosphonate, sulfato, ═O, ═S, or other R-groups. Unless otherwise indicated, an optionally substituted group may have a substituent at each substitutable position of a group. Combinations of substituents contemplated herein are preferably those that result in the formation of stable (e.g., not substantially altered for a week or longer when kept at a temperature of 40° C. or lower in the absence of moisture or other chemically reactive conditions), or chemically feasible, compounds.

“Hydroxy”, “thiol”, “cyano”, “nitro”, and “formyl” refer, respectively, to —OH, —SH, —CN, —NO₂, and —CHO.

“Acyloxy” refers to a RC(═O)O— radical, wherein R is alkyl, cycloalkyl, aryl, heteroalkyl, heteroaryl, or heterocycloalkyl, which are as described herein. In some embodiments, it is a C₁-C₄ acyloxy radical, which refers to the total number of chain or ring atoms of the alkyl, cycloalkyl, aryl, heteroalkyl, heteroaryl, or heterocycloalkyl portion of the acyloxy group plus the carbonyl carbon of acyl, i.e., the other ring or chain atoms plus carbonyl. If the R radical is heteroaryl or heterocycloalkyl, the hetero ring or chain atoms contribute to the total number of chain or ring atoms.

“Alkyl” refers to a group of 1-18, 1-16, 1-12, 1-10, preferably 1-8, more preferably 1-6 unsubstituted or substituted hydrogen-saturated carbons connected in linear, branched, or cyclic fashion, including the combination in linear, branched, and cyclic connectivity. Non-limiting examples include methyl, ethyl, propyl, isopropyl, butyl, and pentyl.

“Cycloalkyl” refers to a monocyclic or polycyclic non-aromatic radical that contains carbon and hydrogen, and may be saturated, or partially unsaturated. Cycloalkyl groups include groups having from 3 to 10 ring atoms (e.g., C₃-C₁₀ cycloalkyl). Whenever it appears herein, a numerical range such as “3 to 10” refers to each integer in the given range; e.g., “3 to 10 carbon atoms” means that the cycloalkyl group may consist of 3 carbon ring atoms, 4 carbon ring atoms, 5 carbon ring atoms, etc., up to and including 10 carbon ring atoms. In some embodiments, it is a C₃-C₈ cycloalkyl radical. In some embodiments, it is a C₃-C₅ cycloalkyl radical. Examples of cycloalkyl group include, but are not limited to, cyclopropyl, cyclobutyl, cyclopentyl, cyclopentenyl, cyclohexyl, cyclohexenyl, cycloseptyl, cyclooctyl, cyclononyl, cyclodecyl, and norbornyl. The term “cycloalkyl” also refers to spiral ring system, in which the cycloalkyl rings share one carbon atom.

“Heterocycloalkyl” refers to a 3- to 18-membered nonaromatic ring (e.g., C₃-C₁₈ heterocycloalkyl) radical that comprises two to twelve ring carbon atoms and from one to six heteroatoms selected from nitrogen, oxygen and sulfur. Whenever it appears herein, a numerical range such as “3 to 18” refers to each integer in the given range; e.g., “3 to 18 ring atoms” means that the heterocycloalkyl group may consist of 3 ring atoms, 4 ring atoms, etc., up to and including 18 ring atoms. In some embodiments, it is a C₅-C₁₀ heterocycloalkyl. In some embodiments, it is a C₄-C₁₀ heterocycloalkyl. In some embodiments, it is a C₃-C₁₀ heterocycloalkyl. The heterocycloalkyl radical may be a monocyclic, bicyclic, tricyclic or tetracyclic ring system, which may include fused or bridged ring systems. The heteroatoms in the heterocycloalkyl radical may be optionally oxidized. One or more nitrogen atoms, if present, may optionally be quaternized. The heterocycloalkyl radical may be partially or fully saturated. The heterocycloalkyl may be attached to the rest of the molecule through any atom of the ring(s). Examples of such heterocycloalkyl radicals include, but are not limited to, 6,7-dihydro-5H-cyclopenta[b]pyridine, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. In some embodiments, the heterocycloalkyl group is aziridinyl, azetidinyl, pyrrolidinyl, piperidinyl, homopiperidinyl, morpholinyl, thiomorpholinyl, piperazinyl, tetrahydrofuranyl, tetrahydrothienyl, dihydrooxazolyl, tetrahydropyranyl, tetrahydrothiopyranyl, indolinyl, tetrahydroquinolyl, tetrahydroisoquinolin and benzoxazinyl, preferably dihydrooxazolyl and tetrahydrofuranyl.

“Halo” refers to any of halogen atoms fluorine (F), chlorine (Cl), bromine (Br), or iodine (I). A particular example of such halo groups is fluorine.

“Haloalkyl” refers to an alkyl substituted by one or more halo(s).

“Alkenyl” refers to a group of unsubstituted or substituted hydrocarbons containing 2-18, 2-16, 2-12, 2-10, for example, 2-8 (e.g., 2-6) carbons, which are linear, branched, cyclic, or in combination thereof, with at least one carbon-to-carbon double bond.

“Haloalkenyl” refers to an alkenyl substituted by one or more halo(s).

“Alkynyl” refers to a group of unsubstituted or substituted hydrocarbons containing 2-18, 2-16, 2-12, 2-10, for example, 2-8 (e.g., 2-6) carbons, which are linear, branched, cyclic, or in combination thereof, with at least one carbon-to-carbon triple bond.

“Haloalkynyl” refers to an alkynyl substituted by one or more halo(s).

“Amino protecting group” refers to those groups intended to protect an amino group against undesirable reactions during synthetic procedures and which can later be removed to reveal the amine. Commonly used amino protecting groups are disclosed in Protective Groups in Organic Synthesis, Greene, T. W.; Wuts, P. G. M., John Wiley & Sons, New York, N.Y., (3rd Edition, 1999). Amino protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, alpha-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxy-carbonyl groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, alpha,alpha-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. Amine protecting groups also include cyclic amino protecting groups such as phthaloyl and dithiosuccinimidyl, which incorporate the amino nitrogen into a heterocycle. Typically, amino protecting groups include formyl, acetyl, benzoyl, pivaloyl, t-butylacetyl, phenylsulfonyl, Alloc, Teoc, benzyl, Fmoc, Boc and Cbz.

“Amino” refers to unsubstituted amino and substituted amino groups, for example, primary amines, secondary amines, tertiary amines and quaternary amines. Specifically, “amino” refers to —NRₐR_{b}, wherein Rₐ and R_{b}, both directly connected to the N, can be independently selected from hydrogen, deuterium, halo, hydroxy, cyano, formyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, a nitrogen protective group, —(CO)-alkyl, —(CO)—O-alkyl, or —S(O)ₙR_{c} (n=0 to 2, R_{c} is directly connected to S), wherein R_{c} is independently selected from hydrogen, deuterium, halo, amino, hydroxy, thiol, cyano, formyl, alkyl, haloalkyl, alkenyl, haloalkenyl, alkynyl, haloalkynyl, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio.

“Aryl” refers to a C₆-C₁₄ aromatic hydrocarbon. For example, aryl can be phenyl, napthyl, or fluorenyl.

“Heteroaryl” refers to a C₆-C₁₄ aromatic hydrocarbon having one or more heteroatoms, such as N, O or S. The heteroaryl can be substituted or unsubstituted. Examples of a heteroaryl include, but are not limited to, azepinyl, acridinyl, benzimidazolyl, benzindolyl, 1,3-benzodioxolyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzofurazanyl, benzothiazolyl, benzothienyl, benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). In some embodiments, the heteroaryl can be dithiazinyl, furyl, imidazolyl, indolyl, isoquinolinyl, isoxazolyl, oxadiazolyl (e.g., (1,3,4)-oxadiazolyl, or (1,2,4)-oxadiazolyl), oxazolyl, pyrazinyl, pyrazolyl, pyrazyl, pyridazinyl, pyridinyl, pyrimidinyl, pyrimidyl, pyrrolyl, quinolinyl, tetrazolyl, thiazolyl, thienyl, triazinyl, (1,2,3)-triazolyl, (1,2,4)-triazolyl, 1,3,4-oxadiazolyl, 1,2,4-oxadiazolyl, 1,2,4-triazolyl, 1,3,4-thiadiazolyl, 5-amino-1,2,4-oxadiazolyl, 5-amino-1,3,4-oxadiazolyl, 5-amino-1,3,4-oxadiazolyl, 3-methyl-1,2,4-oxadiazolyl, 5-methyl-1,2,4-oxadiazolyl, 5-(trifluoromethyl)-1,2,4-oxadiazolyl, 5-(methylamino)-1,2,4-oxadiazolyl, 5-(aminomethyl)-1,2,4-oxadiazolyl, 5-(aminomethyl)-1,3,4-oxadiazolyl, 5-amino-4-cyanooxazolyl, 5,6-dichloro-1H-indolyl, 5,6-difluoro-1H-indolyl, 5-chloro-1H-indolyl, 5,6-dibromo-1H-indolyl, 5-fluoro-1H-indolyl, 5-methoxy-1H-indolyl, 7-fluoro-1H-indolyl, 6-cyano-1H-indolyl, 5-cyano-1H-indolyl, 4-fluoro-1H-indolyl, 5,6-difluoro-1H-indolyl, 6-fluoro-1H-indolyl, or 5,7-difluoro-1H-indolyl.

The substituent on the heteroaryl group can be alkyl (e.g., C1-C6 alkyl), amino, cyano, halo (e.g., fluoro, bromo, and chloro), alkylamino (e.g., C1-C6 alkylamino), methyleneamino, nitro, or hydroxyl. The heteroaryl group can have two, three or four substituents.

“Carbocycle” refers to a C₆-C₁₄ cyclic hydrocarbon. For example, aryl can be phenyl, napthyl, or fluorenyl.

“Heterocycle” refers to a C₆-C₁₄ cyclic hydrocarbon having one or more heteroatoms, such as N, O or S.

“Alkoxy” refers to an alkyl connected to an oxygen atom (—O—-alkyl).

“Haloalkoxy” refers to a haloalkyl connected to an oxygen atom (—O-haloalkyl).

“Thioalkoxy” refers to an alkyl connected to a sulfur atom (—S-alkyl).

“Halothioalkoxy” refers to a haloalkyl connected to a sulfur atom (—S-haloalkyl).

“Carbonyl” refers to —(CO)—, wherein (CO) indicates that the oxygen is connected to the carbon with a double bond.

“Alkanoyl” or “acyl” refers to an alkyl connected to a carbonyl group [—(CO)-alkyl].

“Haloalkanoyl” or “haloacyl” refers to a haloalkyl connected to a carbonyl group [—(CO)-haloalkyl].

“Thiocarbonyl” refers to —(CS)—, wherein (CS) indicates that the sulfur is connected to the carbon with a double bond.

“Thioalkanoyl (or thioacyl)” refers to an alkyl connected to a thiocarbonyl group [—(CS)-alkyl].

“Halothioalkanoyl” or “halothioacyl” refers to a haloalkyl connected to a thiocarbonyl group [—(CS)-haloalkyl].

“Carbonyloxy” refers to an alkanoyl (or acyl) connected to an oxygen atom [—O—(CO)-alkyl].

“Halocarbonyloxy” refers to a haloalkanoyl (or haloacyl) connected to an oxygen atom [—O—(CO)-haloalkyl].

“Carbonylthio” refers to an alkanoyl (or acyl) connected to a sulfur atom [—S—(CO)-alkyl].

“Halocarbonylthio” refers to a haloalkanoyl (or haloacyl) connected to a sulfur atom [—S—(CO)-haloalkyl].

“Thiocarbonyloxy” refers to a thioalkanoyl (or thioacyl) connected to an oxygen atom [—O—(CS)-alkyl].

“Halothiocarbonyloxy” refers to a halothioalkanoyl (or halothioacyl) connected to an oxygen atom [—O—(CS)-haloalkyl].

“Thiocarbonylthio” refers to a thioalkanoyl (or thioacyl) connected to a sulfur atom [—S—(CS)-alkyl].

“Halothiocarbonylthio” refers to a halothioalkanoyl (or halothioacyl) connected to a sulfur atom [—S—(CS)-haloalkyl].

The present disclosure provides methods of treating cancer in a patient. In some embodiments, the method includes administering to the patient (1) a therapeutically effective amount of an indole compound, or an enantiomer, diastereomer, or pharmaceutically acceptable salt thereof, and (2) a therapeutically effective amount of an inhibitor of an immune checkpoint protein. In other embodiments, the method includes administering to the patient (1) a therapeutically effective amount of a phosphate derivative of an indole compound, and (2) a therapeutically effective amount of an inhibitor of an immune checkpoint protein. In a particular embodiment, the phosphate derivative of an indole compound is an indolo-phosphoramidate analog (IPA).

### Indole Compounds

The indole compounds in the disclosed methods can modulate human aryl hydrocarbon receptor (AhR). These compounds bind specifically to AhR. Without wishing to be bound by theory, it is contemplated that AhR bound by one of the present compounds is agonized with respect to the receptor's immune-stimualtory activity. In some embodiments, the indole compounds are those described in U.S. Provisional Patent Application No. 62/717,387, filed Aug. 10, 2018, and U.S. Provisional Patent Application No. 62/588,751, filed Nov. 20, 2017, each of which is incorporated by reference in its entirety.

In some embodiments, the compound has the structure of formula 2, or an enantiomer, diastereomer, or pharmaceutically acceptable salt thereof:

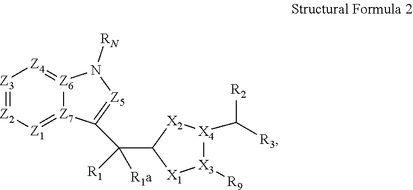

wherein:

X₁ is N (nitrogen), O (oxygen), S (sulfur), or C (carbon); X₂ is N (nitrogen), O (oxygen) S (sulfur), or C (carbon); X₃ is N (nitrogen), O (oxygen), S (sulfur) or C (carbon); and X₄ is N (nitrogen) O (oxygen), S (sulfur), or C (carbon), such that at least one of X₁, X₂, X₃ and X₄ is N, each of X₁, X₂, X₃ and X₄ is optionally selected to form a heteroaromatic, wherein the bond between X₁ and the adjacent carbon, between X₂ and the adjacent carbon, between X₁ and X₄, between X₂ and X₃, and between X₃ and X₄ can be a single bond or a double bond and the valence of X₁, X₂, X₃ and X₄ is completed with H or C₁-C₆ alkyl (i.e., the ring can be aromatic, partially saturated, or saturated);

Z₁ is N or CR₄, Z₂ is N or CR₅, Z₃ is N or CR₆, Z₄ is N or CR₇, Z₅ is N or CR₈, Z₆ is N or C, Z₇ is N or C, wherein no more than two of Z₁, Z₂, Z₃, Z₄, Z₅, Z₆, and Z₇ are N;

R₂ and R₃ are together selected from the group consisting of ═O, ═S, or ═NRₐ (Rₐ is H, C₁-C₆ alkyl, C₁-C₆ acyl, or —OR, R is H, C₁-C₆ alkyl, or C₁-C₆ acyl), or

R₂ and R₃ are each independently selected from the group consisting of —NRₐR_{b} (Rₐ and R_{b} are each independently H, C₁-C₆ alkyl, or C₁-C₆ acyl), hydrogen, deuterium, halo, amino, hydroxy, cyano, formyl, furyl, nitro, alkyl, haloalkyl, alkenyl, haloalkenyl, alkynyl, haloalkynyl, C₁-C₆ acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₀ (n=0 to 2, R₁₀ is directly connected to S), wherein R₁₀ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;

R₄, R₅, R₆, R₇, R₈, and R₉ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;

R_{N} is H, CN, C₁-C₆ alkyl, —OH, —(CO)—OR, or —OR, wherein R is H, C₁-C₆ alkyl, or C₁-C₆ acyl; and

R₁ and R₁ₐ are taken together to form ═NR_{b}, wherein R_{b} is H, C₁-C₆ alkyl, hydroxy, C₁-C₆ alkoxy (—O-alkyl), C₁-C₆ acyloxy, amino, or C₁-C₆ acyl, R₂ preferably can be ═O, R₃ preferably can be —OR, wherein R is H or C₁-C₆ alkyl, or

R₁ and R₁ₐ are taken together to form ═CR_{b}R_{c}, wherein R_{b} and R_{c} are each independently H, C₁-C₆ alkyl, alkoxy (—O-alkyl), thioalkoxy (—S-alkyl), cyano (—CN), or amino, R₂ preferably can be ═O, R₃ preferably can be —OR, wherein R is H or C₁-C₆ alkyl, or

R₁ and R₁ₐ are taken together to form ═O, ═NORₐ, or ═S, R₂ and R₃ preferably can be each independently —OR or —NRₐR_{b}, wherein R, Rₐ, and R_{b} are each independently H, C₁-C₆ alkyl, or C₁-C₆ acyl, or

R₁ and R₁ₐ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₂ (n=0 to 2, R₁₂ is directly connected to S), wherein R₁₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio; and

optionally, adjacent R groups, together, can form a six- to twelve-membered ring.

In certain embodiments, Z₁ is CR₄, Z₂ is CR₅, Z₃ is CR₆, Z₄ is CR₇, Z₅ is CR₈, Z₆ is C, Z₇ is C, wherein R₄ is halo, cyano, formyl, or nitro and each of R₅, R₆, R₇, and R₈ is H. In certain embodiments, at least one of R₄, R₅, R₆, and R₇ is halo, e.g., F, Cl or Br.

In some embodiments, the compound has the structure of formula 2a, or an enantiomer, diastereomer, or pharmaceutically acceptable salt thereof:

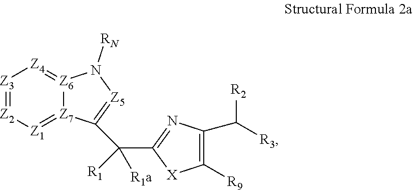

wherein:

X is either O (oxygen) or S (sulfur);

Z₁ is N or CR₄, Z₂ is N or CR₅, Z₃ is N or CR₆, Z₄ is N or CR₇, Z₅ is N or CR₈, Z₆ is N or C, Z₇ is N or C, wherein no more than two of Z₁, Z₂, Z₃, Z₄, Z₅, Z₆, and Z₇ are N;

R₂ and R₃ are together selected from the group consisting of ═O, ═S, or ═NRₐ (Rₐ is H, C₁-C₆ alkyl, C₁-C₆ acyl, or —OR, R is H, C₁-C₆ alkyl, or C₁-C₆ acyl), or

R₂ and R₃ are each independently selected from the group consisting of —NRₐR_{b} (Rₐ and R_{b} are each independently H, C₁-C₆ alkyl, or C₁-C₆ acyl), hydrogen, deuterium, halo, amino, hydroxy, cyano, formyl, furyl, nitro, alkyl, haloalkyl, alkenyl, haloalkenyl, alkynyl, haloalkynyl, C₁-C₆ acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₀ (n=0 to 2, R₁₀ is directly connected to S), wherein R₁₀ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;

R₄, R₅, R₆, R₇, R₈, and R₉ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;

R_{N} is H, CN, C₁-C₆ alkyl, —OH, —(CO)—OR, or —OR, wherein R is H, C₁-C₆ alkyl, or C₁-C₆ acyl; and

R₁ and R₁ₐ are taken together to form ═NR_{b}, wherein R_{b} is H, C₁-C₆ alkyl, hydroxy, C₁-C₆ alkoxy (—O-alkyl), C₁-C₆ acyloxy, amino, or C₁-C₆ acyl, R₂ preferably can be ═O, R₃ preferably can be —OR, wherein R is H or C₁-C₆ alkyl, or

R₁ and R₁ₐ are taken together to form ═CR_{b}R_{c}, wherein R_{b} and R_{c} are each independently H, C₁-C₆ alkyl, alkoxy (—O-alkyl), thioalkoxy (—S-alkyl), cyano (—CN), or amino, R₂ preferably can be ═O, R₃ preferably can be —OR, wherein R is H or C₁-C₆ alkyl, or

R₁ and R₁ₐ are taken together to form ═O, ═NORₐ, or ═S, R₂ and R₃ preferably can be each independently —OR or —NRₐR_{b}, wherein R, Rₐ, and R_{b} are each independently H, C₁-C₆ alkyl, or C₁-C₆ acyl, or

R₁ and R₁ₐ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₂ (n=0 to 2, R₁₂ is directly connected to S), wherein R₁₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio; and

optionally, adjacent R groups, together, can form a six- to twelve-membered ring.

In certain embodiments, the carbon-carbon double bond of the five-membered nitrogen-containing ring can be saturated. The compounds described herein include stereoisomers or diastereomers of the saturated carbon atoms. The saturation can be hydrogen or C₁-C₆ alkyl groups added to the carbon-carbon bond. In certain embodiments, Z₁ is CR₄, Z₂ is CR₅, Z₃ is CR₆, Z₄ is CR₇, Z₅ is CR₈, Z₆ is C, Z₇ is C, wherein R₄ is halo, cyano, formyl, or nitro and each of R₅, R₆, R₇, and R₈ is H. In certain embodiments, at least one of R₄, R₅, R₆, and R₇ is halo, e.g., F, Cl or Br.

In some embodiments, the compound has the structure of formula 3, or an enantiomer, diastereomer, or pharmaceutically acceptable salt thereof:

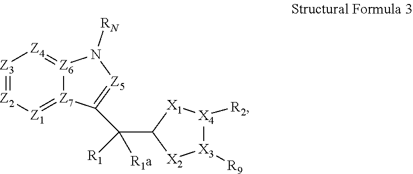

wherein:

X₁ is N (nitrogen), O (oxygen), S (sulfur), or C (carbon); X₂ is N (nitrogen), O (oxygen) S (sulfur), or C (carbon); X₃ is N (nitrogen), O (oxygen), S (sulfur) or C (carbon); and X₄ is N (nitrogen) O (oxygen), S (sulfur), or C (carbon), such that at least one of X₁, X₂, X₃ and X₄ is N, each of X₁, X₂, X₃ and X₄ is optionally selected to form a heteroaromatic, wherein the bond between X₁ and the adjacent carbon, between X₂ and the adjacent carbon, between X₁ and X₄, between X₂ and X₃, and between X₃ and X₄ can be a single bond or a double bond and the valence of X₁, X₂, X₃ and X₄ is completed with H or C₁-C₆ alkyl (i.e., the ring can be aromatic, partially saturated, or saturated);

Z₁ is N or CR₄, Z₂ is N or CR₅, Z₃ is N or CR₆, Z₄ is N or CR₇, Z₅ is N or CR₈, Z₆ is N or C, Z₇ is N or C, wherein no more than two of Z₁, Z₂, Z₃, Z₄, Z₅, Z₆, and Z₇ are N;

R₁ and R₁ₐ are taken together to form ═NR_{b}, wherein R_{b} is H, C₁-C₆ alkyl, hydroxy, C₁-C₆ alkoxy (—O-alkyl), C₁-C₆ acyloxy, amino, or C₁-C₆ acyl, or

R₁ and R₁ₐ are taken together to form ═CR_{b}R_{c}, wherein R_{b} and R_{c} are each independently H, C₁-C₆ alkyl, alkoxy (—O-alkyl), thioalkoxy (—S-alkyl), cyano (—CN), or amino, or

R₁ and R₁ₐ are taken together to form ═O, ═NORₐ, or ═S, wherein Rₐ is H, C₁-C₆ alkyl, or C₁-C₆ acyl, or

R₁ and R₁ₐ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₂ (n=0 to 2, R₁₂ is directly connected to S), wherein R₁₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio; and

R₂ and R₉ are each independently selected from the group consisting of hydrogen, aryl, heteroaryl, cycloalkyl, heterocycloalkyl, alkyl, —NR₂ₐC(O)OR_{2b}, —NR₂ₐC(O)R_{2b}, —(C₀-C₆ alkyl)-CONHSO₂R₂ₐ, —(C₀-C₆ alkyl)-CONHSO₂NR₂ₐR_{2b}, —(C₀-C₆ alkyl)-SO₂NHCOR₂ₐ, —(C₀-C₆ alkyl)-SO₂NHR₂ₐ, —(C₀-C₆ alkyl)-CONR₂ₐOR_{2b},

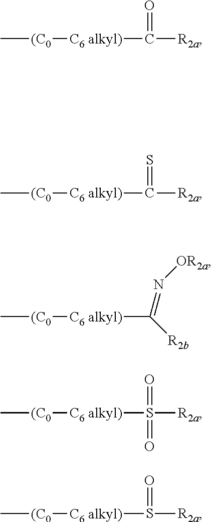

deuterium, halo, amino, hydroxy, cyano, formyl, nitro, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₀ (n=0 to 2, R₁₀ is directly connected to S), wherein R₁₀ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio,

wherein R₂ₐ and R_{2b} are each independently H, C₁-C₆ alkyl, alkoxy (—O-alkyl), hydroxy, thioalkoxy (—S-alkyl), cyano (—CN), or amino;

R₄, R₅, R₆, R₇, and R₈ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;

R_{N} is H, CN, C₁-C₆ alkyl, —OH, —(CO)—OR, or —OR, wherein R is H, C₁-C₆ alkyl, or C₁-C₆ acyl; and

optionally, adjacent R groups, together, can form a six- to twelve-membered ring.

In certain embodiments, Z₁ is CR₄, Z₂ is CR₅, Z₃ is CR₆, Z₄ is CR₇, Z₅ is CR₈, Z₆ is C, Z₇ is C, wherein R₄ is halo, cyano, formyl, or nitro and each of R₅, R₆, R₇, and R₈ is H. In certain embodiments, at least one of R₄, R₅, R₆, and R₇ is halo, e.g., F, Cl or Br. In certain embodiments, R₂ₐ is substituted amino. Substituted amino can include alkyl amino, for example, unsubstituted alkylamino, hydroxyalkylamino or alkoxyalkylamino, or cycloalkyl amino, for example, —NRₐR_{b} where Rₐ and R_{b} together form a 3, 4, 5, 6, 7, or 8 member alkylene ring. The alkylene ring can be unsubstituted or substituted, for example, with halo, hydroxyl, alkoxy, or alkyl (including substituted alkyl) groups.

In some embodiments, the compound has the structure of formula 3c, or an enantiomer, diastereomer, or pharmaceutically acceptable salt thereof:

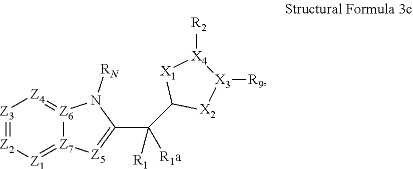

wherein:

X₁ is N (nitrogen), O (oxygen), S (sulfur), or C (carbon); X₂ is N (nitrogen), O (oxygen) S (sulfur), or C (carbon); X₃ is N (nitrogen), O (oxygen), S (sulfur) or C (carbon); and X₄ is N (nitrogen) O (oxygen), S (sulfur), or C (carbon), such that at least one of X₁, X₂, X₃ and X₄ is N, each of X₁, X₂, X₃ and X₄ is optionally selected to form a heteroaromatic, wherein the bond between X₁ and the adjacent carbon, between X₂ and the adjacent carbon, between X₁ and X₄, between X₂ and X₃, and between X₃ and X₄ can be a single bond or a double bond and the valence of X₁, X₂, X₃ and X₄ is completed with H or C₁-C₆ alkyl (i.e., the ring can be aromatic, partially saturated, or saturated);

Z₁ is N or CR₄, Z₂ is N or CR₅, Z₃ is N or CR₆, Z₄ is N or CR₇, Z₅ is N or CR₈, Z₆ is N or C, Z₇ is N or C, wherein no more than two of Z₁, Z₂, Z₃, Z₄, Z₅, Z₆, and Z₇ are N;

R₁ and R₁ₐ are taken together to form ═NR_{b}, wherein R_{b} is H, C₁-C₆ alkyl, hydroxy, C₁-C₆ alkoxy (—O-alkyl), C₁-C₆ acyloxy, amino, or C₁-C₆ acyl, or

R₁ and R₁ₐ are taken together to form ═CR_{b}R_{c}, wherein R_{b} and R_{c} are each independently H, C₁-C₆ alkyl, alkoxy (—O-alkyl), thioalkoxy (—S-alkyl), cyano (—CN), or amino, or

R₁ and R₁ₐ are taken together to form ═O, ═NORₐ, or ═S, wherein Rₐ is H, C₁-C₆ alkyl, or C₁-C₆ acyl, or

R₁ and R₁ₐ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₂ (n=0 to 2, R₁₂ is directly connected to S), wherein R₁₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio; and

R₂ and R₉ are each independently selected from the group consisting of hydrogen, aryl, heteroaryl, cycloalkyl, heterocycloalkyl, alkyl, —NR₂ₐC(O)OR_{2b}, —NR₂ₐC(O)R_{2b}, —(C₀-C₆ alkyl)-CONHSO₂R₂ₐ, —(C₀-C₆ alkyl)-CONHSO₂NR₂ₐR_{2b}, —(C₀-C₆ alkyl)-SO₂NHCOR₂ₐ, —(C₀-C₆ alkyl)-SO₂NHR₂ₐ, —(C₀-C₆ alkyl)-CONR₂ₐOR_{2b},

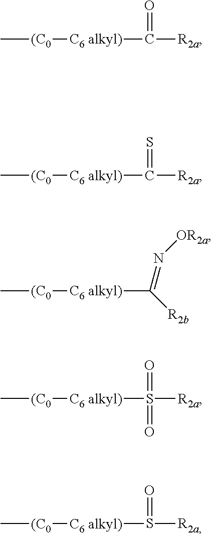

deuterium, halo, amino, hydroxy, cyano, formyl, nitro, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₀ (n=0 to 2, R₁₀ is directly connected to S), wherein R₁₀ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio, wherein R₂ₐ and R_{2b} are each independently H, C₁-C₆ alkyl, alkoxy (—O-alkyl), hydroxy, thioalkoxy (—S-alkyl), cyano (—CN), or amino;

R₄, R₅, R₆, R₇, and R₈ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;

R_{N} is H, CN, C₁-C₆ alkyl, —OH, —(CO)—OR, or —OR, wherein R is H, C₁-C₆ alkyl, or C₁-C₆ acyl; and

optionally, adjacent R groups, together, can form a six- to twelve-membered ring.

In certain embodiments, Z₁ is CR₄, Z₂ is CR₅, Z₃ is CR₆, Z₄ is CR₇, Z₅ is CR₈, Z₆ is C, Z₇ is C, wherein R₄ is halo, cyano, formyl, or nitro and each of R₅, R₆, R₇, and R₈ is H. In certain embodiments, at least one of R₄, R₅, R₆, and R₇ is halo, e.g., F, Cl or Br. In certain embodiments, R₂ₐ is substituted amino. Substituted amino can include alkyl amino, for example, unsubstituted alkylamino, hydroxyalkylamino or alkoxyalkylamino, or cycloalkyl amino, for example, —NRₐR_{b} where Rₐ and R_{b} together form a 3, 4, 5, 6, 7, or 8 member alkylene ring. The alkylene ring can be unsubstituted or substituted, for example, with halo, hydroxyl, alkoxy, or alkyl (including substituted alkyl) groups.

In some embodiments, the compound has the structure of formula 3a, or an enantiomer, diastereomer, or pharmaceutically acceptable salt thereof:

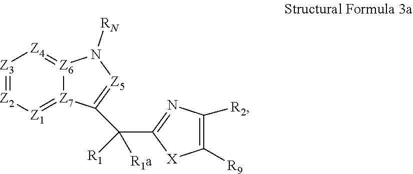

wherein:

X is either O (oxygen) or S (sulfur);

Z₁ is N or CR₄, Z₂ is N or CR₅, Z₃ is N or CR₆, Z₄ is N or CR₇, Z₅ is N or CR₈, Z₆ is N or C, Z₇ is N or C, wherein no more than two of Z₁, Z₂, Z₃, Z₄, Z₅, Z₆, and Z₇ are N;

R₁ and R₁ₐ are taken together to form ═NR_{b}, wherein R_{b} is H, C₁-C₆ alkyl, hydroxy, C₁-C₆ alkoxy (—O-alkyl), C₁-C₆ acyloxy, amino, or C₁-C₆ acyl, or

R₁ and R₁ₐ are taken together to form ═CR_{b}R_{c}, wherein R_{b} and R_{c} are each independently H, C₁-C₆ alkyl, alkoxy (—O-alkyl), thioalkoxy (—S-alkyl), cyano (—CN), or amino, or

R₁ and R₁ₐ are taken together to form ═O, ═NORₐ, or ═S, wherein Rₐ is H, C₁-C₆ alkyl, or C₁-C₆ acyl; or

R₁ and R₁ₐ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₂ (n=0 to 2, R₁₂ is directly connected to S), wherein R₁₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio; and

R₂ and R₉ are each independently selected from the group consisting of hydrogen, aryl, heteroaryl, cycloalkyl, heterocycloalkyl, alkyl, —NR₂ₐC(O)OR_{2b}, —NR₂ₐC(O)R_{2b}, —(C₀-C₆ alkyl)-CONHSO₂R₂ₐ, —(C₀-C₆ alkyl)-CONHSO₂NR₂ₐR_{2b}, —(C₀-C₆ alkyl)-SO₂NHCOR₂ₐ, —(C₀-C₆ alkyl)-SO₂NHR₂ₐ, —(C₀-C₆ alkyl)-CONR₂ₐOR_{2b},

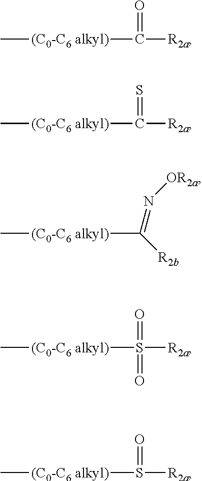

deuterium, halo, amino, hydroxy, cyano, formyl, nitro, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₀ (n=0 to 2, R₁₀ is directly connected to S), wherein R₁₀ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio, wherein R₂ₐ and R_{2b} are each independently H, C₁-C₆ alkyl, alkoxy (—O-alkyl), hydroxy, thioalkoxy (—S-alkyl), cyano (—CN), or amino;

R₄, R₅, R₆, R₇, and R₈ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;

R_{N} is H, CN, C₁-C₆ alkyl, —OH, —(CO)—OR, or —OR, wherein R is H, C₁-C₆ alkyl, or C₁-C₆ acyl; and

optionally, adjacent R groups, together, can form a six- to twelve-membered ring.

In certain embodiments, the carbon-carbon double bond of the five-membered nitrogen-containing ring can be saturated. The compounds described herein include stereoisomers or diastereomers of the saturated carbon atoms. The saturation can be hydrogen or C₁-C₆ alkyl groups added to the carbon-carbon bond. In certain embodiments, Z₁ is CR₄, Z₂ is CR₅, Z₃ is CR₆, Z₄ is CR₇, Z₅ is CR₈, Z₆ is C, Z₇ is C, wherein R₄ is halo, cyano, formyl, or nitro and each of R₅, R₆, R₇, and R₈ is H. In certain embodiments, R₂ₐ is substituted amino. Substituted amino can include alkyl amino, for example, unsubstituted alkylamino, hydroxyalkylamino or alkoxyalkylamino, or cycloalkyl amino, for example, —NRₐR_{b} where Rₐ and R_{b} together form a 3, 4, 5, 6, 7, or 8 member alkylene ring. The alkylene ring can be unsubstituted or substituted, for example, with halo, hydroxyl, alkoxy, or alkyl (including substituted alkyl) groups. In certain embodiments, at least one of R₄, R₅, R₆, and R₇ is halo, e.g., F, Cl or Br.

In some embodiments, the compound has the structure of formula 3b, or an enantiomer, diastereomer, or pharmaceutically acceptable salt thereof:

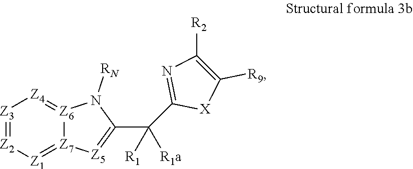

wherein:

X is either O (oxygen) or S (sulfur);

Z₁ is N or CR₄, Z₂ is N or CR₅, Z₃ is N or CR₆, Z₄ is N or CR₇, Z₅ is N or CR₈, Z₆ is N or C, Z₇ is N or C, wherein no more than two of Z₁, Z₂, Z₃, Z₄, Z₅, Z₆, and Z₇ are N;

R₁ and R₁ₐ are taken together to form ═NR_{b}, wherein R_{b} is H, C₁-C₆ alkyl, hydroxy, C₁-C₆ alkoxy (—O-alkyl), C₁-C₆ acyloxy, amino, or C₁-C₆ acyl, or

R₁ and R₁ₐ are taken together to form ═CR_{b}R_{c}, wherein R_{b} and R_{c} are each independently H, C₁-C₆ alkyl, alkoxy (—O-alkyl), thioalkoxy (—S-alkyl), cyano (—CN), or amino, or

R₁ and R₁ₐ are taken together to form ═O, ═NORₐ, or ═S, wherein Rₐ is H, C₁-C₆ alkyl, or C₁-C₆ acyl, or

R₁ and R₁ₐ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₂ (n=0 to 2, R₁₂ is directly connected to S), wherein R₁₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio; and

R₂ and R₉ are each independently selected from the group consisting of hydrogen, aryl, heteroaryl, cycloalkyl, heterocycloalkyl, alkyl, —NR₂ₐC(O)OR_{2b}, —NR₂ₐC(O)R_{2b}, —(C₀-C₆ alkyl)-CONHSO₂R₂ₐ, —(C₀-C₆ alkyl)-CONHSO₂NR₂ₐR_{2b}, —(C₀-C₆ alkyl)-SO₂NHCOR₂ₐ, —(C₀-C₆ alkyl)-SO₂NHR₂ₐ, —(C₀-C₆ alkyl)-CONR₂ₐOR_{2b},

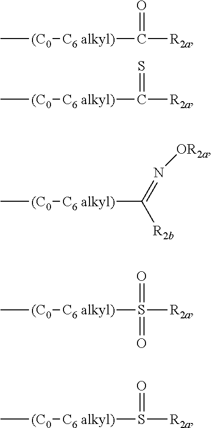

deuterium, halo, amino, hydroxy, cyano, formyl, nitro, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₀ (n=0 to 2, R₁₀ is directly connected to S), wherein R₁₀ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio, wherein R₂ₐ and R_{2b} are each independently H, C₁-C₆ alkyl, alkoxy (—O-alkyl), hydroxy, thioalkoxy (—S-alkyl), cyano (—CN), or amino;

R₄, R₅, R₆, R₇, and R₈ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;

R_{N} is H, CN, C₁-C₆ alkyl, —OH, —(CO)—OR, or —OR, wherein R is H, C₁-C₆ alkyl, or C₁-C₆ acyl; and

optionally, adjacent R groups, together, can form a six- to twelve-membered ring.

In certain embodiments, the carbon-carbon double bond of the five-membered nitrogen-containing ring can be saturated. The compounds described herein include stereoisomers or diastereomers of the saturated carbon atoms. The saturation can be hydrogen or C₁-C₆ alkyl groups added to the carbon-carbon bond. In certain embodiments, Z₁ is CR₄, Z₂ is CR₅, Z₃ is CR₆, Z₄ is CR₇, Z₅ is CR₈, Z₆ is C, Z₇ is C, wherein R₄ is halo, cyano, formyl, or nitro and each of R₅, R₆, R₇, and R₈ is H. In certain embodiments, R₂ₐ is substituted amino. Substituted amino can include alkyl amino, for example, unsubstituted alkylamino, hydroxyalkylamino or alkoxyalkylamino, or cycloalkyl amino, for example, —NRₐR_{b} where Rₐ and R_{b} together form a 3, 4, 5, 6, 7, or 8 member alkylene ring. The alkylene ring can be unsubstituted or substituted, for example, with halo, hydroxyl, alkoxy, or alkyl (including substituted alkyl) groups. In certain embodiments, at least one of R₄, R₅, R₆, and R₇ is halo, e.g., F, Cl or Br.

In some embodiments, the compound has the structure of formula 4, or an enantiomer, diastereomer, or pharmaceutically acceptable salt thereof:

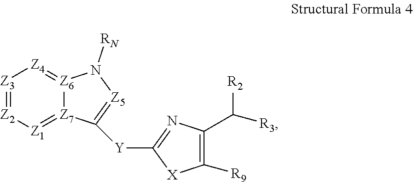

wherein:

X is O (oxygen) or S (sulfur);

Y is a bond, O (oxygen), S (sulfur), or

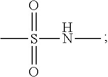

Z₁ is N or CR₄, Z₂ is N or CR₅, Z₃ is N or CR₆, Z₄ is N or CR₇, Z₅ is N or CR₈, Z₆ is N or C, Z₇ is N or C, wherein no more than two of Z₁, Z₂, Z₃, Z₄, Z₅, Z₆, and Z₇ are N;

R₄, R₅, R₆, R₇, R₈, and R₉ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₀ (n=0 to 2, R₁₀ is directly connected to S), wherein R₁₀ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;

R_{N} is H, CN, C₁-C₆ alkyl, —OH, —(CO)—OR, or —OR, wherein R is H, C₁-C₆ alkyl, or C₁-C₆ acyl;

R₂ and R₃ are together selected from the group consisting of ═O, ═S, or ═NRₐ (Rₐ is H, C₁-C₆ alkyl, C₁-C₆ acyl, or —OR, R is H, C₁-C₆ alkyl, or C₁-C₆ acyl), or

R₂ and R₃ are each independently selected from the group consisting of —NRₐR_{b} (Rₐ and R_{b} are each independently H, C₁-C₆ alkyl, or C₁-C₆ acyl), hydrogen, deuterium, halo, amino, hydroxy, cyano, formyl, furyl, nitro, alkyl, haloalkyl, alkenyl, haloalkenyl, alkynyl, haloalkynyl, C₁-C₆ acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio; and

optionally, adjacent R groups, together, can form a six- to twelve-membered ring.

In certain embodiments, the carbon-carbon double bond of the five-membered nitrogen-containing ring can be saturated. The compounds described herein include stereoisomers or diastereomers of the saturated carbon atoms. The saturation can be hydrogen or C₁-C₆ alkyl groups added to the carbon-carbon bond. In certain embodiments, Z₁ is CR₄, Z₂ is CR₅, Z₃ is CR₆, Z₄ is CR₇, Z₅ is CR₈, Z₆ is C, Z₇ is C, wherein R₄ is halo, cyano, formyl, or nitro and each of R₅, R₆, R₇, and R₈ is H. In certain embodiments, at least one of R₄, R₅, R₆, and R₇ is halo, e.g., F, Cl or Br.

In some embodiments, the compound has the structure of formula 5, or an enantiomer, diastereomer, or pharmaceutically acceptable salt thereof:

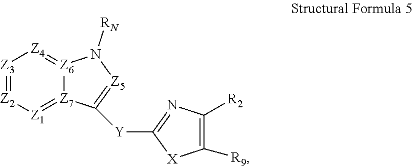

wherein:

X is O (oxygen) or S (sulfur);

Y is a bond, O (oxygen), S (sulfur), or

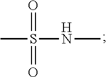

Z₁ is N or CR₄, Z₂ is N or CR₅, Z₃ is N or CR₆, Z₄ is N or CR₇, Z₅ is N or CR₈, Z₆ is N or C, Z₇ is N or C, wherein no more than two of Z₁, Z₂, Z₃, Z₄, Z₅, Z₆, and Z₇ are N;

R₂ and R₉ are each independently selected from the group consisting of hydrogen, aryl, heteroaryl, cycloalkyl, heterocycloalkyl, alkyl, —NR₂ₐC(O)OR_{2b}, —NR₂ₐC(O)R_{2b}, —(C₀-C₆ alkyl)-CONHSO₂R₂ₐ, —(C₀-C₆ alkyl)-CONHSO₂NR₂ₐR_{2b}, —(C₀-C₆ alkyl)-SO₂NHCOR₂ₐ, —(C₀-C₆ alkyl)-SO₂NHR₂ₐ, —(C₀-C₆ alkyl)-CONR₂ₐOR_{2b},

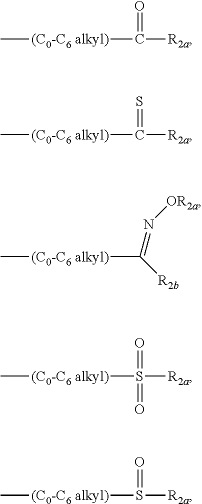

deuterium, halo, amino, hydroxy, cyano, formyl, nitro, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₀ (n=0 to 2, R₁₀ is directly connected to S), wherein R₁₀ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio, wherein R₂ₐ and R_{2b} are each independently H, C₁-C₆ alkyl, alkoxy (—O-alkyl), hydroxy, thioalkoxy (—S-alkyl), cyano (—CN), or amino;

R₄, R₅, R₆, R₇, and R₈ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;

R_{N} is H, CN, C₁-C₆ alkyl, —OH, —(CO)—OR, or —OR, wherein R is H, C₁-C₆ alkyl, or C₁-C₆ acyl; and

optionally, adjacent R groups, together, can form a six- to twelve-membered ring.

In certain embodiments, the carbon-carbon double bond of the five-membered nitrogen-containing ring can be saturated. The compounds described herein include stereoisomers or diastereomers of the saturated carbon atoms. The saturation can be hydrogen or C1-C6 alkyl groups added to the carbon-carbon bond. In certain embodiments, Z₁ is CR₄, Z₂ is CR₅, Z₃ is CR₆, Z₄ is CR₇, Z₅ is CR₈, Z₆ is C, Z₇ is C, wherein R₄ is halo, cyano, formyl, or nitro and each of R₅, R₆, R₇, and R₈ is H. In certain embodiments, at least one of R₄, R₅, R₆, and R₇ is halo, e.g., F, Cl or Br. In certain embodiments, R₂ₐ is substituted amino. Substituted amino can include alkyl amino, for example, unsubstituted alkylamino, hydroxyalkylamino or alkoxyalkylamino, or cycloalkyl amino, for example, —NRₐR_{b} where Rₐ and R_{b} together form a 3, 4, 5, 6, 7, or 8 member alkylene ring. The alkylene ring can be unsubstituted or substituted, for example, with halo, hydroxyl, alkoxy, or alkyl (including substituted alkyl) groups.

In still another embodiment, the compound has structural formula 6, or an enantiomer, diastereomer, or pharmaceutically acceptable salt thereof:

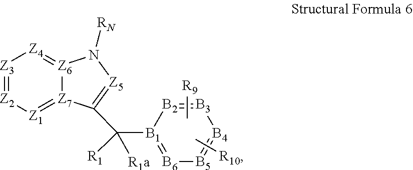

wherein:

R₁ and R₁ₐ are taken together to form ═NR_{b}, wherein R_{b} is H, C₁-C₆ alkyl, hydroxy, C₁-C₆ alkoxy (—O-alkyl), C₁-C₆ acyloxy, amino, or C₁-C₆ acyl, or

R₁ and R₁ₐ are taken together to form ═CR_{b}R_{c}, wherein R_{b} and R_{c} are each independently H, C₁-C₆ alkyl, alkoxy (—O-alkyl), thioalkoxy (—S-alkyl), cyano (—CN), or amino, or

R₁ and R₁ₐ are taken together to form ═O, ═NORₐ, or ═S, wherein Rₐ is H, C₁-C₆ alkyl, or C₁-C₆ acyl, or

R₁ and R₁ₐ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₄ (n=0 to 2, R₁₄ is directly connected to S), wherein R₁₄ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;

Z₁ is N or CR₄, Z₂ is N or CR₅, Z₃ is N or CR₆, Z₄ is N or CR₇, Z₅ is N or CR₈, Z₆ is N or C, Z₇ is N or C, wherein no more than two of Z₁, Z₂, Z₃, Z₄, Z₅, Z₆, and Z₇ are N;

R₄, R₅, R₆, R_{7,} and R₈ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;

R_{N} is H, CN, C₁-C₆ alkyl, —OH, —(CO)—OR, or —OR, wherein R is H, C₁-C₆ alkyl, or C₁-C₆ acyl;

B₁, B₂, B₃, B₄, B₅, and B₆ are each independently C or N;

R₉ and R₁₀, the number of which, together, complete the valence of each of B₁, B₂, B₃, B₄, B₅, and B₆, are each independently selected from the group consisting of hydrogen, aryl, heteroaryl, cycloalkyl, heterocycloalkyl, alkyl, —NR₂ₐC(O)OR_{2b}, —NR₂ₐC(O)R_{2b}, —(C₀-C₆ alkyl)-CONHSO₂R₂ₐ, —(C₀-C₆ alkyl)-CONHSO₂NR₂ₐR_{2b}, —(C₀-C₆ alkyl)-SO₂NHCOR₂ₐ, —(C₀-C₆ alkyl)-SO₂NHR₂ₐ, —(C₀-C₆ alkyl)-CONR₂ₐOR_{2b},

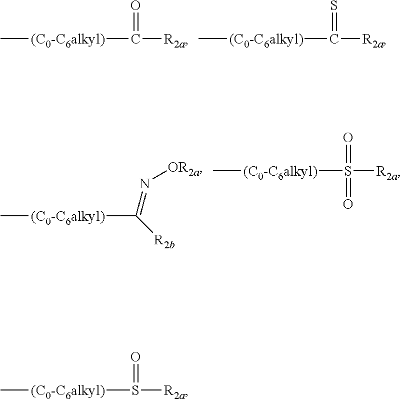

deuterium, halo, amino, hydroxy, cyano, formyl, nitro, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₂ (n=0 to 2, R₁₂ is directly connected to S), wherein R₁₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and

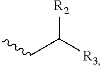

wherein R₂ₐ and R_{2b} are each independently H, C₁-C₆ alkyl, alkoxy (—O-alkyl), hydroxy, thioalkoxy (—S-alkyl), cyano (—CN), or amino;

wherein R₂ and R₃ are together selected from the group consisting of ═O, ═S, or ═NRₐ (Rₐ is H, C₁-C₆ alkyl, C₁-C₆ acyl, or —OR, R is H, C₁-C₆ alkyl, or C₁-C₆ acyl), or

R₂ and R₃ are each independently selected from the group consisting of —NRₐR_{b} (Rₐ and R_{b} are each independently H, C₁-C₆ alkyl, or C₁-C₆ acyl), hydrogen, deuterium, halo, amino, hydroxy, cyano, formyl, furyl, nitro, alkyl, haloalkyl, alkenyl, haloalkenyl, alkynyl, haloalkynyl, C₁-C₆ acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₃ (n=0 to 2, R₁₃ is directly connected to S), wherein R₁₃ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio; and

optionally, adjacent R groups, together, can form a six- to twelve-membered ring.

In certain embodiments, Z₁ is CR₄, Z₂ is CR₅, Z₃ is CR₆, Z₄ is CR₇, Z₅ is CR₈, Z₆ is C, Z₇ is C, wherein R₄ is halo, cyano, formyl, or nitro and each of R₅, R₆, R₇, and R₈ is H. In certain embodiments, at least one of R₄, R₅, R₆, and R₇ is halo, e.g., F, Cl or Br. In certain embodiments, R₂ₐ is substituted amino. Substituted amino can include alkyl amino, for example, unsubstituted alkylamino, hydroxyalkylamino or alkoxyalkylamino, or cycloalkyl amino, for example, —NRₐR_{b} where Rₐ and R_{b} together form a 3, 4, 5, 6, 7, or 8 member alkylene ring. The alkylene ring can be unsubstituted or substituted, for example, with halo, hydroxyl, alkoxy, or alkyl (including substituted alkyl) groups.

In some embodiments, the compound has structural formula 7, or an enantiomer, diastereomer, or pharmaceutically acceptable salt thereof:

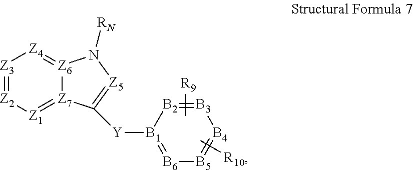

wherein:

Y is a bond, O (oxygen), S (sulfur), or

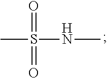

Z₁ is N or CR₄, Z₂ is N or CR₅, Z₃ is N or CR₆, Z₄ is N or CR₇, Z₅ is N or CR₈, Z₆ is N or C, Z₇ is N or C, wherein no more than two of Z₁, Z₂, Z₃, Z₄, Z₅, Z₆, and Z₇ are N;

R₄, R₅, R₆, R₇, and R₈ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;

R_{N} is H, CN, C₁-C₆ alkyl, —OH, —(CO)—OR, or —OR, wherein R is H, C₁-C₆ alkyl, or C₁-C₆ acyl;

B₁, B₂, B₃, B₄, B₅, and B₆ are each independently C or N;

R₉ and R₁₀, the number of which, together, complete the valence of each of B₁, B₂, B₃, B₄, B₅, and B₆, are each independently selected from the group consisting of hydrogen, aryl, heteroaryl, cycloalkyl, heterocycloalkyl, alkyl, —NR₂ₐC(O)OR_{2b}, —NR₂ₐC(O)R_{2b}, —(C₀-C₆ alkyl)-CONHSO₂R₂ₐ, —(C₀-C₆ alkyl)-CONHSO₂NR₂ₐR_{2b}, —(C₀-C₆ alkyl)-SO₂NHCOR₂ₐ, —(C₀-C₆ alkyl)-SO₂NHR₂ₐ, —(C₀-C₆ alkyl)-CONR₂ₐOR_{2b},

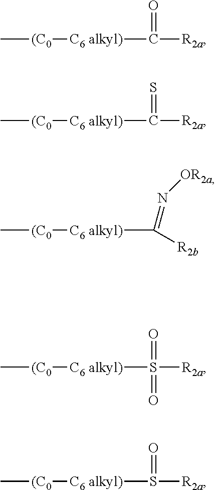

deuterium, halo, amino, hydroxy, cyano, formyl, nitro, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₂ (n=0 to 2, R₁₂ is directly connected to S), wherein R₁₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and

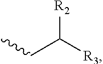

wherein R₂ₐ and R_{2b} are each independently H, C₁-C₆ alkyl, alkoxy (—O-alkyl), hydroxy, thioalkoxy (—S-alkyl), cyano (—CN), or amino;

wherein R₂ and R₃ are together selected from the group consisting of ═O, ═S, or ═NRₐ (Rₐ is H, C₁-C₆ alkyl, C₁-C₆ acyl, or —OR, R is H, C₁-C₆ alkyl, or C₁-C₆ acyl), or

R₂ and R₃ are each independently selected from the group consisting of —NRₐR_{b} (Rₐ and R_{b} are each independently H, C₁-C₆ alkyl, or C₁-C₆ acyl), hydrogen, deuterium, halo, amino, hydroxy, cyano, formyl, furyl, nitro, alkyl, haloalkyl, alkenyl, haloalkenyl, alkynyl, haloalkynyl, C₁-C₆ acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₃ (n=0 to 2, R₁₃ is directly connected to S), wherein R₁₃ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio; and

optionally, adjacent R groups, together, can form a six- to twelve-membered ring.

In certain embodiments, Z₁ is CR₄, Z₂ is CR₅, Z₃ is CR₆, Z₄ is CR₇, Z₅ is CR₈, Z₆ is C, Z₇ is C, wherein R₄ is halo, cyano, formyl, or nitro and each of R₅, R₆, R₇, and R₈ is H. In certain embodiments, at least one of R₄, R₅, R₆, and R₇ is halo, e.g., F, Cl or Br. In certain embodiments, R₂ₐ is substituted amino. Substituted amino can include alkyl amino, for example, unsubstituted alkylamino, hydroxyalkylamino or alkoxyalkylamino, or cycloalkyl amino, for example, —NRₐR_{b} where Rₐ and R_{b} together form a 3, 4, 5, 6, 7, or 8 member alkylene ring. The alkylene ring can be unsubstituted or substituted, for example, with halo, hydroxyl, alkoxy, or alkyl (including substituted alkyl) groups.

In each of the formulae, in some embodiments, each of R₄, R₅, R₆, and R₇ is hydrogen. In other embodiments, at least one of R₄, R₅, R₆, and R₇ can be F, Cl or Br and the others of R₄, R₅, R₆, and R₇ are hydrogen. In still other embodiments, at least two of R₄, R₅, R₆, and R₇, independently, can be F, Cl or Br and the others of R₄, R₅, R₆, and R₇ are hydrogen. The F, Cl or Br can be at the indole ring carbon 5, 6, or 7.

In each of formulae 3, 3a, 3b, 3c, and 5, in certain embodiments, R₉ can be hydrogen. R₂ can be acyl, cyano, hydroxyl-substituted C1-C6 alkyl, amino-substituted C1-C6 alkyl, aryl, or heteroaryl. The aryl or heteroaryl can be substituted or unsubstituted. The substituted aryl or heteroaryl can be substituted with halo, amino, hydroxyl, or C1-C6 alkyl. The amino can be unsubstituted.

In each of formulae 2, 2a, and 4, in certain embodiments, R₂ can be hydroxyl or amino and R₃ can be alkyl, aryl, nitro, or cyano. R₉ can be hydrogen. The amino can be substituted or unsubstituted.

In some embodiments, the compound has structural formula 8, or an enantiomer, diastereomer, or pharmaceutically acceptable salt thereof:

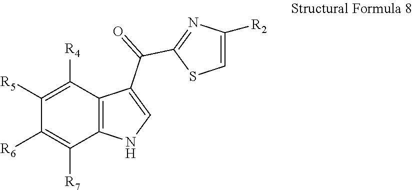

wherein R₂ is selected from the group consisting of substituted alkyl, heteroaryl, and

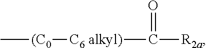

wherein R₂ₐ is H, C1-C6 alkyl, alkoxy (—O-alkyl), hydroxy, thioalkoxy (—S-alkyl), cyano (—CN), or amino; and

R₄, R₅, R₆, and R₇, are each independently selected from the group consisting of hydrogen and halo.

In some embodiments, R₂ is substituted alkyl, e.g., a C1-C6 alkyl substituted with one or more hydroxyl, amino, nitro, or cyano. In some embodiments, R₂ is heteroaryl, e.g., oxadiazolyl or thiadiazolyl, optionally substituted with one or more hydroxyl, amino, nitro, cyano, C1-C6 alkyl, or C1-C6 alkyl amino. In some embodiments, R₂ is —C(O)—R₂ₐ, and R₂ₐ is C1-C6 alkyl.

In one embodiment of the compound of structural formula 8, at least one of R₄, R₅, R₆, and R₇ is F, Cl or Br and the others of R₄, R₅, R₆, and R₇ are hydrogen. In another embodiment, at least two of R₄, R₅, R₆, and R₇ are F, Cl or Br and the others of R₄, R₅, R₆, R₇ are hydrogen.

In one embodiment, R₅ is F and R₄, R₆, and R₇ are hydrogen. In another embodiment, R₆ is F and R₄, R₅, and R₇ are hydrogen. In still another embodiment, R₇ is F and R₄, R₅, and R₆ are hydrogen.

In one embodiment, R₅ is Cl and R₄, R₆, and R₇ are hydrogen. In another embodiment, R₆ is Cl and R₄, R₅, and R₇ are hydrogen. In still another embodiment, R₇ is Cl and R₄, R₅, and R₆ are hydrogen.

In one embodiment, R₅ and R₆ are F and R₄ and R₇ are hydrogen. In another embodiment, R₅ and R₇ are F and R₄ and R₆ are hydrogen. In still another embodiment, R₆ and R₇ are F and R₄ and R₅ are hydrogen.

In one embodiment, R₅ and R₆ are Cl and R₄ and R₇ are hydrogen. In another embodiment, R₅ and R₇ are Cl and R₄ and R₆ are hydrogen. In still another embodiment, R₆ and R₇ are Cl and R₄ and R₅ are hydrogen.

In some embodiments, each of R₄, R₅, R₆ and R₇ is hydrogen.

Exemplary indole compounds that can be used in the disclosed methods are shown in Tables 1 and 2.

TABLE 1
Representative Indole Compounds

| ARI-# | Structural Formula | ¹H NMR Data | Mass Characterization |
|---|---|---|---|
| 001 | 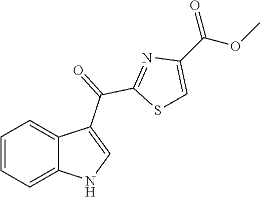 [SMILES: COC(=O)c1csc(C(=O)c2c[nH]c3ccccc23)n1] | | |
| 002 | 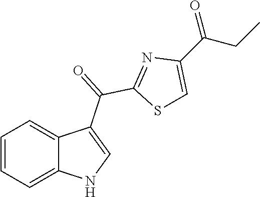 [SMILES: CCC(=O)c1csc(C(=O)c2c[nH]c3ccccc23)n1] | | |
| 003 | 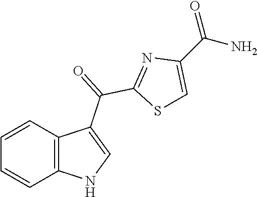 [SMILES: NC(=O)c1csc(C(=O)c2c[nH]c3ccccc23)n1] | | |
| 004 | 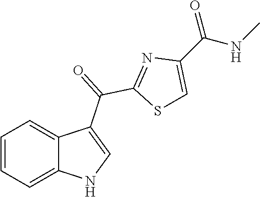 [SMILES: CNC(=O)c1csc(C(=O)c2c[nH]c3ccccc23)n1] | | |
| 005 | 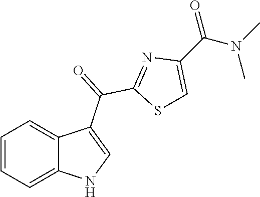 [SMILES: CN(C)C(=O)c1csc(C(=O)c2c[nH]c3ccccc23)n1] | | |
| 006 | 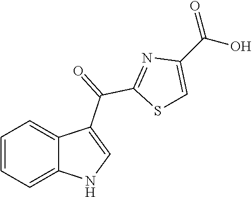 [SMILES: O=C(O)c1csc(C(=O)c2c[nH]c3ccccc23)n1] | | |
| 007 | 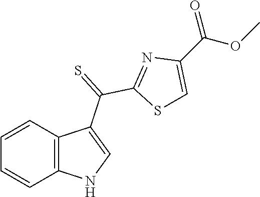 [SMILES: COC(=O)c1csc(C(=S)c2c[nH]c3ccccc23)n1] | ¹H NMR (500 MHz, DMSO-d₆) δ 12.81 (s, 1H), 9.12 (s, 1H), 8.89-8.64 (m, 1H), 8.77 (s, 1H), 7.62-7.59 (m, 1H), 7.38-7.35 (m, 2H), 3.90 (s, 3H). | ESI MS m/z 303 [M + H]⁺ |
| 008 | 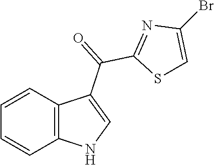 [SMILES: O=C(c1nc(Br)cs1)c1c[nH]c2ccccc12] | 1H NMR (500 MHz, DMSO-d₆) δ 12.32 (s, 1H), 8.99 (d, J = 3.5 Hz, 1H), 8.30-8.28 (m, 2H), 7.60-7.57 (m, 1H), 7.32-7.28 (m, 2H). | ESI MS m/z 307 [M +H]⁺ |
| 009 | 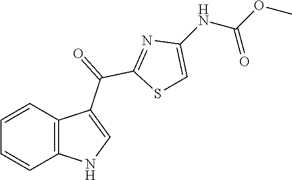 [SMILES: COC(=O)Nc1csc(C(=O)c2c[nH]c3ccccc23)n1] | ¹H NMR (500 MHz, DMSO-d₆) δ 12.81 (s, 1H), 10.77 (s, 1H), 9.15 (s, 1H), 8.31 (dd, J = 6.5, 2.0 Hz, 1H), 7.60 (s, 1H), 7.55 (dd, J = 6.0, 1.5 Hz, 1H), 7.30-7.55 (m, 2H), 3.73 (s, 3H). | ESI MS m/z 302 [M + H]⁺ |
| 011 | 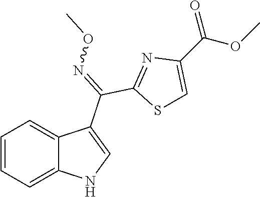 [SMILES: CON=C(c1nc(C(=O)OC)cs1)c1c[nH]c2ccccc12] | ¹H NMR (500 MHz, CDCl₃, 1.4:1 mixture of oxime (E), (Z)- isomers) δ 8.77 (s, 1H), 8.51 (d, J = 3.0 Hz, 0.7H), 8.45 (s, 0.7H), 8.39 (s, 0.7H), 8.35-8.34 (m, 0.7H), 8.21 (s, 1H), 7.82 (d, J = 2.5 Hz, 1H), 7.47-7.38 (m, 1.7H), 7.32-7.30 (m, 1H), 7.24-7.20 (m, 1.4H), 7.17-7.14 (m, 1H), 7.11-7.08 (m, 1H), 4.29 (s, 2.1H), 4.13 (s, 3H), 3.97 (s, 2.1H), 3.89 (s, 3H). | ESI MS m/z 316 [M + H]⁺ |
| 013 | 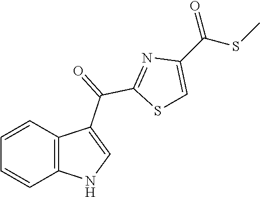 [SMILES: CSC(=O)c1csc(C(=O)c2c[nH]c3ccccc23)n1] | ¹H NMR (500 MHz, DMSO-d₆) δ 12.43 (s, 1H), 9.12 (d, J = 3.0 Hz, 1H), 8.86 (s, 1H), 8.33-8.31 (m, 1H), 7.61-7.59 (m, 1H), 7.33-7.30 (m, 2H), 2.48 (s, 3H). | ESI MS m/z 303 [M + H]⁺ |
| 014 | 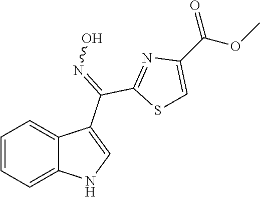 [SMILES: COC(=O)c1csc(C(=NO)c2c[nH]c3ccccc23)n1] | ¹H NMR (500 MHz DMSO-d₆, 3.8:1 mixture of oxime (E), (Z)- isomers) δ 12.81 (s, 1H), 12.06 (s, 0.26H), 11.68 (s, 0.26H), 11.47 (s, 1H), 8.77 (s, 1H), 8.56 (s, 0.26H), 8.40 (d, J = 3.0 Hz, 1H), 8.14 (d, J = 8.0 Hz, 1H), 7.87 (d, J = 2.5 Hz, 0.26H), 7.46-7.44 (m, 1.26H), 7.29 (d, J = 8.0 Hz, 0.26H), 7.18-7.08 (m, 2.26H), 7.00 (t, J = 8.0 Hz, 0.26H), 3.89 (s, 3H), 3.80 (s, 0.82H). | ESI MS m/z 302 [M + H]⁺ |
| 015 | 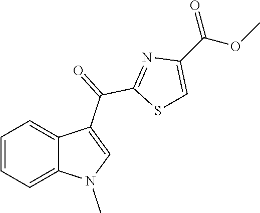 [SMILES: COC(=O)c1csc(C(=O)c2cn(C)c3ccccc23)n1] | ¹H NMR (500 MHz, CDCl₃) δ 9.07 (s, 1H), 8.53-8.51 (m, 1H), 8.40 (s, 1H), 7.42-7.36 (m, 3H), 3.04 (s, 3H), 3.94 (s, 3H). | ESI MS m/z 301 [M + H]⁺ |
| 016 | 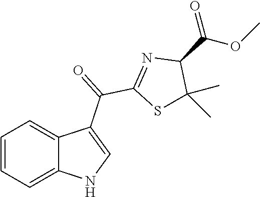 [SMILES: COC(=O)[C@@H]1N=C(C(=O)c2c[nH]c3ccccc23)SC1(C)C] | ¹H NMR (500 MHz, CDCl₃) δ 8.76 (d, , J = 3.0 Hz, 1H), 8.70 (s, 1H), 8.44-8.43 (m, 1H), 7.43-7.42 (m, 1H), 7.34-7.32 (m, 2H), 5.0 (s, 1H), 3.85 (s, 3H), 1.79 (s, 3H), 1.4 (s, 3H). | ESI MS m/z 317 [M + H]⁺ |
| 017 | 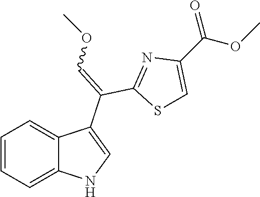 [SMILES: COC=C(c1nc(C(=O)OC)cs1)c1c[nH]c2ccccc12] | ¹H NMR (500 MHz, CDCl₃, 2.3:1 mixture of (E), (Z)-isomers) δ 8.32, 8.29 (s, 1.26H), 8.16 (s, 1H), 7.94 (s, 0.4H), 7.85 (s, 0.4H), 7.60-7.57 (m, 2H), 7.43-7.36 (m, 2.5H), 7.24-7.17 (m, 1.7H), 7.12-7.08 (m, 1.48H), 6.98 (s, 1H), 4.02 (s, 3H), 1.29 (s, 1.29H), 3.88 (s, 4.28 H). | ESI MS m/z 315 [M + H]⁺ |
| 018 | 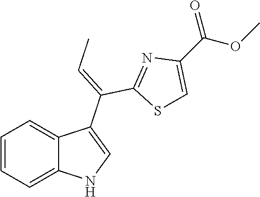 [SMILES: C/C=C(\c1nc(C(=O)OC)cs1)c1c[nH]c2ccccc12] | ¹H NMR (500 MHz, CDCl₃, 1.8:1 mixture of (E), (Z)-isomers) δ 8.34 (bs, 0.89H), 8.17-7.16 (m, 1H), 8.01 (s, 0.94H), 7.50-7.44 (m, 1.86H), 7.40-7.28 (m, 4.10H), 7.25-7.17 (m, 2.37H), 7.13-7.08 (m, 1.61H), 6.40 (q, J = 7.0 Hz, 0.52H), 3.97 (s, 1.67H), 3.96 (s, 3H), 2.19 (d, J = 7.0 Hz, 1.68H), 1.80 (d, J = 7.0 Hz, 3.0H). | ESI MS m/z 299 [M + H]⁺ |
| 019 | 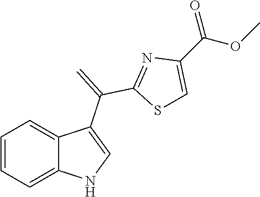 [SMILES: C=C(c1nc(C(=O)OC)cs1)c1c[nH]c2ccccc12] | ¹H NMR (500 MHz, CDCl₃, key protons reported) δ 8.30 (bs, 1H, indole NH), 6.26 (d, J = 0.8 Hz, 1H, olefin), 5.83 (bs, 1H, olefin), 3.96 (s, 3H, methyl ester). | ESI MS m/z 285 [M + H]⁺ |
| 020 | 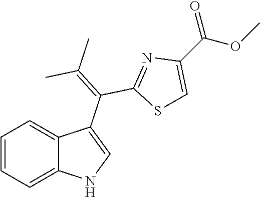 [SMILES: COC(=O)c1csc(C(=C(C)C)c2c[nH]c3ccccc23)n1] | ¹H NMR (500 MHz, CDCl₃) δ 8.23 (s, 1H), 8.01 (s, 1H), 7.41-7.39 (m, 1H), 7.34-7.32 (m, 1H), 7.22-7.19 (m, 1H), 7.17 (d, J = 2.5 Hz, 1H), 7.10-7.07, (m, 1H), 3.95 (s, 3H), 2.37 (s, 3H), 1,83 (s, 3H). | ESI MS m/z 313 [M + H]⁺ |
| 021 | 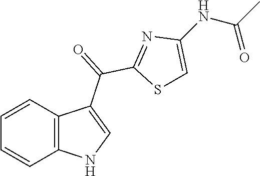 [SMILES: CC(=O)Nc1csc(C(=O)c2c[nH]c3ccccc23)n1] | ¹H NMR (500 MHz, DMSO-d₆) δ 12.40 (s, 1H), 11.43 (s, 1H), 9.11 (s, 1H), 8.32-8.30 (m, 1H), 7.91 (s, 1H), 7.57-7.55 (m, 1H), 7.31-7.26 (m, 2H), 2.12 (s, 3H). | ESI MS m/z 284 [M − H]⁻ |
| 022 | 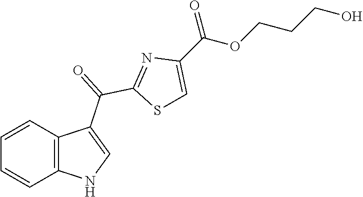 [SMILES: O=C(OCCCO)c1csc(C(=O)c2c[nH]c3ccccc23)n1] | ¹H NMR (500 MHz, DMSO-d₆) δ 12.38 (s, 1H), 9.10 (s, 1H), 8.87 (s, 1H), 8.32-8.30 (m, 1H), 7.60-7.58 (m, 1H), 7.32-7.29 (m, 2H), 4.61 (t, , J = 5.0 Hz, 1H), 4.40 (app t, J = 6.5 Hz, 2H), 3.58 (app q, J = 6.1, Hz, 2H), 1.90 (quint, J = 6.4 Hz, 2H). | ESI MS m/z 331 [M + H]⁺ |
| 023 | 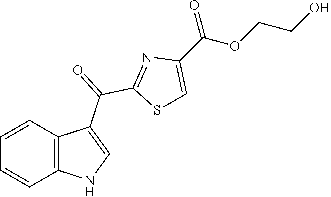 [SMILES: O=C(OCCO)c1csc(C(=O)c2c[nH]c3ccccc23)n1] | ¹H NMR (500 MHz, DMSO-d₆) δ 12.30 (s, 1H), 9.11 (s, 1H), 8.89 (s, 1H), 8.32-8.30 (m, 1H), 7.60-7.58 (m, 1H), 7.32-7.28 (m, 2H), 4.96 (t, , J = 5.0 Hz, 1H), 4.36 (app t, J = 6.0 Hz, 2H), 3.73 (app q, J = 5.5, Hz, 2H). | ESI MS m/z 317 [M + H]⁺ |
| 024 | 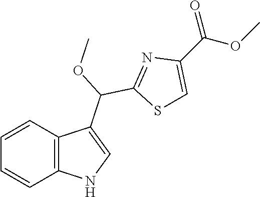 [SMILES: COC(=O)c1csc(C(OC)c2c[nH]c3ccccc23)n1] | ¹H NMR (500 MHz, DMSO-d₆) δ 11.20 (s, 1H), 8.51 (s, 1H), 7.45 (d, , J = 2.5 Hz, 1H), 7.40 (d, J = 8.0 Hz, 1H), 7.37 (d, , J = 8.0 Hz, 1H), 7.09-7.06 (m, 1H), 6.95-6.92 (m, 1H), 5.89 (s, 1H), 3.76 (s, 3H), 3.34 (s, 3H). | ESI MS m/z 303 [M + H]⁺ ESI MS m/z 271 [M + H − CH₃OH]⁺ |
| 025 | 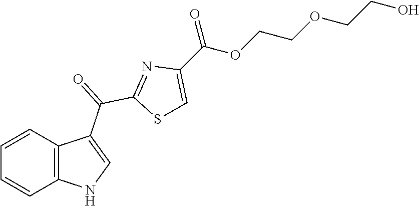 [SMILES: O=C(OCCOCCO)c1csc(C(=O)c2c[nH]c3ccccc23)n1] | ¹H NMR (500 MHz, CDCl₃) δ 9.35 (d, J = 1.5 Hz, 1H), 9.11 (s, 1H), 8.52 (d, J = 2 Hz, 1H), 8.45 (s, 1H), 7.46 (dd, J = 7, 1.5 Hz, 1H), 7.37-7.31 (m, 2H), 4.58-4.57 (m, 2H), 3.91-3.89 (m, 2H), 3.85 (app dd, J = 4.0, 2.0 Hz, 2H), 3.74 (app dd, J = 6.0, 3.0 Hz, 2H). | ESI MS m/z 361 [M + H]⁺ |
| 026 | 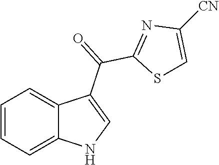 [SMILES: N#Cc1csc(C(=O)c2c[nH]c3ccccc23)n1] | ¹H NMR (500 MHz, DMSO-d₆) δ 12.39 (s, 1H), 9.18 (s, 1H), 9.03 (d, J = 3 Hz, 1H), 8.29-8.28 (m, 1H), 7.60-7.58 (m, 1H), 7.33-7.27 (m, 2H). | ESI MS m/z 254 [M + H]⁺ |
| 028 | 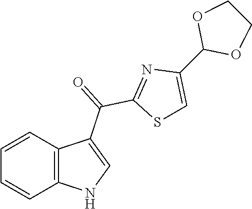 [SMILES: O=C(c1nc(C2OCCO2)cs1)c1c[nH]c2ccccc12] | ¹H NMR (500 MHz, CDCl₃) δ 9.17 (1, , J = 3.0 Hz, 1H), 8.74 (s, 1H), 8.53 (dd, J = 8.5, 1.5 Hz, 1H), 7.73 (s, 1H), 7.46-7.44 (m, 1H), 7.37-7.30 (m, 2H), 6.11 (s, 1H), 4.20-4.10 (m, 4H). | ESI MS m/z 301 [M + H]⁺ |
| 029 | 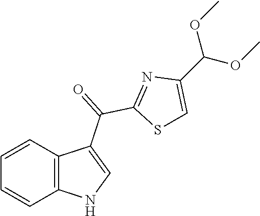 [SMILES: COC(OC)c1csc(C(=O)c2c[nH]c3ccccc23)n1] | ¹H NMR (500 MHz, DMSO-d₆) δ 12.26 (s, 1H), 9.04 (s, 1H), 8.32-8.30 (m, 1H), 8.03 (s, 1H), 7.58-7.57 (m, 1H), 7.31-7.25 (m, 2H), 5.68 (d, J = 1.0 Hz, 1H), 3.34 (s, 3H), 3.31 (s, 3H). | ESI MS m/z 303 [M + H]⁺ |
| 030 | 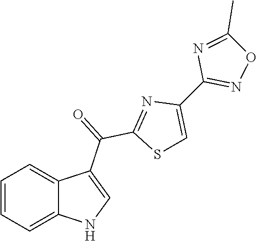 [SMILES: Cc1nc(-c2csc(C(=O)c3c[nH]c4ccccc34)n2)no1] | ¹H NMR (500 MHz, DMSO-d₆) δ 12.35 (s, 1H), 9.14 (s, 1H), 8.78 (s, 1H), 8.34-8.31 (m, 1H), 7.61-7.59 (m, 1H), 7.33-7.28 (m, 2H), 2.72 (s, 3H). | ESI MS m/z 309 [M − H]⁻ |
| 031 | 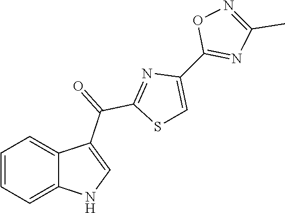 [SMILES: Cc1noc(-c2csc(C(=O)c3c[nH]c4ccccc34)n2)n1] | ¹H NMR (500 MHz, DMSO-d₆) δ 12.36 (s, 1H), 9.14 (s, 1H), 9.10 (s, 1H), 8.33-8.31 (m, 1H), 7.62-7.60 (m, 1H), 7.33-7.29 (m, 2H), 2.48 (s, 3H). | ESI MS m/z 309 [M − H]⁻ |
| 032 | 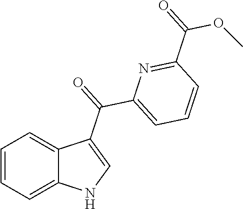 [SMILES: COC(=O)c1cccc(C(=O)c2c[nH]c3ccccc23)n1] | ¹H NMR (500 MHz, DMSO-d₆) δ 12.20 (s, 1H), 9.11 (s, 1H), 8.43-8.39 (m, 1H), 8.30-8.22 (m, 3H), 7.56-7.52 (m, 1H), 7.29-7.26 (m, 2H), 3.98 (s, 3H). | ESI MS m/z 279 [M − H]⁻ |
| 033 | 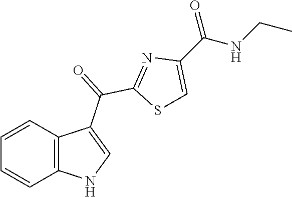 [SMILES: CCNC(=O)c1csc(C(=O)c2c[nH]c3ccccc23)n1] | ¹H NMR (500 MHz, DMSO-d₆) δ 12.35 (s, 1H), 9.43 (d, J = 3.0 Hz, 1H), 8.79 (t, J = 5.5 Hz, 1H), 8.59 (s, 1H), 8.35-8.31 (m, 1H), 7.59-7.55 (m, 1H), 7.32-7.26 (m, 2H), 3.41-3.36 (m, 2H), 1.19 (t, J = 7.0 Hz, 3H). | ESI MS m/z 300 [M + H]⁺ |
| 034 | 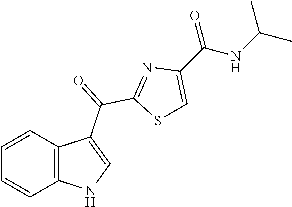 [SMILES: CC(C)NC(=O)c1csc(C(=O)c2c[nH]c3ccccc23)n1] | ¹H NMR (500 MHz, DMSO-d₆) δ 12.42 (s, 1H), 9.38 (s, 1H), 8.59 (s, 1H), 8.42 (d, J = 3.0 Hz, 1H), 8.34-8.30 (m, 1H), 7.57-7.54 (m, 1H), 7.31-7.26 (m, 2H), 4.21-4.14 (m, 1H), 1.26 (s, 3H), 1.25 (s, 3H). | ESI MS m/z 314 [M + H]⁺ |
| 035 | 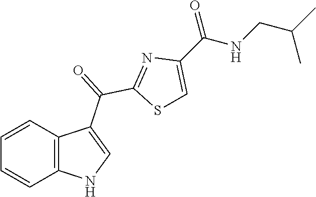 [SMILES: CC(C)CNC(=O)c1csc(C(=O)c2c[nH]c3ccccc23)n1] | ¹H NMR (500 MHz, DMSO-d₆) δ 12.36 (s, 1H), 9.41 (s, 1H), 8.73 (t, J = 6.0 Hz, 1H), 8.59 (s, 1H), 8.33-8.31 (m, 1H), 7.57-7.56 (m, 1H), 7.32-7.28 (m, 2H), 3.18 (t, J = 6.5 Hz, 2H), 1.96-1.90 (m, 1H), 0.93 (s, 3H), 0.92 (s, 3H). | ESI MS m/z 328 [M + H]⁺ |
| 036 | 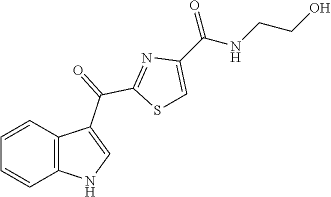 [SMILES: O=C(NCCO)c1csc(C(=O)c2c[nH]c3ccccc23)n1] | ¹H NMR (500 MHz, DMSO-d₆) δ 12.36 (s, 1H), 9.40 (s, 1H), 8.69 (t, J = 5.5 Hz, 1H), 8.61 (s, 1H), 8.33-8.31 (m, 1H), 7.58-7.56 (m, 1H), 7.32-7.26 (m, 2H), 4.81 (t, J = 5.5 Hz, 1H), 3.58 (dd, J = 12.0, 6.5 Hz, 2H), 3.43 (dd, J = 12.0, 6.0 Hz, 2H). | ESI MS m/z 316 [M + H]⁺ |
| 037 | 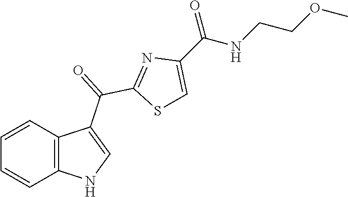 [SMILES: COCCNC(=O)c1csc(C(=O)c2c[nH]c3ccccc23)n1] | ¹H NMR (500 MHz, DMSO-d₆) δ 12.36 (s, 1H), 9.40 (s, 1H), 8.76 (bs, 1H), 8.61 (s, 1H), 8.33-8.31 (m, 1H), 7.58-7.55 (m, 1H), 7.32-7.26 (m, 2H), 3.52-3.51 (m, 4H), 3.30 (s, 3H). | ESI MS m/z 330 [M + H]⁺ |
| 038 | 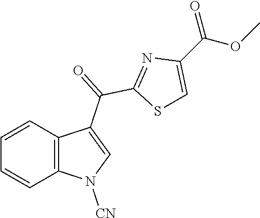 [SMILES: COC(=O)c1csc(C(=O)c2cn(C#N)c3ccccc23)n1] | ¹H NMR (500 MHz, DMSO-d₆) δ 9.32 (s, 1H), 9.01 (s, 1H), 8.38 (d, J = 7.0 Hz, 1H), 7.81 (d, J = 8.0 Hz, 1H), 7.63-7.55 (m, 2H), 3.94 (s, 3H). | ESI MS m/z 312 [M + H]⁺ |
| 039 | 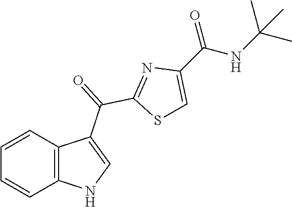 [SMILES: CC(C)(C)NC(=O)c1csc(C(=O)c2c[nH]c3ccccc23)n1] | ¹H NMR (500 MHz, DMSO-d₆) δ 12.44 (s, 1H), 9.28 (d, J = 3.0 Hz, 1H), 8.57 (s, 1H), 8.32-8.29 (m, 1H), 7.81 (bs, 1H), 7.57-7.53 (m, 1H), 7.31-7.26 (m, 2H), 1.46 (s, 9H). | ESI MS m/z 328 [M + H]⁺ |
| 040 | 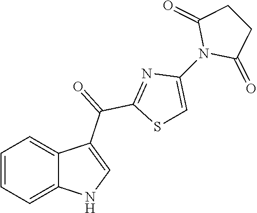 [SMILES: O=C(c1nc(N2C(=O)CCC2=O)cs1)c1c[nH]c2ccccc12] | ¹H NMR (500 MHz, DMSO-d₆) δ 12.32 (s, 1H), 8.97 (s, 1H), 8.32-8.30 (m, 1H), 8.07 (s, 1H), 7.58-7.56 (m, 1H), 7.31-7.27 (m, 2H), 2.88 (s, 4H). | ESI MS m/z 324 [M − H]⁻ |
| 041 | 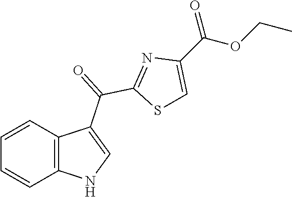 [SMILES: CCOC(=O)c1csc(C(=O)c2c[nH]c3ccccc23)n1] | ¹H NMR (400 MHz, DMSO-d₆) δ 12.38 (s, 1H), 9.10 (s, 1H), 8.87 (s, 1H), 8.30-8.35 (m, 1H), 7.55-7.62 (m, 1H), 7.28-7.33 (m, 2H), 4.39 (q, J = 7.2 Hz, 2H), 1.37 (t, J = 7.2 Hz, 3H). | ESI MS m/z 301 [M + H]⁺ |
| 042 | 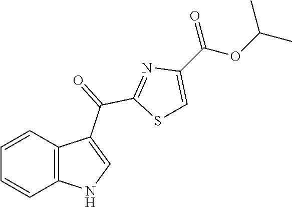 [SMILES: CC(C)OC(=O)c1csc(C(=O)c2c[nH]c3ccccc23)n1] | ¹H NMR (400 MHz, DMSO-d₆) δ 12.38 (s, 1H), 9.10 (s, 1H), 8.83 (s, 1H), 8.30-8.33 (m, 1H), 7.57-7.61 (m, 1H), 7.26-7.34 (m, 2H), 5.15-5.24 (m, 1H), 1.23 (s, 6H). | ESI MS m/z 315 [M + H]⁺ |
| 043 | 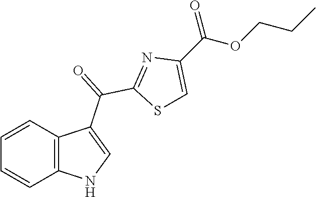 [SMILES: CCCOC(=O)c1csc(C(=O)c2c[nH]c3ccccc23)n1] | ¹H NMR (400 MHz, DMSO-d₆) δ 12.41 (s, 1H), 9.11 (s, 1H), 8.89 (s, 1H), 8.30-8.33 (m, 1H), 7.57-7.62 (m, 1H), 7.28-7.35 (m, 2H), 4.30 (t, J = 6.4 Hz, 2H), 1.72-1.80 (m, 2H), 1.0 (t, J = 7.2 Hz, 3H). | ESI MS m/z 315 [M + H]⁺ |
| 044 | 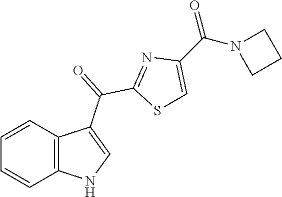 [SMILES: O=C(c1nc(C(=O)N2CCC2)cs1)c1c[nH]c2ccccc12] | ¹H NMR (500 MHz, DMSO-d₆) δ 12.30 (s, 1H), 9.00 (d, J = 3.5 Hz, 1H), 8.65 (s, 1H), 8.31-8.29 (m, 1H), 7.58-7.55 (m, 1H), 7.32-7.26 (m, 2H), 4.73 (d, J = 7.5 Hz, 2H), 4.12 (d, J = 7.5 Hz, 2H), 2.38-2.31 (m, 2H). | ESI MS m/z 312 [M + H]⁺ |
| 045 | 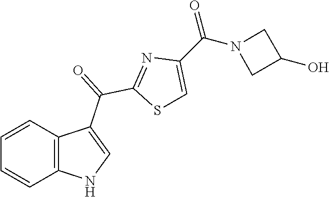 [SMILES: O=C(c1nc(C(=O)N2CC(O)C2)cs1)c1c[nH]c2ccccc12] | ¹H NMR (500 MHz, DMSO-d₆) δ 12.36 (s, 1H), 8.99 (d, J = 3.5 Hz, 1H), 8.66 (s, 1H), 8.31-8.29 (m, 1H), 7.58-7.56 (m, 1H), 7.32-7.30 (m, 2H), 5.82 (d, J = 7.0 Hz, 1H), 4.92-4.89 (m, 1H), 4.60-4.55 (m, 1H), 4.43-4.40 (m, 1H), 4.34-4.31 (m, 1H), 3.85 (dd, J = 10.5, 3.5 Hz, 1H). | ESI MS m/z 328 [M + H]⁺ |
| 046 | 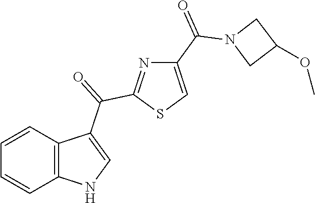 [SMILES: COC1CN(C(=O)c2csc(C(=O)c3c[nH]c4ccccc34)n2)C1] | ¹H NMR (500 MHz, DMSO-d₆) δ 12.34 (s, 1H), 9.00 (d, J = 3.0 Hz, 1H), 8.68 (s, 1H), 8.31-8.28 (m, 1H), 7.59-7.56 (m, 1H), 7.32-7.28 (m, 2H), 4.90-4.87 (m, 1H), 4.51 (dd, J = 10.0, 2.5 Hz, 1H), 4.34-4.28 (m, 2H), 3.94-3.90 (m, 1H), 3.28 (s, 3H). | ESI MS m/z 342 [M + H]⁺ |
| 047 | 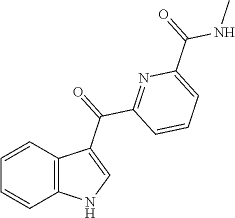 [SMILES: CNC(=O)c1cccc(C(=O)c2c[nH]c3ccccc23)n1] | ¹H NMR (500 MHz, DMSO-d₆) δ 12.14 (s, 1H), 8.63 (q, J = 4.5 Hz, 1H), 8.57 (d, J = 3.0 Hz, 1H), 8.38-8.35 (m, 1H), 8.22-8.17 (m, 2H), 8.11 (dd, J = 7.0, 2.5 Hz, 1H), 7.55-7.52 (m, 1H), 7.29-7.25 (m, 2H), 2.87 (d, J = 4.5 Hz, 3H). | ESI MS m/z 280 [M + H]⁺ |
| 048 | 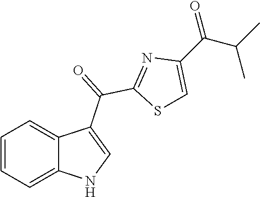 [SMILES: CC(C)C(=O)c1csc(C(=O)c2c[nH]c3ccccc23)n1] | ¹H NMR (400 MHz, DMSO-d₆) δ 12.35 (bs, 1H), 9.12 (s, 1H), 8.88 (s, 1H), 8.30-8.35 (m, 1H), 7.57-7.62 (m, 1H), 7.27-7.35 (m, 2H), 3.80-3.88 (m, 1H), 1.22 (d, J = 6.8 Hz, 6H). | ESI MS m/z 299 [M + H]⁺ |
| 049 | 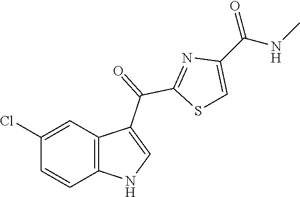 [SMILES: CNC(=O)c1csc(C(=O)c2c[nH]c3ccc(Cl)cc23)n1] | ¹H NMR (400 MHz, DMSO-d₆) δ 12.52 (bs, 1H), 9.50 (s, 1H), 8.74 (bs, 1H), 8.62 (s, 1H), 8.30 (d, J = 2.0 Hz, 1H), 7.60 (d, J = 8.8 Hz, 1H), 7.30-7.35 (m, 2H), 2.87 (d, J = 4.8 Hz, 3H). | ESI MS m/z 318 [M − H]⁻ |
| 050 | 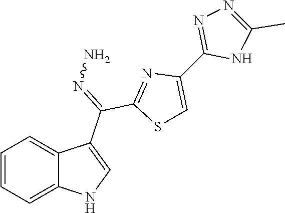 [SMILES: Cc1nnc(-c2csc(C(=NN)c3c[nH]c4ccccc34)n2)[nH]1] | ¹H NMR (500 MHz, DMSO-d₆) δ 14.42 (s, 1H), 13.88 (s, 1H), 9.47-9.18 (m, 1H), 8.51 (bs, 1H), 8.36 (dd, J = 7.0, 1.0 Hz, 1H), 7.67 (d, J = 7.5 Hz, 1H), 7.38-7.34 (m, 2H), 4.02 (s, 3H), 2.41 (s, 2H). | ESI MS m/z 324 [M + H]⁺ |
| 052 | 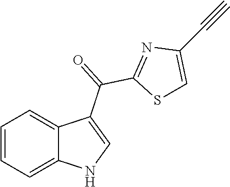 [SMILES: C#Cc1csc(C(=O)c2c[nH]c3ccccc23)n1] | ¹H NMR (500 MHz, CDCl₃) δ 9.20 (d, J = 3.5 Hz, 1H), 8.71 (bs, 1H), 8.53 (dd, J = 7.0, 2.0 Hz, 1H), 7.79 (s, 1H), 7.48-7.46 (m, 1H), 7.38-7.32 (m, 2H), 3.27 (s, 1H). | ESI MS m/z 253 [M + H]⁺ |
| 053 | 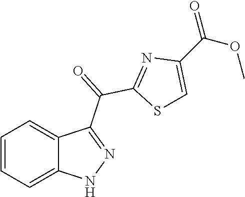 [SMILES: COC(=O)c1csc(C(=O)c2n[nH]c3ccccc23)n1] | ¹H NMR (500 MHz, DMSO-d₆) δ 14.37 (s, 1H), 8.95 (s, 1H), 8.30 (d, J = 8.5 Hz, 1H), 7.78 (d, J = 8.5, Hz, 1H), 7.55 (dt, J = 6.0, 1.0 Hz, 1H), 7.43 (dt, J = 8.0, 0.5 Hz, 1H), 3.91 (s, 3H). | ESI MS m/z 288 [M + H]⁺ |
| 054 | 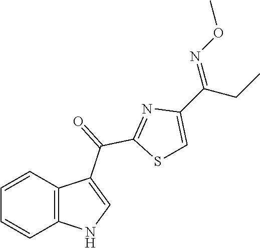 [SMILES: CC/C(=N\OC)c1csc(C(=O)c2c[nH]c3ccccc23)n1] | ¹H NMR (400 MHz, DMSO-d₆) δ 12.32 (bs, 1H), 9.07 (s, 1H), 8.31-8.34 (m, 2H), 7.56-7.59 (m, 1H), 7.27-7.31 (m, 1H), 3.97 (s, 3H), 2.90 (q, J = 7.6 Hz, 2H), 1.14-1.25 (m, 3H). | ESI MS m/z 314 [M + H]⁺ |
| 055 | 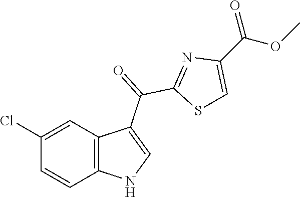 [SMILES: COC(=O)c1csc(C(=O)c2c[nH]c3ccc(Cl)cc23)n1] | ¹H NMR (400 MHz, DMSO-d₆) δ 12.52 (s, 1H), 9.12 (s, 1H), 8.92 (s, 1H), 8.28 (d, J = 2.0 Hz, 1H), 7.63 (d, J = 8.4 Hz, 1H), 7.32-7.36 (m, 1H), 3.94 (s, 3H). | ESI MS m/z 319 [M − H]⁻ |
| 056 | 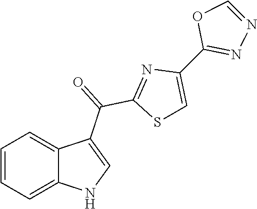 [SMILES: O=C(c1nc(-c2nnco2)cs1)c1c[nH]c2ccccc12] | ¹H NMR (500 MHz, DMSO-d₆) δ 12.39 (s, 1H), 9.45 (s, 1H), 9.14 (d, J = 3.5 Hz, 1H), 8.96 (s, 1H), 8.33-8.32 (m, 1H), 7.62-7.59 (m, 1H), 7.34-7.28 (m, 2H). | ESI MS m/z 297 [M + H]⁺ |
| 057 | 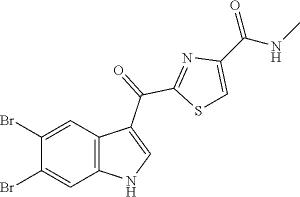 [SMILES: CNC(=O)c1csc(C(=O)c2c[nH]c3cc(Br)c(Br)cc23)n1] | ¹H NMR (400 MHz, DMSO-d₆) δ 12.58 (bs, 1H), 9.49 (d, J = 3.2 Hz, 1H), 8.70-8.74 (m, 1H), 8.62-8.85 (m, 2H), 7.95 (s, 1H), 2.87-2.89 (s, 3H). | ESI MS m/z 440 [M − H]⁻ |
| 058 | 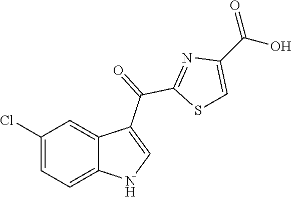 [SMILES: O=C(O)c1csc(C(=O)c2c[nH]c3ccc(Cl)cc23)n1] | ¹H NMR (400 MHz, DMSO-d₆) δ 13.46 (bs, 1H), 12.76 (s, 1H), 9.17 (s, 1H), 8.83 (s, 1H), 8.28 (d, J = 2.0 Hz, 1H), 7.64 (d, J = 8.4 Hz, 1H), 7.31-7.35 (m, 1H). | ESI MS m/z 305 [M − H]⁻ |
| 059 | 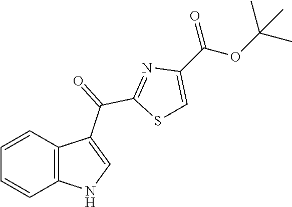 [SMILES: CC(C)(C)OC(=O)c1csc(C(=O)c2c[nH]c3ccccc23)n1] | ¹H NMR (400 MHz, DMSO-d₆) δ 12.40 (bs, 1H), 9.12 (s, 1H), 8.76 (s, 1H), 8.30-8.33 (m, 1H), 7.58-7.61 (m, 1H), 7.28-7.33 (m, 2H), 1.60 (s, 9H). | ESI MS m/z 329 [M + H]⁺ |
| 060 | 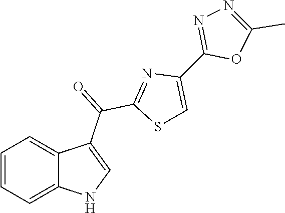 [SMILES: Cc1nnc(-c2csc(C(=O)c3c[nH]c4ccccc34)n2)o1] | ¹H NMR (500 MHz, DMSO-d₆) δ 12.38 (s, 1H), 9.12 (d, J = 3.0 Hz, 1H), 8.87 (s, 1H), 8.34-8.31 (m, 1H), 7.62-7.59 (m, 1H), 7.33-7.28 (m, 2H), 2.64 (s, 3H). | ESI MS m/z 311 [M + H]⁺ |
| 061 | 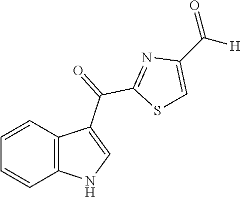 [SMILES: O=Cc1csc(C(=O)c2c[nH]c3ccccc23)n1] | ¹H NMR (500 MHz, DMSO-d₆) δ 12.39 (s, 1H), 10.08 (s, 1H), 9.15 (s, 1H), 9.03 (s, 1H), 8.32-8.30 (m, 1H), 7.60-7.58 (m, 1H), 7.33-7.28 (m, 2H). | ESI MS m/z 257 [M + H]⁺ |
| 062 | 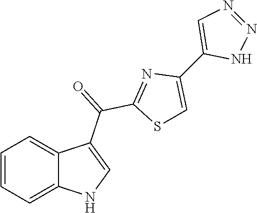 [SMILES: O=C(c1nc(-c2cnn[nH]2)cs1)c1c[nH]c2ccccc12] | ¹H NMR (500 MHz, DMSO-d₆) δ 15.51, 15.19 (bs, 1H), 12.27 (s, 1H), 9.29 (s, 1H), 8.70, 8.40 (bs, 2H), 8.36-8.32 (m, 1H), 7.60-7.57 (m, 1H), 7.32-7.26 (m, 2H). | ESI MS m/z 296 [M + H]⁺ |
| 063 | 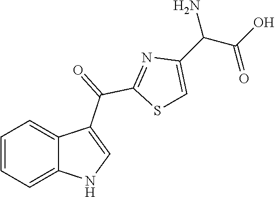 [SMILES: NC(C(=O)O)c1csc(C(=O)c2c[nH]c3ccccc23)n1] | ¹H NMR (500 MHz, DMSO-d₆) δ 12.34 (s, 1H), 9.31 (d, J = 2.5 Hz, 1H), 8.34-8.30 (m, 1H), 8.20 (bs, 3H), 8.00 (d, J = 0.5 Hz, 1H), 7.57-7.53 (m, 1H), 7.30-7.26 (m, 2H), 4.58 (s, 1H). | ESI MS m/z 302 [M + H]⁺ |
| 064 | 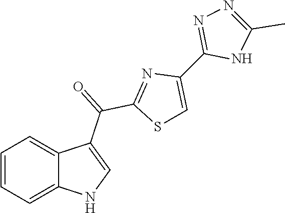 [SMILES: Cc1nnc(-c2csc(C(=O)c3c[nH]c4ccccc34)n2)[nH]1] | ¹H NMR (500 MHz, DMSO-d₆) δ 14.46, 13.82 (bs, 1H), 12.40, 12.30 (bs, 1H), 8.57, 8.41 (bs, 1H), 8.33 (br d, J = 8.5 Hz, 1H), 7.59 (br d, J = 6.5 Hz, 1H), 7.32-7.28 (m, 2H), 2.45, 2.36 (s, 3H). | ESI MS m/z 310 [M + H]⁺ |
| 065 | 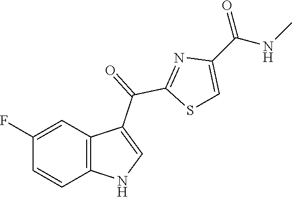 [SMILES: CNC(=O)c1csc(C(=O)c2c[nH]c3ccc(F)cc23)n1] | ¹H NMR (400 MHz, DMSO-d₆) δ 12.52 (bs, 1H), 9.50 (s, 1H), 8.74 (bs, 1H), 8.62 (s, 1H), 7.98-7.99 (m, 1H), 7.58-7.62 (m, 1H), 7.14-7.20 (m, 1H), 2.89 (d, J = 4.8 Hz, 3H). | ESI MS m/z 304 [M + H]⁺ |
| 066 | 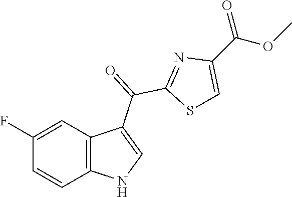 [SMILES: COC(=O)c1csc(C(=O)c2c[nH]c3ccc(F)cc23)n1] | ¹H NMR (400 MHz, DMSO-d₆) δ 12.47 (s, 1H), 9.13 (s, 1H), 8.92 (s, 1H), 7.96-8.00 (m, 1H), 7.61-7.65 (m, 1H), 7.17-7.21 (m, 1H), 3.95 (s, 3H). | ESI MS m/z 303 [M − H]⁻ |
| 067 | 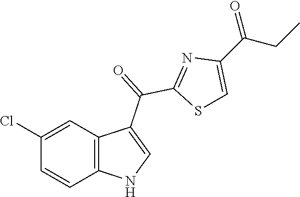 [SMILES: CCC(=O)c1csc(C(=O)c2c[nH]c3ccc(Cl)cc23)n1] | ¹H NMR (400 MHz, DMSO-d₆) δ 12.51 (bs, 1H), 9.19 (s, 1H), 8.86 (s, 1H), 8.29 (d, J = 2.0 Hz, 1H), 7.62 (d, J = 8.4 Hz, 1H), 7.31-7.36 (m, 1H), 3.25 (q, J = 7.2 Hz, 2H), 1.15 (t, J = 7.2 Hz, 3H). | ESI MS m/z 317 [M − H]⁻ |
| 068 | 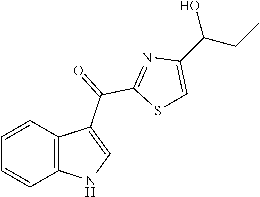 [SMILES: CCC(O)c1csc(C(=O)c2c[nH]c3ccccc23)n1] | ¹H NMR (400 MHz, DMSO-d₆) δ 12.25 (bs, 1H), 9.15 (d, J = 2.0 Hz, 1H), 8.30-8.34 (m, 1H), 7.82 (s, 1H), 7.55-7.60 (m, 1H), 7.26-7.30 (m, 2H), 5.47 (d, J = 5.2 Hz, 1H), 4.71-4.77 (m, 1H), 1.90-2.00 (m, 1H), 1.75-1.88 (m, 1H), 0.94 (t, J = 5.4 Hz, 3H). | ESI MS m/z 287 [M + H]⁺ |
| 069 | 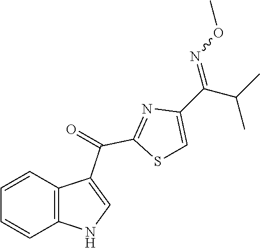 [SMILES: CON=C(c1csc(C(=O)c2c[nH]c3ccccc23)n1)C(C)C] | ¹H NMR (400 MHz, DMSO-d₆) δ 12.32 (bs, 1H), 9.02 (s, 1H), 8.74 (s, 1H), 8.31-8.33 (m, 1H), 7.56-7.59 (m, 1H), 7.28-7.31 (m, 2H), 3.95 (s, 3H), 3.50-3.55 (m, 1H), 1.25 (d, J = 6.8 Hz, 6H). | ESI MS m/z 326 [M − H]⁻ |
| 070 | 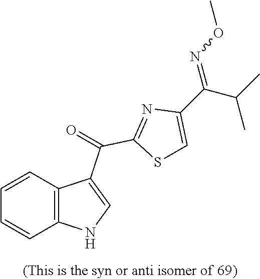 [SMILES: CON=C(c1csc(C(=O)c2c[nH]c3ccccc23)n1)C(C)C] | ¹H NMR (400 MHz, DMSO-d₆) δ 12.36 (bs, 1H), 9.09 (s, 1H), 8.30-8.34 (m, 1H), 8.21 (s, 1H), 7.55-7.60 (m, 1H), 7.26-7.30 (m, 2H), 3.95 (s, 3H), 3.65-3.69 (m, 1H), 1.24-1.32 (m, 6H). | ESI MS m/z 328 [M + H]⁺ |
| 071 | 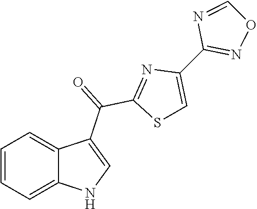 [SMILES: O=C(c1nc(-c2ncon2)cs1)c1c[nH]c2ccccc12] | ¹H NMR (500 MHz, DMSO-d₆) δ 12.37 (s, 1H), 9.82 (s, 1H), 9.15 (d, J = 3.0 Hz, 1H), 8.85 (s, 1H), 8.35-8.31 (m, 1H), 7.62-7.59 (m, 1H), 7.33-7.28 (m, 2H). | ESI MS m/z 297 [M + H]⁺ |
| 072 | 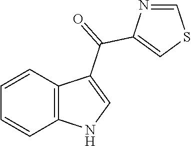 [SMILES: O=C(c1cscn1)c1c[nH]c2ccccc12] | ¹H NMR (500 MHz, DMSO-d₆) δ 12.07 (s, 1H), 9.28 (d, J = 2.0 Hz, 1H), 8.94 (d, J = 3.0 Hz, 1H), 8.51 (d, J = 2.5 Hz, 1H), 8.38-8.36 (m, 1H), 7.54-7.52 (m, 1H), 7.27-7.22 (m, 2H). | ESI MS m/z 229 [M + H]⁺ |
| 073 | 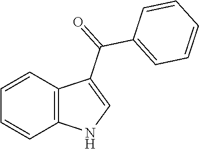 [SMILES: O=C(c1ccccc1)c1c[nH]c2ccccc12] | ¹H NMR (500 MHz, DMSO-d₆) δ 12.06 (s, 1H), 8.26-8.24 (m, 1H), 7.93 (s, 1H), 7.79-7.77 (m, 2H), 7.62-7.59 (m, 1H), 7.55-7.51 (m, 3H), 7.28-7.22 (m, 2H). | ESI MS m/z 222 [M + H]⁺ |
| 074 | 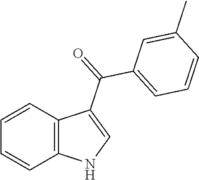 [SMILES: Cc1cccc(C(=O)c2c[nH]c3ccccc23)c1] | ¹H NMR (500 MHz, DMSO-d₆) δ 12.02 (s, 1H), 8.24 (dd, J = 6.5, 1.5 Hz, 1H), 7.92 (d, J = 3.0 Hz, 1H), 7.58-7.56 (m, 2H), 7.52-7.50 (m, 1H), 7.42-7.41 (m, 2H), 7.27-7.21 (m, 2H), 2.41 (s, 3H). | ESI MS m/z 236 [M + H]⁺ |
| 075 | 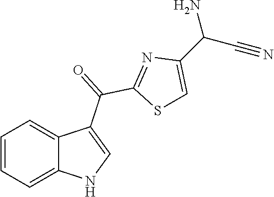 [SMILES: N#CC(N)c1csc(C(=O)c2c[nH]c3ccccc23)n1] | ¹H NMR (500 MHz, DMSO-d₆) δ 12.34 (s, 1H), 9.16 (s, 1H), 8.37-8.30 (m, 1H), 7.92 (d, J = 1.0 Hz, 1H), 7.58-7.55 (m, 1H), 7.31-7.27 (m, 2H), 5.35 (t, J = 8.0 Hz, 1H), 3.04 (d, J = 8.0 Hz, 2H). | ESI MS m/z 283 [M + H]⁺ |
| 076 | 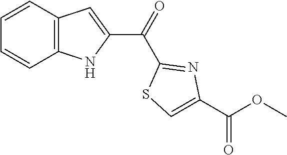 [SMILES: COC(=O)c1csc(C(=O)c2cc3ccccc3[nH]2)n1] | ¹H NMR (400 MHz, DMSO-d₆) δ 12.18 (s, 1H), 9.00 (s, 1H), 8.22 (d, J = 1.2 Hz, 1H), 7.84 (d, J = 8.0 Hz, 1H), 7.50-7.53 (m, 1H), 7.37-7.40 (m, 1H), 7.13-7.16 (m, 1H), 3.95 (s, 3H). | ESI MS m/z 287 [M + H]⁺ |
| 077 | 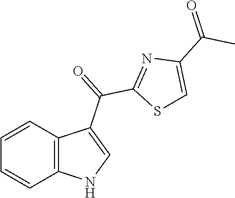 [SMILES: CC(=O)c1csc(C(=O)c2c[nH]c3ccccc23)n1] | ¹H NMR (400 MHz, DMSO-d₆) δ 12.35 (bs, 1H), 9.18 (s, 1H), 8.86 (s, 1H), 8.31-8.34 (m, 1H), 7.58-7.61 (m, 1H), 7.27-7.34 (m, 2H), 2.74 (s, 3H). | ESI MS m/z 270.4 [M + H]⁺ |
| 078 | 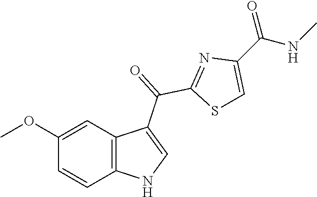 | ¹H NMR (400 MHz, DMSO-d₆) δ 12.25 (bs, 1H), 9.39 (s, 1H), 8.71-8.73 (m, 1H), 8.58 (s, 1H), 7.85 (d, J = 2.4 Hz, 1H), 7.47 (d, J = 8.8 Hz, 1H), 6.91-6.95 (m, 1H), 3.83 (s, 3H), 2.89 (d, J = 4.8 Hz, 3H). | ESI MS m/z 316 [M + H]⁺ |
| 079 | 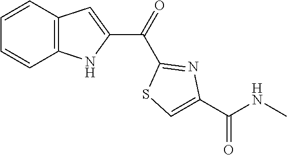 [SMILES: CNC(=O)c1csc(C(=O)c2cc3ccccc3[nH]2)n1] | ¹H NMR (400 MHz, DMSO-d₆) δ 12.08 (bs, 1H), 8.72-8.80 (m, 1H), 8.72 (s, 1H), 8.47 (s, 1H), 7.81 (d, J = 8.0 Hz, 1H), 7.54 (d, J = 8.4 Hz, 1H), 7.35-7.40 (m, 1H), 7.13-7.18 (m, 1H), 2.92 (d, J = 4.8 Hz, 3H). | ESI MS m/z 286 [M + H]⁺ |
| 080 | 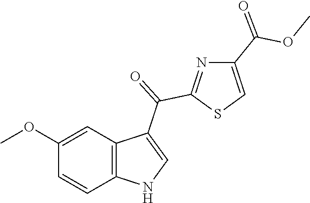 | ¹H NMR (400 MHz, DMSO-d₆) δ 12.26 (bs, 1H), 9.03 (s, 1H), 8.88 (s, 1H), 7.83 (d, J = 2.4 Hz, 1H), 7.49 (d, J = 5.6 Hz, 1H), 6.91-6.95 (m, 1H), 3.92 (s, 3H), 3.83 (s, 3H). | ESI MS m/z 317 [M + H]⁺ |
| 081 | 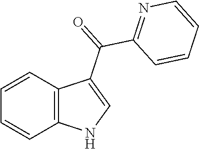 [SMILES: O=C(c1ccccn1)c1c[nH]c2ccccc12] | ¹H NMR (500 MHz, DMSO-d₆) δ 12.08 (s, 1H), 8.81 (d, J = 3.0 Hz, 1H), 8.76-8.75 (m, 1H), 8.39-8.37 (m, 1H), 8.04-8.02 (m, 2H), 7.64-7.11 (m, 1H), 7.54-7.51 (m, 1H), 7.27-7.23 (m, 2H). | ESI MS m/z 223 [M + H]⁺ |
| 082 | 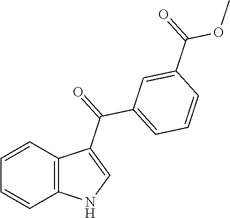 [SMILES: COC(=O)c1cccc(C(=O)c2c[nH]c3ccccc23)c1] | ¹H NMR (500 MHz, DMSO-d₆) δ 12.13 (s, 1H), 8.29 (t, J = 1.5 Hz, 1H), 8.24 (dd, J = 8.5, 1.5 Hz, 1H), 8.17 (dt, J = 8.0, 1.5 Hz, 1H), 8.06 (dt, J = 8.0, 1.5 Hz, 1H), 7.98 (d, J = 3.0 Hz, 1H), 7.70 (t, J = 8.0 Hz, 1H), 7.54-7.53 (m, 1H), 7.30-7.24 (m, 2H), 3.89 (s, 3H). | ESI MS m/z 280 [M + H]⁺ |
| 083 | 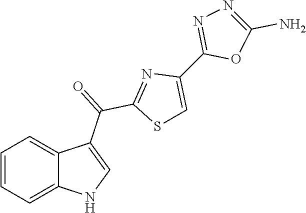 [SMILES: Nc1nnc(-c2csc(C(=O)c3c[nH]c4ccccc34)n2)o1] | ¹H NMR (500 MHz, DMSO-d₆) δ 12.39 (s, 1H), 9.08 (s, 1H), 8.54 (s, 1H), 8.33-8.31 (m, 1H), 7.61-7.59 (m, 1H), 7.41 (bs, 2H), 7.33-7.28 (m, 2H). | ESI MS m/z 312 [M + H]⁺ |
| 085 | 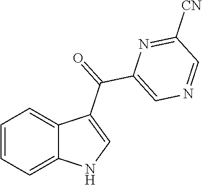 [SMILES: N#Cc1cncc(C(=O)c2c[nH]c3ccccc23)n1] | ¹H NMR (400 MHz, DMSO-d₆) δ 12.30 (bs, 1H), 9.40-9.44 (m, 2H), 8.59 (s, 1H), 8.32-8.35 (m, 1H), 7.56-7.60 (m, 1H), 7.29-7.32 (m, 2H). | ESI MS m/z 249 [M + H]⁺ |
| 086 | 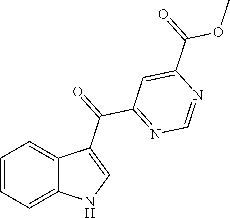 [SMILES: COC(=O)c1cc(C(=O)c2c[nH]c3ccccc23)ncn1] | ¹H NMR (400 MHz, DMSO-d₆) δ 12.34 (bs, 1H), 9.58 (d, J = 1.6 Hz, 1H), 8.80 (s, 1H), 8.41 (s, 1H), 8.33-8.35 (m, 1H), 7.55-7.58 (m, 1H), 7.28-7.32 (m, 2H), 3.97 (s, 3H). | ESI MS m/z 280 [M − H]⁻ |
| 087 | 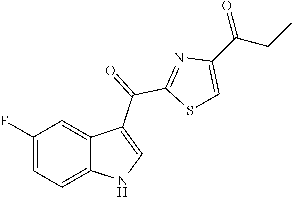 [SMILES: CCC(=O)c1csc(C(=O)c2c[nH]c3ccc(F)cc23)n1] | ¹H NMR (400 MHz, DMSO-d₆) δ 12.42 (bs, 1H), 9.20 (s, 1H), 8.86 (s, 1H), 7.97-8.00 (m, 1H), 7.60-7.63 (m, 1H), 7.16-7.19 (m, 1H), 3.24 (q, J = 7.2 Hz, 2H), 1.15 (t, J = 7.2 Hz, 3H). | ESI MS m/z 303 [M + H]⁺ |
| 088 | 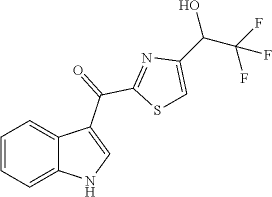 [SMILES: O=C(c1nc(C(O)C(F)(F)F)cs1)c1c[nH]c2ccccc12] | ¹H NMR (500 MHz, DMSO-d₆) δ 12.32 (s, 1H), 9.09 (s, 1H), 8.30-8.32 (m, 1H), 8.18 (s, 1H), 7.57-7.55 (m, 1H), 7.31-7.25 (m, 2H), 7.15 (d, J = 6.0 Hz, 1H), 5.50 (7, J = 7.0 Hz, 1H). | ESI MS m/z 327 [M + H]⁺ |
| 089 | 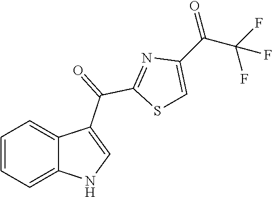 [SMILES: O=C(c1nc(C(=O)C(F)(F)F)cs1)c1c[nH]c2ccccc12] | ¹H NMR (500 MHz, CD₃OD, partial CD₃O-adduct) δ 9.25, 9.17 (s, 1H), 8.39-8.36 (m, 1H), 8.12, 8.07 (s, 1H), 7.51-7.48 (m, 1H), 7.30-7.25 (m, 2H). | As hydrate, ESI MS m/z 343 [M + H + H₂O]⁺ |
| 090 | 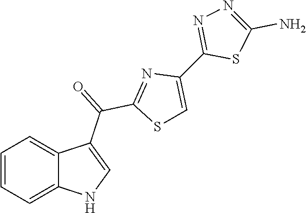 [SMILES: Nc1nnc(-c2csc(C(=O)c3c[nH]c4ccccc34)n2)s1] | ¹H NMR (500 MHz, DMSO-d₆) δ 12.38 (bs, 1H), 9.02 (d, J = 3.5 Hz, 1H), 8.53 (s, 1H), 8.34-8.30 (m, 1H), 7.61-7.57 (m, 1H), 7.52 (bs, 2H), 7.33-7.27 (m, 2H). | ESI MS m/z 328 [M + H]⁺ |
| 091 | 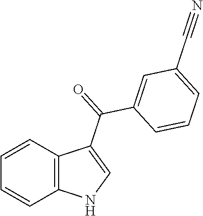 [SMILES: N#Cc1cccc(C(=O)c2c[nH]c3ccccc23)c1] | ¹H NMR (500 MHz, DMSO-d₆) δ 12.18 (s, 1H), 8.25 (dd, J = 5.0, 1.5 Hz, 1H), 8.18-8.17 (m, 1H), 8.09-8.03 (m, 3H), 7.75 (dt, J = 8.0, 0.5 Hz, 1H), 7.54-7.52 (m, 1H), 7.30-7.24 (m, 2H). | ESI MS m/z 247 [M + H]⁺ |
| 092 | 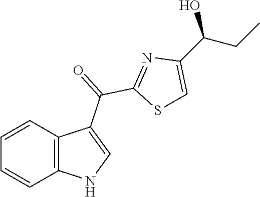 [SMILES: CC[C@H](O)c1csc(C(=O)c2c[nH]c3ccccc23)n1] | ¹H NMR (400 MHz, DMSO-d₆) δ 12.21 (bs, 1H), 9.10 (s, 1H), 8.30-8.34 (m, 1H), 7.82 (s, 1H), 7.55-7.60 (m, 1H), 7.24-7.31 (m, 2H), 5.45 (d, J = 6.8 Hz, 1H), 4.70-7.77 (m, 1H), 1.88-1.95 (m, 1H), 1.75-1.85 (m, 1H), 0.93 (t, J = 6.0 Hz, 3H). | ESI MS m/z 287 [M + H]⁺ |
| 093 | 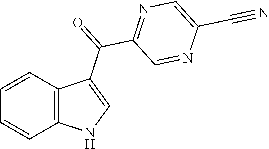 [SMILES: N#Cc1cnc(C(=O)c2c[nH]c3ccccc23)cn1] | ¹H NMR (400 MHz, DMSO-d₆) δ 12.36 (bs, 1H), 9.37 (s, 1H), 9.31 (s, 1H), 8.36 (s, 1H), 8.33-8.37 (m, 1H), 7.55-7.59 (m, 1H), 7.29-7.33 (m, 2H). | ESI MS m/z 248.4 [M + H]⁺ |
| 094 | 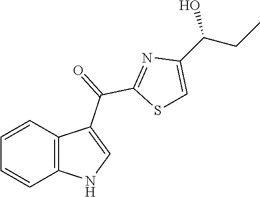 [SMILES: CC[C@@H](O)c1csc(C(=O)c2c[nH]c3ccccc23)n1] | ¹H NMR (400 MHz, DMSO-d₆) δ 12.22 (bs, 1H), 9.10 (s, 1H), 8.30-8.34 (m, 1H), 7.82 (s, 1H), 7.55-7.60 (m, 1H), 7.24-7.31 (m, 2H), 5.45 (d, J = 6.0 Hz, 1H), 4.70-4.77 (m, 1H), 1.88-2.05 (m, 1H), 1.74-1.85 (m, 1H), 0.95 (t, J = 6.0 Hz, 3H). | ESI MS m/z 287 [M + H]⁺ |
| 095 | 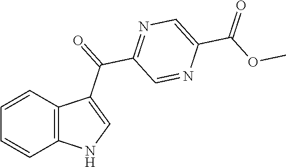 [SMILES: COC(=O)c1cnc(C(=O)c2c[nH]c3ccccc23)cn1] | ¹H NMR (400 MHz, DMSO-d₆) δ 12.33 (bs, 1H), 9.34 (d, J = 1.2 Hz, 1H), 9.28 (d, J = 1.2 Hz, 1H), 8.68 (s, 1H), 8.35-8.38 (m, 1H), 7.55-7.58 (m, 1H), 7.29-7.32 (m, 2H). | ESI MS m/z 280 [M − H]⁻ |
| 096 | 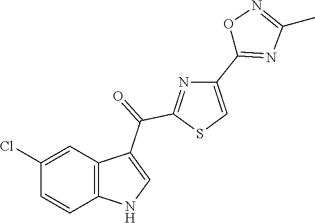 [SMILES: Cc1noc(-c2csc(C(=O)c3c[nH]c4ccc(Cl)cc34)n2)n1] | ¹H NMR (500 MHz, DMSO-d₆) δ 12.49 (s, 1H), 9.16 (d, J = 1.5 Hz, 1H), 9.11 (d, J = 2.0 Hz, 1H), 8.29 (m, 1H), 7.65 (d, J = 8.5 Hz, 1H), 7.35-7.33 (m, 1H), 2.48 (s, 3H). | ESI MS m/z 343 [M − H]⁻ |
| 097 | 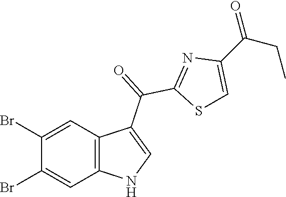 [SMILES: CCC(=O)c1csc(C(=O)c2c[nH]c3cc(Br)c(Br)cc23)n1] | ¹H NMR (400 MHz, DMSO-d₆) δ 12.49 (bs, 1H), 9.18 (s, 1H), 8.87 (s, 1H), 8.61 (s, 1H), 8.01 (s, 1H), 3.23-3.26 (m, 2H), 1.13-1.16 (m, 3H). | ESI MS m/z 440 [M + H]⁺ |
| 099 | 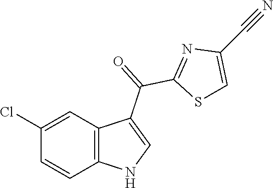 [SMILES: N#Cc1csc(C(=O)c2c[nH]c3ccc(Cl)cc23)n1] | ¹H NMR (500 MHz, DMSO-d₆) δ 12.54 (s, 1H), 9.20 (s, 1H), 9.08 (d, J = 3.0 Hz, 1H), 8.26 (d, J = 2.0 Hz, 1H), 7.62 (d, J = 9.0 Hz, 1H), 7.34 (dd, J = 9.0, 5.0 Hz, 1H). | ESI MS m/z 286 [M − H]⁻ |
| 100 | 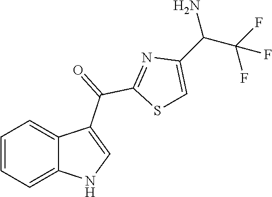 [SMILES: NC(c1csc(C(=O)c2c[nH]c3ccccc23)n1)C(F)(F)F] | ¹H NMR (500 MHz, DMSO-d₆) δ 12.27 (s, 1H), 9.13 (d, J = 3.0 Hz, 1H), 8.32-8.30 (m, 1H), 8.15 (s, 1H), 7.57-7.55 (m, 1H), 7.31-7.55 (m, 2H), 4.86-4.81 (m, 1H), 2.71 (d, J = 8.0 Hz, 2H). | ESI MS m/z 326 [M + H]⁺ |
| 101 | 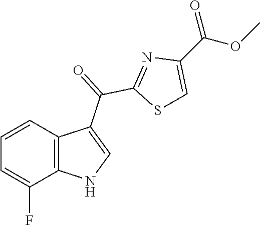 [SMILES: COC(=O)c1csc(C(=O)c2c[nH]c3c(F)cccc23)n1] | ¹H NMR (400 MHz, DMSO-d₆) δ 12.97 (bs, 1H), 9.11 (s, 1H), 8.92 (s, 1H), 8.12 (d, J = 7.6 Hz, 1H), 7.26-7.30 (m, 1H), 7.15-7.25 (m, 1H), 3.96 (s, 3H). | ESI MS m/z 305 [M + H]⁺ |
| 102 | 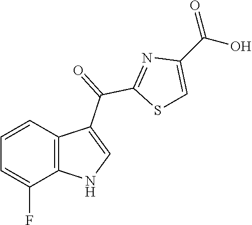 [SMILES: O=C(O)c1csc(C(=O)c2c[nH]c3c(F)cccc23)n1] | ¹H NMR (400 MHz, DMSO-d₆) δ 13.47 (bs, 1H), 12.96 (s, 1H), 9.17 (s, 1H), 8.83 (s, 1H), 8.13 (d, J = 8.0 Hz, 1H), 7.27-7.30 (m, 1H), 7.17-7.20 (m, 1H). | ESI MS m/z 289 [M − H]⁻ |
| 103 | 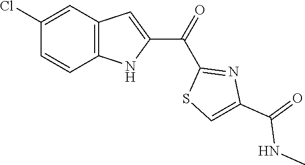 [SMILES: CNC(=O)c1csc(C(=O)c2cc3cc(Cl)ccc3[nH]2)n1] | ¹H NMR (400 MHz, DMSO-d₆) δ 12.29 (bs, 1H), 8.74 (s, 1H), 8.72-8.74 (m, 1H), 8.42 (d, J = 1.2 Hz, 1H), 7.84 (d, J = 2.0 Hz, 1H), 7.56 (d, J = 8.0 Hz, 1H), 7.36-7.40 (m, 1H), 2.92 (d, J = 4.8 Hz, 3H). | ESI MS m/z 320 [M + H]⁺ |
| 104 | 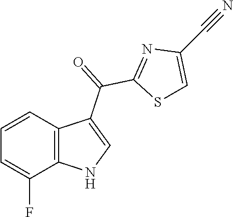 [SMILES: N#Cc1csc(C(=O)c2c[nH]c3c(F)cccc23)n1] | ¹H NMR (400 MHz, DMSO-d₆) δ 13.05 (bs, 1H), 9.21 (s, 1H), 9.02 (s, 1H), 8.10 (d, J = 8.0 Hz, 1H), 7.26-7.33 (m, 1H), 7.16-7.20 (m, 1H). | ESI MS m/z 270 [M − H]⁻ |
| 105 | 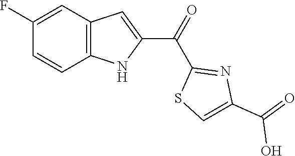 [SMILES: O=C(O)c1csc(C(=O)c2cc3cc(F)ccc3[nH]2)n1] | ¹H NMR (400 MHz, DMSO-d₆) δ 13.57 (bs, 1H), 12.27 (s, 1H), 8.92 (s, 1H), 8.23 (s, 1H), 7.65 (m, 1H), 7.51 (m, 1H), 7.25 (m, 1H). | ESI MS m/z 289 [M − H]⁻ |
| 106 | 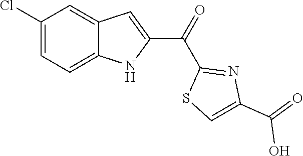 [SMILES: O=C(O)c1csc(C(=O)c2cc3cc(Cl)ccc3[nH]2)n1] | ¹H NMR (400 MHz, DMSO-d₆) δ 13.56 (bs, 1H), 12.35 (s, 1H), 8.92 (s, 1H), 8.20 (s, 1H), 7.97 (d, J = 4.5 Hz, 1H), 7.53 (d, J = 8.8 Hz, 1H), 7.35-7.39 (m, 1H). | ESI MS m/z 305 [M − H]⁻ |
| 107 | 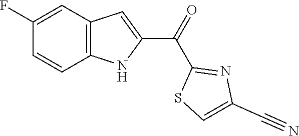 [SMILES: N#Cc1csc(C(=O)c2cc3cc(F)ccc3[nH]2)n1] | ¹H NMR (400 MHz, DMSO-d₆) δ 12.31 (bs, 1H), 9.30 (s, 1H), 8.14 (s, 1H), 7.63-7.70 (m, 1H), 7.51-7.55 (m, 1H), 7.23-7.30 (m, 1H) | ESI MS m/z 270 [M − H]⁻ |
| 108 | 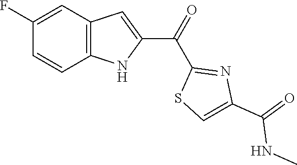 [SMILES: CNC(=O)c1csc(C(=O)c2cc3cc(F)ccc3[nH]2)n1] | ¹H NMR (400 MHz, DMSO-d₆) δ 12.21 (bs, 1H), 8.70-8.80 (m, 2H), 8.43 (s, 1H), 7.50-8.60 (m, 2H), 7.26 (m, 1H), 2.92 (d, J = 4.8 Hz, 3H). | ESI MS m/z 302 [M − H]⁻ |
| 109 | 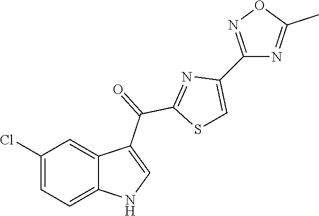 [SMILES: Cc1nc(-c2csc(C(=O)c3c[nH]c4ccc(Cl)cc34)n2)no1] | ¹H NMR (500 MHz, DMSO-d₆) δ 12.50 (d, J = 2.0 Hz, 1H), 9.16 (d, J = 3.5 Hz, 1H), 8.81 (s, 1H), 8.30 (d, J = 2.0 Hz, 1H), 7.64 (d, J = 8.5 Hz, 1H), 7.34 (dd, J = 8.5, 2.0 Hz, 1H), 2.72 (s, 3H). | ESI MS m/z 343 [M − H]⁻ |
| 110 | 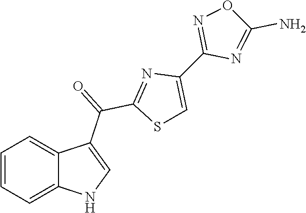 [SMILES: Nc1nc(-c2csc(C(=O)c3c[nH]c4ccccc34)n2)no1] | ¹H NMR (500 MHz, DMSO-d₆) δ 12.34 (s, 1H), 9.12 (s, 1H), 8.55 (s, 1H), 8.33-8.32 (m, 1H), 8.06 (s, 2H), 7.60-7.59 (m, 1H), 7.32-7.27 (m, 2H). | ESI MS m/z 312 [M + H]⁺ |
| 111 | 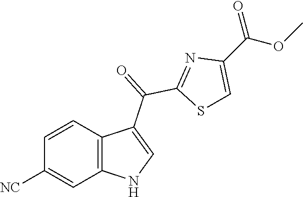 [SMILES: COC(=O)c1csc(C(=O)c2c[nH]c3cc(C#N)ccc23)n1] | ¹H NMR (400 MHz, DMSO-d₆) δ 12.80 (bs, 1H), 9.26 (s, 1H), 8.94 (s, 1H), 8.41-8.44 (m, 1H), 8.15 (s, 1H), 7.65-7.69 (m, 1H), 3.93 (s, 3H). | ESI MS m/z 310 [M − H]⁻ |
| 112 | 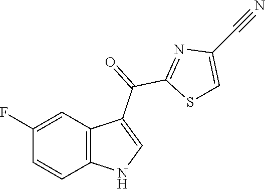 [SMILES: N#Cc1csc(C(=O)c2c[nH]c3ccc(F)cc23)n1] | ¹H NMR (400 MHz, DMSO-d₆) δ 12.51 (bs, 1H), 9.21 (s, 1H), 9.08 (s, 1H), 7.93-7.98 (m, 1H), 7.60-7.65 (m, 1H), 7.15-7.22 (m, 1H). | ESI MS m/z 270 [M − H]⁻ |
| 113 | 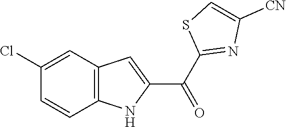 [SMILES: N#Cc1csc(C(=O)c2cc3cc(Cl)ccc3[nH]2)n1] | ¹H NMR (400 MHz, DMSO-d₆) δ 12.38 (bs, 1H), 9.30 (s, 1H), 8.12 (s, 1H), 7.96 (s, 1H), 7.52 (d, J = 8.8 Hz, 1H), 7.37 (d, J = 8.4 Hz, 1H). | ESI MS m/z 286 [M − H]⁻ |
| 114 | 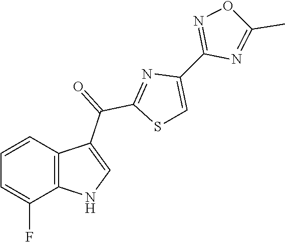 [SMILES: Cc1nc(-c2csc(C(=O)c3c[nH]c4c(F)cccc34)n2)no1] | ¹H NMR (400 MHz, DMSO-d₆) δ 12.98 (bs, 1H), 9.16 (s, 1H), 8.82 (s, 1H), 8.14 (d, J = 8.0 Hz, 1H), 7.28-7.31 (m, 1H), 7.16-7.22 (m, 1H), 2.73 (s, 3H). | ESI MS m/z 327 [M − H]⁻ |
| 116 | 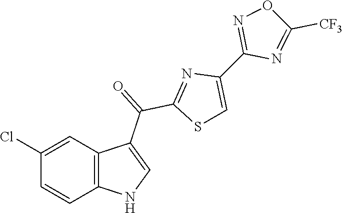 [SMILES: O=C(c1nc(-c2noc(C(F)(F)F)n2)cs1)c1c[nH]c2ccc(Cl)cc12] | ¹H NMR (500 MHz, DMSO-d₆) δ 12.52 (s, 1H), 9.15 (s, 1H), 9.04 (s, 1H), 8.30 (m, 1H), 7.65 (dd, J = 8.5, 0.5 Hz, 1H), 7.34 (dd, J = 9.0, 2.5 Hz, 1H). | ESI MS m/z 397 [M − H]⁻ |
| 117 | 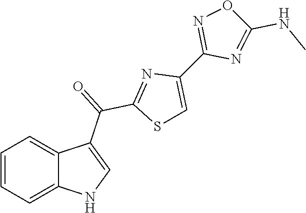 [SMILES: CNc1nc(-c2csc(C(=O)c3c[nH]c4ccccc34)n2)no1] | ¹H NMR (500 MHz, DMSO-d₆) δ 12.35 (s, 1H), 9.12 (d, J = 3.0 Hz, 1H), 8.59 (s, 1H), 8.46-8.44 (m, 1H), 8.33-8.31 (m, 1H), 7.60-7.58 (m, 1H), 7.32-7.27 (m, 2H), 2.96 (d, J = 4.5 Hz, 3H). | ESI MS m/z 326 [M + H]⁺ |
| 118 | 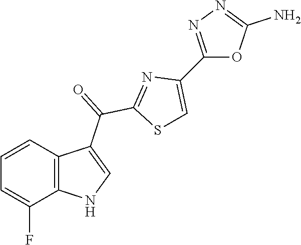 [SMILES: Nc1nnc(-c2csc(C(=O)c3c[nH]c4c(F)cccc34)n2)o1] | ¹H NMR (400 MHz, DMSO-d₆) δ 13.03 (bs, 1H), 9.10 (s, 1H), 8.58 (s, 1H), 8.14 (d, J = 8.0 Hz, 1H), 7.47 (s, 2H), 7.29 (d, J = 5.2 Hz, 1H), 7.16-7.22 (m, 1H). | ESI MS m/z 328 [M − H]⁻ |
| 119 | 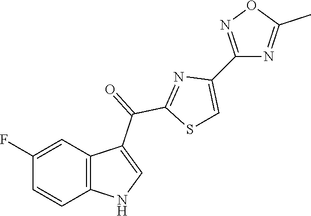 [SMILES: Cc1nc(-c2csc(C(=O)c3c[nH]c4ccc(F)cc34)n2)no1] | ¹H NMR (400 MHz, DMSO-d₆) δ 12.48 (bs, 1H), 9.17 (s, 1H), 8.80 (s, 1H), 7.97-8.01 (m, 1H), 7.62-7.66 (m, 1H), 7.17-7.19 (m, 1H), 2.72 (s, 3H). | ESI MS m/z 327 [M − H]⁻ |
| 120 | 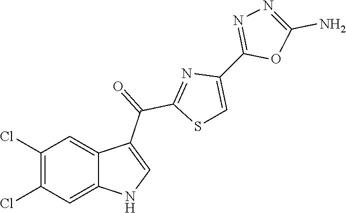 [SMILES: Nc1nnc(-c2csc(C(=O)c3c[nH]c4cc(Cl)c(Cl)cc34)n2)o1] | ¹H NMR (500 MHz, DMSO-d₆) δ 12.56 (d, J = 2.5 Hz, 1H), 9.12 (d, J = 3.0 Hz, 1H), 8.57 (s, 1H), 8.45 (s, 1H), 7.89 (s, 1H), 7.39 (s, 2H). | ESI MS m/z 378 [M − H]⁻ |
| 121 | 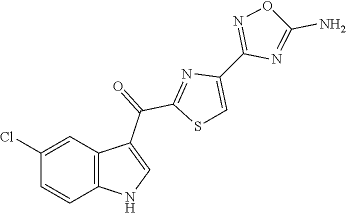 [SMILES: Nc1nc(-c2csc(C(=O)c3c[nH]c4ccc(Cl)cc34)n2)no1] | ¹H NMR (500 MHz, DMSO-d₆) δ 12.47 (s, 1H), 9.14 (s, 1H), 8.56 (s, 1H), 8.30 (d, J = 2.0 Hz, 1H), 8.05 (s, 2H), 7.63 (d, J = 8.5 Hz, 1H), 7.32 (dd, J = 8.5, 2.0 Hz, 1H). | ESI MS m/z 344 [M − H]⁻ |
| 123 | 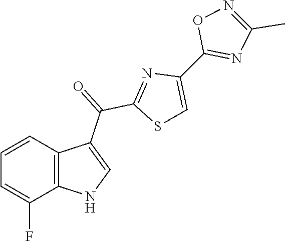 [SMILES: Cc1noc(-c2csc(C(=O)c3c[nH]c4c(F)cccc34)n2)n1] | ¹H NMR (400 MHz, DMSO-d₆) δ 13.01 (bs, 1H), 9.13 (d, J = 8.0 Hz, 1H), 8.13 (d, J = 8.0 Hz, 1H), 7.27-7.33 (m, 1H), 7.17-7.23 (m, 1H), 2.51 (s, 3H). | ESI MS m/z 327 [M − H]⁻ |
| 124 | 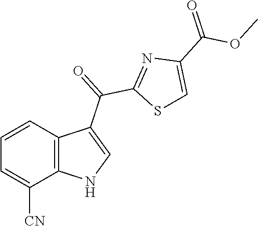 [SMILES: COC(=O)c1csc(C(=O)c2c[nH]c3c(C#N)cccc23)n1] | ¹H NMR (400 MHz, DMSO-d₆) δ 13.27 (bs, 1H), 9.15 (s, 1H), 8.95 (s, 1H), 8.63 (d, J = 7.6 Hz, 1H), 7.84 (d, J = 7.2 Hz, 1H), 7.45-7.48 (m, 1H), 3.93 (s, 3H). | ESI MS m/z 310 [M − H]⁻ |
| 125 | 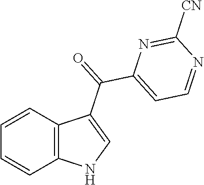 [SMILES: N#Cc1nccc(C(=O)c2c[nH]c3ccccc23)n1] | ¹H NMR (400 MHz, DMSO-d₆) δ 12.38 (bs, 1H), 9.27 (d, J = 4.8 Hz, 1H), 8.66 (d, J = 2.4 Hz, 1H), 8.30-8.33 (m, 1H), 8.24-8.27 (m, 1H), 7.57-7.60 (m, 1H), 7.30-7.33 (m, 2H). | ESI MS m/z 247 [M − H]⁻ |
| 126 | 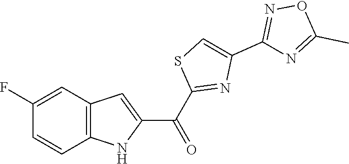 [SMILES: Cc1nc(-c2csc(C(=O)c3cc4cc(F)ccc4[nH]3)n2)no1] | ¹H NMR (400 MHz, DMSO-d₆) 12.31 (bs, 1H), 8.91 (s, 1H), 8.24 (s, 1H), 7.68 (d, J = 8.8 Hz, 1H), 7.52-7.56 (m, 1H), 7.25-7.28 (m, 1H), 2.74 (s, 3H). | ESI MS m/z 327 [M − H]⁻ |
| 127 | 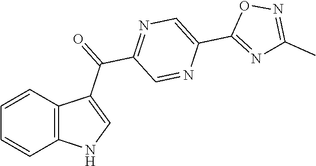 [SMILES: Cc1noc(-c2cnc(C(=O)c3c[nH]c4ccccc34)cn2)n1] | ¹H NMR (400 MHz, DMSO-d₆) δ 12.35 (bs, 1H), 9.50 (s, 1H), 9.35 (s, 1H), 8.72 (d, J = 3.2 Hz, 1H), 8.37-8.40 (m, 1H), 7.56-7.59 (m, 1H), 7.28-7.34 (m, 2H), 2.53 (s, 3H). | ESI MS m/z 304 [M − H]⁻ |
| 128 | 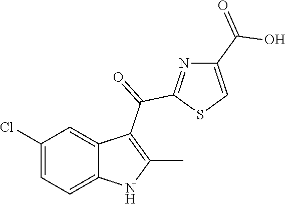 [SMILES: Cc1[nH]c2ccc(Cl)cc2c1C(=O)c1nc(C(=O)O)cs1] | ¹H NMR (400 MHz, DMSO-d₆) δ 13.35 (bs, 1H), 12.43 (s, 1H), 8.83 (s, 1H), 8.12 (s, 1H), 7.44 (d, J = 8.4 Hz, 1H), 7.20-7.23 (m, 1H), 2.63 (s, 3H). | ESI MS m/z 319 [M − H]⁻ |
| 129 | 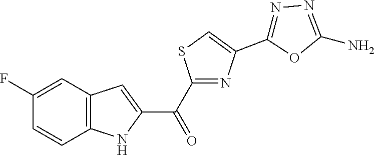 [SMILES: Nc1nnc(-c2csc(C(=O)c3cc4cc(F)ccc4[nH]3)n2)o1] | ¹H NMR (400 MHz, DMSO-d₆) δ 12.31 (bs, 1H), 8.67 (s, 1H), 8.20 (s, 1H), 7.63 (d, J = 9.6 Hz, 1H), 7.50-7.55 (m, 1H), 7.48 (s, 2H), 7.26-7.30 (m, 1H). | ESI MS m/z 328 [M − H]⁻ |
| 130 | 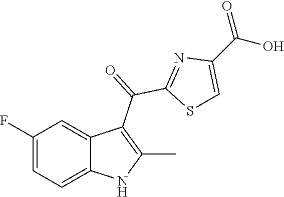 [SMILES: Cc1[nH]c2ccc(F)cc2c1C(=O)c1nc(C(=O)O)cs1] | ¹H NMR (400 MHz, DMSO-d₆) δ 13.36 (bs, 1H), 12.37 (s, 1H), 8.83 (s, 1H), 7.87 (dd, J = 10.8, 1.6 Hz, 1H), 7.40-7.46 (m, 1H), 7.00-7.08 (m, 1H), 2.63 (s, 3H). | ESI MS m/z 303 [M − H]⁻ |
| 131 | 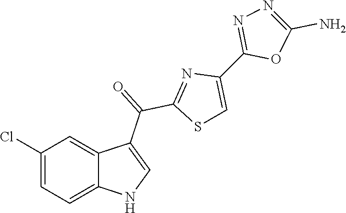 [SMILES: Nc1nnc(-c2csc(C(=O)c3c[nH]c4ccc(Cl)cc34)n2)o1] | ¹H NMR (400 MHz, DMSO-d₆) δ 12.38 (bs, 1H), 8.67 (s, 1H), 8.18 (s, 1H), 7.93 (s, 1H), 7.48-7.56 (m, 3H), 7.36-7.40 (m, 1H). | ESI MS m/z 344 [M − H]⁻ |
| 132 | 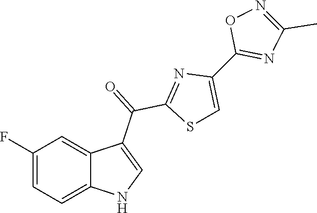 [SMILES: Cc1noc(-c2csc(C(=O)c3c[nH]c4ccc(F)cc34)n2)n1] | ¹H NMR (400 MHz, DMSO-d₆) δ 12.47 (bs, 1H), 9.17 (s, 1H), 9.12 (s, 1H), 7.96-8.00 (m, 1H), 7.62-7.67 (m, 1H), 7.15-7.22 (m, 1H), 2.48 (s, 3H). | ESI MS m/z 327 [M − H]⁻ |
| 133 | 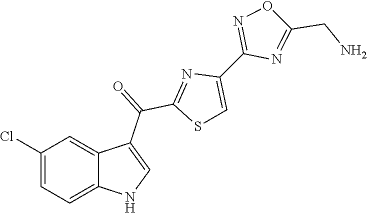 [SMILES: NCc1nc(-c2csc(C(=O)c3c[nH]c4ccc(Cl)cc34)n2)no1] | ¹H NMR (400 MHz, DMSO-d₆) δ 12.60 (bs, 1H), 9.17 (s, 1H), 8.81 (s, 1H), 8.30 (s, 1H), 7.64 (d, J = 8.8 Hz, 1H), 7.32-7.35 (m, 2H), 4.08 (s, 2H), 2.32 (s, 2H). | ESI MS m/z 358 [M − H]⁻ |
| 134 | 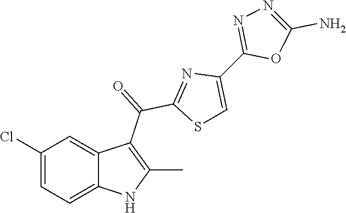 [SMILES: Cc1[nH]c2ccc(Cl)cc2c1C(=O)c1nc(-c2nnc(N)o2)cs1] | ¹H NMR (400 MHz, DMSO-d₆) δ 12.42 (bs, 1H), 8.58 (s, 1H), 8.01 (s, 1H), 7.45 (d, J = 8.4 Hz, 1H), 7.38 (s, 2H), 7.21 (d, J = 8.4 Hz, 1H), 2.62 (s, 3H). | ESI MS m/z 358 [M − H]⁻ |
| 135 | 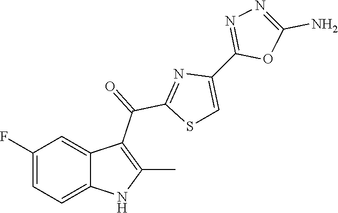 [SMILES: Cc1[nH]c2ccc(F)cc2c1C(=O)c1nc(-c2nnc(N)o2)cs1] | ¹H NMR (400 MHz, DMSO-d₆) δ 12.36 (bs, 1H), 8.57 (s, 1H), 7.72-7.76 (m, 1H), 7.39-7.45 (m, 3H), 7.04-7.06 (m, 1H), 2.62 (s, 3H). | ESI MS m/z 342 [M − H]⁻ |
| 137 | 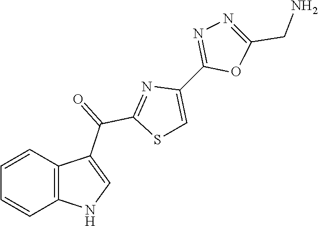 [SMILES: NCc1nnc(-c2csc(C(=O)c3c[nH]c4ccccc34)n2)o1] | ¹H NMR (400 MHz, DMSO-d₆) δ 12.42 (bs, 1H), 9.14 (s, 1H), 8.90 (s, 1H), 8.32-8.35 (m, 1H), 7.60-7.63 (m, 1H), 7.29-7.34 (m, 2H), 4.04 (s, 2H), 1.99 (s, 2H). | ESI MS m/z 326 [M + H]⁺ |
| 138 | 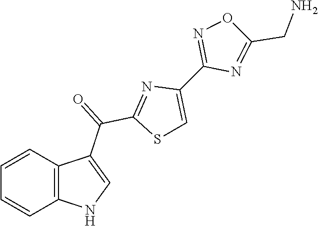 [SMILES: NCc1nc(-c2csc(C(=O)c3c[nH]c4ccccc34)n2)no1] | ¹H NMR (400 MHz, DMSO-d₆) δ 12.39 (bs, 1H), 9.15 (s, 1H), 8.80 (s, 1H), 8.32-8.35 (m, 1H), 7.61 (d, J = 5.6 Hz, 1H), 7.30-7.32 (m, 2H), 4.08 (s, 2H), 2.23 (s, 2H). | ESI MS m/z 326 [M + H]⁺ |
| 139 | 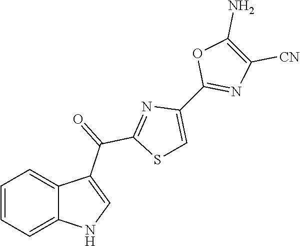 [SMILES: N#Cc1nc(-c2csc(C(=O)c3c[nH]c4ccccc34)n2)oc1N] | ¹H NMR (400 MHz, DMSO-d₆) δ 12.38 (bs, 1H), 9.09 (d, J = 2.8 Hz, 1H), 8.48 (s, 1H), 8.30-9.34 (m, 1H), 8.17 (s, 1H), 7.60 (d, J = 6.4 Hz, 1H), 7.29-7.33 (m, 2H). | ESI MS m/z 334 [M − H]⁻ |
| 140 | 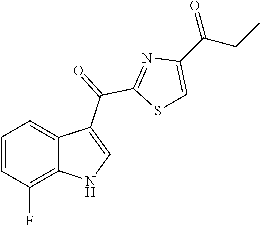 [SMILES: CCC(=O)c1csc(C(=O)c2c[nH]c3c(F)cccc23)n1] | ¹H NMR (400 MHz, DMSO-d₆) δ 12.96 (bs, 1H), 9.17 (s, 1H), 8.88 (s, 1H), 8.13 (d, J = 8.0 Hz, 1H), 7.27-7.30 (m, 1H), 7.17-7.21 (m, 1H), 3.24 (q, J = 7.2 Hz, 2H), 1.15 (t, J = 7.26 Hz, 3H). | ESI MS m/z 301 [M − H]⁻ |
| 141 | 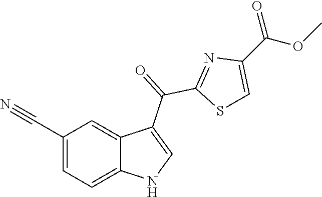 [SMILES: COC(=O)c1csc(C(=O)c2c[nH]c3ccc(C#N)cc23)n1] | ¹H NMR (400 MHz, DMSO-d₆) δ 12.78 (bs, 1H), 9.22 (s, 1H), 8.95 (s, 1H), 8.66 (s, 1H), 7.78-7.82 (d, J = 8.4 Hz, 1H), 7.68-7.72 (d, J = 8.4 Hz, 1H), 3.93 (s, 3H). | LC-MS: m/z 310.1 [M − H]⁻ |
| 142 | 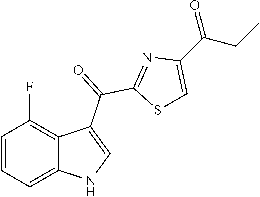 [SMILES: CCC(=O)c1csc(C(=O)c2c[nH]c3cccc(F)c23)n1] | ¹H NMR (400 MHz, DMSO-d₆) δ 12.57 (bs, 1H), 9.13 (s, 1H), 8.86 (s, 1H), 7.42 (d, J = 8.0 Hz, 1H), 7.29-7.34 (m, 1H), 7.00-7.06 (m, 1H), 3.22 (q, J = 7.2 Hz, 2H), 1.14 (t, J = 7.2 Hz, 3H). | ESI MS m/z 301 [M − H]⁻ |
| 143 | 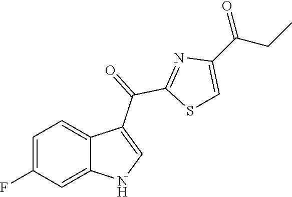 [SMILES: CCC(=O)c1csc(C(=O)c2c[nH]c3cc(F)ccc23)n1] | ¹H NMR (400 MHz, DMSO-d₆) δ 12.35 (bs, 1H), 9.17 (d, J = 2.8 Hz, 1H), 8.86 (s, 1H), 8.27-8.33 (m, 1H), 7.39-7.43 (m, 1H), 7.16-7.18 (m, 1H), 3.24 (q, J = 7.2 Hz, 2H), 1.15 (t, J = 7.2 Hz, 3H). | ESI MS m/z 301 [M − H]⁻ |
| 144 | 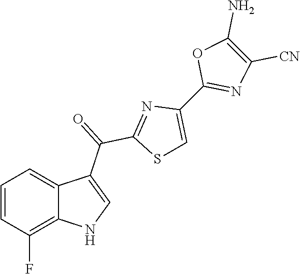 [SMILES: N#Cc1nc(-c2csc(C(=O)c3c[nH]c4c(F)cccc34)n2)oc1N] | | |
| 145 | 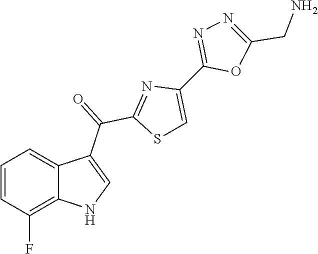 [SMILES: NCc1nnc(-c2csc(C(=O)c3c[nH]c4c(F)cccc34)n2)o1] | ¹H NMR (400 MHz, DMSO-d₆) δ 9.15 (s, 1H), 8.91 (s, 1H), 8.12-8.15 (d, J = 7.6 Hz, 1H), 7.26-7.30 (m, 1H), 7.16-7.21 (m, 1H), 4.03 (s, 2H). | LC/MS (m/z) 342.3 [M + H]− |
| 146 | 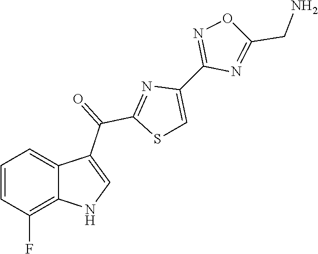 [SMILES: NCc1nc(-c2csc(C(=O)c3c[nH]c4c(F)cccc34)n2)no1] | ¹H NMR (400 MHz, DMSO-d₆) δ 9.16 (s, 1H), 8.83 (s, 1H), 8.13-8.16 (d, J = 8.0 Hz, 1H), 7.26-7.31 (m, 1H), 7.16-7.22 (m, 1H), 4.08 (s, 2H). | LC/MS (m/z) 342.4 [M − H]⁻ |
| 147 | 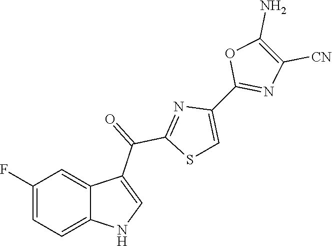 [SMILES: N#Cc1nc(-c2csc(C(=O)c3c[nH]c4ccc(F)cc34)n2)oc1N] | ¹H NMR (400 MHz, DMSO-d₆) δ 12.48 (bs, 1H), 9.12 (s, 1H), 8.49 (s, 1H), 8.17 (s, 2H), 7.96-8.00 (m, 1H), 7.61-7.66 (m, 1H), 7.15-7.21 (m, 1H). | ESI MS m/z 352 [M − H]⁻ |
| 148 | 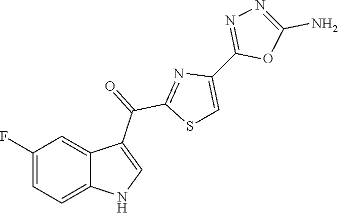 [SMILES: Nc1nnc(-c2csc(C(=O)c3c[nH]c4ccc(F)cc34)n2)o1] | ¹H NMR (400 MHz, DMSO-d₆) δ 12.50 (bs, 1H), 9.12 (s, 1H), 8.56 (s, 1H), 7.97-8.01 (m, 1H), 7.60-7.65 (m, 1H), 7.49 (s, 2H), 7.16-7.19 (m, 1H). | ESI MS m/z 328 [M − H]⁻ |
| 149 | 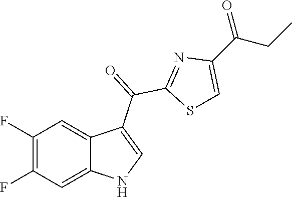 [SMILES: CCC(=O)c1csc(C(=O)c2c[nH]c3cc(F)c(F)cc23)n1] | ¹H NMR (400 MHz, DMSO-d₆) δ 12.43 (bs, 1H), 9.19 (s, 1H), 8.87 (s, 1H), 8.13-8.19 (m, 1H), 7.64-7.70 (m, 1H), 3.24 (q, J = 7.2 Hz, 2H), 1.15 (t, J = 7.2 Hz, 3H). | ESI MS m/z 319 [M − H]⁻ |
| 150 | 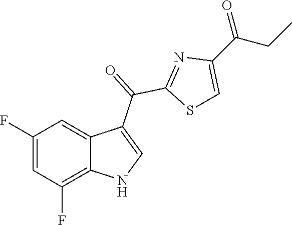 [SMILES: CCC(=O)c1csc(C(=O)c2c[nH]c3c(F)cc(F)cc23)n1] | ¹H NMR (400 MHz, DMSO-d₆) δ 13.11 (bs, 1H), 9.19 (s, 1H), 8.88 (s, 1H), 7.83-7.87 (m, 1H), 7.24-7.31 (m, 1H), 3.23-3.28 (q, J = 7.2 Hz, 2H), 1.13-1.18 (t, J = 7.2 Hz, 3H). | LC-MS: m/z 318.9 [M − H]⁻ |
| 154 | 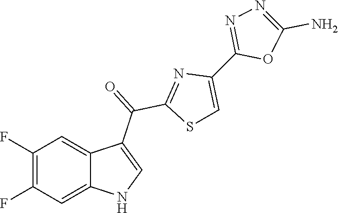 [SMILES: Nc1nnc(-c2csc(C(=O)c3c[nH]c4cc(F)c(F)cc34)n2)o1] | ¹H NMR (400 MHz, DMSO-d₆) δ 12.55 (bs, 1H), 9.11 (s, 1H), 8.58 (s, 1H), 8.13-8.16 (m, 1H), 7.67-7.70 (m, 1H), 7.44 (s, 2H). | LC-MS: m/z 346.0 [M − H]⁻ |

TABLE 2
Additional Exemplary Indole Compounds

| ARI-# | Structural Formula |
|---|---|
| 1001 | 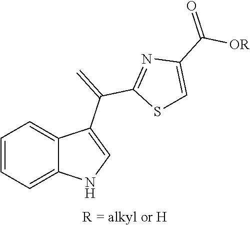 |
| 1002 | 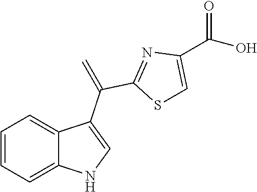 [SMILES: C=C(c1nc(C(=O)O)cs1)c1c[nH]c2ccccc12] |
| 1003 | 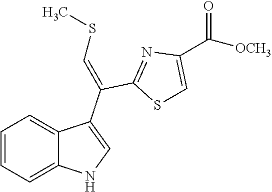 [SMILES: COC(=O)c1csc(/C(=C\SC)c2c[nH]c3ccccc23)n1] |
| 1004 | 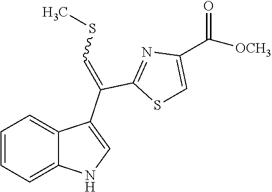 [SMILES: COC(=O)c1csc(C(=CSC)c2c[nH]c3ccccc23)n1] |
| 1005 | 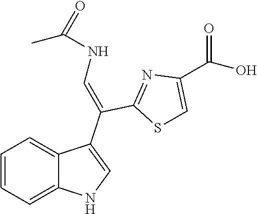 [SMILES: CC(=O)N/C=C(\c1nc(C(=O)O)cs1)c1c[nH]c2ccccc12] |
| 1006 | 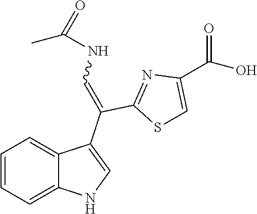 [SMILES: CC(=O)NC=C(c1nc(C(=O)O)cs1)c1c[nH]c2ccccc12] |
| 1007 | 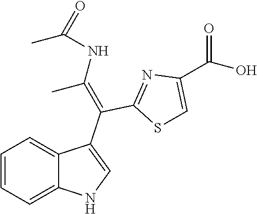 [SMILES: CC(=O)N/C(C)=C(\c1nc(C(=O)O)cs1)c1c[nH]c2ccccc12] |
| 1008 | 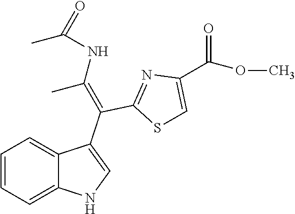 [SMILES: COC(=O)c1csc(/C(=C(/C)NC(C)=O)c2c[nH]c3ccccc23)n1] |
| 1009 | 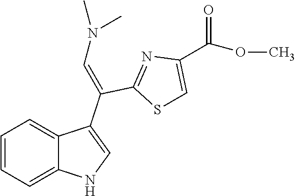 [SMILES: COC(=O)c1csc(/C(=C\N(C)C)c2c[nH]c3ccccc23)n1] |
| 1010 | 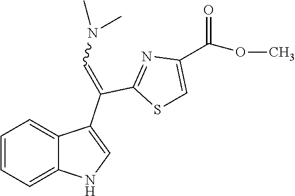 [SMILES: COC(=O)c1csc(C(=CN(C)C)c2c[nH]c3ccccc23)n1] |
| 1011 | 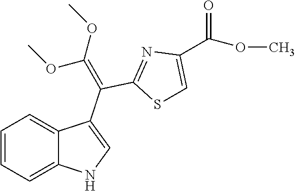 [SMILES: COC(=O)c1csc(C(=C(OC)OC)c2c[nH]c3ccccc23)n1] |
| 1012 | 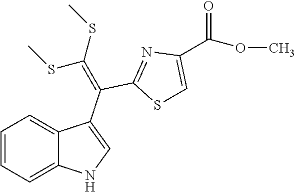 [SMILES: COC(=O)c1csc(C(=C(SC)SC)c2c[nH]c3ccccc23)n1] |
| 1013 | 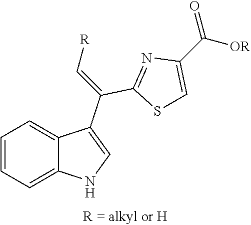 |
| 1014 | 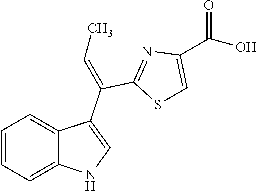 [SMILES: C/C=C(\c1nc(C(=O)O)cs1)c1c[nH]c2ccccc12] |
| 1015 | 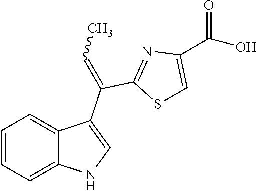 |
| 1016 | 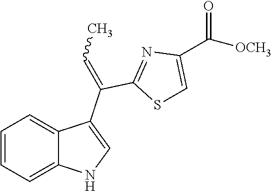 |
| 1017 | 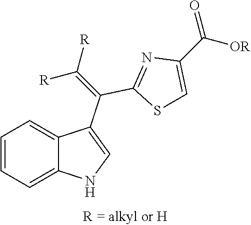 |
| 1018 | 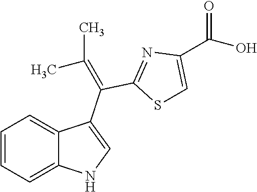 [SMILES: CC(C)=C(c1nc(C(=O)O)cs1)c1c[nH]c2ccccc12] |
| 1019 | 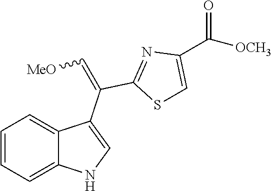 [SMILES: COC=C(c1nc(C(=O)OC)cs1)c1c[nH]c2ccccc12] |
| 1020 | 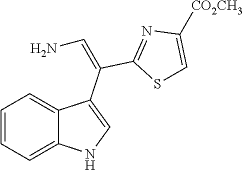 [SMILES: COC(=O)c1csc(/C(=C/N)c2c[nH]c3ccccc23)n1] |
| 1021 | 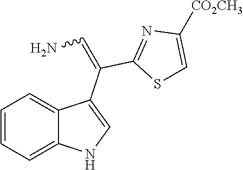 [SMILES: COC(=O)c1csc(C(=CN)c2c[nH]c3ccccc23)n1] |
| 1022 | 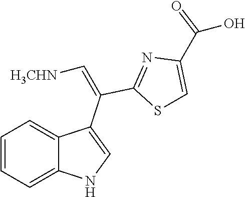 |
| 1023 | 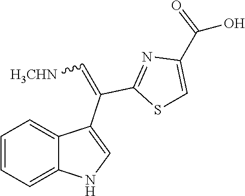 |
| 1024 | 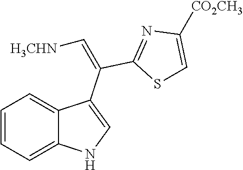 |
| 1025 | 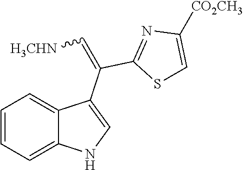 |
| 1026 | 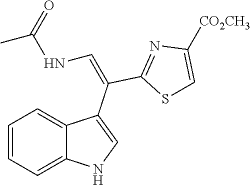 [SMILES: COC(=O)c1csc(/C(=C/NC(C)=O)c2c[nH]c3ccccc23)n1] |
| 1027 | 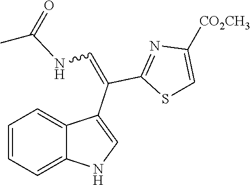 [SMILES: COC(=O)c1csc(C(=CNC(C)=O)c2c[nH]c3ccccc23)n1] |
| 1028 | 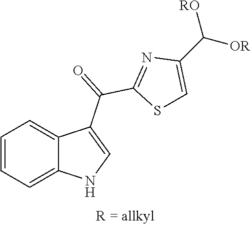 |
| 1029 | 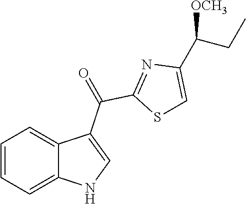 [SMILES: CC[C@H](OC)c1csc(C(=O)c2c[nH]c3ccccc23)n1] |
| 1030 | 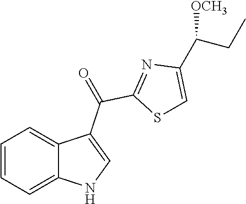 [SMILES: CC[C@@H](OC)c1csc(C(=O)c2c[nH]c3ccccc23)n1] |
| 1031 | 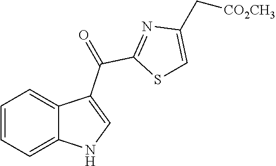 [SMILES: COC(=O)Cc1csc(C(=O)c2c[nH]c3ccccc23)n1] |
| 1032 | 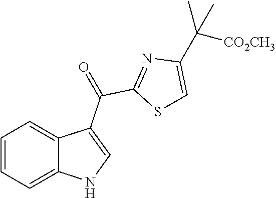 [SMILES: COC(=O)C(C)(C)c1csc(C(=O)c2c[nH]c3ccccc23)n1] |
| 1033 | 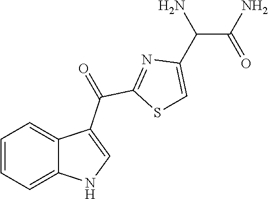 [SMILES: NC(=O)C(N)c1csc(C(=O)c2c[nH]c3ccccc23)n1] |
| 1034 | 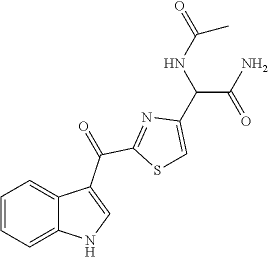 [SMILES: CC(=O)NC(C(N)=O)c1csc(C(=O)c2c[nH]c3ccccc23)n1] |
| 1035 | 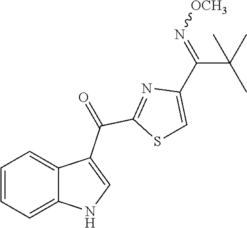 [SMILES: CON=C(c1csc(C(=O)c2c[nH]c3ccccc23)n1)C(C)(C)C] |
| 1036 | 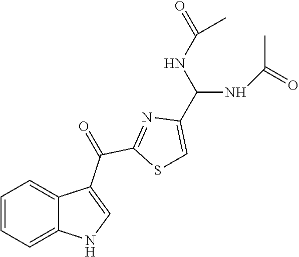 [SMILES: CC(=O)NC(NC(C)=O)c1csc(C(=O)c2c[nH]c3ccccc23)n1] |
| 1037 | 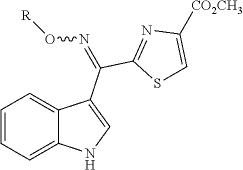 |
| 1038 | 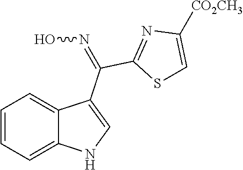 [SMILES: COC(=O)c1csc(C(=NO)c2c[nH]c3ccccc23)n1] |
| 1039 | 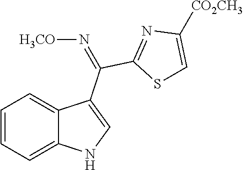 [SMILES: CO/N=C(/c1nc(C(=O)OC)cs1)c1c[nH]c2ccccc12] |
| 1040 | 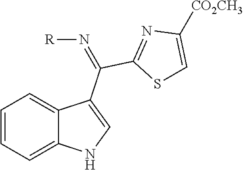 |
| 1041 | 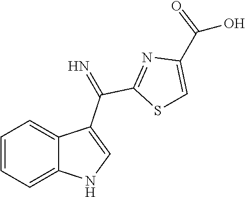 [SMILES: N=C(c1nc(C(=O)O)cs1)c1c[nH]c2ccccc12] |
| 1042 | 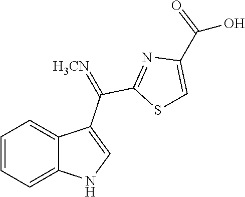 |
| 1043 | 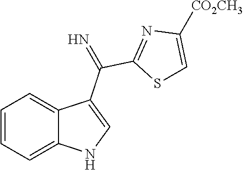 [SMILES: COC(=O)c1csc(C(=N)c2c[nH]c3ccccc23)n1] |
| 1044 | 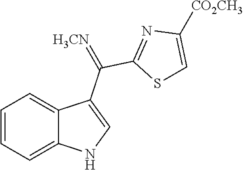 |
| 1045 | 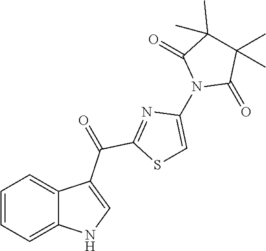 [SMILES: CC1(C)C(=O)N(c2csc(C(=O)c3c[nH]c4ccccc34)n2)C(=O)C1(C)C] |
| 1046 | 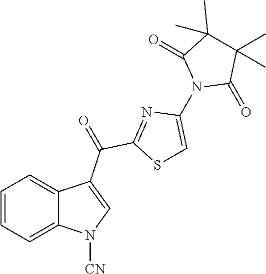 [SMILES: CC1(C)C(=O)N(c2csc(C(=O)c3cn(C#N)c4ccccc34)n2)C(=O)C1(C)C] |
| 1047 | 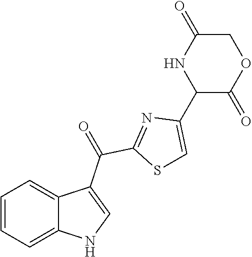 [SMILES: O=C1COC(=O)C(c2csc(C(=O)c3c[nH]c4ccccc34)n2)N1] |
| 1048 | 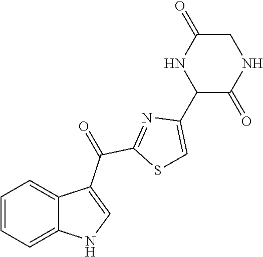 [SMILES: O=C1CNC(=O)C(c2csc(C(=O)c3c[nH]c4ccccc34)n2)N1] |
| 1049 | 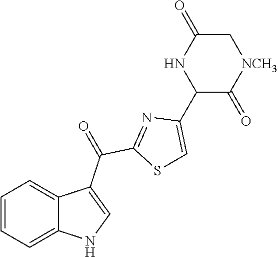 [SMILES: CN1CC(=O)NC(c2csc(C(=O)c3c[nH]c4ccccc34)n2)C1=O] |
| 1050 | 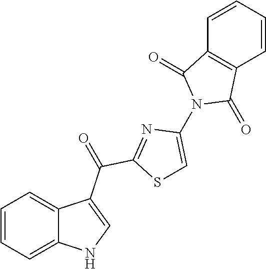 [SMILES: O=C(c1nc(N2C(=O)c3ccccc3C2=O)cs1)c1c[nH]c2ccccc12] |
| 1051 | 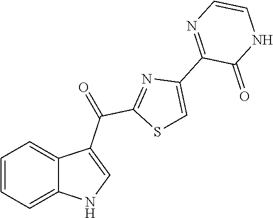 [SMILES: O=C(c1nc(-c2ncc[nH]c2=O)cs1)c1c[nH]c2ccccc12] |
| 1052 | 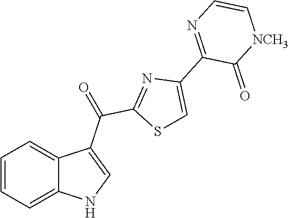 [SMILES: Cn1ccnc(-c2csc(C(=O)c3c[nH]c4ccccc34)n2)c1=O] |
| 1053 | 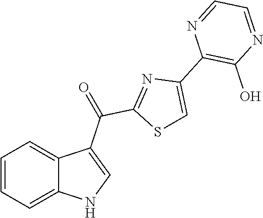 [SMILES: O=C(c1nc(-c2nccnc2O)cs1)c1c[nH]c2ccccc12] |
| 1054 | 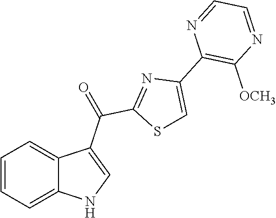 [SMILES: COc1nccnc1-c1csc(C(=O)c2c[nH]c3ccccc23)n1] |
| 1055 | 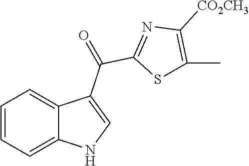 [SMILES: COC(=O)c1nc(C(=O)c2c[nH]c3ccccc23)sc1C] |
| 1056 | 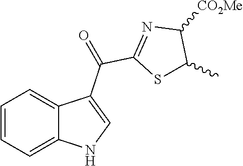 [SMILES: COC(=O)C1N=C(C(=O)c2c[nH]c3ccccc23)SC1C] |
| 1057 | 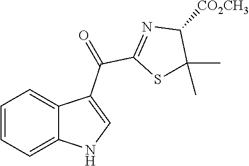 [SMILES: COC(=O)[C@H]1N=C(C(=O)c2c[nH]c3ccccc23)SC1(C)C] |
| 1058 | 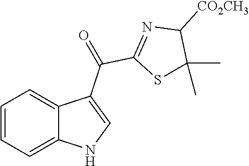 [SMILES: COC(=O)C1N=C(C(=O)c2c[nH]c3ccccc23)SC1(C)C] |
| 1059 | 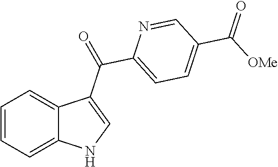 [SMILES: COC(=O)c1ccc(C(=O)c2c[nH]c3ccccc23)nc1] |
| 1060 | 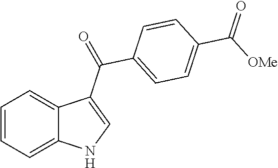 [SMILES: COC(=O)c1ccc(C(=O)c2c[nH]c3ccccc23)cc1] |
| 1061 | 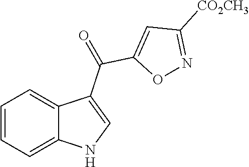 [SMILES: COC(=O)c1cc(C(=O)c2c[nH]c3ccccc23)on1] |
| 1062 | 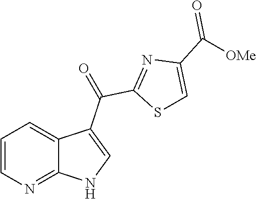 [SMILES: COC(=O)c1csc(C(=O)c2c[nH]c3ncccc23)n1] |
| 1063 | 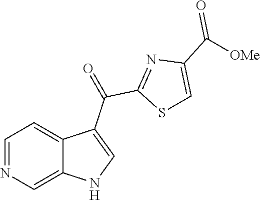 [SMILES: COC(=O)c1csc(C(=O)c2c[nH]c3cnccc23)n1] |
| 1064 | 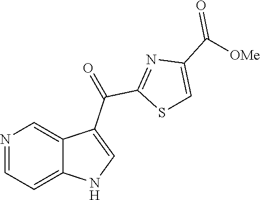 [SMILES: COC(=O)c1csc(C(=O)c2c[nH]c3ccncc23)n1] |
| 1065 | 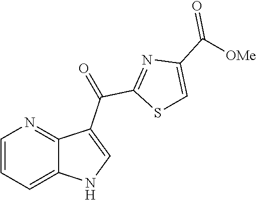 [SMILES: COC(=O)c1csc(C(=O)c2c[nH]c3cccnc23)n1] |
| 1066 | 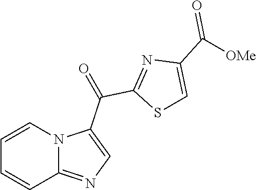 [SMILES: COC(=O)c1csc(C(=O)c2cnc3ccccn23)n1] |
| 1067 | 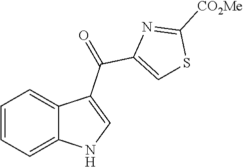 [SMILES: COC(=O)c1nc(C(=O)c2c[nH]c3ccccc23)cs1] |
| 1068 | 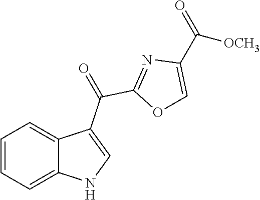 [SMILES: COC(=O)c1coc(C(=O)c2c[nH]c3ccccc23)n1] |
| 1069 | 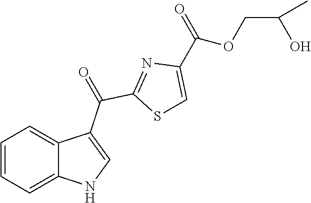 [SMILES: CC(O)COC(=O)c1csc(C(=O)c2c[nH]c3ccccc23)n1] |
| 1070 | 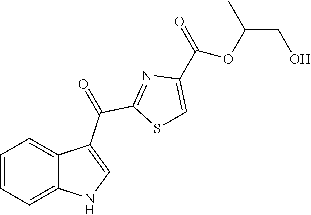 [SMILES: CC(CO)OC(=O)c1csc(C(=O)c2c[nH]c3ccccc23)n1] |
| 1071 | 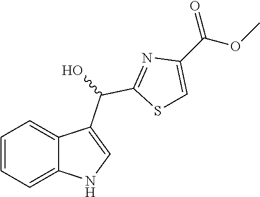 [SMILES: COC(=O)c1csc(C(O)c2c[nH]c3ccccc23)n1] |
| 1072 | 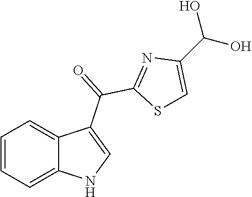 [SMILES: O=C(c1nc(C(O)O)cs1)c1c[nH]c2ccccc12] |
| 1073 | 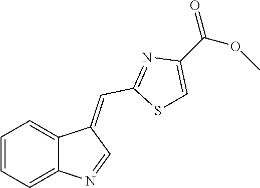 [SMILES: COC(=O)c1csc(/C=C2\C=Nc3ccccc32)n1] |

Single stereochemical isomers, enantiomers, diastereomers, and pharmaceutically acceptable salts of the above exemplified compounds are also within the scope of the present disclosure. Pharmaceutically acceptable salts may be, for example, derived from suitable inorganic and organic acids and bases.

Acid addition salts can be prepared by reacting the purified compound in its free-based form, if possible, with a suitable organic or inorganic acid and isolating the salt thus formed. Examples of pharmaceutically acceptable acid addition salts include, without limitations, salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid.

Base addition salts can be prepared by reacting the purified compound in its acid form with a suitable organic or inorganic base and isolating the salt thus formed. Such salts include, without limitations, alkali metal (e.g., sodium, lithium, and potassium), alkaline earth metal (e.g., magnesium and calcium), ammonium and N⁺(C₁₋₄alkyl)₄ salts.

Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, and valerate salts.

In some embodiments, the indole compound is selected from ARI-017, ARI-018, ARI-019, ARI-020, ARI-031, ARI-060, ARI-083, ARI-087, ARI-090, ARI-118, ARI-120, ARI-140, ARI-143, ARI-145, ARI-146, ARI-148, ARI-149, or ARI-150, or an enantiomer, diastereomer, or pharmaceutically acceptable salt thereof. In some embodiments, the compound is selected from ARI-087, ARI-140, ARI-143, ARI-149, and ART-150, or an enantiomer, diastereomer, or pharmaceutically acceptable salt thereof. In some embodiments, the compound is selected from ART-031, ART-060, ART-083, ARI-090, ART-118, ART-120, ART-145, ARI-146, and ART-148, or an enantiomer, diastereomer, or pharmaceutically acceptable salt thereof.

The indole compounds of the present disclosure may be synthesized by methods known in the art or by methods illustrated in the Examples of the present application below as well as in the Examples in U.S. Provisional Patent Application No. 62/717,387, filed Aug. 10, 2018, and U.S. Provisional Patent Application No. 62/588,751, filed Nov. 20, 2017, each of which is incorporated herein by reference in its entirety.

### Synthesis of Indole Compounds

The compounds disclosed herein can be prepared by using one or more of the following general synthetic schemes exemplified below. These general synthetic schemes as well as the examples that follow are for purposes of illustration only and are not to be construed as limiting the scope of the invention in any manner.

Unless otherwise noted, reagents and solvents were used as received from commercial suppliers. Non-commercial indole carboxylic acids were prepared by the methods of A. S. Katner; Organic Preparations and Procedures 2(4):297-303 (1970); J Med Chem 57(17):7293-7316 (2014); and Chem. Eur. J 17(26):7298-7303 (2011). Non-commercial key intermediates methyl 2-(1H-indole-3-carbonyl)thiazole-4-carboxylate and 2-(1H-indole-3-carbonyl)thiazole-4-carboxylic acid were obtained from the literature preparation. The preparation of tert-butyl 3-(methoxy(methyl)carbamoyl)-1H-indazole-1-carboxylate is known. See Crestey, Francois; Stiebing, Silvia; Legay, Remi; Collot, Valerie; Rault, Sylvain; Tetrahedron 63(2):419-428 (2007).

All non-aqueous reactions were carried out under an atmosphere of dry nitrogen (unless otherwise noted). Proton nuclear magnetic resonance spectra were obtained on a Bruker Avance III 400 MHz NMR with autosampler, a Bruker Avance II 300 MHz NMR, or a Bruker Ascend 500 spectrometer at 500 MHz. Spectra are given in ppm (δ) and coupling constants, J values, are reported in hertz (Hz). Tetramethylsilane was used as an internal standard for proton nuclear magnetic resonance. Mass spectra and LCMS analyses were obtained using a Waters Acquity UPLC-H Class LC-MS system or a Shimadzu 2020 single quadrupole mass spectrometer (DUIS, UP-LCMS). HPLC analyses were performed using a Waters Separations Module 2695/2998 PDA detector.

### Intermediate Preparation

### Preparation 1: 2-bromo-4-((tert-butyldimethylsilyloxy)methyl)thiazole (1)

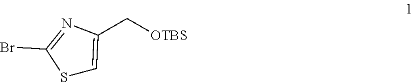 [SMILES: CC(C)(C)[Si](C)(C)OCc1csc(Br)n1]

This compound was prepared according to the procedure described in documents WO2013/163279 and Tetrahedron Lett. 1991, 32, 4263. NaBH₄ (32.0 g, 0.845 mol) was added portionwise to a solution of ethyl 2-bromothiazole-4-carboxylate (100.0 g, 0.424 mol) in EtOH (800 mL) over 0.5 h at <50° C. with stirring. The suspension was heated under reflux for 5 h. The mixture was cooled to room temperature and the solvent was removed under reduced pressure. The residue was dissolved in CH₂Cl₂ (500 mL) and the resulting solution was washed with saturated aqueous NaHCO₃ (300 mL×3) and brine (300 mL×1), dried over anhydrous Na₂SO₄ and concentrated to dryness to afford the corresponding alcohol (70 g).

The alcohol was dissolved in dimethylformamide (DMF) (300 mL) and imidazole (36.8 g, 0.54 mol) was added. Then a solution of TBS-Cl (81.5 g, 0.54 mol) in tetrahydrofuran (THF) (200 mL) was added dropwise at room temperature. The reaction mixture was stirred overnight, and then water (100 mL) was added. The resulting mixture was extracted with EtOAc (100 mL×3). The combined organic phases were washed with aqueous 5% KHSO₄ (200 mL×3), saturated aqueous NaHCO₃ (200 mL×3) and brine (200 mL×1), dried over anhydrous Na₂SO₄ and concentrated to dryness. The residue was purified by distillation under reduced pressure to afford compound 1 (bp130˜140 C/13.3 pa, 96.1 g, 74% yield) as an oil.

### Preparation 2: 5-fluoro-1H-indole-3-carboxylic acid (2)

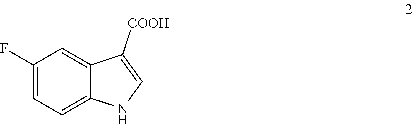 [SMILES: O=C(O)c1c[nH]c2ccc(F)cc12]

This compound was prepared according to the procedure described in the Journal of Medicinal Chemistry 2014, 57(17), 7293-7316. Trifluoroacetic anhydride (38 mL, 56.0 g, 0.27 mol) was added dropwise to a solution of 5-fluoro-1H-indole (30.0 g, 0.22 mol) in DMF (300 mL) over 0.5 h at 0° C. The reaction mixture was allowed to warm to room temperature and stirred overnight. The mixture was quenched with water (1 L), after which solids began to form, the mixture was stirred for 0.5 h, then filtered. The solid was collected, washed with water (200 mL×3), then added to aqueous sodium hydroxide (20%, 150 mL, 0.75 mol) and heated under reflux for 8 h. The reaction mixture was cooled and acidified with aqueous 3N HCl to pH of 3 whereupon a precipitate was produced. The solid was collected by filtration, washed with water (200 mL×3), dried to afford compound 2 (27.1 g, 68% yield) as off-white solid.

### Preparation 3: 7-fluoro-1H-indole-3-carboxylic acid (3)

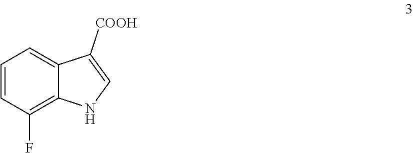 [SMILES: O=C(O)c1c[nH]c2c(F)cccc12]

This compound was synthesized according to the protocol described in Preparation 2 from 7-fluoro-1H-indole to give title compound in the form of a yellow solid (75% yield).

### Preparation 4: 1H-Indole-5-methoxy-3-carboxylic acid (4)

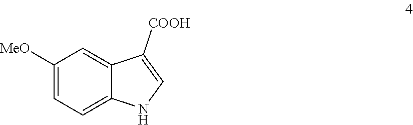 [SMILES: COc1ccc2[nH]cc(C(=O)O)c2c1]

This compound was synthesized according to the protocol described in Preparation 2 from 5-methoxy-1H-indole to give title compound in the form of a yellow solid (65% yield).

### Preparation 5: 5-Bromo-1H-Indole-3-carboxylic acid (5)

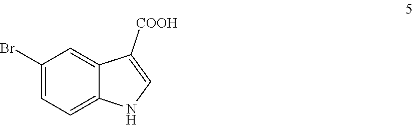 [SMILES: O=C(O)c1c[nH]c2ccc(Br)cc12]

This compound was synthesized according to the protocol described in Preparation 2 from 5-bromo-1H-indole to give title compound in the form of a yellow solid (70% yield).

### Preparation 6: 6-Bromo-1H-Indole-3-carboxylic acid (6)

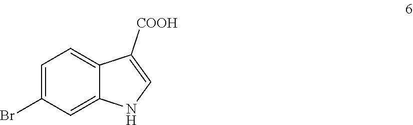 [SMILES: O=C(O)c1c[nH]c2cc(Br)ccc12]

This compound was synthesized according to the protocol described in Preparation 2 from 6-bromo-1H-indole to give title compound in the form of a yellow solid (55% yield).

### Preparation 7: 7-Bromo-1H-Indole-3-carboxylic acid (7)

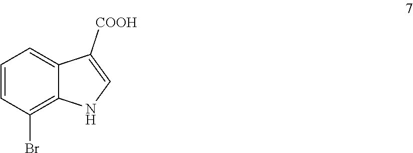 [SMILES: O=C(O)c1c[nH]c2c(Br)cccc12]

This compound was synthesized according to the protocol described in Preparation 2 from 7-bromo-1H-indole to give title compound in the form of a yellow solid (63% yield).

### Preparation 8: 5-chloro-2-methyl-1H-indole-3-carboxylic acid (8)

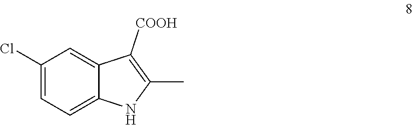

This compound was prepared according to the procedure described in Chemistry-A European Journal, 2011, 17(26), 7298-7303. InBr₃ (5 mg, cat.) and anhydrous MgSO₄ (24.0 g, 0.2 mol) were added to a solution of 4-chloroaniline (24.0 g, 0.21 mol) and methyl acetoacetate (28.0 g, 0.24 mol) in dichloromethane (DCM) (200 mL) at room temperature. The reaction mixture was stirred overnight, then filtered. The filtrate was concentrated to dryness. The residue was dissolved in DMF (200 mL), and Pd(OAc)₂ (2.2 g, 10 mmol), Cu(OAc)₂ (110.0 g, 0.61 mol), K₂CO₃ (83.0 g, 0.60 mol) were added. The resulting mixture was heated to 140° C. and stirred for 5 h. The mixture was cooled to room temperature, quenched with water (500 mL) and then extracted with EtOAc (300 mL×3). The combined organic phases were washed with water (500 mL×3), saturated aqueous NaHCO₃ (500 mL×3) and brine (500 mL×1), dried (Na₂SO₄), filtered and then concentrated to dryness. The residue was purified by flash column chromatography on silica gel (EtOAc:Hexane-1:20 to 1:10) to afford methyl 5-chloro-2-methyl-1H-indole-3-carboxylate (10.1 g, 21% yield).

The above methyl ester (10.0 g, 45 mmol) was added to aqueous sodium hydroxide (10%, 100 mL, 0.25 mol) and heated under reflux for 8 h. The reaction mixture was cooled and acidified with aqueous 3N HCl to a pH of 3 whereupon a precipate began to form. The solid was collected by filtration, washed with water (20 mL×3), dried to afford compound 8 (6.7 g, 71% yield) as an off-white solid.

### Preparation 9: 5-fluoro-2-methyl-1H-indole-3-carboxylic acid (9)

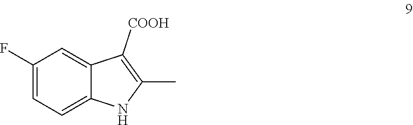

This compound was synthesized according to the protocol described in Preparation 8 from 4-fluoroaniline to give title compound in the form of a yellow solid (22% yield).

### Preparation of the Key Intermediates Int-A, Int-B and Int-C

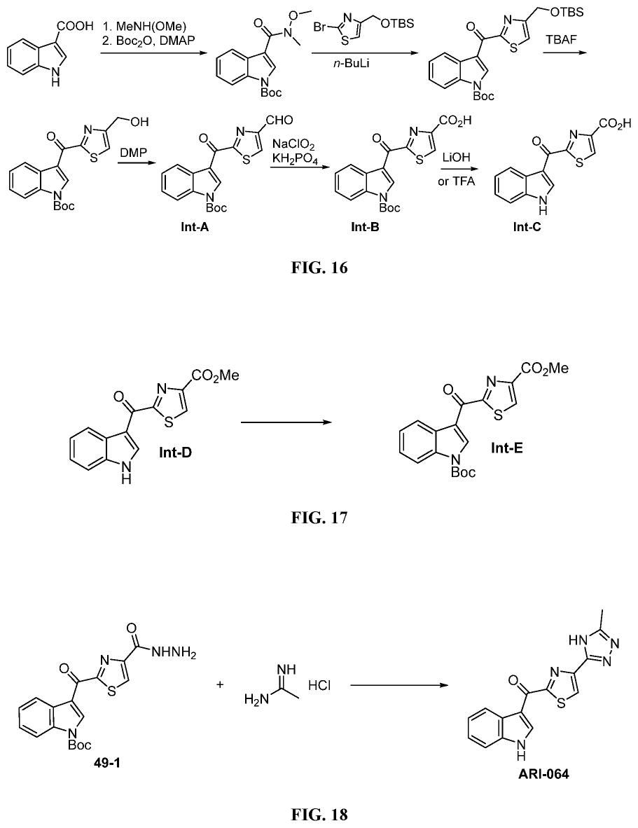

The key intermediates Int-A, Int-B and Int-C were synthesized according to the scheme of FIG. 16 and by the following steps:

Step 1: Oxalyl chloride (473.3 g, 3.73 mol) was added dropwise to a suspension of indol-3-carboxylic acid (400 g, 2.48 mol) in DCM (4 L) at 0° C. over 1 h. The mixture was allowed to warm to room temperature and stirred overnight. The mixture was concentrated to dryness to afford 1H-indole-3-carbonyl chloride (446.0 g).

The above 1H-indole-3-carbonyl chloride (446.0 g) was added portion-wise to a suspension of N,O-dimethylhydroxylamine hydrochloride (266.0 g, 2.73 mol) and TEA (551.1 g, 5.46 mol) in DCM (5 L) at room temperature over 1 h. The mixture was stirred overnight, then quenched with water (2 L). The organic phase was collected and washed with water (2 L×2), saturated aqueous NaHCO₃ (2 L×2), and brine (2 L×1), dried (Na₂SO₄), filtered and concentrated to dryness. The residue and DMAP (15.1 g, 0.124 mol) was dissolved in DMF (1 L) and DCM (4 L), cooled to 0° C. Boc₂O (540.64 g, 2.48) and DMAP (15.1 g, 0.124 mol) were added dropwise to over 1 h.

The resulting mixture was allowed to warm to room temperature and stirred overnight. The mixture was quenched with water (2 L). The organic phase was separated and washed with water (2 L×2), saturated aqueous NaHCO₃ (2 L×2), and brine (2 L×1), dried (Na₂SO₄), filtered and concentrated to dryness. The residue was triturated with EtOAc/hexane (1:5, 1 L), filtered and dried to afford tert-butyl 3-(methoxy(methyl)carbamoyl)-1H-indole-1-carboxylate (557.9 g, 75% yield) as off-white solid. ¹H-NMR (400 MHz, DMSO-d6): δ 8.25 (s, 1H), 8.22 (d, J=8.0 Hz, 1H), 8.13 (d, J=7.6 Hz, 1H), 7.35-7.45 (m, 1H), 7.30-7.35 (m, 1H), 3.74 (s, 3H), 3.32 (s, 3H), 1.67 (s, 9H).

Step 2: A solution of 2-bromo-4-((tert-butyldimethylsilyloxy)methyl)thiazole (135.0 g, 0.44 mol) in THF (1.5 L) was cooled to −78° C., and n-BuLi (1.6 M solution in hexane, 385 mL, 0.62 mol) was added dropwise at −78° C. over 1 h. The mixture was stirred for 0.5 h at this temperature, then a solution of tert-butyl 3-(methoxy(methyl)carbamoyl)-1H-indole-1-carboxylate (120.0 g, 0.4 mol) in THF (500 mL) was added dropwise over 1 h. The mixture was stirred at −78° for 1 h then allowed to warm to 0° C. and quenched with aqueous 10% NH₄Cl (1 L). The organic phase was collected and washed with water (1 L×2), saturated aqueous NaHCO₃ (1 L×2), and brine (1 L×1), dried (Na₂SO₄), filtered and concentrated to dryness. The residue was triturated with EtOAc/hexane (1:5, 500 mL), filtered and dried to afford tert-butyl 3-(4-((tert-butyldimethylsilyloxy)methyl)thiazole-2-carbonyl)-1H-indole-1-carboxylate (132.0 g, 70% yield) as off-white solid. ¹H-NMR (400 MHz, DMSO-d6): δ 9.42 (bs, 1H), 8.39 (d, J=7.6 Hz, 1H), 8.14 (d, J=8.0 Hz, 1H), 8.01 (s, 1H), 7.40-7.52 (m, 2H), 4.93 (s, 2H), 1.69 (s, 9H), 0.92 (s, 9H), 0.14 (s, 6H).

Step 3: A solution of tert-butyl 3-(4-((tert-butyldimethylsilyloxy)methyl)thiazole-2-carbonyl)-1H-indole-1-carboxylate (91.0 g, 0.19 mol) in THF (500 mL) and pyridine (50 mL) was cooled to 0° C., and HF-pyridine (30% , 50 mL) was added dropwise over 10 min. The mixture was stirred for 0.5 h at this temperature, then allowed to warm to room temperature and stirred overnight. The mixture was quenched with aqueous 10% NH₄Cl (1 L) and EtOAc (500 mL). The organic phase was collected and washed with water (500 mL×2), saturated aqueous NaHCO₃ (500 mL×2), and brine (500 mL×1), dried (Na₂SO₄), filtered and concentrated to dryness. The residue was triturated with EtOAc/hexane (1:5, 100 mL), filtered and dried to afford tert-butyl 3-(4-(hydroxymethyl)thiazole-2-carbonyl)-1H-indole-1-carboxylate (49.6 g, 73% yield) as off-white solid. ¹H-NMR (400 MHz, DMSO-d6): δ 9.43 (s, 1H), 8.35-8.40 (m, 1H), 8.13 (d, J=8.0 Hz, 1H), 7.97 (s, 1H), 7.42-7.50 (m, 2H), 5.5557 (t, J=5.6 Hz, 1H), 4.75 (d, J=5.6 Hz, 2H), 1.99 (s, 9H). ESI MS: m/z 359 [M+H]⁺.

Step 4: To a mixture of tert-butyl 3-(4-(hydroxymethyl)thiazole-2-carbonyl)-1H-indole-1-carboxylate (1.9 g, 5.30 mmol) in DCM (53.0 ml) in a water bath was added 1,1,1-tris(acetyloxy)-1,1-dihydro-1,2-benziodoxol-3-(1H)-one (2.473 g, 5.83 mmol). After 1 h, saturated NaHCO₃ (aq) and 10% Na₂S₂O₃ (aq) were added then the mixture stirred for 30 min. The layers were separated and the organic phase was washed with bicarbonate, dried (Na₂SO₄), filtered and concentrated. Chromatography (silica gel, heptane to CH₂Cl₂) gave tert-butyl 3-(4-formylthiazole-2-carbonyl)-1H-indole-1-carboxylate (1.63 g) as a white solid. ESI MS m/z 357 [M+H]⁺.

Step 5: A solution of NaClO₂ (19.0 g, 210 mmol) and KH₂PO₄ (46.7 g, 0.336 mmol) in H₂O (200 mL) was added dropwise to a solution of tert-butyl 3-(4-formylthiazole-2-carbonyl)-1H-indole-1-carboxylate (15.0 g, 42 mmol) in tBuOH/H₂O/DCM (300 mL/60 mL/60 mL) at room temperature over 0.5 h. The mixture was stirred for 5 h. The mixture was extracted with EtOAc (300 mL×4), the combined organic phases were washed with aqueous 5% KHSO₄ (500 mL×3) and brine (500 mL×1), dried (Na₂SO₄), filtered and concentrated to dryness. The residue was triturated with EtOAc/hexane (1:2, 50 mL), filtered and dried to afford 2-(1-(tert-butoxycarbonyl)-1H-indole-3-carbonyl)thiazole-4-carboxylic acid (13.5 g, 86% yield) as off-white solid. ¹H-NMR (400 MHz, DMSO-d6): δ 13.48 (bs, 1H), 9.62 (s, 1H), 8.89 (s, 1H), 8.38 (m 1H), 8.18 (d, J=8.0 Hz, 1H), 7.48 (m, 2H), 1.69 (s, 9H). ESI MS m/z 371 [M−H]⁻.

### Alternate Preparation of the Key Intermediate Int-B

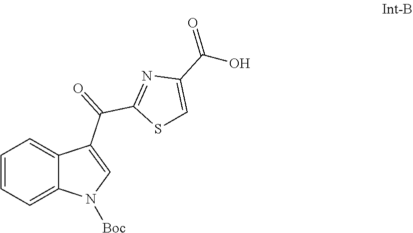 [SMILES: CC(C)(C)OC(=O)n1cc(C(=O)c2nc(C(=O)O)cs2)c2ccccc21]

To a suspension of methyl 2-(1H-indole-3-carbonyl)thiazole-4-carboxylate (5.00 g, 17.46 mmol) and di-tert-butyl dicarbonate (5.27 ml, 22.70 mmol) in acetonitrile (175 ml) was added DMAP (0.640 g, 5.24 mmol). Upon completion, the reaction mixture was concentrated. Chromatography (silica gel, 1% to 10% MeOH in DCM) gave methyl 2-(1-(tert-butoxycarbonyl)-1H-indole-3-carbonyl)thiazole-4-carboxylate (6.30 g) as an off white solid. ESI MS m/z 373 [M+H]⁺.

### Preparation of the Key Intermediate Int-E

The key intermediate Int-E was synthesized according to the scheme of FIG. 17 and by the following method:

To a suspension of methyl 2-(1H-indole-3-carbonyl)thiazole-4-carboxylate (200 mg, 0.699 mmol) and di-tert-butyl dicarbonate (198 mg, 0.908 mmol) in acetonitrile (6986 μl) was added 4-(dimethylamino)pyridine (25.6 mg, 0.210 mmol). Upon completion, the reaction solvent was concentrated. Chromatography (silica gel, heptane to % MeOH/CH₂Cl₂) gave methyl 2-(1-(tert-butoxycarbonyl)-1H-indole-3-carbonyl)thiazole-4-carboxylate (269 mg) as a white solid. ESI MS m/z 387 [M+H]⁺

### Phosphate Derivatives of Indole Compounds

Phosphate derivatives of indole compounds can also be used in the disclosed methods. The indole compounds can bind specifically to and modulate human aryl hydrocarbon receptor (AhR). Without wishing to be bound by theory, it is contemplated that AhR bound by one of the indole compounds is agonized with respect to the receptor's immune-stimualtory activity. The phosphate derivative of an indole compound can include a phosphate moiety, which can be a phosphate salt. The phosphate moiety can include an alkoxy group. The phosphate salt can have one or more counter ions, which can be an alkali metal ion, an alkaline earth metal ion, or an organic amine cation. In a particular embodiment, the phosphate derivative of an indole compound is an indolo-phosphoramidate analog (IPA). The indolo-phosphoramidate analog can have a nitrogen-phosphorous (N-P) bond. In certain embodiments, the indolo-phosphoramidate analog can include a labile linker between the indole nitrogen and the phosphate phosphorus. The linker can form a phosphate. Alternatively, the linker can be non-labile, such as a phosphonate. The labile linker can be of the formula —(CR₂R₃—O)ₓ—, where x is 0, 1, 2, 3, 4, 5, or 6 and each of R₂ and R₃ can be, independently, H, or C1-C6 alkyl. The carbon of the CR₂R₃—O— group can be bonded to the indole nitrogen. In certain embodiments, x is 0 or 1. In certain embodiments, each of R₂ and R₃ can be, independently, H. In some embodiments, the phosphate derivatives as well as their synthesis methods are those described in U.S. Provisional Patent Application No. 62/734,989, filed Sep. 21, 2018, which is incorporated herein by reference in its entirety.

In one embodiment, the phosphate derivative is a compound of Formula I:

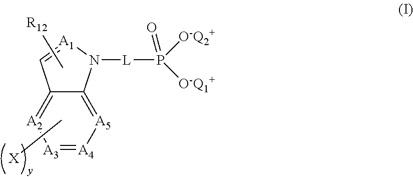

R₁₂ can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio.

Each of A₁, A₂, A₃, A₄, and A₅, independently, can be CR₂ or N.

L can be —(CR₂R₃—O)ₙ— or a bond.

R₂ can be H or C1-C6 alkyl, R₃ can be H or C1-C6 alkyl, or, together, R₂ and R₃ form a C3-C8 cycloalkyl.

n can be 0, 1, 2, 3, 4, 5, or 6.

y can be 0, 1, 2, 3, or 4.

Each X, independently, can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio.

Q₁⁺ and Q₂⁺ can be each, independently, a monocation, or together can be a dication or one of Q₁⁺ or Q₂⁺ can be C1-C6 alkyl, benzyl, allyl or —(CR₂R₃—O)—R₂₃, and R₂₃ can be H or C1-C6 alkyl. The alkyl can be a substituted alkyl, for example an alkoxy alkyl, amino alkyl, alkyl ester, alkyl carbamate or alkyl carbonate.

In some embodiments, the phosphate derivative has a structure Formula II:

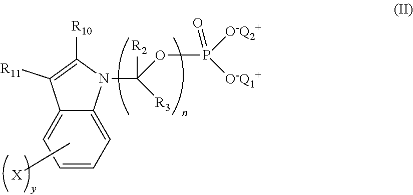

R₁₀ can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio.

R₁₁ can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio.

One of R₁₀ and R₁₁ is H or C1-C6 alkyl.

R₂ can be H or C1-C6 alkyl, R₃ can be H or C1-C6 alkyl, or, together, R₂ and R₃ can form a C3-C8 cycloalkyl.

y can be 0, 1, 2, 3, or 4.

Each X, independently, can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio.

Q₁⁺ and Q₂⁺ can be each, independently, a monocation, or together are a dication.

n can be 0, 1, 2, 3, 4, 5, or 6. For example, n can be 0 or n can be 1.

In certain circumstances, the phosphate derivative can be of Formula III:

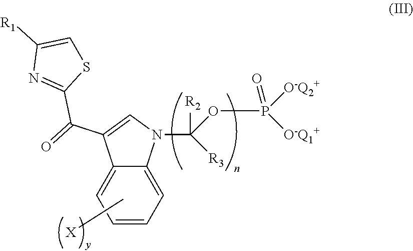

wherein:

R₁ can be —C(═O)—R₄, cyano, an oxadiazole, or a thiadiazole.

R₂ and R₃ can be each, independently, hydrogen, or C₁-C₆ alkyl.

R₄ can be selected from the group consisting of —NRₐR_{b} (Rₐ and R_{b} are each independently H, C₁-C₆ alkyl, or C₁-C₆ acyl), hydrogen, deuterium, halo, amino, hydroxy, cyano, formyl, furyl, nitro, alkyl, haloalkyl, alkenyl, haloalkenyl, alkynyl, haloalkynyl, unsubstituted or substituted C₁-C₆ acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₘR₂₂ (m=0 to 2, R₂₂ is directly connected to S), wherein R₂₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carbonyloxy, halocarbonyloxy, carbonylthio, halocarbonylthio, thiocarbonyloxy, halothiocarbonyloxy, thiocarbonylthio, and halothiocarbonylthio.

In certain circumstances, the phosphate derivative can be of Formula IV:

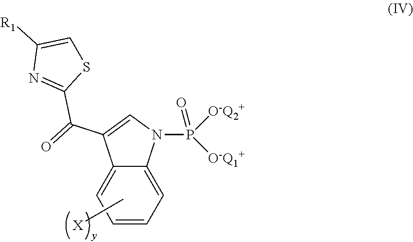

wherein:

R₁ can be —C(═O)—R₄, cyano, an oxadiazole, or a thiadiazole.

R₄ can be selected from the group consisting of —NRₐR_{b} (Rₐ and R_{b} are each independently H, C₁-C₆ alkyl, or C₁-C₆ acyl), hydrogen, deuterium, halo, amino, hydroxy, cyano, formyl, furyl, nitro, alkyl, haloalkyl, alkenyl, haloalkenyl, alkynyl, haloalkynyl, unsubstituted or substituted C₁-C₆ acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₘR₂₂ (m=0 to 2, R₂₂ is directly connected to S), wherein R₂₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carbonyloxy, halocarbonyloxy, carbonylthio, halocarbonylthio, thiocarbonyloxy, halothiocarbonyloxy, thiocarbonylthio, and halothiocarbonylthio.

Each X, independently, can be H or halogen.

In certain circumstances, the phosphate derivative can be of Formula V:

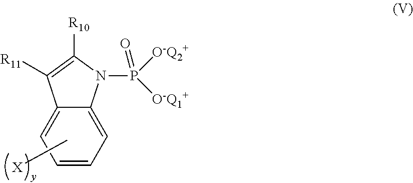

wherein:

R₁₀ can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio.

R₁₁ can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio, wherein one of R₁₀ and R₁₁ can be H or C1-C6 alkyl.

y can be 0, 1, 2, 3, or 4.

Each X, independently, can be H or halogen.

In certain circumstances, Q₁⁺ and Q₂⁺ can be each, independently, an alkali metal.

In certain circumstances, Q₁⁺ and Q₂⁺ can be each, independently, selected from the group consisting of lithium, sodium, and potassium.

In certain circumstances, Q₁⁺ and Q₂⁺ can be each, independently, selected from the group consisting of ammonium and alkyl ammonium. For example, the alkyl ammonium can be a hydroxyalkyl ammonium.

In certain circumstances, Q₁⁺ and Q₂⁺ together can be selected from the group consisting of an alkaline earth metal salt.

In certain circumstances, Q₁⁺ and Q₂⁺ can be each independently selected from the group consisting of zinc, calcium and magnesium.

In certain circumstances, R₁ can be —C(═O)—R₄, and R₄ is C₁-C₆ alkyl or C₁-C₆ alkoxy.

In certain circumstances, R₁ can be an oxadiazole or a thiadiazole. The oxadiazole or thiadiazole can be substituted, for example, with a C1-C6 alkyl, haloalkyl, halo, amino, or hydroxy. The oxadiazole or thiadiazole can be a 1,3,4, 1,2,4 or 1,2,3 heterocycle.

In certain circumstances, n can be 0, 1, or 2.

In certain circumstances, Q₁⁺ and Q₂⁺ can be each independently lithium, sodium, or potassium, y can be 0, 1 or 2, and X can be F, Cl, or Br.

In certain circumstances, the phosphate derivative can be selected from the group consisting of:

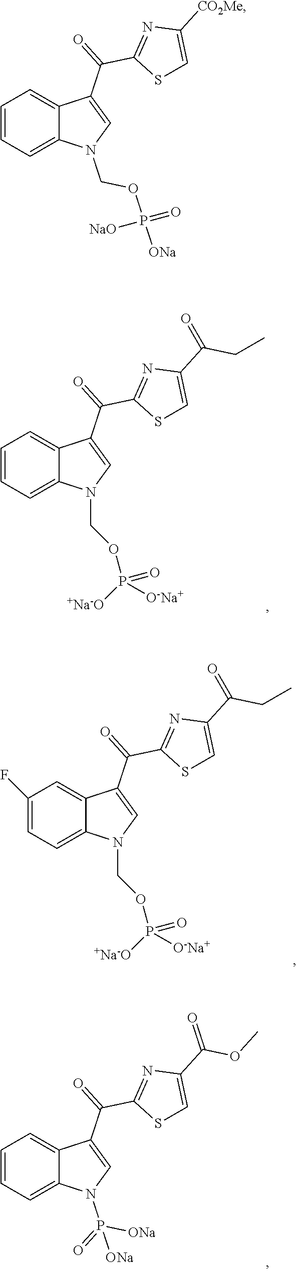

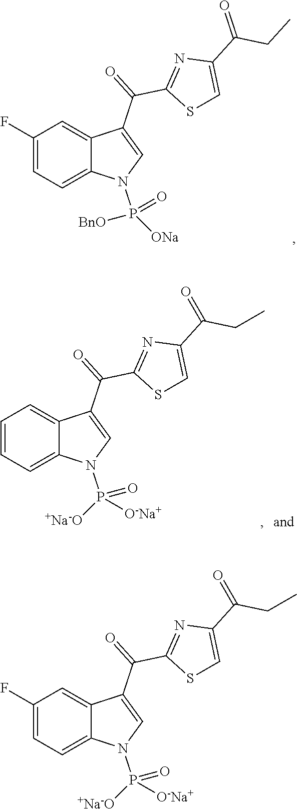

In certain circumstances, R₁ can be an unsubstituted or substituted oxadiazole.

In certain circumstances, n can be 0.

In certain circumstances, Q₁⁺ and Q₂⁺ each can be lithium, sodium, or potassium.

In certain circumstances, the phosphate derivative can be selected from the group consisting of:

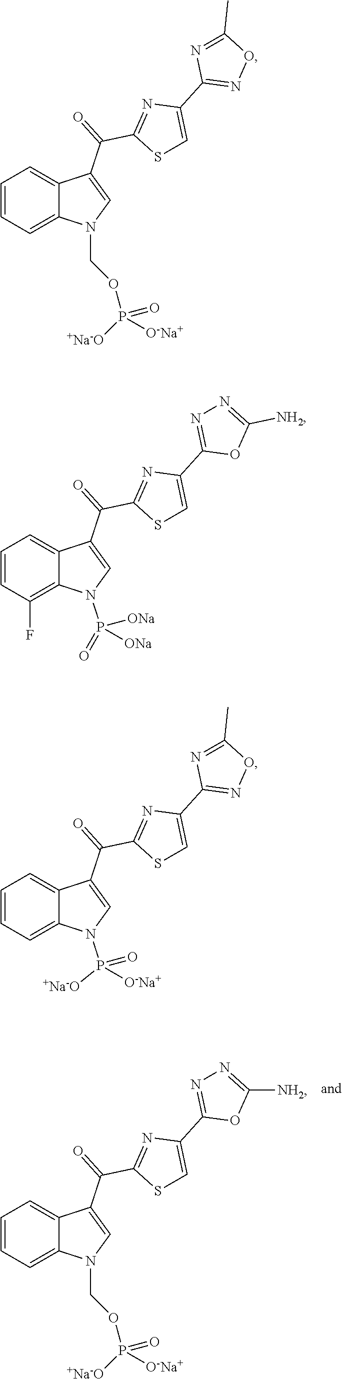

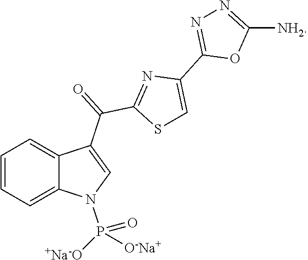

In certain circumstances, the phosphate derivative can be of Formula VI:

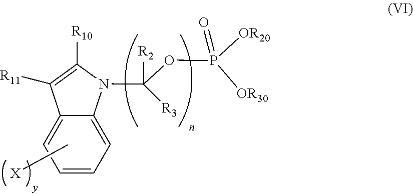

R₁₀ can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio.

R₁₁ can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio, wherein one of R₁₀ and R₁₁ can be H or C1-C6 alkyl.

R₂ can be H or C1-C6 alkyl, R₃ can be H or C1-C6 alkyl, or, together, R₂ and R₃ form a C3-C8 cycloalkyl.

y can be 0, 1, 2, 3, or 4.

Each X, independently, can be H or halogen.

R₂₀ and R₃₀ each, independently, can be H, C1-C6 alkyl or benzyl, or one of R₂₀ or R₃₀ is H, C1-C6 alkyl, allyl or benzyl and the other of R₂₀ or R₃₀ is a cation.

n can be 0, 1, 2, 3, 4, 5, or 6. Preferably, n can be 0 or 1.

Exemplary phosphate derivatives of indole compounds for the disclosed methods are shown in Table 3.

TABLE 3
Representative phosphate derivatives of indole compounds

| Compound | Structural Formula |
|---|---|
| A | 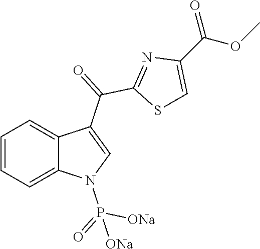 |
| B | 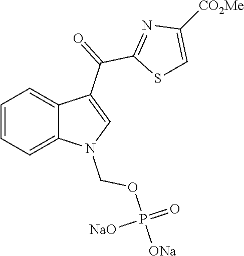 |
| C | 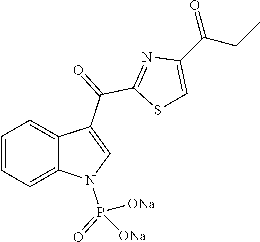 |
| D | 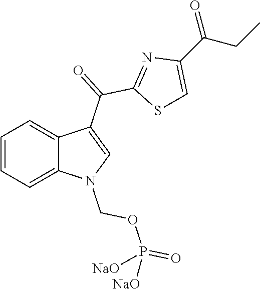 |
| E | 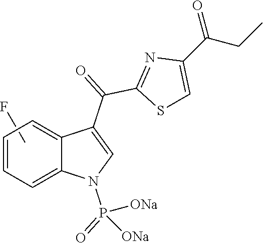 |
| F | 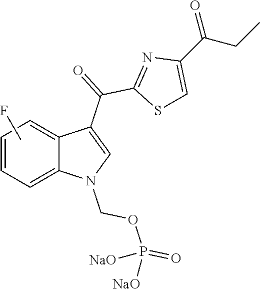 |
| G | 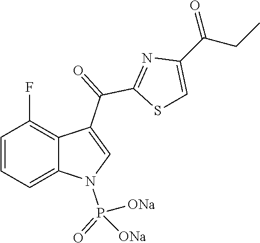 |
| H | 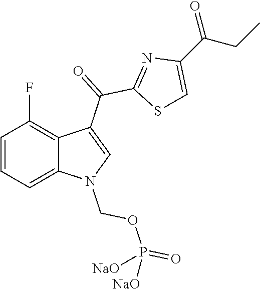 |
| I | 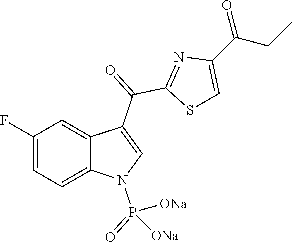 |
| J | 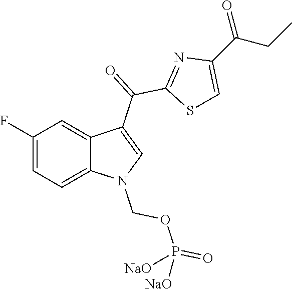 |
| K | 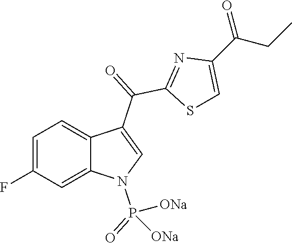 |
| L | 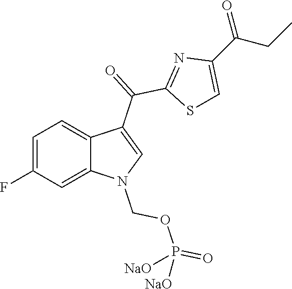 |
| M | 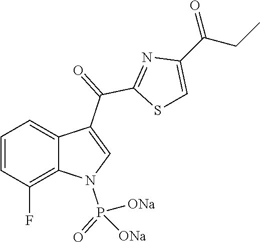 |
| N | 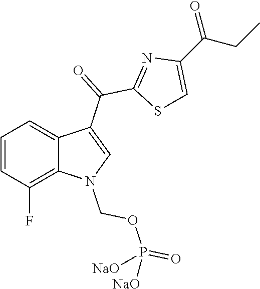 |
| O | 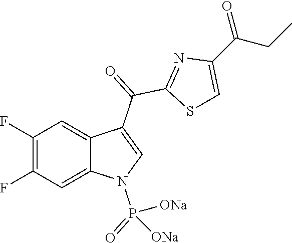 |
| P | 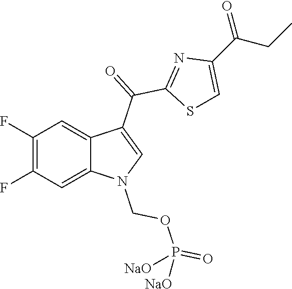 |
| Q | 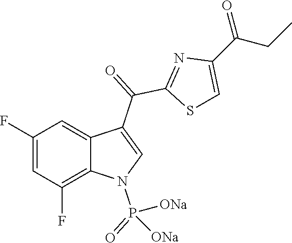 |
| R | 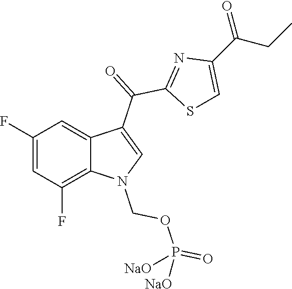 |
| S | 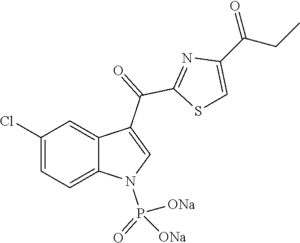 |
| T | 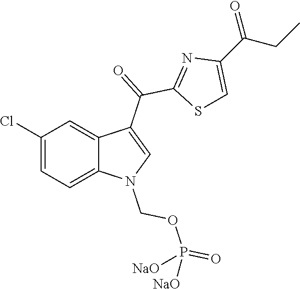 |
| U | 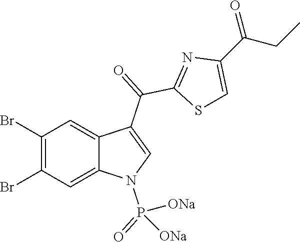 |
| V | 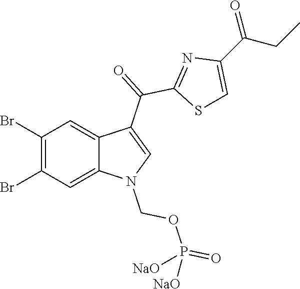 |
| W | 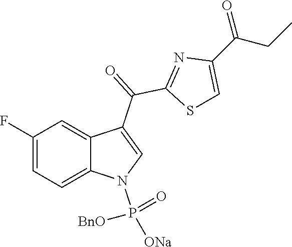 |
| X | 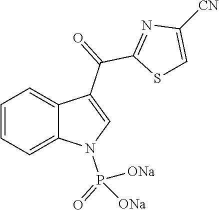 |
| Y | 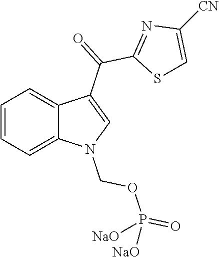 |
| Z | 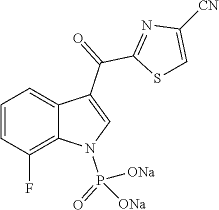 |
| AA | 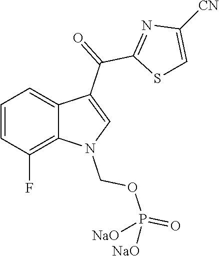 |
| AB | 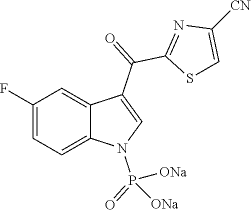 |
| AC | 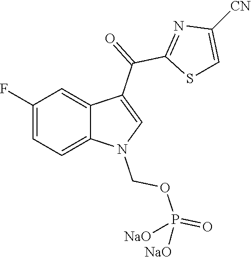 |
| AD | 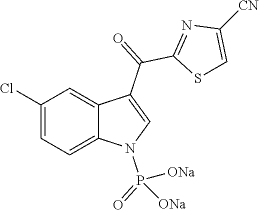 |
| AE | 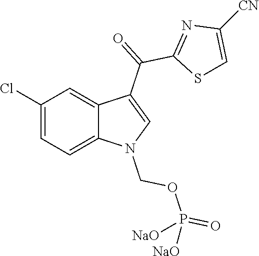 |
| AF | 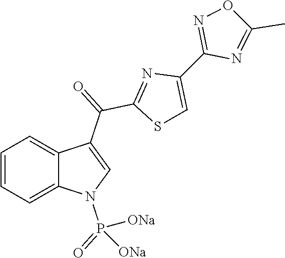 |
| AG | 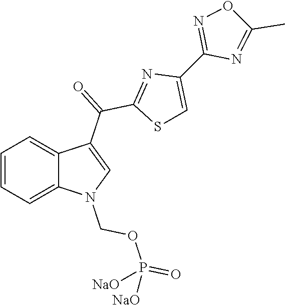 |
| AH | 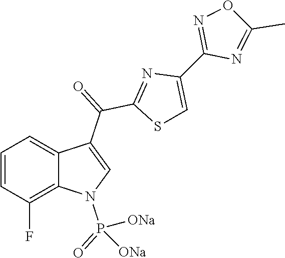 |
| AI | 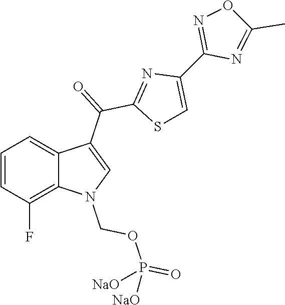 |
| AJ | 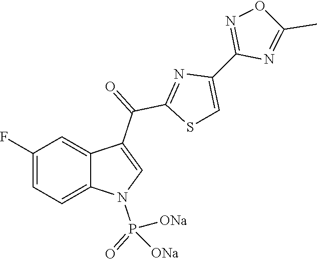 |
| AK | 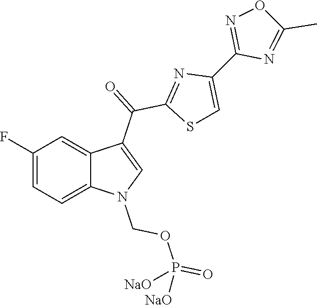 |
| AL | 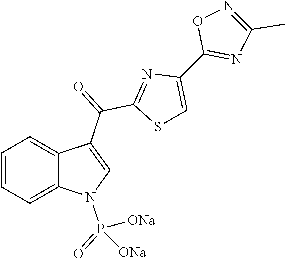 |
| AM | 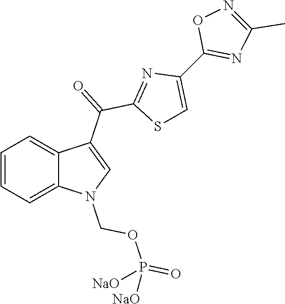 |
| AN | 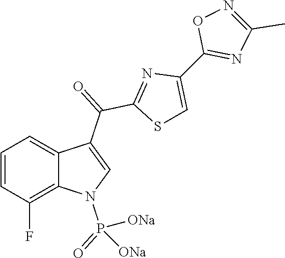 |
| AO | 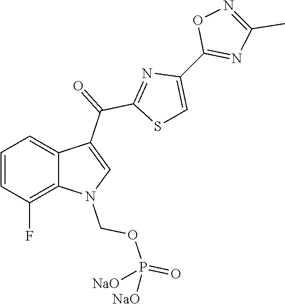 |
| AP | 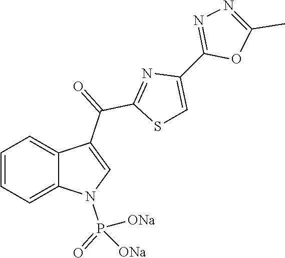 |
| AQ | 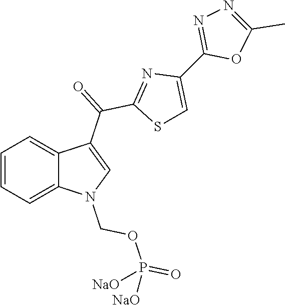 |
| AR | 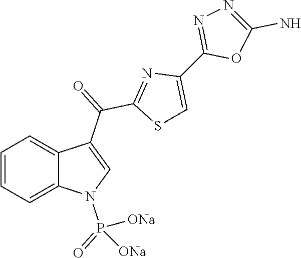 |
| AS | 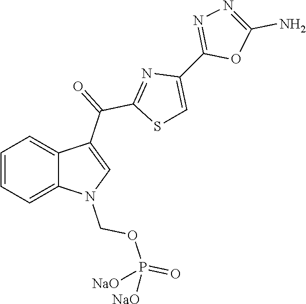 |
| AT | 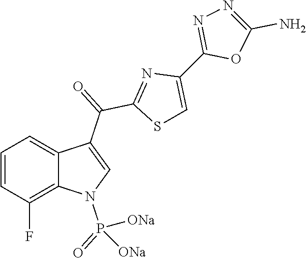 |
| AU | 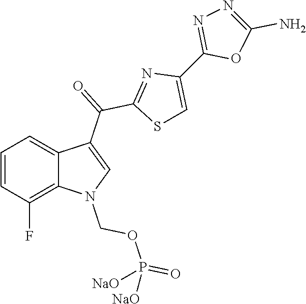 |
| AV | 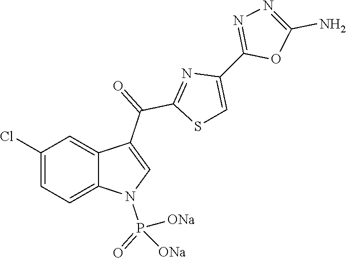 |
| AW | 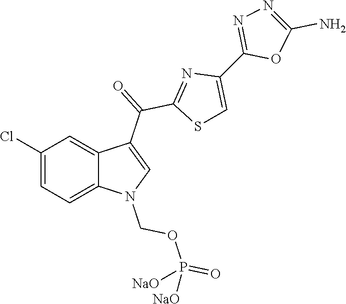 |
| AX | 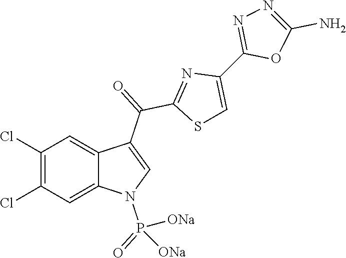 |
| AY | 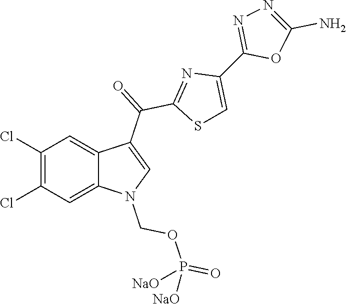 |
| AZ | 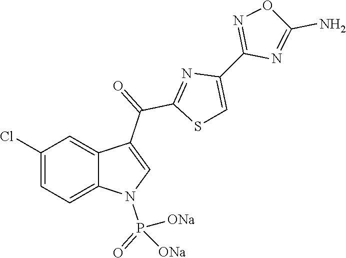 |
| BA | 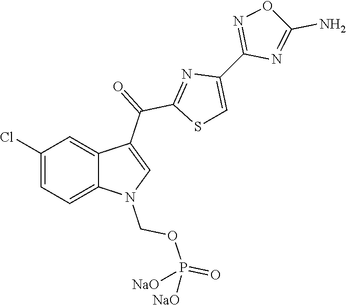 |
| BB | 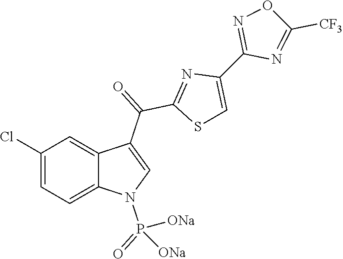 |
| BC | 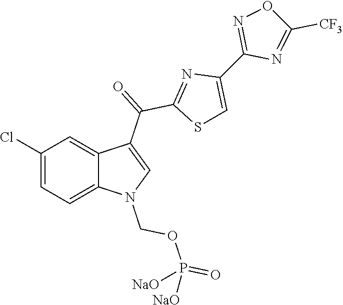 |
| BD | 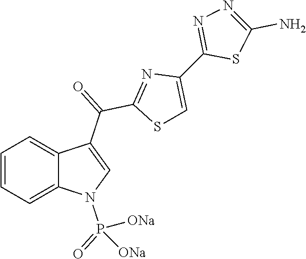 |
| BE | 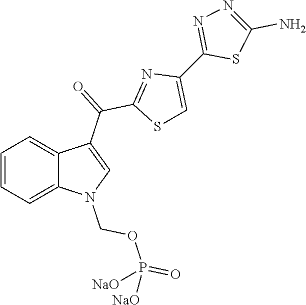 |
| BF | 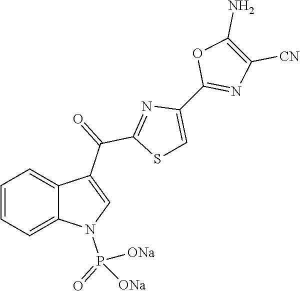 |
| BG | 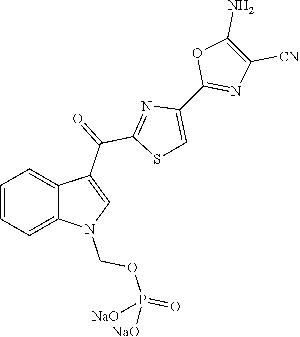 |
| BH | 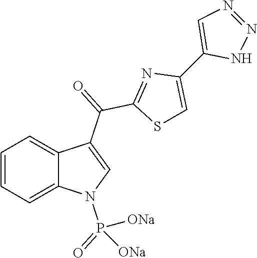 |
| BI | 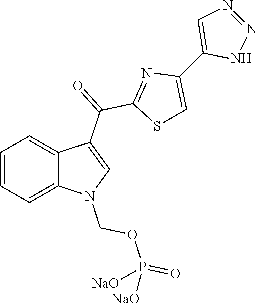 |
| BJ | 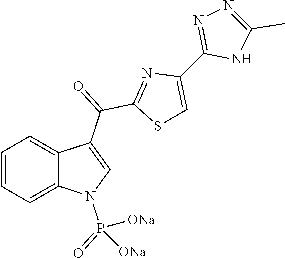 |
| BK | 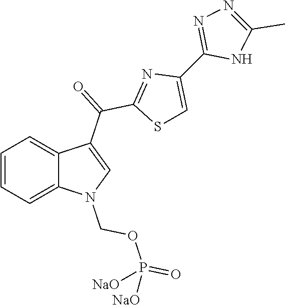 |
| BL | 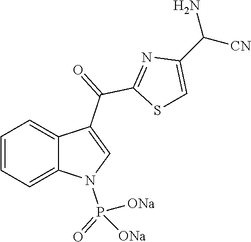 |
| BM | 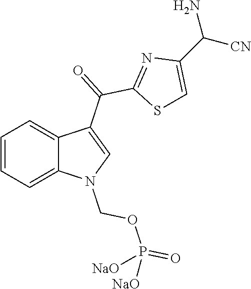 |
| BN | 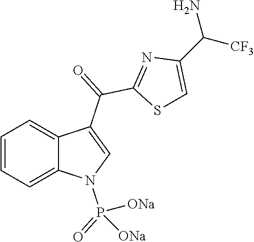 |
| BO | 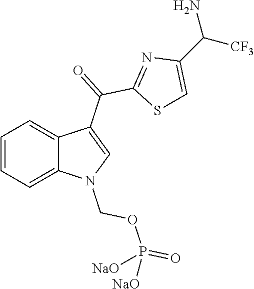 |
| BP | 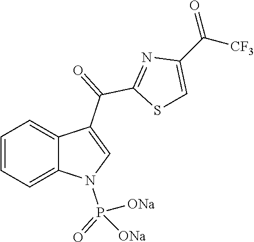 |
| BQ | 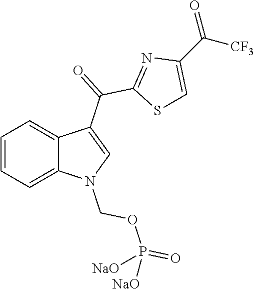 |

Compound A had the following spectroscopic characteristics: LC/MS ESI MS m/z 365 [M−H]⁺, ¹H NMR (500 MHz, D₂O) δ 9.01 (d, J=3.5 Hz, 1H), 8.66 (s, 1H), 8.22 (dd, J=6.5, 2.0 Hz, 1H), 7.96 (dd, J=6.0, 2.5 Hz, 1H), 7.39-7.33 (m, 2H), 3.93 (s, 3H).

Compound B had the following spectroscopic characteristics: LC/MS ESI MS m/z 395 [M−H]⁻, ¹H NMR (500 MHz, D₂O) δ 9.05 (d, J=3.5 Hz, 1H), 8.67-8.65 (m, 1H), 8.28-8.25 (m, 1H), 7.79 (d, J=8.0, Hz, 1H), 7.45-7.39 (m, 2H), 5.88 (dd, J=6.5, 2.5 Hz, 2H), 3.94 (s, 3H).

Compound C had the following spectroscopic characteristics: LC/MS ESI MS m/z 363 [M−H]⁻, ESI MS m/z 365 [M+H]⁺, ¹H NMR (500 MHz, D₂O) δ 9.13 (d, J=3.5 Hz, 1H), 8.66 (s, 1H), 8.25-8.24 (m, 1H), 7.97-7.95 (m, 1H), 7.38-7.34 (m, 2H), 3.23 (q, J=7.0 Hz, 2H), 1.15 (t, J=7.0 Hz, 3H). ³¹P NMR (202 MHz, D₂O) δ−4.56.

Compound I had the following spectroscopic characteristics: LC/MS ESI MS m/z 381 [M−J]⁻, ¹H NMR (500 MHz, D₂O) δ 9.13 (d, J=3.5 Hz, 1H), 8.62 (s, 1H), 7.92-7.88 (m, 2H), 7.11 (dt, J=9.5, 3.0 Hz, 1H), 3.21 (q, J=7.0 Hz, 2H), 1.15 (t, J=7.0 Hz, 3H); ¹⁹F NMR (470 MHz, D₂O) δ−120.60; ³¹P NMR (202 MHz, D₂O) δ−4.55.

Compound W had the following spectroscopic characteristics: LC/MS ESI MS m/z 471 [M−H]⁻, ¹H NMR (500 MHz, D₂O) δ 8.72 (d, J=3.5 Hz, 1H), 8.00 (s, 1H), 7.61 (dd, J=9.0, 4.5 Hz, 1H), 7.44 (dd, J=9.5, 2.0 Hz, 1H), 6.96 (dt, J=9.0, 2.5 Hz, 1H), 6.91-6.82 (m, 5H), 4.77 (d, J=11.0 Hz, 2H), 2.97 (q, J=7.0 Hz, 2H), 1.07 (t, J=7.5 Hz, 3H); ¹⁹F NMR (470 MHz, D₂O) δ−119.99; ³¹P NMR (202 MHz, D₂O) δ−8.19.

Compound AF had the following spectroscopic characteristics: LC/MS ESI MS m/z 390 [M−H]⁻, ¹H NMR (500 MHz, D₂O) δ 9.15 (d, J=3.5 Hz, 1H), 8.38 (d, J=0.5 Hz, 1H), 8.24-8.22 (m, 1H), 7.96-7.95 (m, 1H), 7.38-7.33 (m, 2H); ³¹P NMR (202 MHz, D₂O) δ−4.67.

Compound AQ had the following spectroscopic characteristics: LC/MS ESI MS m/z 391 [M+H]⁺, ¹H NMR (500 MHz, D₂O) δ 9.18 (d, J=4.0 Hz, 1H), 8.52 (s, 1H), 8.27-8.25 (m, 1H), 7.97-7.95 (m, 1H), 7.39-7.34 (m, 2H), 2.66 (s, 3H). ³¹P NMR (202 MHz, D₂O) δ−4.60.

Single stereochemical isomers, enantiomers, diastereomers, and pharmaceutically acceptable salts of the above exemplified phosphate derivatives are also within the scope of the present disclosure. Pharmaceutically acceptable salts may be, for example, derived from suitable inorganic and organic acids and bases.

Acid addition salts can be prepared by reacting the purified compound in its free-based form with a suitable organic or inorganic acid and isolating the salt thus formed. Examples of pharmaceutically acceptable acid addition salts include, without limitations, salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid.

Base addition salts can be prepared by reacting the purified compound in its acid form with a suitable organic or inorganic base and isolating the salt thus formed. Such salts include, without limitations, alkali metal (e.g., sodium, lithium, and potassium), alkaline earth metal (e.g., magnesium and calcium), ammonium, alkylammonium, substituted alkylammonium and N⁺(C₁₋₄alkyl)₄ salts. The alkyl can be a hydroxyalkyl.

Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, and valerate salts.

The phosphate derivatives of the present disclosure may be synthesized by methods known in the art or by methods illustrated in the Examples disclosed in U.S. Provisional Patent Application No. 62/734,989, filed Sep. 21, 2018.

### Inhibitors of Immune Checkpoint Proteins

Immune checkpoint proteins, such as cytotoxic T-lymphocyte-associated protein 4 (CTLA-4), programmed cell death protein 1 (PD-1) and its ligands PD-L1 and PD-L2, inhibit the immune system. The present treatment methods use a combination of an indole compound described herein and an inhibitor of any one of the above immune checkpoint proteins.

In particular embodiments, the inhibitor of an immune checkpoint protein is an anti-PD-1 antibody. Examples of anti-PD-1 antibodies are nivolumab, pembrolizumab, pidilizumab, MEDI0608 (formerly AMP-514; see, e.g., WO 2012/145493 and U.S. Pat. No. 9,205,148), PDR001 (see, e.g., WO 2015/112900), PF-06801591 (see, e.g., WO 2016/092419), BGB-A317 (see, e.g., WO 2015/035606), and cemiplimab (see, e.g.,WO 2015/112800).

In another particular embodiment, the inhibitor of an immune checkpoint protein is an anti-CTLA-4 antibody. Non-limiting examples of anti-CTLA-4 antibodies include ipilimumab and tremelimumab.

In another particular embodiment, the inhibitor of an immune checkpoint protein is an anti-PD-L1 antibody. Non-limiting examples of anti-PD-L1 antibodies include atezolizumab, avelumab, durvalumab, LY3300054, and BMS-936559.

### Pharmaceutical Compositions and Use

An aspect of the present disclosure relates to pharmaceutical compositions comprising one or more indole compounds, one or more phosphate derivatives of the indole compounds, or one or more inhibitors of immune checkpoint proteins disclosed herein formulated with one or more pharmaceutically acceptable excipients or carriers (carrier system). Combinations of the indole compounds or their phosphate derivatives and the inhibitors of immune checkpoint proteins may be co-formulated with one or more pharmaceutically acceptable excipients or carriers (carrier system).

The carrier system may include, for example, solvents, diluents, or other liquid vehicle, dispersion or suspension aids, surface active agents, isotonic agents, thickening or emulsifying agents, preservatives, fillers, extenders, disintegrating agents, solid binders, absorbents, lubricants, wetting agents, and the like. The pharmaceutical compositions can be administered to patients, for example, orally, or parenterally (e.g., subcutaneously, intravenously, or intramuscularly), intranasally, or topically. The pharmaceutical compositions may be provided, for example, in a form of cream, capsules, tablets, lozenges, or injectables.

When the inhibitors of immune checkpoint proteins are antibodies, e.g., anti-PD-1 antibodies, anti-CTLA-4 antibodies, and anti-PD-L1 antibodies, the antibodies can be formulated for suitable storage stability. For example, an antibody can be lyophilized or stored or reconstituted for use using pharmaceutically acceptable excipients.

The choice of excipient(s) will to a large extent depend on factors such as the particular mode of administration, the effect of the excipient on solubility and stability, and the nature of the dosage form. “Pharmaceutically acceptable excipient” includes any and all solvents, dispersion media, coatings, antibacterial and antifungal agents, isotonic and absorption delaying agents, and the like that are physiologically compatible. Some examples of pharmaceutically acceptable excipients are water, saline, phosphate buffered saline, dextrose, glycerol, ethanol and the like, as well as combinations thereof. In some cases, isotonic agents, for example, sugars, polyalcohols such as mannitol, sorbitol, or sodium chloride will be included in the composition. Additional examples of pharmaceutically acceptable substances are wetting agents or minor amounts of auxiliary substances such as wetting or emulsifying agents, preservatives or buffers.

The pharmaceutical compositions are typically suitable for parenteral administration, particularly when they comprise an antibody. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intratumoral, and intrasynovial injection or infusions; and kidney dialytic infusion techniques. Regional perfusion is also contemplated. Preferred embodiments may include the intravenous and the subcutaneous routes.

The individual indole compounds, phosphate derivatives of the indole compounds, and inhibitors of immune checkpoint proteins in the combination therapy of the present disclosure can be administered separately to the patient, in any order as deemed appropriate for the patient by the healthcare provider. They can also be administered simultaneously. The inhibitors of immune checkpoint proteins and the indole compounds or phosphate derivatives of the indole compounds in the combination therapy can be formulated in separate pharmaceutical compositions, or co-formulated in a single pharmaceutical composition or provided in a pharmaceutical kit.

In some embodiments of the disclosed methods, the patient has diffuse large B-cell lymphoma, marginal zone lymphoma, chronic lymphocytic leukemia, small lymphocytic lymphoma, prolymphocytic leukemia, acute lymphocytic leukemia, Waldenström's Macroglobulinemia, follicular lymphoma, mantle cell lymphoma, Hodgkin lymphoma, non-Hodgkin lymphoma, multiple myeloma, prostate cancer, ovarian cancer, fallopian tube cancer, cervical cancer, breast cancer, lung cancer such as non-small cell lung cancer, skin cancer such as melanoma, colon cancer, colorectal cancer, stomach cancer, pancreatic cancer, liver cancer, kidney cancer, bladder cancer, soft tissue cancer, glioma, or head and neck cancer. In a particular embodiment, the patient has colon cancer, breast cancer, pancreatic cancer, lung cancer, prostate cancer, kidney cancer, and melanoma. In another particular embodiment, the patient has a cancer refractory to an anti-PD-1 antibody treatment, such as colon cancer, breast cancer, lung cancer, and melanoma which are refractory to an anti-PD-1 antibody treatment.

“Treat”, “treating” and “treatment” refer to a method of alleviating or abrogating a biological disorder and/or at least one of its attendant symptoms. As used herein, to “alleviate” a disease, disorder or condition means reducing the severity and/or occurrence frequency of the symptoms of the disease, disorder, or condition. Further, references herein to “treatment” include references to curative, palliative and prophylactic treatment. Treatment of cancer encompasses inhibiting cancer growth (including causing partial or complete cancer regression), inhibiting cancer progression or metastasis, preventing cancer recurrence or residual disease, and/or prolonging the patient's survival. “A therapeutically effective amount” is an amount of the medication that can achieve the desired curative, palliative, or prophylactic effect for the treated condition.

In some embodiments, the effective dose range of an indole compound, a phosphate derivative of an indole compound, and an inhibitor of an immune checkpoint protein is determined by measuring the patient's blood concentration of the above agents under a specified dosing regimen to establish a concentration-time profile, consulting with an established correlation between the concentration-time profiles and effects on cancer inhibition or eradication obtained during a trial, and balancing the therapeutic effects achievable with possible toxicity to the patient, with further consideration of the health condition or physical durability of the patient. The dosing frequency of the compound may be determined similarly. The dosing may be continued until the patient is free from the cancer.

In some embodiments, an effective amount for tumor therapy may be measured by its ability to stabilize disease progression and/or ameliorate symptoms in a patient, and preferably to reverse disease progression, e.g., by reducing tumor size. In some embodiments, a maintenance dosing may be provided after the patient is free of cancer to ensure its complete elimination or eradication, or prevention of residual disease. The duration of the maintenance dosing can be determined based on clinical trial data.

It is contemplated that a suitable dose of an indole compound, a phosphate derivative of an indole compound, or an inhibitor of an immune checkpoint protein of the present disclosure may be in the range of 0.1-100 mg/kg, such as about 0.5-50 mg/kg, e.g., about 1-20 mg/kg. The compound may for example be administered in a dosage of at least 0.25 mg/kg, e.g., at least 0.5 mg/kg, such as at least 1 mg/kg, e.g., at least 1.5 mg/kg, such as at least 2 mg/kg, e.g., at least 3 mg/kg, such as at least 4 mg/kg, e.g., at least 5 mg/kg; and e.g., up to at most 50 mg/kg, such as up to at the most 30 mg/kg, e.g., up to at the most 20 mg/kg, such as up to at the most 15 mg/kg. Administration will normally be repeated at suitable intervals, e.g., twice a day, thrice a day, once a day, once every week, once every two weeks, or once every three weeks, and for as long as deemed appropriate by the responsible doctor, who may optionally increase or decrease the dosage as necessary.

In order that this invention may be better understood, the following examples are set forth. These examples are for purposes of illustration only and are not to be construed as limiting the scope of the invention in any manner.

### EXAMPLES

### Example 1: Preparation of methyl 2-(1H-indole-3-carbonothioyl)thiazole-4-carboxylate (ARI-007)

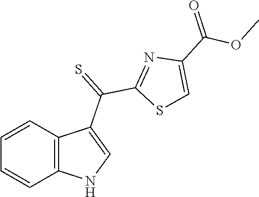 [SMILES: COC(=O)c1csc(C(=S)c2c[nH]c3ccccc23)n1]

Methyl 2-(1H-indole-3-carbonyl)thiazole-4-carboxylate (0.200 g, 0.699 mmol) and Lawesson's Reagent (0.113 g, 0.279 mmol) in THF (6.99 ml) were combined and the mixture heated to 65° C. Upon completion, the reaction was concentrated onto silica gel. Chromatography (silica gel, 0 to 100% heptane to ethyl acetate) followed by reverse phase chromatography (C18, 10% ACN/H₂O to ACN) gave methyl 2-(1H-indole-3-carbonothioyl)thiazole-4-carboxylate (75 mg) as a brown solid.

### Example 2: Preparation of (4-bromothiazol-2-yl)(1H-indol-3-yl)methanone (ARI-008)

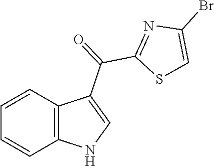 [SMILES: O=C(c1nc(Br)cs1)c1c[nH]c2ccccc12]

Step 1. Conducted by analogy to Org. Lett. 2016, 18, 3918-3921. To a solution of 1H-indole (360 mg, 3.07 mmol) in 3 mL of anhydrous THF was added potassium tert-butoxide (1 M in THF) (3.38 mL, 3.38 mmol). After stirring for 30 min, triethylborane (1 M in hexanes) (3.38 mL, 3.38 mmol) was added. After 30 min, the solution was cannulated slowly into an ice-cold mixture of 4-bromothiazole-2-carbonyl chloride (763 mg, 3.37 mmol) in THF (3 mL). Upon completion, the reaction was quenched with saturated NH₄Cl (aq). This mixture was extracted 3× with EtOAc. The combined organics were dried over Na₂SO₄, filtered and concentrated. Chromatography (silica gel, heptane to 10% MeOH/CH₂Cl₂) gave (4-bromothiazol-2-yl)(1H-indol-3-yl)methanone (760 mg) as an impure orange solid. ESI MS m/z 307 [M+H]⁺.

Step 2. To a suspension of (4-bromothiazol-2-yl)(1H-indol-3-yl)methanone (0.218 g, 0.710 mmol) and di-tert-butyl dicarbonate (0.214 ml, 0.923 mmol) in acetonitrile (7.10 ml) was added DMAP (0.026 g, 0.213 mmol). Upon completion, the reaction mixture was concentrated under reduced pressure onto silica gel. Chromatography (silica gel, 0 to 50% DCM/heptane) gave tert-butyl 3-(4-bromothiazole-2-carbonyl)-1H-indole-1-carboxylate (0.177 g) as a white solid. ESI MS m/z 407 [M+H]⁺.

Step 3. To a solution of tert-butyl 3-(4-bromothiazole-2-carbonyl)-1H-indole-1-carboxylate (0.100 g, 0.246 mmol) in dichloromethane (2.5 ml) was added trifluoroacetic acid (TFA) (0.500 ml). Upon completion, the reaction mixture was concentrated. Chromatography (silica gel, heptane to 40% ethyl acetate/heptane) gave (4-bromothiazol-2-yl)(1H-indol-3-yl)methanone (0.060 g) as a yellow solid.

### Example 3: Preparation of methyl (2-(1H-indole-3-carbonyl)thiazol-4-yl)carbamate (ARI-009)

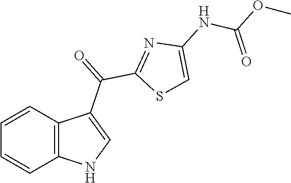 [SMILES: COC(=O)Nc1csc(C(=O)c2c[nH]c3ccccc23)n1]

Triethylamine (0.410 ml, 2.94 mmol) and diphenylphosphoryl azide (0.950 ml, 4.41 mmol) were added to an ice-cold mixture of 2-(1H-indole-3-carbonyl)thiazole-4-carboxylic acid (0.400 g, 1.469 mmol) in dioxane (2.94 ml) at 0° C. After 15 min, the ice bath was removed then methanol (16 ml, 395 mmol) was added dropwise over 10 minutes once gas evolution had ceased. The reaction mixture was stirred overnight. Water was added and the mixture was extracted with ethyl acetate 2×75 mL, then washed with brine, dried over sodium sulfate, filtered and concentrated onto silica gel. Chromatography (silica gel, heptane to 50% ethyl acetate/heptane) gave methyl (2-(1H-indole-3-carbonyl)thiazol-4-yl)carbamate (0.021 g) as a yellow solid.

### Example 4: Preparation of methyl 2-((1H-indol-3-yl)(methoxyimino)methyl)thiazole-4-carboxylate (ARI-011)

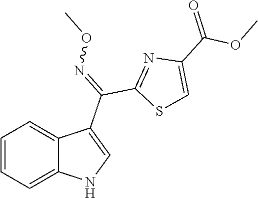 [SMILES: CON=C(c1nc(C(=O)OC)cs1)c1c[nH]c2ccccc12]

A mixture of 2-(1H-indole-3-carbonyl)thiazole-4-carboxylic acid (314 mg, 1.153 mmol) and O-methylhydroxylamine hydrochloride (450 mg, 5.39 mmol) in MeOH was heated in a microwave to 140° C. for 30 min. Upon completion, the mixture was concentrated to dryness. Chromatography (silica gel, heptane to DCM then to 10% MeOH/DCM) gave methyl 2-((1H-indol-3-yl)(methoxyimino)methyl)thiazole-4-carboxylate (163.8, mg) as a mixture of E/Z isomers and as an orange solid after lyophilization from acetonitrile/H₂O.

### Example 5: Preparation of S-methyl 2-(1H-indole-3-carbonyl)thiazole-4-carbothioate (ARI-013)

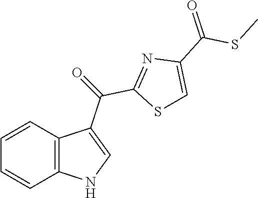 [SMILES: CSC(=O)c1csc(C(=O)c2c[nH]c3ccccc23)n1]

To a suspension of 2-(1H-indole-3-carbonyl)thiazole-4-carboxylic acid (0.500 g, 1.836 mmol) and di-tert-butyl dicarbonate (0.554 ml, 2.387 mmol) in acetonitrile (18.36 ml) was added DMAP (0.067 g, 0.551 mmol) and triethylamine (0.256 ml, 1.836 mmol). After consumption of starting material, sodium methyl mercaptide (0.167 g, 2.387 mmol) was added and the reaction mixture stirred overnight. The reaction mixture was then concentrated under reduced pressure and redissolved in EtOAc, then washed with saturated NH₄Cl. The organic layer was dried over magnesium sulfate, then absorbed onto silica gel. Chromatography (silica gel, 12 g, solid load, 0-80% CH₂Cl₂/hepatane) gave tert-butyl 3-(4-((methylthio)carbonyl)thiazole-2-carbonyl)-1H-indole-1-carboxylate (0.21 g, 0.522 mmol, 28.4% yield). ESI MS: m/z 403 [M+H]+.

To a solution of tert-butyl 3-(4-((methylthio)carbonyl)thiazole-2-carbonyl)-1H-indole-1-carboxylate (0.207 g, 0.514 mmol) in DCM (4 ml) was added TFA (1.8 ml). Upon completion, the reaction mixture was concentrated under reduced pressure and precipitated overnight from methanol to give S-methyl 2-(1H-indole-3-carbonyl)thiazole-4-carbothioate (0.116 g).

### Example 6: Preparation of methyl 2-((hydroxyimino)(1H-indol-3-yl)methyl)thiazole-4-carboxylate (ARI-014)

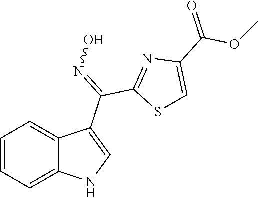

Methyl 2-(1H-indole-3-carbonyl)thiazole-4-carboxylate (0.300 g, 1.048 mmol) and hydroxylamine hydrochloride (0.218 g, 3.14 mmol) were combined with pyridine (11 ml). The reaction mixture was sealed and heated in the microwave at 130° C. Upon completion, the reaction mixture was concentrated onto silica gel. Chromatography (DCM to 1% MeOH/DCM) gave methyl 2-((hydroxyimino)(1H-indol-3-yl)methyl)thiazole-4-carboxylate (214.1, mg) as a yellow glass and as a mixture of E/Z isomers.

### Example 7: Preparation of methyl 2-(1-methyl-1H-indole-3-carbonyl)thiazole-4-carboxylate (ARI-015)

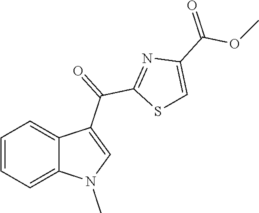 [SMILES: COC(=O)c1csc(C(=O)c2cn(C)c3ccccc23)n1]

To a suspension of methyl 2-(1H-indole-3-carbonyl)thiazole-4-carboxylate (111 mg, 0.388 mmol) in ice-cold THF (3877 μl) was added potassium hexamethyldisilazide (0.5 M in toluene) (814 μl, 0.407 mmol). DMF (500 uL) was added to improve solubility, the mixture stirred 10 min, then iodomethane (25.3 μl, 0.407 mmol) was added. The reaction mixture was quenched by the addition of anhydrous MeOH, then concentrated to dryness. Chromatography (silica gel, CH₂Cl₂ to 1% MeOH/CH₂Cl₂) gave methyl 2-(1-methyl-1H-indole-3-carbonyl)thiazole-4-carboxylate (66.7 mg) as a pale orange solid.

### Example 8: Preparation of methyl (S)-2-(1H-indole-3-carbonyl)-5,5-dimethyl-4,5-dihydrothiazole-4-carboxylatecarboxylate (ARI-016)

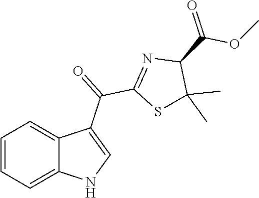 [SMILES: COC(=O)[C@@H]1N=C(C(=O)c2c[nH]c3ccccc23)SC1(C)C]

Step 1. 1H-indole-3-carbonyl cyanide (213 mg, 1.252 mmol) and (S)-2-amino-3-mercapto-3-methylbutanoic acid (187 mg, 1.252 mmol) were combined with DMF (12 mL) then the mixture treated with 1,8-diazabicyclo[5.4.0]undec-7-ene (18.72 μl, 0.125 mmol). The reaction mixture was heated to 40° C. Chromatography (silica gel, heptane to EtOAc+0.1% AcOH) gave (S)-2-(1H-indole-3-carbonyl)-5,5-dimethyl-4,5-dihydrothiazole-4-carboxylic acid (97 mg) as a white solid, ESI MS m/z 303 [M+H]⁺. Treatment of the solid with sodium methoxide (16.97 mg, 0.314 mmol) in MeOH (10 ml) gave sodium (S)-2-(1H-indole-3-carbonyl)-5,5-dimethyl-4,5-dihydrothiazole-4-carboxylate after the solvent was removed to dryness. The material was used as is.

Step 2. To a solution of sodium (S)-2-(1H-indole-3-carbonyl)-5,5-dimethyl-4,5-dihydrothiazole-4-carboxylate (102 mg, 0.314 mmol) in DMF (6280 μl) was added iodomethane (19.55 μl, 0.314 mmol). After the reaction was complete, the reaction was concentrated to dryness, then partitioned between EtOAc and water. The organic layer was dried with brine, filtered and concentrated. Chromatography (silica gel, CH₂Cl₂ to 6% MeOH/CH₂Cl₂) gave methyl (S)-2-(1H-indole-3-carbonyl)-5,5-dimethyl-4,5-dihydrothiazole-4-carboxylatecarboxylate (48.7 mg) as a light yellow solid.

### Example 9: Preparation of methyl 2-(1-(1H-indol-3-yl)-2-methoxyvinyl)thiazole-4-carboxylate (ARI-017)

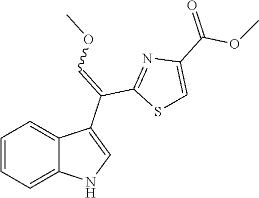 [SMILES: COC=C(c1nc(C(=O)OC)cs1)c1c[nH]c2ccccc12]

Step 1. To an ice-cold solution of (methoxymethyl)triphenylphosphonium chloride (322 mg, 0.939 mmol) in THF (8 mL) was added potassium hexamethyldisilazide (0.5 M in toluene) (1.708 mL, 0.854 mmol). After 30 min, solid methyl 2-(1-(tert-butoxycarbonyl)-1H-indole-3-carbonyl)thiazole-4-carboxylate (300 mg, 0.776 mmol) was added then allowed to slowly warm to room temperature. Upon completion, saturated NH₄Cl was added then after 15 min the reaction mixture was partitioned between EtOAc and saturated NH₄Cl. The organic layer was dried with brine and Na₂SO₄, and filtered. Chromatography (silica gel, heptane to 25% EtOAc/heptane) gave methyl 2-(1-(1-(tert-butoxycarbonyl)-1H-indol-3-yl)-2-methoxyvinyl)thiazole-4-carboxylate (276.9 mg) as a pale yellow solid. ESI MS m/z 415 [M+H]⁺.

Step 2. A mixture of methyl 2-(1-(1-(tert-butoxycarbonyl)-1H-indol-3-yl)-2-methoxyvinyl)thiazole-4-carboxylate (80 mg, 0.193 mmol) and K₂CO₃ (53.4 mg, 0.386 mmol) was stirred in MeOH (10 mL) with heating to 50° C. The reaction mixture was concentrated. Chromatography (silica gel, CH₂Cl₂ to 5% MeOH/CH₂Cl₂) gave methyl 2-(1-(1H-indol-3-yl)-2-methoxyvinyl)thiazole-4-carboxylate (5.7, mg) as an off-white solid and as a mixture of E/Z isomers.

### Example 10: Preparation of methyl 2-(1-(1H-indol-3-yl)prop-1-en-1-yl)thiazole-4-carboxylate (ARI-018)

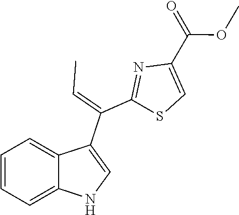 [SMILES: C/C=C(\c1nc(C(=O)OC)cs1)c1c[nH]c2ccccc12]

Prepared according to the method described in Example 9 except that (ethyl)triphenylphosphonium bromide was used instead of (methoxymethyl)triphenylphosphonium chloride.

### Example 11: Preparation of methyl 2-(1-(1H-indol-3-yl)vinyl)thiazole-4-carboxylate (ARI-019)

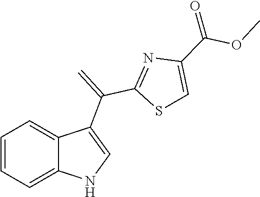 [SMILES: C=C(c1nc(C(=O)OC)cs1)c1c[nH]c2ccccc12]

Prepared according to the method described in Example 9 except that methyltriphenylphosphonium bromide was used instead of (methoxymethyl)triphenylphosphonium chloride.

### Example 12: Preparation of methyl 2-(1-(1H-indol-3-yl)-2-methylprop-1-en-1-yl)thiazole-4-carboxylate (ARI-020)

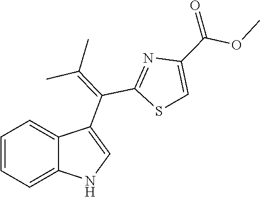 [SMILES: COC(=O)c1csc(C(=C(C)C)c2c[nH]c3ccccc23)n1]

Prepared according to the method described in Example 9 except that isopropyltriphenylphosphonium iodide was used instead of (methoxymethyl)triphenylphosphonium chloride.

### Example 13: Preparation of N-(2-(1H-indole-3-carbonyl)thiazol-4-yl)acetamide (ARI-021)

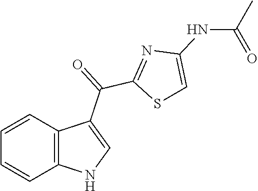 [SMILES: CC(=O)Nc1csc(C(=O)c2c[nH]c3ccccc23)n1]

Step 1. Diphenylphosphoryl azide (0.231 ml, 1.071 mmol) was added to a solution of 2-(1-(tert-butoxycarbonyl)-1H-indole-3-carbonyl)thiazole-4-carboxylic acid (0.266 g, 0.714 mmol) and triethylamine (0.199 ml, 1.429 mmol) in DMF (40 ml) at ambient temperature, then stirred for 30 min. After this time, water (2 ml) was added and the resulting mixture was heated to 80° C. for one hour. The reaction mixture was cooled to ambient temperature, and water was added (50 mL), and the resulting mixture was then extracted with ethyl acetate (3×50 mL). The organic layers were pooled, washed with brine, and dried over sodium sulfate, then filtered and concentrated onto silica gel under reduced pressure. Chromatography (silica gel, heptane to 30% ethyl acetate/heptane) gave tert-butyl 3-(4-aminothiazole-2-carbonyl)-1H-indole-1-carboxylate (0.116 g). ESI MS m/z 344 [M+H]⁺.

Step 2. Acetyl chloride (0.025 ml, 0.352 mmol) was added to an ice-cold solution of tert-butyl 3-(4-aminothiazole-2-carbonyl)-1H-indole-1-carboxylate (0.110 g, 0.320 mmol) and triethylamine (0.067 ml, 0.480 mmol) in dichloromethane (21 ml). Upon completion, potassium carbonate (0.144 g, 0.320 mmol) and methanol (10.50 ml) were added to remove the Boc group. Upon completion, water was added to the reaction and the mixture extracted with ethyl acetate. The organic was washed with brine wash, dried over magnesium sulfate, filtered and the crude was concentrated onto silica gel. Chromatography (silica gel, heptane to 80% ethyl acetate/heptane) gave N-(2-(1H-indole-3-carbonyl)thiazol-4-yl)acetamide (44.2 mg).

### Example 14: Preparation of 3-hydroxypropyl 2-(1H-indole-3-carbonyl)thiazole-4-carboxylate (ARI-022)

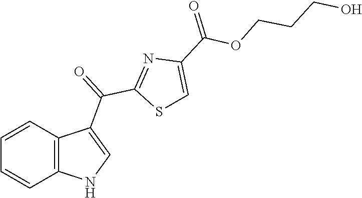 [SMILES: O=C(OCCCO)c1csc(C(=O)c2c[nH]c3ccccc23)n1]

To an ice-cold solution of 2-(1H-indole-3-carbonyl)thiazole-4-carboxylic acid (0.300 g, 1.102 mmol) and 4-(dimethylamino)pyridine (0.013 g, 0.110 mmol) in tetrahydrofuran (11.02 ml) was sequentially added triethylamine (0.192 ml, 1.377 mmol), 1,3-propanediol (0.735 ml, 11.02 mmol), and 3-(((ethylimino)methylene)amino)-N,N-dimethylpropan-1-amine hydrochloride (0.232 g, 1.212 mmol). The reaction mixture was warmed to ambient temperature and stirred. Upon completion, 1M HCl (aq) was added and the subsequent mixture extracted with EtOAc. The combined organics were washed with water, sodium bicarbonate and brine. The crude was filtered and concentrated onto silica gel. Chromatography (silica gel, DCM to 5% MeOH/DCM) gave 3-hydroxypropyl 2-(1H-indole-3-carbonyl)thiazole-4-carboxylate (0.126 g) as a yellow solid.

### Example 15: Preparation of 2-hydroxyethyl 2-(1H-indole-3-carbonyl)thiazole-4-carboxylate (ARI-023)

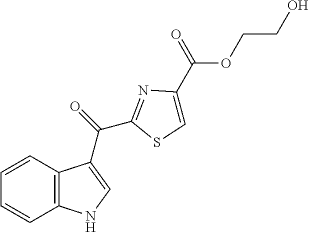 [SMILES: O=C(OCCO)c1csc(C(=O)c2c[nH]c3ccccc23)n1]

Prepared according to the method described in Example 14 except that ethylene glycol was used instead of 1,3-propanediol.

### Example 16: Preparation of methyl 2-((1H-indol-3-yl)(methoxy)methyl)thiazole-4-carboxylate (ARI-024)

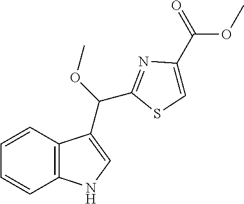 [SMILES: COC(=O)c1csc(C(OC)c2c[nH]c3ccccc23)n1]

Sodium borohydride (0.092 g, 2.445 mmol) was added portionwise to a mixture of methyl 2-(1H-indole-3-carbonyl)thiazole-4-carboxylate (0.200 g, 0.699 mmol) in tetrahydrofuran (6.99 mL) and methanol (6.99 mL). Upon completion, the reaction mixture was quenched with 1M HCl then extracted with DCM. The organic was washed with brine, and dried over sodium sulfate then filtered. Chromatography (silica gel, DCM to 5% MeOH/DCM) gave methyl 2-((1H-indol-3-yl)(methoxy)methyl)thiazole-4-carboxylate (0.035 g) as a pink solid.

### Example 17: Preparation of 2-(2-hydroxyethoxy)ethyl 2-(1H-indole-3-carbonyl)thiazole-4-carboxylate (ARI-025)

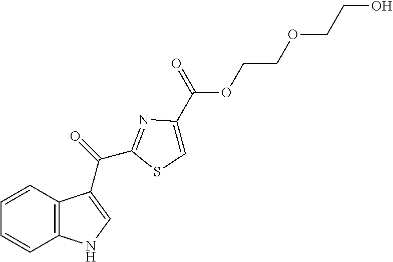 [SMILES: O=C(OCCOCCO)c1csc(C(=O)c2c[nH]c3ccccc23)n1]

Prepared according to the method described in Example 14 except that diethylene glycol was used instead of 1,3-propanediol.

### Example 18: Preparation of 2-(1H-indole-3-carbonyl)thiazole-4-carbonitrile (ARI-026)

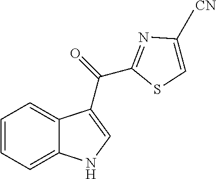

Triethylamine (2.57 ml, 18.43 mmol) was added to an ice-cold suspension of 2-(1H-indole-3-carbonyl)thiazole-4-carboxamide (see WO2018121434A1) (1.00 g, 3.69 mmol) in tetrahydrofuran (36.9 ml). Subsequently trifluoroacetic anhydride (1.302 ml, 9.22 mmol) was added dropwise. The ice bath was removed. Upon completion, the reaction mixture was poured over ice and diluted with ethyl acetate. The organic layer was washed with 2M Na₂CO₃ and brine, dried over sodium sulfate, filtered and concentrated onto silica gel. Chromatography (silica gel, heptane to 50% EtOAc/heptane) gave 2-(1H-indole-3-carbonyl)thiazole-4-carbonitrile (0.720 g) as a yellow solid.

### Example 19: Preparation of (4-(1,3-dioxolan-2-yl)thiazol-2-yl)(1H-indol-3-yl)methanone (ARI-028)

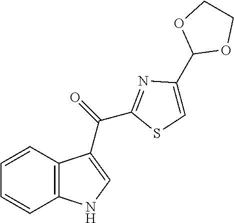 [SMILES: O=C(c1nc(C2OCCO2)cs1)c1c[nH]c2ccccc12]

To a solution of tert-butyl 3-(4-formylthiazole-2-carbonyl)-1H-indole-1-carboxylate (0.200 g, 0.561 mmol) and ethylene glycol (0.094 ml, 1.684 mmol) in dioxane (5.61 ml) was added p-toluenesulfonic acid monohydrate (1.067 mg, 5.61 μmol) and the mixture heated to 50° C. Upon completion, potassium carbonate (0.116 g, 0.842 mmol) and MeOH was added to remove the Boc group. Upon completion, water was added and the pH of the solution was adjusted to 8 with 1M HCl. The mixture was extracted with ethyl acetate and the organic layer was washed with brine, then concentrated onto silica gel. Chromatography (silica gel, heptane to 50% EtOAc/heptane) gave (4-(1,3-dioxolan-2-yl)thiazol-2-yl)(1H-indol-3-yl)methanone (73.4 mg) as a yellow solid.

### Example 20: Preparation of (4-(dimethoxymethyl)thiazol-2-yl)(1H-indol-3-yl)methanone (ARI-029)

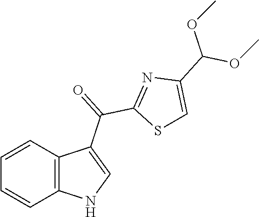 [SMILES: COC(OC)c1csc(C(=O)c2c[nH]c3ccccc23)n1]

Prepared according to the method described in Example 19 except that methanol was used instead of ethylene glycol.

### Example 21: Preparation of (1H-indol-3-yl)(4-(5-methyl-1,2,4-oxadiazol-3-yl)thiazol-2-yl)methanone (ARI-030)

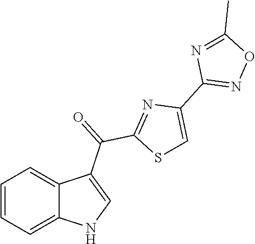 [SMILES: Cc1nc(-c2csc(C(=O)c3c[nH]c4ccccc34)n2)no1]

A mixture of 2-(1H-indole-3-carbonyl)thiazole-4-carbonitrile (50 mg, 0.197 mmol), K₃PO₄ (126 mg, 0.592 mmol) and hydroxylamine hydrochloride (34.3 mg, 0.494 mmol) in DMF (2.5 ml) was heated to 100° C. with a microwave for 30 min. Acetyl chloride (0.028 ml, 0.395 mmol) was added and the reaction heated to 110° C. for 2 hr. The DMF was removed under high vacuum. Added water, sonicated then collected the solid by filtration. The solid was rinsed with H₂O, then dried at 50° C. under high vacuum to give (1H-indol-3-yl)(4-(5-methyl-1,2,4-oxadiazol-3-yl)thiazol-2-yl)methanone (34 mg) as a yellow solid.

### Example 22: Preparation of (1H-indol-3-yl)(4-(3-methyl-1,2,4-oxadiazol-5-yl)thiazol-2-yl)methanone (ARI-031)

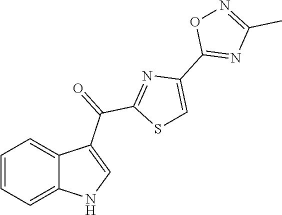 [SMILES: Cc1noc(-c2csc(C(=O)c3c[nH]c4ccccc34)n2)n1]

Step 1. To 2-(1-(tert-butoxycarbonyl)-1H-indole-3-carbonyl)thiazole-4-carboxylic acid (200 mg, 0.537 mmol), N-hydroxyacetamidine (39.8 mg, 0.537 mmol) and triethylamine (299 μl, 2.148 mmol) in ethyl acetate (2685 μl) was added 1-propanephosphonic acid cyclic anhydride (50 wt % in EtOAc) (799 μl, 1.343 mmol) dropwise. The mixture was heated to 80° C. Upon completion, saturated NaHCO₃ (aq) was added and the solid collected by filtration. Washed the solid with H₂O, and then with a minimum of EtOAc. The solid was dried under high vacuum at 50° C. to give tert-butyl 3-(4-(3-methyl-1,2,4-oxadiazol-5-yl)thiazole-2-carbonyl)-1H-indole-1-carboxylate as an off-white solid. ESI MS m/z 411 [M+H]⁺.

Step 2. To tert-butyl 3-(4-(3-methyl-1,2,4-oxadiazol-5-yl)thiazole-2-carbonyl)-1H-indole-1-carboxylate (99 mg, 0.241 mmol) was added K₂CO₃ (33.3 mg, 0.241 mmol) and MeOH (5 ml). Upon reaction completion, silica gel was added and the mixture concentrated. Chromatography (silica gel, heptane to 40% EtOAc/heptane) gave (1H-indol-3-yl)(4-(3-methyl-1,2,4-oxadiazol-5-yl)thiazol-2-yl)methanone (64 mg) as a yellow solid after drying overnight at 50° C. in the vacuum oven.

### Example 23: Preparation of methyl 6-(1H-indole-3-carbonyl)picolinate (ARI-032)

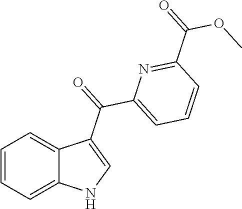 [SMILES: COC(=O)c1cccc(C(=O)c2c[nH]c3ccccc23)n1]

Prepared according to the method described in Example 2 except that methyl 6-(chlorocarbonyl)picolinate hydrochloride was used instead of 4-bromothiazole-2-carbonyl chloride in step 1 and the Boc deprotection was effected as follows. Methyl 6-(1H-indole-3-carbonyl)picolinate (1197 mg, 4.27 mmol) was treated with sodium sulfate (606 mg, 4.27 mmol) and stirred in anhydrous MeOH (50 ml) for 30 min then K₂CO₃ (177 mg, 1.281 mmol) was added. Upon completion, the mixture was filtered through Celite, silica gel was added and concentrated to dryness. Chromatography (silica gel, heptane to EtOAc) and then reverse phase (C18, H₂O to CH₃CN) gave methyl 6-(1H-indole-3-carbonyl)picolinate as an off-white solid.

### Example 24: Preparation of N-ethyl-2-(1H-indole-3-carbonyl)thiazole-4-carboxamide (ARI-033)

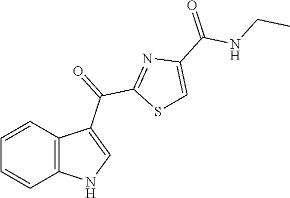 [SMILES: CCNC(=O)c1csc(C(=O)c2c[nH]c3ccccc23)n1]

Propylphosphonic anhydride (0.352 ml, 0.591 mmol, 50 wt % in EtOAc) was added to a solution of 2-(1-(tert-butoxycarbonyl)-1H-indole-3-carbonyl)thiazole-4-carboxylic acid (0.200 g, 0.537 mmol), ethanamine (0.322 ml, 0.644 mmol), and DIPEA (0.141 ml, 0.806 mmol) in N,N-dimethylformamide (5.37 ml). Upon completion, the Boc group was removed by adding potassium carbonate (0.300 g, 2.171 mmol) and 10 mL of MeOH and heating the mixture to 50° C. Upon completion, the mixture was concentrated under reduced pressure then to it added 20 mL of water, followed by neutralization with 1M HCl to pH 7. This mixture was extracted with 3×40 mL ethyl acetate. The combined organic layers were washed with 50 mL of 5% LiCl and brine, then concentrated under reduced pressure onto silica gel. Chromatography (silica gel, DCM to 5% MeOH/DCM) gave N-ethyl-2-(1H-indole-3-carbonyl)thiazole-4-carboxamide (89.5 mg) as a yellow solid.

### Example 25: Preparation of 2-(1H-indole-3-carbonyl)-N-isopropylthiazole-4-carboxamide (ARI-034)

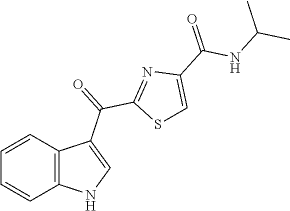 [SMILES: CC(C)NC(=O)c1csc(C(=O)c2c[nH]c3ccccc23)n1]

Prepared according to the method described in Example 24 except that isopropylamine was used instead of ethylamine.

### Example 26: Preparation of 2-(1H-indole-3-carbonyl)-N-isobutylthiazole-4-carboxamide (ARI-035)

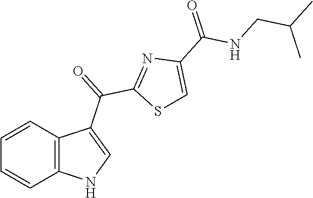 [SMILES: CC(C)CNC(=O)c1csc(C(=O)c2c[nH]c3ccccc23)n1]

Prepared according to the method described in Example 24 except that isobutylamine was used instead of ethylamine.

### Example 27: Preparation of N-(2-hydroxyethyl)-2-(1H-indole-3-carbonyl)thiazole-4-carboxamide (ARI-036)

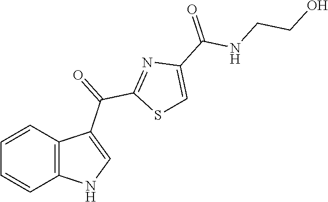 [SMILES: O=C(NCCO)c1csc(C(=O)c2c[nH]c3ccccc23)n1]

To a solution of 2-(1H-indole-3-carbonyl)thiazole-4-carboxylic acid (0.200 g, 0.735 mmol) and hexafluorophosphate azabenzotriazole tetramethyl uronium(HATU) (0.335 g, 0.881 mmol) in N,N-dimethylformamide (7.35 ml) was added N,N-diisopropylethylamine (DIPEA) (0.385 ml, 2.204 mmol) and then ethanolamine (0.222 ml, 3.67 mmol). Upon completion, water was added and the reaction mixture was concentrated under reduced pressure to remove DMF. The reaction mixture was partitioned between EtOAc and H₂O. The organic phase was successively washed with 1M HCl, H₂O, saturated NaHCO₃, and brine, then dried over Na₂SO₄. After filtration, the crude solution was concentrated onto silica gel. Chromatography (DCM to 25% 80:18:2 CH₂Cl₂/MeOH/conc. NH4OH) gave N-(2-hydroxyethyl)-2-(1H-indole-3-carbonyl)thiazole-4-carboxamide (0.164 g) as a yellow solid.

### Example 28: Preparation of 2-(1H-indole-3-carbonyl)-N-(2-methoxyethyl)thiazole-4-carboxamide (ARI-037)

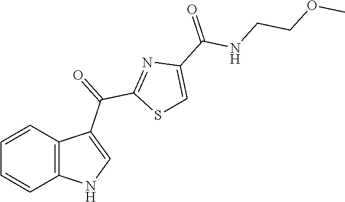 [SMILES: COCCNC(=O)c1csc(C(=O)c2c[nH]c3ccccc23)n1]

Prepared according to the method described in Example 27 except that 2-methoxyethan-1-amine was used instead of ethanolamine.

### Example 29: Preparation of methyl 2-(1-cyano-1H-indole-3-carbonyl)thiazole-4-carboxylate (ARI-038)

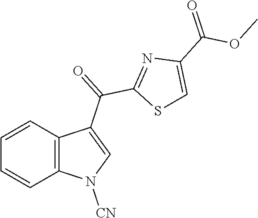 [SMILES: COC(=O)c1csc(C(=O)c2cn(C#N)c3ccccc23)n1]

Cyanogen bromide (0.740 g, 6.99 mmol) was added to an ice cold solution of methyl 2-(1H-indole-3-carbonyl)thiazole-4-carboxylate (0.500 g, 1.746 mmol) and cesium carbonate (0.683 g, 2.096 mmol) in acetonitrile (17.46 ml). The reaction suspension was filtered and rinsed with acetonitrile. The filtrate was concentrated under reduced pressure and then treated with boiling DCM and filtered. The filtrate was directly loaded onto a silica gel column. Chromatography (heptane to 50% EtOAc/heptane) gave methyl 2-(1-cyano-1H-indole-3-carbonyl)thiazole-4-carboxylate (0.046 g) as a white solid.

### Example 30: Preparation of N-(tert-butyl)-2-(1H-indole-3-carbonyl)thiazole-4-carboxamide (ARI-039)

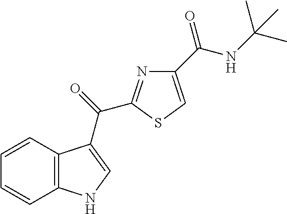 [SMILES: CC(C)(C)NC(=O)c1csc(C(=O)c2c[nH]c3ccccc23)n1]

Prepared according to the method described in Example 24 except that tert-butylamine was used instead of ethylamine.

### Example 31: Preparation of 1-(2-(1H-indole-3-carbonyl)thiazol-4-yl)pyrrolidine-2,5-dione (ARI-040)

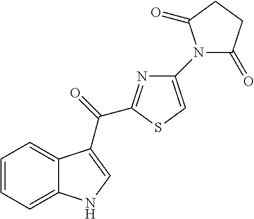 [SMILES: O=C(c1nc(N2C(=O)CCC2=O)cs1)c1c[nH]c2ccccc12]

To a mixture of (4-bromothiazol-2-yl)(1H-indol-3-yl)methanone (217 mg, 0.533 mmol) and succinimide (79 mg, 0.799 mmol) in 2,4,6-trimethylpyridine (1 ml, 0.533 mmol) was added cuprous oxide (45.7 mg, 0.320 mmol). The mixture was then heated in a microwave to 175° C. for 7 hrs. The mixture was diluted with CH₂Cl₂ and then 5% H₂SO₄ (aq) was added. The solid was removed by filtration. The filtrate was treated with 5% H₂SO₄ (aq) then extracted with 2× with CH₂Cl₂. The combined organic extracts were dried over Na₂SO₄, filtered and concentrated. Chromatography (silica gel, DCM to 1% MeOH/DCM, dry loaded on Celite) gave 1-(2-(1H-indole-3-carbonyl)thiazol-4-yl)pyrrolidine-2,5-dione (7 mg) as a yellow solid.

### Example 32: Preparation of (2-(1H-indole-3-carbonyl)thiazol-4-yl)(azetidin-1-yl)methanone (ARI-044)

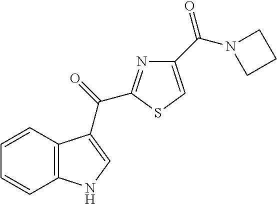 [SMILES: O=C(c1nc(C(=O)N2CCC2)cs1)c1c[nH]c2ccccc12]

Prepared according to the method described in Example 27 except that azetidine hydrochloride was used instead of ethanolamine.

### Example 33: Preparation of (2-(1H-indole-3-carbonyl)thiazol-4-yl)(3-methoxyazetidin-1-yl)methanone (ARI-046)

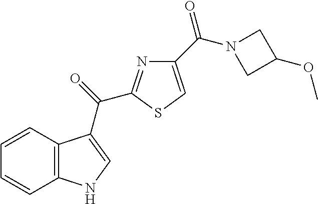 [SMILES: COC1CN(C(=O)c2csc(C(=O)c3c[nH]c4ccccc34)n2)C1]

Prepared according to the method described in Example 27 except that 3-methoxyazetidine hydrochloride was used instead of ethanolamine.

### Example 34: Preparation of 6-(1H-indole-3-carbonyl)-N-methylpicolinamide (ARI-047)

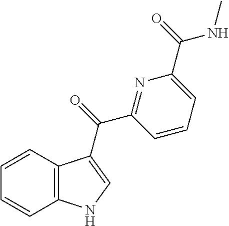 [SMILES: CNC(=O)c1cccc(C(=O)c2c[nH]c3ccccc23)n1]

Step 1. Sodium hydroxide (0.813 ml, 0.813 mmol) was added to a stirring solution of methyl 6-(1H-indole-3-carbonyl)picolinate (0.228 g, 0.813 mmol) in tetrahydrofuran (4.5 ml) and water (3.7 ml). Upon completion, the reaction mixture was diluted with water and extracted with 30 mL of EtOAc to remove unreacted ester. The aqueous layer was adjusted to pH 5 with 1M HCl, then extracted with EtOAc. The organic was washed with brine and dried over Na₂SO₄, and filtered. The crude was concentrated onto silica gel. Chromatography (C18, H₂O to 60% ACN/water) gave 6-(1H-indole-3-carbonyl)picolinic acid (0.185 g, 0.695 mmol, 85% yield) as a yellow solid. ESI MS m/z 267 [M+H]⁺.

Step 2. Prepared according to the method described in Example 27 except that methylamine in THF was used instead of ethanolamine.

### Example 35: Preparation of 2-(hydrazineylidene(1H-indol-3-yl)methyl)-4-(5-methyl-4H-1,2,4-triazol-3-yl)thiazole (ARI-050)

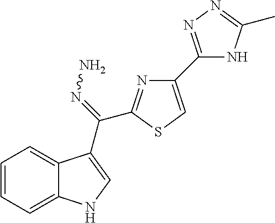 [SMILES: Cc1nnc(-c2csc(C(=NN)c3c[nH]c4ccccc34)n2)[nH]1]

Step 1. To a mixture of 2-(1-(tert-butoxycarbonyl)-1H-indole-3-carbonyl)thiazole-4-carboxylic acid (60 mg, 0.161 mmol), ammonium chloride (60.3 mg, 1.128 mmol), HOBt (37.0 mg, 0.242 mmol), and ethylene dichloride (EDC) (93 mg, 0.483 mmol) was added N,N-dimethylformamide (1.6 mL) and then DIPEA (0.169 mL, 0.967 mmol). Upon completion, the reaction mixture was diluted with water and saturated NaHCO₃. The precipitate was collected, washed with water, and dried in vacuo to provide tert-butyl 3-(4-carbamoylthiazole-2-carbonyl)-1H-indole-1-carboxylate (56.6 mg, 0.152 mmol) as a yellow solid. ESI MS m/z 372 [M+H]⁺.

Step 2. A mixture of tert-butyl 3-(4-carbamoylthiazole-2-carbonyl)-1H-indole-1-carboxylate (56.6 mg, 0.152 mmol) and 1,1-dimethoxy-N,N-dimethylethan-1-amine (1.0 mL, 6.84 mmol) was stirred at 80° C. Upon completion, the reaction mixture was cooled to room temperature and concentrated to a dark brown viscous oil. The oil was dissolved in acetic acid (1.0 ml) then hydrazine hydrate (24 μL, 0.762 mmol) was added. The reaction mixture was stirred at 80° C. for 1 h. The reaction mixture was concentrated to dryness. Chromatography (C18, H₂O to 60% MeCN) gave 2-(hydrazineylidene(1H-indol-3-yl)methyl)-4-(5-methyl-4H-1,2,4-triazol-3-yl)thiazole (18 mg) as a yellow solid.

### Example 36: Preparation of (4-ethynylthiazol-2-yl)(1H-indol-3-yl)methanone (ARI-052)

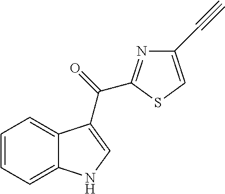 [SMILES: C#Cc1csc(C(=O)c2c[nH]c3ccccc23)n1]

To a stirred solution of tert-butyl 3-(4-formylthiazole-2-carbonyl)-1H-indole-1-carboxylate (71 mg, 0.200 mmol) in anhydrous methanol (2.0 mL) was added K₂CO₃ (55.3 mg, 0.400 mmol) followed by dimethyl (1-diazo-2-oxopropyl)phosphonate (53.8 mg, 0.280 mmol). Upon completion, the reaction mixture was concentrated to dryness. The residue was treated with brine and EtOAc. Organic layer was separated and the aqueous layer was extracted twice with EtOAc. The combined organic layers were dried (Na₂SO₄), filtered, and concentrated. Chromatography (silica gel, heptane to 50% EtOAc/heptane) gave (4-ethynylthiazol-2-yl)(1H-indol-3-yl)methanone (28 mg) as a yellow solid.

### Example 37: Preparation of methyl 2-(1H-indazole-3-carbonyl)thiazole-4-carboxylate (ARI-053)

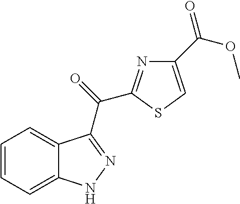 [SMILES: COC(=O)c1csc(C(=O)c2n[nH]c3ccccc23)n1]

To a −78° C. suspension of methyl 2-bromothiazole-4-carboxylate (0.775 g, 3.49 mmol) in THF (8.19 ml) was added iPrMgCl-LiCl (1.3 M in THF) (2.52 ml, 3.28 mmol). After 15 min, a solution of tert-butyl 3-(methoxy(methyl)carbamoyl)-1H-indazole-1-carboxylate (1.0 g, 3.28 mmol) in THF (8.19 ml) was added dropwise to the wine colored Grignard. After 1.5 hrs, 1 N HCl (aq) was added with the bath temperature held at −10° C. The mixture was extracted with EtOAc then washed with saturated NaHCO₃ (aq), and brine, dried (Na₂SO₄), filtered and concentrated to a solid. Chromatography (silica gel, heptane to 20% EtOAc/heptane) gave impure methyl 2-(1-(tert-butoxycarbonyl)-1H-indazole-3-carbonyl)thiazole-4-carboxylate (317 mg, 0.818 mmol). To remove the Boc group, this was treated with anhydrous MeOH (10 mL) and then K₂CO₃ (113 mg, 0.818 mmol) was added. Upon reaction completion, 1 N HCl was added to acidify then the mixture was extracted with EtOAc. The extract was washed with saturated sodium bicarbonate then brine, dried over Na₂SO₄, filtered and concentrated. Chromatography (C18, H₂O to CH₃CN both with 0.1% TFA modifier) gave a solid that was triturated with hot MeOH then dried under vacuum at 50° C. to give methyl 2-(1H-indazole-3-carbonyl)thiazole-4-carboxylate (64, mg) as a yellow solid.

### Example 38: Preparation of (4-(1,3,4-oxadiazol-2-yl)thiazol-2-yl)(1H-indol-3-yl)methanone (ARI-056)

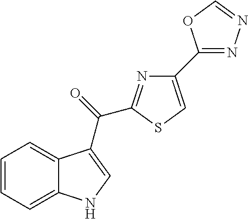 [SMILES: O=C(c1nc(-c2nnco2)cs1)c1c[nH]c2ccccc12]

Step 1. A solution of 2-(1-(tert-butoxycarbonyl)-1H-indole-3-carbonyl)thiazole-4-carboxylic acid (200 mg, 0.537 mmol) and carbonyldiimidazole (113 mg, 0.698 mmol) in tetrahydrofuran (2.0 mL) was stirred at room temperature for 3 h. A precipitate had formed. The crude was carried forward. The mixture was cooled in an ice bath then hydrazine hydrate (78 μL, 1.612 mmol) was added. The reaction was allowed to warm to room temperature overnight then concentrated. The crude was carried forward. ESI MS m/z 387 [M+H]⁺.

Step 2. A mixture of crude tert-butyl 3-(4-(hydrazinecarbonyl)thiazole-2-carbonyl)-1H-indole-1-carboxylate (208 mg, 0.538 mmol), triethyl orthoformate (2.7 mL, 16.21 mmol), and acetic acid (1.0 mL, 17.47 mmol) was stirred at 100° C. A yellow precipitate formed. Upon completion, the reaction mixture was cooled to room temperature, diluted with CH₂Cl₂ and the mixture sonicated. The precipitate was collected, washed with CH₂Cl₂, and dried in vacuo to provide of (4-(1,3,4-oxadiazol-2-yl)thiazol-2-yl)(1H-indol-3-yl)methanone (98 mg) as a yellow solid.

### Example 39: Preparation of (1H-indol-3-yl)(4-(5-methyl-1,3,4-oxadiazol-2-yl)thiazol-2-yl)methanone (ARI-060)

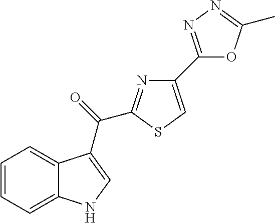 [SMILES: Cc1nnc(-c2csc(C(=O)c3c[nH]c4ccccc34)n2)o1]

Step 1. To a stirred suspension of 2-(1-(tert-butoxycarbonyl)-1H-indole-3-carbonyl)thiazole-4-carboxylic acid (200 mg, 0.537 mmol) in dichloromethane (7.0 mL) at room temperature was added HATU (408 mg, 1.074 mmol) followed by DIPEA (0.141 mL, 0.806 mmol). N,N-Dimethylformamide (0.7 mL) was added to aid solubility. After 10 min, acetohydrazide (47.7 mg, 0.644 mmol) was added. Upon completion, the reaction mixture was absorbed on silica gel. Chromatography (silica gel, CH₂Cl₂ to 10% MeOH/CH₂Cl₂) gave tert-butyl 3-(4-(2-acetylhydrazine-1-carbonyl)thiazole-2-carbonyl)-1H-indole-1-carboxylate as an off-white solid (347, mg). ESI MS m/z 427 [M−H]⁻.

Step 2. To a stirred suspension of tert-butyl 3-(4-(2-acetylhydrazine-1-carbonyl)thiazole-2-carbonyl)-1H-indole-1-carboxylate (230 mg, 0.537 mmol) in dichloromethane (20 mL) at room temperature was added triethylamine (0.374 mL, 2.68 mmol) followed by tosyl-chloride (307 mg, 1.610 mmol). The reaction mixture was heated to 65° C. with stirring for 3 h. A clear solution formed. The reaction mixture was then absorbed on silica gel. Chromatography (silica gel, heptane to 65% EtOAc/heptane) gave tert-butyl 3-(4-(5-methyl-1,3,4-oxadiazol-2-yl)thiazole-2-carbonyl)-1H-indole-1-carboxylate (172, mg) as an off-white solid. ESI MS m/z 411[M+H]⁺.

Step 3. To a stirred suspension of tert-butyl 3-(4-(5-methyl-1,3,4-oxadiazol-2-yl)thiazole-2-carbonyl)-1H-indole-1-carboxylate (162 mg, 0.395 mmol) in methanol (8 mL) at room temperature was added potassium carbonate (164 mg, 1.184 mmol). Upon completion, the mixture was cooled in an ice-water bath, and neutralized with 2 M HCl. The precipitate was collected by filtration, washed with water and methanol, and dried in vacuo to provide (1H-indol-3-yl)(4-(5-methyl-1,3,4-oxadiazol-2-yl)thiazol-2-yl)methanone (130 mg) as a yellow solid.

### Example 40: Preparation of 2-(1H-indole-3-carbonyl)thiazole-4-carbaldehyde (ARI-061)

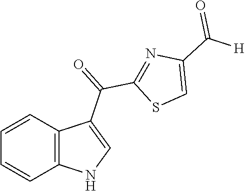 [SMILES: O=Cc1csc(C(=O)c2c[nH]c3ccccc23)n1]

Potassium carbonate (0.175 g, 1.263 mmol) and tert-butyl 3-(4-formylthiazole-2-carbonyl)-1H-indole-1-carboxylate (0.150 g, 0.421 mmol) were suspended in methanol (4.21 ml). Upon completion, the reaction mixture was acidified with 1M HCl and extracted with EtOAc. The organic was then concentrated onto silica gel. Chromatography (silica gel, heptane to 50% EtOAc/heptane) gave 2-(1H-indole-3-carbonyl)thiazole-4-carbaldehyde (0.090 g) as a yellow solid.

### Example 41: Preparation of (4-(1H-1,2,3-triazol-5-yl)thiazol-2-yl)(1H-indol-3-yl)methanone (ARI-062)

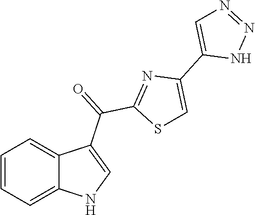 [SMILES: O=C(c1nc(-c2cnn[nH]2)cs1)c1c[nH]c2ccccc12]

To a stirred solution of (4-ethynylthiazol-2-yl)(1H-indol-3-yl)methanone (150 mg, 0.595 mmol), and copper(I) iodide (5.66 mg, 0.030 mmol) in N,N-dimethylformamide (4.5 mL)/methanol (0.500 mL) was added TMSN₃ (0.118 mL, 0.892 mmol). The reaction mixture was stirred in a sealed reaction vessel at 100° C. for 6 h. The reaction mixture was concentrated in vacuo. The residue was dissolved in hot MeOH/water, filtered and the filtrate concentrated to dryness. The resulting residue was triturated with CH₂Cl₂ and dried in vacuo to provide (4-(1H-1,2,3-triazol-5-yl)thiazol-2-yl)(1H-indol-3-yl)methanone (154 mg) as a yellow solid.

### Example 42: Preparation of 2-(2-(1H-indole-3-carbonyl)thiazol-4-yl)-2-aminoacetic acid (ARI-063)

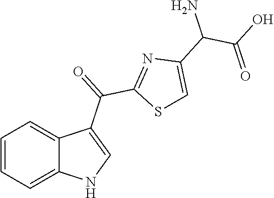 [SMILES: NC(C(=O)O)c1csc(C(=O)c2c[nH]c3ccccc23)n1]

Step 1. To a solution of ammonium acetate (130 mg, 1.684 mmol), sodium cyanide (30.3 mg, 0.617 mmol) and ammonium hydroxide (170 μL, 1.268 mmol) in water (500 μL)/ethanol (500 μL) at room temperature was added tert-butyl 3-(4-formylthiazole-2-carbonyl)-1H-indole-1-carboxylate (200 mg, 0.561 mmol). The cloudy reaction mixture was stirred for 18.5 h. More ethanol (500 μL), ammonium acetate (130 mg, 1.684 mmol), ammonium hydroxide (170 μL, 1.268 mmol), and sodium cyanide (30.3 mg, 0.617 mmol) were added. Stirring was continued for an additional 19.5 h. More ethanol (500 μL), ammonium acetate (130 mg, 1.684 mmol), ammonium hydroxide (170 μL, 1.268 mmol), and tetrabutylammonium cyanide (166 mg, 0.617 mmol) were added. Stirring was continued for 24 h. The reaction mixture was diluted with EtOAc (3 mL), washed with water (1 mL), dried over Na₂SO₄, filtered, and concentrated. The residue was dried in vacuo to an orange-brown sticky solid (293 mg). The crude was carried forward. ESI MS m/z 383 [M+H]⁺.

Step 2. To a stirred solution of crude tert-butyl 3-(4-(amino(cyano)methyl)thiazole-2-carbonyl)-1H-indole-1-carboxylate (215 mg, 0.562 mmol) in acetic acid (4.0 mL) at room temperature was added concentrated hydrochloric acid (2.0 mL, 24.36 mmol). The reaction mixture was stirred at 100° C. for 17 h. The reaction mixture was cooled to room temperature and then concentrated to dryness. Chromatography (C18, H₂O to 50% MeCN/H₂O) gave 2-(2-(1H-indole-3-carbonyl)thiazol-4-yl)-2-aminoacetic acid (26.1 mg) as a light red solid.

### Example 43: Preparation of (1H-indol-3-yl)(4-(5-methyl-4H-1,2,4-triazol-3-yl)thiazol-2-yl)methanone (ARI-064)

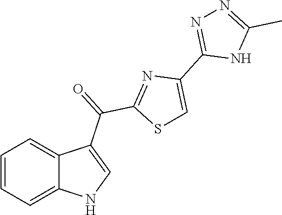 [SMILES: Cc1nnc(-c2csc(C(=O)c3c[nH]c4ccccc34)n2)[nH]1]

Step 1. To a stirred mixture of 2-(1-(tert-butoxycarbonyl)-1H-indole-3-carbonyl)thiazole-4-carboxylic acid (240 mg, 0.644 mmol), ammonium chloride (241 mg, 4.51 mmol), HOBt (148 mg, 0.967 mmol) and EDC (371 mg, 1.933 mmol) were added N,N-dimethylformamide (6.5 mL) and then DIPEA (0.675 mL, 3.87 mmol). Upon completion, the reaction mixture was diluted with water and saturated NaHCO₃ (aq). The precipitate was collected by filtration, washed with water, and dried in vacuo to provide tert-butyl 3-(4-carbamoylthiazole-2-carbonyl)-1H-indole-1-carboxylate (224 mg) as a light yellow solid. ESI MS m/z 372 [M+H]⁺.

Step 2. To an ice-cold, stirred solution of tert-butyl 3-(4-carbamoylthiazole-2-carbonyl)-1H-indole-1-carboxylate (200 mg, 0.538 mmol) in methanol (10.0 mL) was added sodium borohydride (61.1 mg, 1.615 mmol) in two portions. The reaction mixture was stirred at 0° C. for 1 h. Then, the reaction mixture was quenched with 2 M HCl until pH reached 5-6 and then concentrated to dryness. The residue was partitioned between EtOAc and water. The organic was washed with brine, dried (Na₂SO₄), filtered, and concentrated to give tert-butyl 3-((4-carbamoylthiazol-2-yl)(hydroxy)methyl)-1H-indole-1-carboxylate (224 mg) as a colorless syrup. The crude was carried forward. ESI MS m/z 374 [M+H]⁺.

Step 3. To a stirred solution of tert-butyl 3-((4-carbamoylthiazol-2-yl)(hydroxy)methyl)-1H-indole-1-carboxylate (224 mg, 0.538 mmol) and dihydropyran (98 μL, 1.072 mmol) in dichloromethane (5.5 mL) at room temperature was added pyridinium p-toluenesulfonate (6.76 mg, 0.027 mmol). The reaction mixture was stirred for 20.5 h. The reaction mixture was absorbed on silica gel. Chromatography (silica gel, heptane to 70% EtOAc/heptane) gave tert-butyl 3-((4-carbamoylthiazol-2-yl)((tetrahydro-2H-pyran-2-yl)oxy)methyl)-1H-indole-1-carboxylate (244 mg) as a light yellow syrup. ESI MS m/z 458 [M+H]⁺.

Step 4. A mixture of tert-butyl 3-((4-carbamoylthiazol-2-yl)((tetrahydro-2H-pyran-2-yl)oxy)methyl)-1H-indole-1-carboxylate (5 mg, 10.93 μmol) and 1,1-dimethoxy-N,N-dimethylethan-1-amine (150 μL, 1.026 mmol) was stirred at 80° C. for 15 h. The reaction mixture was concentrated and residue dried in vacuo to provide tert-butyl (E)-3-((4-((1-(dimethylamino)ethylidene)carbamoyl)thiazol-2-yl)((tetrahydro-2H-pyran-2-yl)oxy)methyl)-1H-indole-1-carboxylate as a brown viscous oil, which was used in the next step without purification. ESI MS m/z 527 [M+H]⁺.

Step 5. A solution of crude tert-butyl (E)-3-((4-((1-(dimethylamino)ethylidene)carbamoyl)thiazol-2-yl)((tetrahydro-2H-pyran-2-yl)oxy)methyl)-1H-indole-1-carboxylate (261 mg, 0.496 mmol) and hydrazine hydrate (77 μL, 2.478 mmol) in acetic acid (3.5 mL) was stirred at 80° C. Upon completion, the reaction mixture was cooled to room temperature and absorbed onto silica gel. Chromatography (silica gel, CH2C12 to 50% 80:18:2 CH₂Cl₂/MeOH/concentrated NH₄OH) gave tert-butyl 3-(hydroxy(4-(5-methyl-4H-1,2,4-triazol-3-yl)thiazol-2-yl)methyl)-1H-indole-1-carboxylate (41 mg) as a yellow solid. ESI MS m/z 496 [M+H]⁺.

Step 6. To a stirred solution of tert-butyl 3-(hydroxy(4-(5-methyl-4H-1,2,4-triazol-3-yl)thiazol-2-yl)methyl)-1H-indole-1-carboxylate (41 mg, 0.100 mmol) in dichloromethane (2.5 mL) at room temperature was added Dess-Martin periodinane (54.9 mg, 0.130 mmol). Upon completion the reaction was quenched with saturated NaHCO₃ (2 mL) and 10% Na₂S₂O₃ (2 mL). The organic layer was separated. The aqueous layer was extracted with CH₂Cl₂ (2×). The combined organic layers were dried (Na₂SO₄), filtered, and concentrated. Chromatography (silica gel, CH₂Cl₂ to 10% MeOH/CH₂Cl₂) gave tert-butyl 3-(4-(5-methyl-4H-1,2,4-triazol-3-yl)thiazole-2-carbonyl)-1H-indole-1-carboxylate (29.1 mg) as a yellow solid. ESI MS m/z 410 [M+H]⁺.

Step 7. To a stirred suspension of tert-butyl 3-(4-(5-methyl-4H-1,2,4-triazol-3-yl)thiazole-2-carbonyl)-1H-indole-1-carboxylate (29 mg, 0.071 mmol) in methanol (2.4 mL) at room temperature was added potassium carbonate (29.4 mg, 0.212 mmol). Upon completion, the reaction mixture was neutralized with 2 M HCl while cooled in an ice-water bath. The resulting precipitate was collected, washed with water and dried in vacuo to provide (1H-indol-3-yl)(4-(5-methyl-4H-1,2,4-triazol-3-yl)thiazol-2-yl)methanone (19.1 mg) as a light yellow solid.

Alternatively, ARI-064 was synthesized according to the scheme of FIG. 18 and by the following method:

### Step: (1H-indol-3-yl)(4-(5-methyl-4H-1,2,4-triazol-3-yl)thiazol-2-yl)methanone (ARI-064)

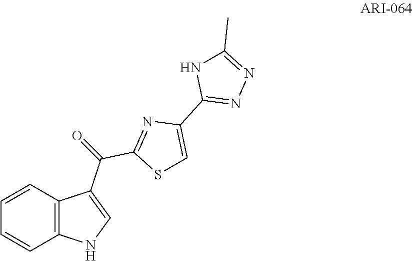 [SMILES: Cc1nnc(-c2csc(C(=O)c3c[nH]c4ccccc34)n2)[nH]1]

A suspension of compound 49-1 (1.45 g, 3.7 mmol), acetimidamide hydrochloride (700 mg, 7.5 mmol) and NaOH (300 mg, 7.5 mmol) in dioxane (20 mL) was stirred for 30 min at 110° C. under microware. After cooled to room temperature, the mixture was filtered, and the solid was collected, washed with EtOAc (20 mL×3) and MeOH (20 mL×3), dried to afford compound ARI-064 (880 mg, 76% yield) in the form of a yellow solid. ¹H-NMR (400 MHz, DMSO-d6): δ 12.39 (bs, 1H), 9.38 (s, 1H), 8.51 (s, 1H), 8.33-8.36 (m, 1H), 7.28-7.62 (d, J=6.4 Hz, 1H), 7.29-7.32 (m, 2H), 2.51 (s, 3H). LC-MS: m/z 308.1 [M−H]⁻.

### Example 44: Preparation of (4-(1,2,4-oxadiazol-3-yl)thiazol-2-yl)(1H-indol-3-yl)methanone (ARI-071)

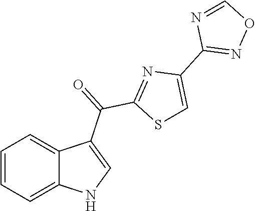 [SMILES: O=C(c1nc(-c2ncon2)cs1)c1c[nH]c2ccccc12]

Step 1. A mixture of 2-(1H-indole-3-carbonyl)thiazole-4-carbonitrile (160 mg, 0.632 mmol), K₃PO₄ (402 mg, 1.895 mmol) and hydroxylamine hydrochloride (110 mg, 1.579 mmol) in DMF (10 mL) was heated to 100° C. in a microwave reactor for 30 min. Triethyl orthoformate (3.16 mL, 18.97 mmol), pyridinium p-toluenesulfonate (PPTS) (31.8 mg, 0.126 mmol) and TFA (0.317 mL, 4.11 mmol) was added. The reaction mixture was further heated to 100° C. on a microwave reactor for 2 h. The reaction mixture was concentrated in vacuo. The residue was triturated with water by sonication. The precipitate was collected, washed with water, dried. Chromatography (silica gel, CH₂Cl₂ to 6% MeOH/CH₂Cl₂) gave (4-(1,2,4-oxadiazol-3-yl)thiazol-2-yl)(1H-indol-3-yl)methanone (69 mg) as a yellow solid.

### Example 45: Preparation of (1H-indol-3-yl)(thiazol-4-yl)methanone (ARI-072)

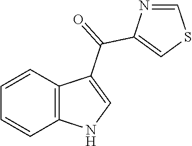 [SMILES: O=C(c1cscn1)c1c[nH]c2ccccc12]

Prepared according to the method described in Example 2 except that ethyl 4-(chlorocarbonyl)thiazole-2-carboxylate was used instead of 4-bromothiazole-2-carbonyl chloride in Step 1 and the Boc deprotection was effected using NaOH in methanol.

Ethyl 4-(chlorocarbonyl)thiazole-2-carboxylate was obtained from commercial ethyl 4-(chlorocarbonyl)thiazole-2-carboxylate as follows. To an ice-cold suspension of 2-(ethoxycarbonyl)thiazole-4-carboxylic acid (1 g, 4.97 mmol) in DCM (9.94 ml) was added 2 drops of DMF then oxalyl chloride (0.505 ml, 5.96 mmol) was added dropwise. The bath was removed and a large bubbler was added. Upon nearing room temperature CO₂ evolution was observed and after 3 h, gas evolution ceased. The solution was concentrated under reduced pressure and used as crude.

### Example 46: Preparation of (1H-indol-3-yl)(phenyl)methanone (ARI-073)

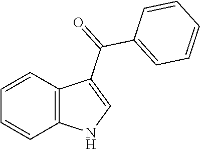 [SMILES: O=C(c1ccccc1)c1c[nH]c2ccccc12]

Prepared according to the method described in Example 2 except that benzoyl chloride was used instead of 4-bromothiazole-2-carbonyl chloride.

### Example 47: Preparation of (1H-indol-3-yl)(m-tolyl)methanone (ARI-074)

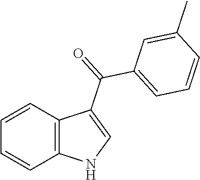 [SMILES: Cc1cccc(C(=O)c2c[nH]c3ccccc23)c1]

Prepared according to the method described in Example 2 except that 3-methylbenzoyl chloride was used instead of 4-bromothiazole-2-carbonyl chloride.

### Example 48: Preparation of 2-(2-(1H-indole-3-carbonyl)thiazol-4-yl)-2-aminoacetonitrile (ARI-075)

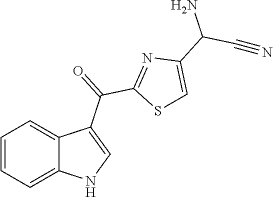 [SMILES: N#CC(N)c1csc(C(=O)c2c[nH]c3ccccc23)n1]

Step 1. A solution of ammonium acetate (195 mg, 2.53 mmol), tetrabutylammonium cyanide (249 mg, 0.926 mmol), and ammonium hydroxide (0.255 mL, 1.902 mmol) in water (1.2 mL) was added to a suspension of tert-butyl 3-(4-formylthiazole-2-carbonyl)-1H-indole-1-carboxylate (300 mg, 0.842 mmol) in ethanol (1.2 mL) at room temperature. The cloudy reaction mixture was stirred for 26 h. The reaction mixture was diluted with EtOAc, washed with water, dried over Na₂SO₄, filtered, and concentrated. Chromatography (silica gel, CH₂Cl₂ to 4.5% MeOH/CH₂Cl₂) and then (C18, H₂O to CH₃CN)) gave 2-(2-(1H-indole-3-carbonyl)thiazol-4-yl)-2-aminoacetonitrile (37.6 mg) as a yellow solid.

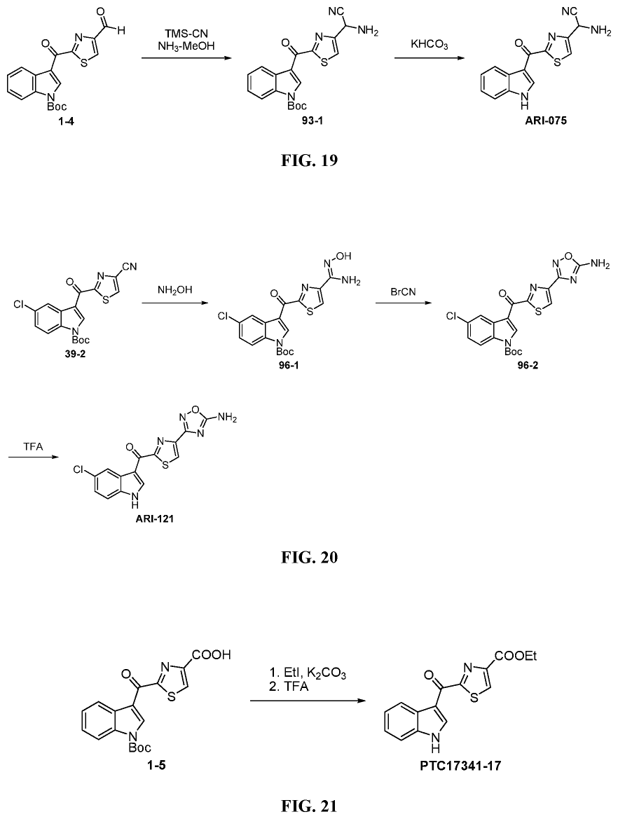

Alternatively, ARI-075 was synthesized according to the scheme of FIG. 19 and by the following method:

### Step 1: tert-Butyl 3-(4-(amino(cyano)methyl)thiazole-2-carbonyl)-1H-indole-1-carboxylate (93-1)

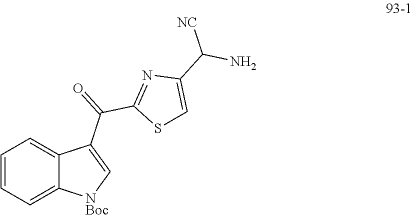 [SMILES: CC(C)(C)OC(=O)n1cc(C(=O)c2nc(C(N)C#N)cs2)c2ccccc21]

Trimethylsilyl cyanide (0.74 mL, 5.5 mmol) was added to a solution of compound 1-4 (1.40 g, 4 mmol) in THF (5 mL) and NH₃-MeOH (7M solution, 20 mL) at room temperature. The mixture was stirred for 2 h, then concentrated to dryness to afford compound 93-1 (2.0 g, ˜100% yield), which was used for next step without further purification.

### Step 2: 2-(2-(1H-indole-3-carbonyl)thiazol-4-yl)-2-aminoacetonitrile (ARI-075)

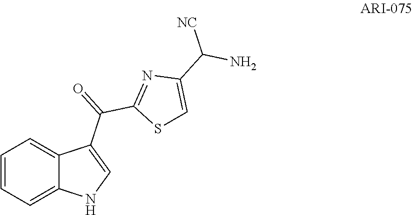 [SMILES: N#CC(N)c1csc(C(=O)c2c[nH]c3ccccc23)n1]

The BOC group of compound 93-1 (2.00 g, 5 mmol) was removed as described in Example 24 (treatment with K₂CO₃ in methanol held at 50° C.) to give title compound ARI-075 in the form of a yellow solid (680 mg, 43% yield). ¹H-NMIR (400 MHz, DMSO-d6): δ 12.37 (bs, 1H), 9.17 (s, 1H), 8.31-8.35 (m, 1H), 8.07 (s, 1H), 7.56˜7.59 (d, J=6.0 Hz, 1H), 7.28˜7.33 (m, 2H), 5.34˜5.39 (t, J=8.0 Hz, 1H), 3.05˜3.08 (d, J=8.0 Hz, 1H). LC-MS: m/z 281.0 [M−H]⁻.

### Example 49: Preparation of (1H-indol-3-yl)(pyridin-2-yl)methanone (ARI-081)

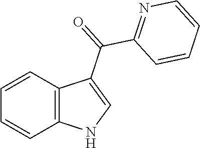 [SMILES: O=C(c1ccccn1)c1c[nH]c2ccccc12]

Prepared according to the method described in Example 2 except that picolinoyl chloride hydrochloride was used instead of 4-bromothiazole-2-carbonyl chloride.

### Example 50: Preparation of methyl 3-(1H-indole-3-carbonyl)benzoate (ARI-082)

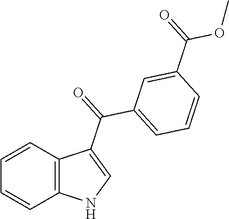 [SMILES: COC(=O)c1cccc(C(=O)c2c[nH]c3ccccc23)c1]

Prepared according to the method described in Example 2 except that methyl 3-(chlorocarbonyl)benzoate was used instead of 4-bromothiazole-2-carbonyl chloride. The carboxylic acid was the primary product which was subsequently esterified by treatment with sulfuric acid in methanol at 100° C.

### Example 51: Preparation of (4-(5-amino-1,3,4-oxadiazol-2-yl)thiazol-2-yl)(1H-indol-3-yl)methanone (ARI-083)

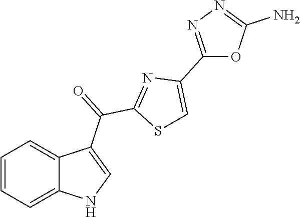 [SMILES: Nc1nnc(-c2csc(C(=O)c3c[nH]c4ccccc34)n2)o1]

Step 1. To a stirred suspension of tert-butyl 3-(4-formylthiazole-2-carbonyl)-1H-indole-1-carboxylate (150 mg, 0.421 mmol) in methanol (0.90 mL)/N,N-dimethylformamide (0.900 mL) at room temperature was added a solution of hydrazinecarboxamide (46.9 mg, 0.421 mmol) and sodium acetate (34.5 mg, 0.421 mmol) in water (0.900 mL). The reaction mixture was stirred for 21.5 h. The reaction mixture was concentrated to dryness and the residue was dried in vacuo to provide tert-butyl 3-(4-((2-carbamoylhydrazono)methyl)thiazole-2-carbonyl)-1H-indole-1-carboxylate as a light yellow solid which was carried forward as crude. ESI MS m/z 414 [M+H]⁺.

Step 2. To a stirred cloudy solution of crude tert-butyl 3-(4-((2-carbamoylhydrazono)methyl)thiazole-2-carbonyl)-1H-indole-1-carboxylate (174 mg, 0.421 mmol) in 1,4-dioxane (30 mL) at room temperature was added potassium carbonate (174 mg, 1.263 mmol) followed by iodine (128 mg, 0.505 mmol). The reaction mixture was stirred at 80° C. for 25 h. The reaction mixture was cooled to room temperature and diluted with water (30 mL). The resulting precipitate was collected, washed with water, and dried in vacuo to provide tert-butyl 3-(4-(5-amino-1,3,4-oxadiazol-2-yl)thiazole-2-carbonyl)-1H-indole-1-carboxylate (155 mg) as a yellow solid. ESI MS m/z 410 [M−H]⁻.

Step 3. To a stirred suspension of tert-butyl 3-(4-(5-amino-1,3,4-oxadiazol-2-yl)thiazole-2-carbonyl)-1H-indole-1-carboxylate (155 mg, 0.377 mmol) in methanol (12.5 mL) at room temperature was added potassium carbonate (156 mg, 1.130 mmol). The reaction mixture was stirred for 15.5 h. The reaction mixture was cooled in an ice-water bath and neutralized with 2M HCl. The resulting precipitate was collected, washed with water, and dried in vacuo to provide (4-(5-amino-1,3,4-oxadiazol-2-yl)thiazol-2-yl)(1H-indol-3-yl)methanone (87.5 mg) as a yellow solid.

### Example 52: Preparation of (1H-indol-3-yl)(4-(2,2,2-trifluoro-1-hydroxyethyl)thiazol-2-yl)methanone (ARI-088)

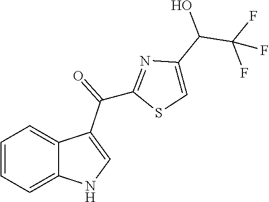 [SMILES: O=C(c1nc(C(O)C(F)(F)F)cs1)c1c[nH]c2ccccc12]

Step 1. To a −35° C. solution of tert-butyl 3-(4-formylthiazole-2-carbonyl)-1H-indole-1-carboxylate (1.63 g, 4.57 mmol) and tetrabutylammonium acetate (0.034 g, 0.114 mmol) in DCM (100 ml) was added trimethyl(trifluoromethyl)silane (0.676 ml, 4.57 mmol) dropwise. The reaction was allowed to slowly warm to room temperature. Upon completion, saturated NaCl was added. The layers were separated and the organic dried (Na₂SO₄), filtered and concentrated. Chromatography (silica gel, heptane to CH₂Cl₂) gave tert-butyl 3-(4-(2,2,2-trifluoro-1-hydroxyethyl)thiazole-2-carbonyl)-1H-indole-1-carboxylate (1.67 g) as a colorless hard film. ESI MS m/z 427 [M+H]⁺.

Step 2. To tert-butyl 3-(4-(2,2,2-trifluoro-1-hydroxyethyl)thiazole-2-carbonyl)-1H-indole-1-carboxylate (2.145 g, 5.03 mmol) was added MeOH (10.06 ml) then 1 M NaOH (aq) (10.06 ml, 10.06 mmol) was added and the mixture heated to 65° C. for 30 min. The solvent was concentrated and the residue partitioned between 1 N HCl and EtOAc. The organic phase was separated, washed with water and then brine, dried (Na₂SO₄), filtered and concentrated onto silica gel. Chromatography (silica gel, heptane to 45% EtOAc/heptane) gave (1H-indol-3-yl)(4-(2,2,2-trifluoro-1-hydroxyethyl)thiazol-2-yl)methanone (1.44, g) as a yellow solid.

### Example 53: Preparation of 1-(2-(1H-indole-3-carbonyl)thiazol-4-yl)-2,2,2-trifluoroethan-1-one (ARI-089)

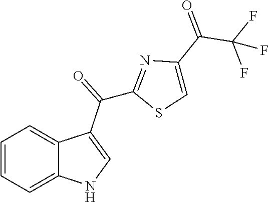 [SMILES: O=C(c1nc(C(=O)C(F)(F)F)cs1)c1c[nH]c2ccccc12]

Step 1. To tert-butyl 3-(4-(2,2,2-trifluoro-1-hydroxyethyl)thiazole-2-carbonyl)-1H-indole-1-carboxylate (417 mg, 0.978 mmol) and 1,1,1-tris(acetyloxy)-1,1-dihydro-1,2-benziodoxol-3-(1H)-one (539 mg, 1.271 mmol) was added CH₂Cl₂ (10 mL). After 1 hr, the reaction was quenched by the addition of saturated NaHCO₃ and 10% Na₂S₂O₃. After stirring 20 min, CH₂Cl₂ was added. After separation, the organic phase was washed with a second portion of bicarbonate, dried over Na₂SO₄, filtered and concentrated. Chromatography (silica gel, heptane to 25% EtOAc/hepane) gave tert-butyl 3-(4-(2,2,2-trifluoro-1,1-dihydroxyethyl)thiazole-2-carbonyl)-1H-indole-1-carboxylate (409.7 mg) as a yellow solid. The mass spectrum for the product shows that the product may exist as the diol ESI MS m/z 443 [M+H+H₂O]⁺.

Step 2. To a solution of tert-butyl 3-(4-(2,2,2-trifluoro-1,1-dihydroxyethyl)thiazole-2-carbonyl)-1H-indole-1-carboxylate (180 mg, 0.407 mmol) in THF (2 ml) was added 2 M NaOH (aq) (1.2 ml, 2.4 mmol) and the mixture was heated to 40° C. Upon completion, the reaction was neutralized with 1 N HCl (aq). Chromatography (C18, H₂O to CH₃CN, liquid load) gave 1-(2-(1H-indole-3-carbonyl)thiazol-4-yl)-2,2,2-trifluoroethan-1-one (80 mg) as a yellow solid.

### Example 54: Preparation of (4-(5-amino-1,3,4-thiadiazol-2-yl)thiazol-2-yl)(1H-indol-3-yl)methanone (ARI-090)

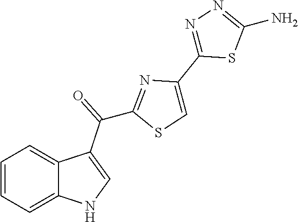 [SMILES: Nc1nnc(-c2csc(C(=O)c3c[nH]c4ccccc34)n2)s1]

Prepared according to the method described in Example 51 except that hydrazinecarbothioamide was used instead of hydrazinecarboxamide.

### Example 55: Preparation of 3-(1H-indole-3-carbonyl)benzonitrile (ARI-091)

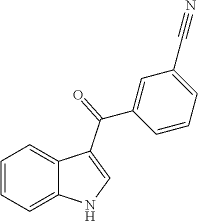 [SMILES: N#Cc1cccc(C(=O)c2c[nH]c3ccccc23)c1]

Step 1. Oxalyl chloride (0.119 ml, 1.357 mmol) was added dropwise to an ice-cold suspension of 3-(1H-indole-3-carbonyl)benzoic acid (0.300 g, 1.131 mmol) in tetrahydrofuran (10 ml). The ice bath was removed and the reaction stirred at ambient temperature. One drop of DMF was added and gas inflow switched from nitrogen inlet to a bubbler. After the bubbling of CO₂ ceased, ammonium hydroxide (0.944 ml, 6.79 mmol) was added at 0° C. Upon completion, the reaction mixture was concentrated under reduced pressure, then triturated with water then dried to give 3-(1H-indole-3-carbonyl)benzamide. The crude solid was used as is. ESI MS m/z 264 [M−H]⁻.

Step 2. A solution of 3-(1H-indole-3-carbonyl)benzamide (0.267 g, 1.010 mmol) and triethylamine (0.704 ml, 5.05 mmol) in tetrahydrofuran (10.10 ml) was stirred in an ice bath for 10 minutes. Trifluoroacetic anhydride (0.357 ml, 2.53 mmol) was added dropwise. Upon completion, the reaction mixture was poured over ice and diluted with ethyl acetate. The organic layer was washed with 2M Na₂CO₃ and brine, then dried over sodium sulfate, filtered and concentrated onto silica gel. Chromatography (silica gel, heptane to 50% EtOAc/heptane) gave 3-(1H-indole-3-carbonyl)benzonitrile (109.7 mg) as an off-white solid.

### Example 56: Preparation of (5-chloro-1H-indol-3-yl)(4-(3-methyl-1,2,4-oxadiazol-5-yl)thiazol-2-yl)methanone (ARI-096)

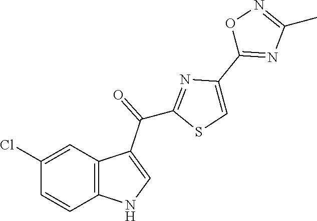 [SMILES: Cc1noc(-c2csc(C(=O)c3c[nH]c4ccc(Cl)cc34)n2)n1]

Prepared according to the method described in Example 22 except that 2-(1-(tert-butoxycarbonyl)-5-chloro-1H-indole-3-carbonyl)thiazole-4-carboxylic acid, derived from 5-chloro-1H-indole-3-carboxylic acid was used instead of 2-(1-(tert-butoxycarbonyl)-1H-indole-3-carbonyl)thiazole-4-carboxylic acid.

### Example 57: Preparation of 2-(5-chloro-1H-indole-3-carbonyl)thiazole-4-carbonitrile (ARI-099)

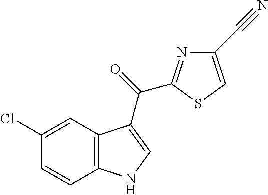 [SMILES: N#Cc1csc(C(=O)c2c[nH]c3ccc(Cl)cc23)n1]

Step 1. Oxalyl chloride (0.129 ml, 1.475 mmol) was added dropwise to an ice-cold suspension of 2-(1-(tert-butoxycarbonyl)-5-chloro-1H-indole-3-carbonyl)thiazole-4-carboxylic acid (0.500 g, 1.229 mmol) in tetrahydrofuran (24 ml). The ice bath was removed and the reaction stirred at ambient temperature. Upon completion, the reaction mixture was concentrated under reduced pressure then resuspended in tetrahydrofuran (24 ml) and chilled in an ice bath. Ammonium hydroxide (1.026 ml, 7.37 mmol) was added at 0° C. Upon completion, the reaction mixture was concentrated under reduced pressure, then triturated with water and concentrated. The solid (0.357 g, 0.880 mmol) was suspended in methanol (8.80 ml) and potassium carbonate (0.365 g, 2.64 mmol) was added. Upon completion, the reaction mixture was concentrated, then suspended in water and adjusted to pH 4 with 1M HCl, the biphasic mixture was filtered and dried to give 2-(5-chloro-1H-indole-3-carbonyl)thiazole-4-carboxamide (0.244 g, 0.798 mmol) as a yellow solid. ESI MS m/z 306 [M+H]⁺.

Step 2. Triethylamine (0.556 ml, 3.99 mmol) was added to an ice-cold suspension of 2-(5-chloro-1H-indole-3-carbonyl)thiazole-4-carboxamide (0.244 g, 0.798 mmol) in tetrahydrofuran (7.98 ml), then stirred for ten minutes. Trifluoroacetic anhydride (0.282 ml, 1.995 mmol) was added dropwise to the reaction mixture. Upon completion, the reaction mixture was poured over ice, then diluted with ethyl acetate. The biphasic mixture was filtered and washed with water to provide 2-(5-chloro-1H-indole-3-carbonyl)thiazole-4-carbonitrile (0.192 g) as a yellow solid.

### Example 58: Preparation of (4-(1-amino-2,2,2-trifluoroethyl)thiazol-2-yl)(1H-indol-3-yl)methanone (ARI-100)

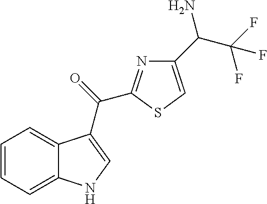 [SMILES: NC(c1csc(C(=O)c2c[nH]c3ccccc23)n1)C(F)(F)F]

Step 1. To an ice-cold solution of tert-butyl 3-(4-(2,2,2-trifluoro-1-hydroxyethyl)thiazole-2-carbonyl)-1H-indole-1-carboxylate (0.305 g, 0.715 mmol) in CH₂Cl₂ (7153 μl) was added triethylamine (299 μl, 2.146 mmol) then methanesulfonylchloride (83 μl, 1.073 mmol) dropwise. Upon completion, the cold reaction mixture was poured into saturated NaHCO₃. The organic phase was separated and then dried (Na2SO4), filtered and concentrated to give tert-butyl 3-(4-(2,2,2-trifluoro-1-((methylsulfonyl)oxy)ethyl)thiazole-2-carbonyl)-1H-indole-1-carboxylate (422 mg) as a yellow gum. Used as is. ESI MS m/z 505 [M+H]⁺.

Step 2. To a mixture of tert-butyl 3-(4-(2,2,2-trifluoro-1-((methylsulfonyl)oxy)ethyl)thiazole-2-carbonyl)-1H-indole-1-carboxylate (0.361 g, 0.715 mmol) and sodium azide (0.279 g, 4.29 mmol) was added DMF. The reaction was heated to 60° C. and stirred overnight. Partial Boc removal was observed. Concentrated the DMF under vacuum. The residue was partitioned between EtOAc and 5% aqueous LiCl. The organic phase was washed with brine, dried (Na₂SO₄), filtered and concentrated to a yellow solid (340 mg). The solid was treated with MeOH (20 mL) and 2 mL of 2N NaOH (aq). then heated the mixture to 50° C. Upon completion, the mixture was neutralized with 1 N HCl then most of MeOH was evaporated. The mixture was then partitioned between EtOAc and H₂O. The organic was dried over Na₂SO₄, and filtered to give (4-(1-azido-2,2,2-trifluoroethyl)thiazol-2-yl)(1H-indol-3-yl)methanone (245 mg) as a yellow solid which was used as is. ESI MS m/z 352 [M+H]⁺.

Step 3. A stirred solution of crude (4-(1-azido-2,2,2-trifluoroethyl)thiazol-2-yl)(1H-indol-3-yl)methanone (242 mg, 0.689 mmol) in a mixture of THF (10 ml) and water (3.33 ml) was heated to 60° C. overnight. The mixture was absorbed onto a SCX-2 5 g column. Eluted with 10% concentrated NH₄OH in MeOH and then further concentrated. Chromatography (C18, H₂O to CH₃CN) gave (4-(1-amino-2,2,2-trifluoroethyl)thiazol-2-yl)(1H-indol-3-yl)methanone (90 mg) as a yellow solid.

### Example 59: Preparation of (5-chloro-1H-indol-3-yl)(4-(5-methyl-1,2,4-oxadiazol-3-yl)thiazol-2-yl)methanone (ARI-109)

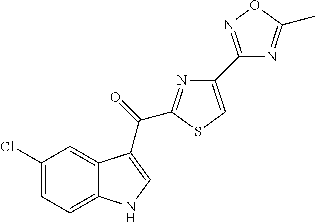 [SMILES: Cc1nc(-c2csc(C(=O)c3c[nH]c4ccc(Cl)cc34)n2)no1]

Step 1. A mixture of tert-butyl 5-chloro-3-(4-cyanothiazole-2-carbonyl)-1H-indole-1-carboxylate (0.860 g, 2.217 mmol) and potassium carbonate (0.919 g, 6.65 mmol) were stirred in methanol (44.3 ml). Upon completion, the reaction mixture was neutralized with 1M HC1, and extracted with ethyl acetate then concentrated onto silica gel. Chromatrography (silica gel, heptane to EtOAc then 20% MeOH/DCM) gave methyl 2-(5-chloro-1H-indole-3-carbonyl)thiazole-4-carbimidate (0.705 g). ESI MS m/z 320 [M+H]⁺.

Step 2. Methyl 2-(5-chloro-1H-indole-3-carbonyl)thiazole-4-carbimidate (0.200 g, 0.625 mmol), potassium phosphate (0.398 g, 1.876 mmol) and hydroxylamine hydrochloride (0.109 g, 1.564 mmol) in N,N-dimethylformamide (7.82 ml) were heated to 100° C. in the microwave for 30 minutes. Acetyl chloride (0.36 ml, 5.06 mmol) was added and the reaction mixture was resubjected to heating to 100° C. in the microwave for 5 h. After this time, the reaction mixture was concentrated under reduced pressure and triturated with water and then with hot methanol to provide (5-chloro-1H-indol-3-yl)(4-(5-methyl-1,2,4-oxadiazol-3-yl)thiazol-2-yl)methanone (144 mg). ESI MS m/z 345 [M+H]⁺.

Step 3. DMAP (0.017 g, 0.136 mmol) was added to a suspension of (5-chloro-1H-indol-3-yl)(4-(5-methyl-1,2,4-oxadiazol-3-yl)thiazol-2-yl)methanone (0.188 g, 0.545 mmol) and Boc₂O (0.165 ml, 0.709 mmol) in acetonitrile (5.45 ml) at ambient temperature. Upon completion, the reaction mixture was concentrated under reduced pressure onto silica gel. Chromatography (silica gel, heptane to 50% EtOAc/heptane) followed by reverse phase chromatography (C18, 5% to 100% acetonitrile/water) gave tert-butyl 5-chloro-3-(4-(5-methyl-1,2,4-oxadiazol-3-yl)thiazole-2-carbonyl)-1H-indole-1-carboxylate (24 mg). ESI MS m/z 445 [M+H]⁺.

Step 4. To a suspension of tert-butyl 5-chloro-3-(4-(5-methyl-1,2,4-oxadiazol-3-yl)thiazole-2-carbonyl)-1H-indole-1-carboxylate (0.024 g, 0.054 mmol) in methanol (0.539 ml) was added potassium carbonate (0.030 g, 0.216 mmol) and the mixture was stirred at ambient temperature overnight. The reaction mixture was concentrated under reduced pressure, suspended in water and acidified with 1 M HCl. The solid was collected by filtration to afford (5-chloro-1H-indol-3-yl)(4-(5-methyl-1,2,4-oxadiazol-3-yl)thiazol-2-yl)methanone (11.7 mg) as an off-white solid.

### Example 60: Preparation of (4-(5-amino-1,2,4-oxadiazol-3-yl)thiazol-2-yl)(1H-indol-3-yl)methanone (ARI-110)

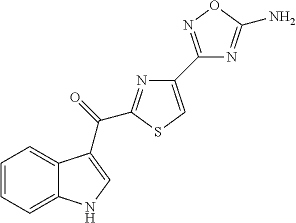 [SMILES: Nc1nc(-c2csc(C(=O)c3c[nH]c4ccccc34)n2)no1]

Step 1. A mixture of 2-(1H-indole-3-carbonyl)thiazole-4-carbonitrile (25 mg, 0.099 mmol), phosphoric acid, potassium salt (62.9 mg, 0.296 mmol) and hydroxylamine hydrochloride (17.15 mg, 0.247 mmol) in DMF (1.1 mL) was heated to 100° C. in a microwave reactor for 30 min. The reaction mixture was concentrated to dryness. The residue was diluted with brine and EtOAc. The precipitate was collected by filtration, washed with water (3×), and then dried in vacuo to provide N′-hydroxy-2-(1H-indole-3-carbonyl)thiazole-4-carboximidamide (23.4 mg) as a yellow solid. ESI MS m/z 287 [M+H]⁺.

Step 2. A mixture of N′-hydroxy-2-(1H-indole-3-carbonyl)thiazole-4-carboximidamide (23.4 mg, 0.082 mmol) and 2,2,2-trichloroacetic anhydride (150 μL, 0.821 mmol) was stirred at 150° C. (bath temperature) for 2 h. The reaction mixture was cooled to room temperature and diluted with water and EtOAc. After stirring for 30 min, the organic layer was separated, washed with saturated NaHCO₃ and brine, dried (Na₂SO₄), filtered and concentrated. Chromatography (silica gel, heptane to 30% EtOAc) gave (1H-indol-3-yl)(4-(5-(trichloromethyl)-1,2,4-oxadiazol-3-yl)thiazol-2-yl)methanone (19 mg). ESI MS m/z 413 [M−H]⁻.

Step 3. Ammonia (7 N) in methanol (2.0 mL, 14.0 mmol) was added to (1H-indol-3-yl)(4-(5-(trichloromethyl)-1,2,4-oxadiazol-3-yl)thiazol-2-yl)methanone (19 mg, 0.046 mmol) at room temperature. The reaction mixture was stirred for 15 h then concentrated to dryness and the residue was dried in vacuo to provide (4-(5-amino-1,2,4-oxadiazol-3-yl)thiazol-2-yl)(1H-indol-3-yl)methanone (15.4 mg) as a yellow solid.

### Example 61: Preparation of (5-chloro-1H-indol-3-yl)(4-(5-(trifluoromethyl)-1,2,4-oxadiazol-3-yl)thiazol-2-yl)methanone (ARI-116)

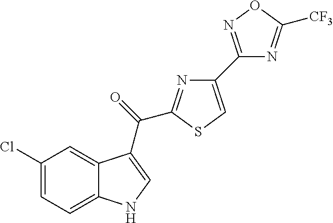 [SMILES: O=C(c1nc(-c2noc(C(F)(F)F)n2)cs1)c1c[nH]c2ccc(Cl)cc12]

A mixture of 2-(5-chloro-1H-indole-3-carbonyl)thiazole-4-carbonitrile (0.250 g, 0.869 mmol), hydroxylamine hydrochloride (0.151 g, 2.172 mmol), and potassium phosphate, tribasic (0.553 g, 2.61 mmol) in N,N-dimethylformamide (10.86 ml) were heated to 100° C. in the microwave for 1 h. Trifluoroacetic anhydride (0.491 ml, 3.48 mmol) was then added to the cooled solution and the reaction was again heated to 100° C. in a microwave reactor for an additional hour. The reaction mixture was concentrated. Chromatography (C18, 0 to100% ACN/water) gave and (5-chloro-1H-indol-3-yl)(4-(5-(trifluoromethyl)-1,2,4-oxadiazol-3-yl)thiazol-2-yl)methanone (41.9 mg) as a yellow solid.

### Example 62: Preparation of (1H-indol-3-yl)(4-(5-(methylamino)-1,2,4-oxadiazol-3-yl)thiazol-2-yl)methanone (ARI-117)

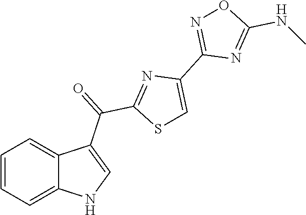 [SMILES: CNc1nc(-c2csc(C(=O)c3c[nH]c4ccccc34)n2)no1]

Methylamine (2.0 M in THF, 15 mL, 30.0 mmol) was added to (1H-indol-3-yl)(4-(5-(trichloromethyl)-1,2,4-oxadiazol-3-yl)thiazol-2-yl)methanone (120 mg, 0.290 mmol) at 0° C. The reaction mixture was stirred for 22 h with gradual warming to room temperature. The reaction mixture was concentrated to dryness and the residue was dried in vacuo to provide (1H-indol-3-yl)(4-(5-(methylamino)-1,2,4-oxadiazol-3-yl)thiazol-2-yl)methanone (79 mg) as a yellow solid.

### Example 63: Preparation of (4-(5-amino-1,3,4-oxadiazol-2-yl)thiazol-2-yl)(5,6-dichloro-1H-indol-3-yl)methanone (ARI-120)

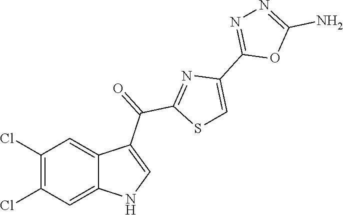 [SMILES: Nc1nnc(-c2csc(C(=O)c3c[nH]c4cc(Cl)c(Cl)cc34)n2)o1]

Prepared according to the method described in Example 131 except that 5,6-dichloro-1H-indole instead of 5,6-difluoro-1H-indole was used in Step 1.

### Example 64: Preparation of (4-(5-amino-1,3,4-oxadiazol-2-yl)thiazol-2-yl)(5,6-dichloro-1H-indol-3-yl)methanone (ARI-121)

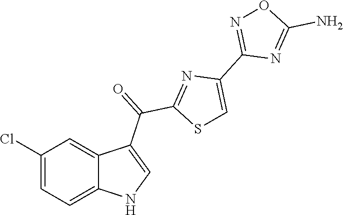 [SMILES: Nc1nc(-c2csc(C(=O)c3c[nH]c4ccc(Cl)cc34)n2)no1]

ARI-121 was synthesized according to the scheme of FIG. 20 and by the following method:

### Step 1: tert-Butyl 5-chloro-3-(4-(N′-hydroxycarbamimidoyl)thiazole-2-carbonyl)-1H-indole-1-carboxylatee (96-1)

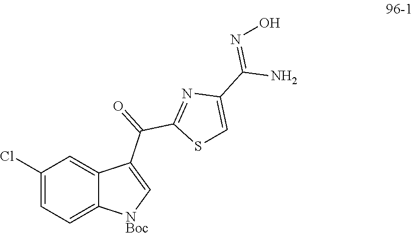 [SMILES: CC(C)(C)OC(=O)n1cc(C(=O)c2nc(/C(N)=N/O)cs2)c2cc(Cl)ccc21]

This compound was synthesized according to the protocol described in Example 21 step 1 from compound 39-2 to give title compound 96-1 in the form of a yellow solid (2.10 g, 83% yield).

### Step 2: tert-Butyl 3-(4-(5-amino-1,2,4-oxadiazol-3-yl)thiazole-2-carbonyl)-5-chloro-1H-indole-1-carboxylate (96-2)

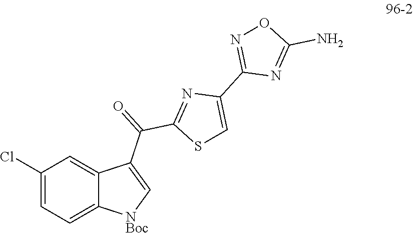 [SMILES: CC(C)(C)OC(=O)n1cc(C(=O)c2nc(-c3noc(N)n3)cs2)c2cc(Cl)ccc21]

BrCN (1.0 g, 9 mmol) was added to a suspension of compound 96-1 (420 mg, 1 mmol) in EtOH (200 mL) and H₂O (50 mL) at room temperature. The mixture was heated to 65° C. and stirred for 20 h. After cooled to room temperature, the mixture was filtered to collect the solid. The solid was washed with EtOH (10 mL×3), dried to afford compound 96-2 (290 mg, 65% yield) as yellow solid.

### Step 3: (4-(5-amino-1,2,4-oxadiazol-3-yl)thiazol-2-yl)(5-chloro-1H-indol-3-yl)methanone (ARI-121)

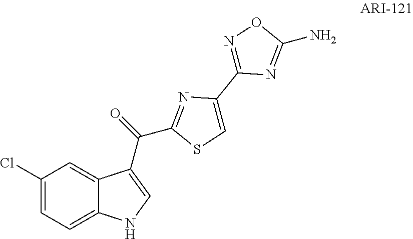 [SMILES: Nc1nc(-c2csc(C(=O)c3c[nH]c4ccc(Cl)cc34)n2)no1]

This compound was synthesized according to the protocol described in Example 116 step 2 from compound 96-2 (380 mg, 0.85 mmol) to give title compound ARI-121 in the form of a yellow solid (228 mg, 78% yield). ¹H-NMR (400 MHz, DMSO-d6): δ 12.53 (bs, 1H), 9.14˜9.16 (d, J=2.4 Hz, 1H), 8.59 (s, 1H), 8.30 (s, 1H), 8.10 (s, 2H), 7.63˜7.66 (d, J=8.4 Hz, 1H), 7.33˜7.36 (m, 1H). LC-MS: m/z 344.3 [M−H]⁻.

### Example 65: Preparation of ethyl 2-(1H-indole-3-carbonyl)thiazole-4-carboxylate (PTC17341-17, ARI-041)

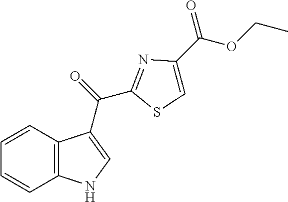 [SMILES: CCOC(=O)c1csc(C(=O)c2c[nH]c3ccccc23)n1]

ARI-041 was synthesized according to the scheme of FIG. 21 and by the following method:

### Step: Ethyl 2-(1H-indole-3-carbonyl)thiazole-4-carboxylate

K₂CO₃ (1.85 g, 13.4 mmol) was added to a solution of compound 1-5 (2.5 g, 6.7 mmol) in DMF (30 mL) at room temperature. The mixture was stirred for 5 min, then iodoethane (1.57 g, 10.1 mmol) was added. The resulting mixture was stirred for 2 h, then quenched with water (200 mL). The mixture was stirred for 0.5 h, then filtered to collect the solid. The solid was washed with water (30 mL×3) and EtOAc (30 mL×3), dried to afford ethyl ester (2.52 g, 94% yield) as off-white solid.

The above ethyl ester (2.50 g, 6.3 mmol) was dissolved in THF/DCM (10 mL/10 mL) at 0° C., and the mixture was allowed to warm to room temperate and was then stirred for 2 h. The mixture was concentrated to dryness. The residue was suspended in saturated aqueous NaHCO₃ (50 mL) and EtOAc (50 mL), stirred for 0.5 h, then filtered to collect the solid. The solid was washed with water (30 mL×3) and EtOAc (30 mL×3), dried to afford ethyl 2-(1H-indole-3-carbonyl)thiazole-4-carboxylate (1.70 g, 91% yield) as yellow solid. ¹H-NMR (400 MHz, DMSO-d6): δ 12.38 (s, 1H), 9.10 (s, 1H), 8.87 (s, 1H), 8.30˜8.35 (m, 1H), 7.55˜7.62 (m, 1H), 7.28˜7.33 (m, 2H), 4.35˜4.44 (q, J=7.2 Hz, 2H), 1.34˜1.40 (t, J=7.2 Hz, 3H). LC-MS: m/z 301.2 [M+H]⁺.

### Example 66: Preparation of isopropyl 2-(1H-indole-3-carbonyl)thiazole-4-carboxylate (ARI-042)

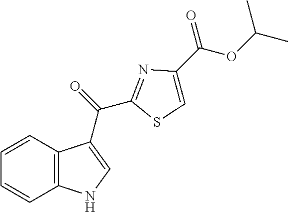 [SMILES: CC(C)OC(=O)c1csc(C(=O)c2c[nH]c3ccccc23)n1]

Prepared according to the method described in Example 14 except that isopropanol was used instead of 1,3-propanediol.

### Example 67: Preparation of propyl 2-(1H-indole-3-carbonyl)thiazole-4-carboxylate (ARI-043)

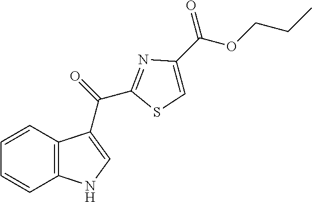 [SMILES: CCCOC(=O)c1csc(C(=O)c2c[nH]c3ccccc23)n1]

Prepared according to the method described in Example 14 except that propanol was used instead of 1,3-propanediol.

### Example 68: Preparation of 2-(5-chloro-1H-indole-3-carbonyl)-N-methylthiazole-4-carboxamide (PTC17341-06) (ARI-049)

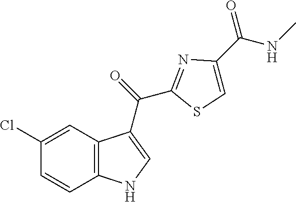 [SMILES: CNC(=O)c1csc(C(=O)c2c[nH]c3ccc(Cl)cc23)n1]

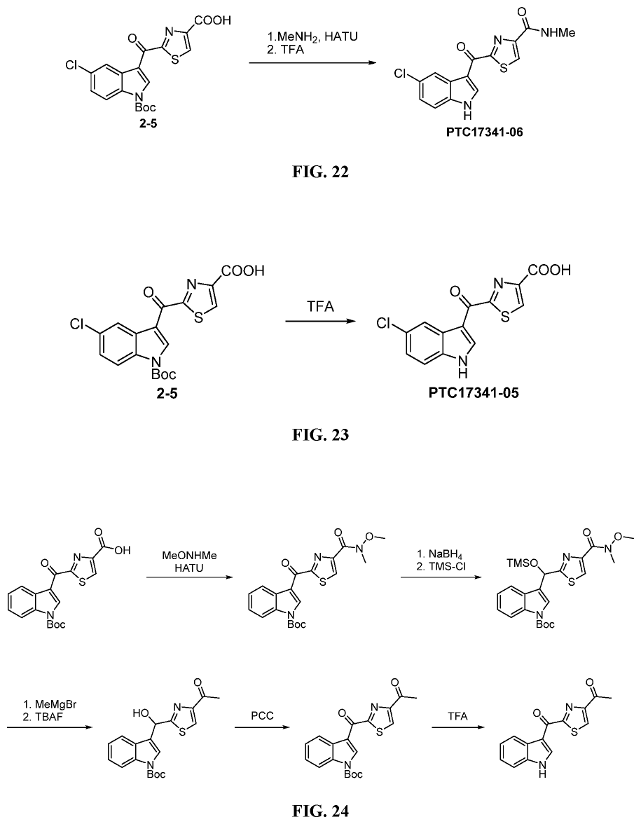

ARI-049 was synthesized according to the scheme of FIG. 22 and by the following method:

### Step: 2-(5-Chloro-1H-indole-3-carbonyl)-N-methylthiazole-4-carboxamide (PTC17341-06, ARI-049)

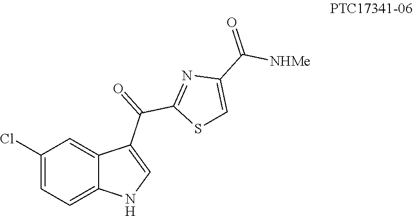 [SMILES: CNC(=O)c1csc(C(=O)c2c[nH]c3ccc(Cl)cc23)n1]

HATU (2.40 g, 6.4 mmol) and DIPEA (1.90 g, 14.7 mmol) were added to a suspension of compound 2-5 (2.00 g, 4.9 mmol) and methylamine hydrochloride (0.50 g, 7.4 mmol) in DMF (20 mL) at room temperature. The mixture was stirred overnight, then quenched with H₂O (100 mL). The mixture was stirred for 0.5 h, then filtered to collect the solid. The solid was washed with water (30 mL×3) and EtOAc (30 mL×3), dried to afford amide (1.20 g, 58% yield) as off-white solid.

The above amide (1.20 g, 2.8 mmol) was dissolved in THF/DCM (10 mL/10 mL) at 0° C., then the mixture was allowed to warm to room temperate and stirred for 2 h. The mixture was concentrated to dryness. The residue was suspended in saturated aqueous NaHCO₃ (50 mL) and EtOAc (50 mL), stirred for 0.5 h, then filtered to collect the solid. The solid was washed with water (30 mL×3) and EtOAc (30 mL×3), dried to afford PTC17341-06 (ARI-049) (820 mg, 89% yield) as yellow solid. ¹H-NMR (400 MHz, DMSO-d6): δ 12.52 (bs, 1H), 9.50 (s, 1H), 8.74 (bs, 1H), 8.62 (s, 1H), 8.30˜8.31 (d, J=2.0 Hz, 1H), 7.58˜7.62 (d, J=8.8 Hz, 1H), 7.30˜7.35 (m, 2H), 2.85˜2.90 (d, J=4.8 Hz, 3H). LC-MS: m/z 318.0 [M−H]⁻.

### Example 69: Preparation of methyl 2-(5-chloro-1H-indole-3-carbonyl)thiazole-4-carboxylate (ARI-055)

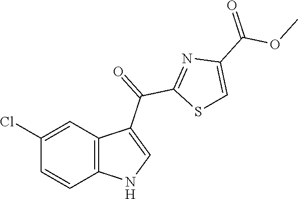 [SMILES: COC(=O)c1csc(C(=O)c2c[nH]c3ccc(Cl)cc23)n1]

Prepared according to the method described in Example 14 except that 2-(5-chloro-1H-indole-3-carbonyl)thiazole-4-carboxylic acid and methanol were used.

### Example 70: Preparation of 2-(5,6-dibromo-1H-indole-3-carbonyl)-N-methylthiazole-4-carboxamide (ARI-057)

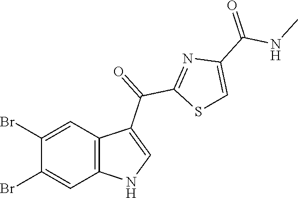 [SMILES: CNC(=O)c1csc(C(=O)c2c[nH]c3cc(Br)c(Br)cc23)n1]

BOC-protected ARI-004 (2.3 g) was dissolved in glacial acetic acid (25 mL). Br₂ (3 eq) was added dropwise. The resulting mixture was stirred at 20˜30° C. for 72 h. The acetic acid was removed under vacuum to afford the crude product as a 3:1 mixture of 5,6 dibromo and monobromo carboxamido products. The crude product mixture was recrystallized from hot glacial aetic acid to afford 2.3 gm of ARI-057 as an off-white solid.

### Example 71: Preparation of 2-(5-chloro-1H-indole-3-carbonyl)thiazole-4-carboxylic acid (PTC17341-05, ARI-058)

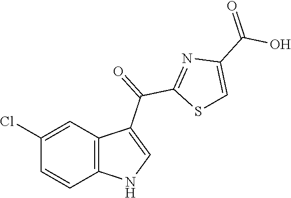 [SMILES: O=C(O)c1csc(C(=O)c2c[nH]c3ccc(Cl)cc23)n1]

ARI-058 was synthesized according to the scheme of FIG. 23 and by the following method:

### Step: 2-(5-chloro-1H-indole-3-carbonyl)thiazole-4-carboxylic acid (PTC17341-05, ARI-058)

A solution of compound 2-5 (1.5 g, 3.7 mmol) in DCM (10 mL) and TFA (10 mL) was stirred at room temperature for 3 h. The mixture was concentrated to dryness. The residue was suspended in EtOAc (20 mL), alkalified by saturated aqueous NaHCO₃ to pH of 7˜8, then acidified by aqueous 1N HCl to pH of 3. The mixture was filtered to collect the solid. The solid was washed with water (10 mL×3) and EtOAc (10 mL×3), dried to afford ARI-058 (HCl salt, 1.1 g, 87% yield) as yellow solid. ¹H-NMR (400 MHz, DMSO-d6): δ 13.46 (bs, 1H), 12.76 (s, 1H), 9.17 (s, 1H), 8.83 (s, 1H), 8.28-8.29 (d, J=2.0 Hz, 1H), 7.63-7.66 (d, J=8.4 Hz, 1H), 7.31˜7.35 (m, 1H) LC-MS: m/z 305.0 [M−H]⁻.

### Example 72: Preparation of tert-butyl 2-(1H-indole-3-carbonyl)thiazole-4-carboxylate (ARI-059)

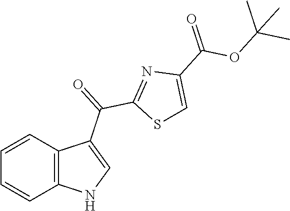 [SMILES: CC(C)(C)OC(=O)c1csc(C(=O)c2c[nH]c3ccccc23)n1]

Prepared according to the method described in Example 14 except that tert-butanol was used instead of 1,3-propanediol.

### Example 73: Preparation of 2-(5-fluoro-1H-indole-3-carbonyl)-N-methylthiazole-4-carboxamide (ARI-065)

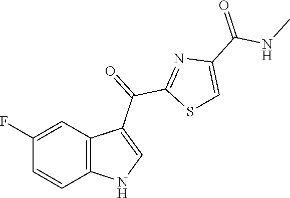 [SMILES: CNC(=O)c1csc(C(=O)c2c[nH]c3ccc(F)cc23)n1]

Prepared according to the method described in Example 24 except that 5-fluoro-1H-indole-3-carboxylic acid and methylamine were used.

### Example 74: Preparation of methyl 2-(5-fluoro-1H-indole-3-carbonyl)thiazole-4-carboxylate (ARI-066)

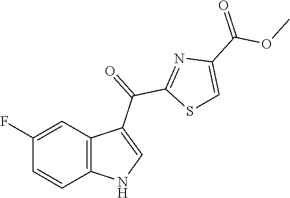 [SMILES: COC(=O)c1csc(C(=O)c2c[nH]c3ccc(F)cc23)n1]

Prepared according to the method described in Example 14 except that methanol was used instead of 1,3-propanediol.

### Example 75: Preparation of 1-(2-(1H-indole-3-carbonyl)thiazol-4-yl)ethan-1-one (ARI-077)

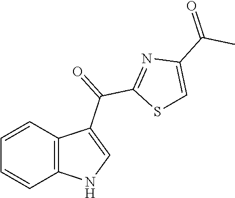 [SMILES: CC(=O)c1csc(C(=O)c2c[nH]c3ccccc23)n1]

ARI-077 was synthesized according to the scheme of FIG. 24 and by the following method:

Step 1. HATU (12.9 g, 34 mmol) and DIPEA (10.1 g, 78 mmol) were added to a suspension of 2-(1-(tert-butoxycarbonyl)-1H-indole-3-carbonyl)thiazole-4-carboxylic acid (10.0 g, 26 mmol) and N,O-dimethylhydroxyamine hydrochloride (3.7 g, 38 mmol) in DMF (50 mL) at room temperature. The mixture was stirred overnight, then quenched with H₂O (200 mL). The mixture was stirred for 0.5 h, then filtered to collect the solid. The solid was washed with water (50 mL×3) and EtOAc (50 mL×3), dried to afford tert-butyl 3-(4-(methoxy(methyl)carbamoyl)thiazole-2-carbonyl)-1H-indole-1-carboxylate (9.1 g, 84% yield) as off-white solid.

Step 2. NaBH₄ (0.54 g, 14 mmol) was added portionwise to a solution of compound tert-butyl 3-(4-(methoxy(methyl)carbamoyl)thiazole-2-carbonyl)-1H-indole-1-carboxylate (5.88 g, 14 mmol) in DCM (50 mL) and EtOH (50 mL) at 0° C. over 10 min. The resulting mixture was stirred for 0.5 h, then quenched with water (100 mL), extracted with DCM (100 mL×3). The combined organic phases were washed with brine (200 mL×2), dried, concentrated to afford alcohol (˜6.0 g) as an oil. The alcohol (6.0 g, 14 mmol) and triethanolamine (TEA) (2.2 g, 28 mmol) were dissolved in THF (60 mL), and cooled to 0° C., then TMSCl (2.2 g, 20 mmol) was added dropwise over 10 min. The resulting mixture was stirred for 2 h, then quenched with water (100 mL), extracted with EtOAc (50 mL×3). The combined organic phases were washed with saturated aqueous NaHCO₃ (50 mL×2) and brine (200 mL×2), dried, concentrated to afford compound tert-butyl 3-((4-(methoxy(methyl)carbamoyl)thiazol-2-yl) (trimethyl silyloxy)methyl)-1H-indole-1-carboxylate (6.9 g, ˜100% yield) as an oil, which was used for next step without further purification.

Step 3. MeMgBr (2 M in Et₂O, 5 mL, 10 mmol) was added portionwise to a solution of compound tert-butyl 3-((4-(methoxy(methyl)carbamoyl)thiazol-2-yl) (trimethyl silyloxy)methyl)-1H-indole-1-carboxylate (2.0 g, 4.1 mmol) in THF (20 mL) at 0° C. over 10 min. The resulting mixture was stirred for 0.5 h, then quenched with saturated aqueous NH₄Cl (50 mL), extracted with EtOAc (50 mL×3). The combined organic phases were washed with brine (100 mL×2), dried, concentrated to afford ketone (˜1.8 g) as an oil.

The above ketone (1.8 g) was dissolved in THF (20 mL), tetrabutylammonium fluoride (TBAF) (1.1 g, 4 mmol) was added. The mixture was stirred for 2 h at room temperature, then quenched with water (50 mL), extracted with EtOAc (50 mL×3). The combined organic phases were washed with brine (100 mL×2), dried, concentrated to dryness. The residue was purified by silica gel chromatography (EtOAc/Hexane=1:3) to afford tert-butyl 3-((4-acetylthiazol-2-yl)(hydroxy)methyl)-1H-indole-1-carboxylate (950 mg, 62% yield).

Step 4. Pyridinium chlorochromate (PCC) (0.8 g, 3.7 mmol) was added to a solution of compound tert-butyl 3-((4-acetylthiazol-2-yl)(hydroxy)methyl)-1H-indole-1-carboxylate (950 mg, 2.6 mmol) in DCM (50 mL) at room temperature. The resulting mixture was stirred overnight, then quenched with water (50 mL). The mixture was filtered, and the filtrate was extracted with DCM (50 mL×3). The combined organic phases were washed with brine (100 mL×2), dried, concentrated to dryness. The residue was purified by silica gel chromatography (EtOAc/Hexane/THF=1:3:1) to afford tert-butyl 3-(4-acetylthiazole-2-carbonyl)-1H-indole-1-carboxylate (670 mg, 69% yield).

Step 5. A solution of tert-butyl 3-(4-acetylthiazole-2-carbonyl)-1H-indole-1-carboxylate (1.5 g, 3.7 mmol) in DCM (10 mL) and TFA (10 mL) was stirred at room temperature. Upon completion, the mixture was concentrated to dryness. The residue was suspended in EtOAc basified with saturated aqueous NaHCO₃ to pH of 7˜8, then acidified by aqueous 1N HCl to pH of 3. The mixture was filtered to collect the solid. The solid was washed with water and EtOAc, dried to afford 1-(2-(1H-indole-3-carbonyl)thiazol-4-yl)ethan-1-one in the form of a yellow solid (660 mg, 90% yield). ¹H-NMR (400 MHz, DMSO-d6): δ 12.35 (bs, 1H), 9.18 (s, 1H), 8.86 (s, 1H), 8.32 (m, 1H), 7.59 (m, 1H), 7.31 (m, 2H), 2.74 (s, 3H). LC-MS: m/z 270 [M+H]⁺.

### Example 76: Preparation of 1-(2-(5-chloro-1H-indole-3-carbonyl)thiazol-4-yl)propan-1-one (ARI-067)

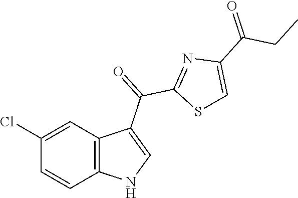 [SMILES: CCC(=O)c1csc(C(=O)c2c[nH]c3ccc(Cl)cc23)n1]

Prepared according to the method described in Example 75 except that 2-(1-(tert-butoxycarbonyl)-5-chloro-1H-indole-3-carbonyl)thiazole-4-carboxylic acid was used.

### Example 77: Synthesis of (4-(1-hydroxypropyl)thiazol-2-yl)(1H-indol-3-yl) methanone (PTC17341-16, ARI-068)

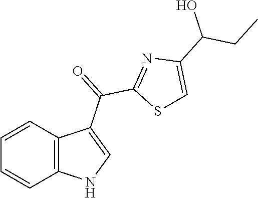 [SMILES: CCC(O)c1csc(C(=O)c2c[nH]c3ccccc23)n1]

### (S)-(4-(1-hydroxypropyl)thiazol-2-yl)(1H-indol-3-yl) methanone (PTC17341-16A, ARI-092) and (R)-(4-(1-hydroxypropyl)thiazol-2-yl) (1H-indol-3-yl)methanone (PTC17341-16B, ARI-094)

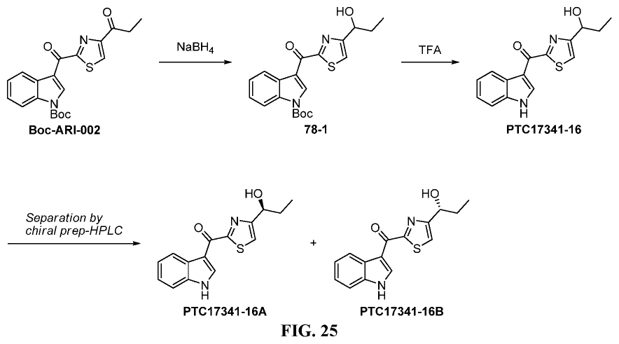

ARI-068 was synthesized according to the scheme of FIG. 25 and by the following method:

### Step 1: tert-Butyl 3-(4-(1-hydroxypropyl)thiazole-2-carbonyl)-1H-indole-1-carboxylate (78-1)

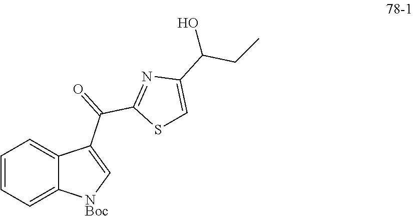 [SMILES: CCC(O)c1csc(C(=O)c2cn(C(=O)OC(C)(C)C)c3ccccc23)n1]

NaBH₄ (60 mg, 1.6 mmol, 0.6 eq) was added portionwise to a solution of compound Boc-ARI-002 (1.0 g, 2.6 mmol) in DCM (30 mL) and MeOH (20 mL) at 0° C. The mixture was stirred for 2 h, then quenched with H₂O (30 mL), extracted with EtOAc (50 mL×3). The combined organic phases were washed with brine (50 mL×3), dried over anhydrous Na₂SO₄, filtered and concentrated in vacuo. The residue was purified by silica gel column chromatography (hexane/EtOAc=2:1) to give compound 78-1 (700 mg, 70% yield) as an oil.

### Step 2: (4-(1-Hydroxypropyl)thiazol-2-yl)(1H-indol-3-yl)methanone (PTC17341-16)

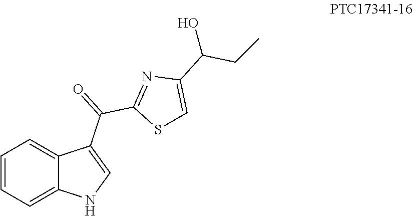 [SMILES: CCC(O)c1csc(C(=O)c2c[nH]c3ccccc23)n1]

This compound was synthesized according to the protocol described in Example 71 from compound 78-1 (2.2 g, 5.7 mmol) to give title compound PTC17341-16 (ARI-068) in the form of a yellow solid (1.4 g, 86% yield). ¹H-NMR (400 MHz, DMSO-d6): δ 12.25 (bs, 1H), 9.15 (d, J=2.0 Hz, 1H), 8.30˜8.34 (m, 1H), 7.82 (s, 1H), 7.55˜7.60 (m, 1H), 7.26˜7.30 (m, 2H), 5.46˜5.48 (d, J=5.2 Hz, 1H), 4.71˜4.77 (m, 1H), 1.90˜2.00 (m, 1H), 1.75˜1.88 (m, 1H), 0.90˜0.99 (t, J=5.4 Hz, 3H). LC-MS: m/z 287.2 [M+H]⁺.

### Step 3: (S)-(4-(1-Hydroxypropyl)thiazol-2-yl)(1H-indol-3-yl)methanone (PTC17341-16A) and (R)-(4-(1-Hydroxypropyl)thiazol-2-yl)(1H-indol-3-yl) methanone (PTC17341-16B)

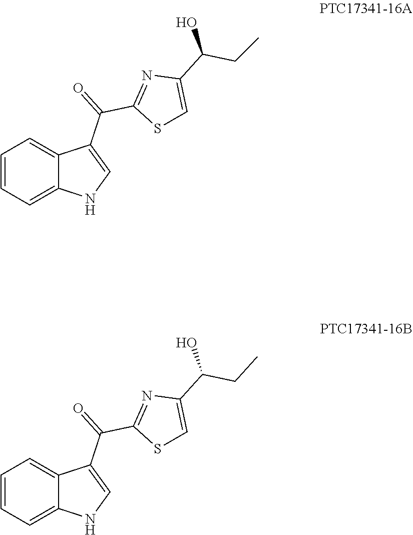 [SMILES: CC[C@H](O)c1csc(C(=O)c2c[nH]c3ccccc23)n1 ; CC[C@@H](O)c1csc(C(=O)c2c[nH]c3ccccc23)n1]

Compound PTC17341-16 (1.0 g, 3.5 mmol) was separated by chiral prep-HPLC to afford compound PTC17341-16A (ARI-092) (140 mg, 14% yield) and PTC17341-16B (ARI-094) (128 mg, 13% yield).

PTC17341-16A (ARI-092): yellow solid, ¹H-NMR (400 MHz, DMSO-d6): δ 12.21 (bs, 1H), 9.10 (s, 1H), 8.30˜8.34 (m, 1H), 7.82 (s, 1H), 7.55˜7.60 (m, 1H), 7.24˜7.31 (m, 2H), 5.44˜5.47 (d, J=6.8 Hz, 1H), 4.70˜7.77 (m, 1H), 1.88˜1.95 (m, 1H), 1.75˜1.85 (m, 1H), 0.90˜0.96 (t, J=6.0 Hz, 3H). LC-MS: m/z 287.2 [M+H]⁺.

PTC17341-16B (ARI-094): yellow solid, ¹H-NMR (400 MHz, DMSO-d6): δ 12.22 (bs, 1H), 9.10 (s, 1H), 8.30˜8.34 (m, 1H), 7.82 (s, 1H), 7.55˜7.60 (m, 1H), 7.24˜7.31 (m, 2H), 5.44˜5.47 (d, J=6.0 Hz, 1H), 4.70˜4.77 (m, 1H), 1.88˜2.05 (m, 1H), 1.74˜1.85 (m, 1H), 0.92˜0.98 (t, J=6.0 Hz, 3H). LC-MS: m/z 287.2 [M+H]⁺.

### Example 78: Synthesis of (E)-(1H-indol-3-yl)(4-(1-(methoxyimino)-2-methyl propyl) thiazol-2-yl)methanone (PTC17341-22-A) and (Z)-(1H-indol -3-yl)(4-(1-(methoxyimino)-2-methylpropyl)thiazol-2-yl)methanone (PTC17341-22-B) (ARI-069 and ARI-070)

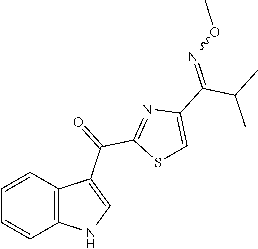

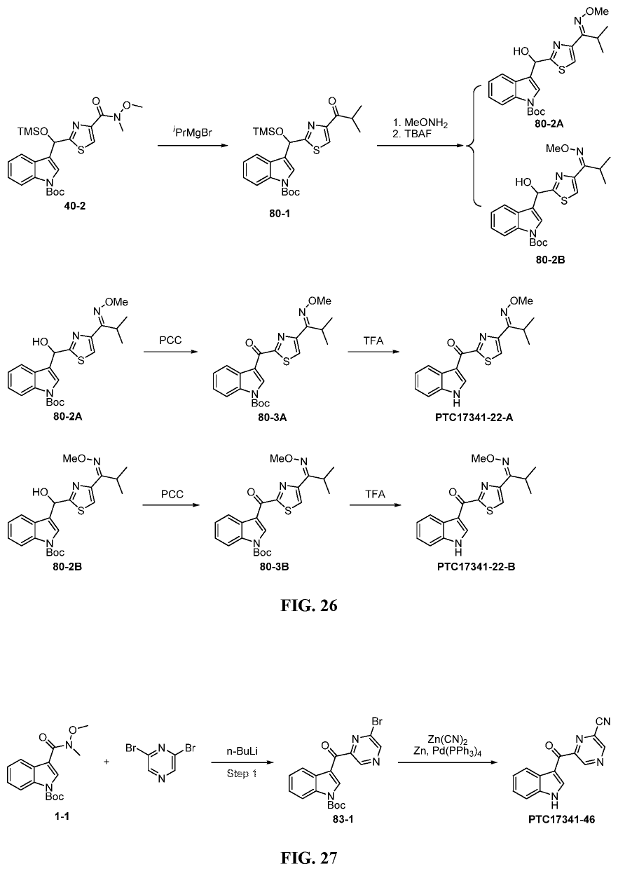

ARI-069 and ARI-070 were synthesized according to the scheme of FIG. 26 and by the following method:

### Step 1: tert-Butyl 3-((4-isobutyrylthiazol-2-yl)(trimethylsilyloxy)methyl)-1H-indole-1-carboxylate (80-1)

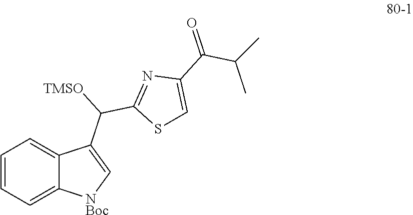

This compound was synthesized according to the protocol described in Example 127 from compound 40-2 (23.0 g, 47 mmol) to give title compound 80-1 (15.3 g, 69% yield).

### Step 2: (E)-tert-Butyl 3-(hydroxy(4-(1-(methoxyimino)-2-methylpropyl)thiazol-2-yl)methyl)-1H-indole-1-carboxylate (80-2A) and (Z)-tert-butyl 3-(hydroxyl (4-(1-(methoxyimino)-2-methylpropyl)thiazol-2-yl)methyl)-1H-indole-1-carboxylate (80-2B)

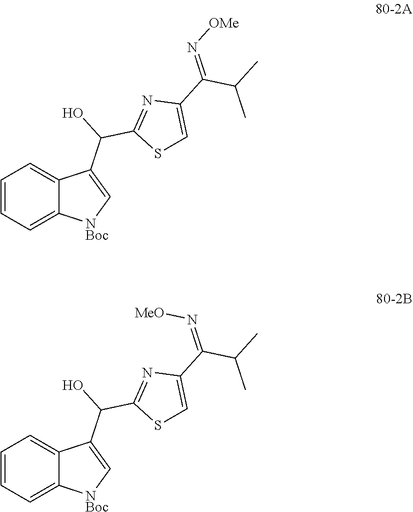 [SMILES: CO/N=C(/c1csc(C(O)c2cn(C(=O)OC(C)(C)C)c3ccccc23)n1)C(C)C ; CO/N=C(\c1csc(C(O)c2cn(C(=O)OC(C)(C)C)c3ccccc23)n1)C(C)C]

NaOAc (2.64 g, 32 mmol) and methoxylamine hydrochloride (1.34 g, 16 mmol) were added to a solution of compound 80-1 (3.80 g, 8 mmol) in EtOH (20 mL) and H₂O (50 mL) at room temperature. The mixture was heated to 70° C. and stirred for 2 h. After cooling to room temperature, the mixture was concentrated in vacuo. The residue was dissolved in THF (20 mL), and TBAF (2.30 g, 8.8 mmol) was added. The mixture was stirred for 2 h at room temperature, then quenched with water (50 mL), extracted with EtOAc (50 mL×3). The combined organic phases were washed with brine (100 mL×2), dried, concentrated to dryness. The residue was purified by silica gel chromatography (EtOAc/Hexane=1:15) and afforded compound 80-2A (600 mg, 17% yield) and 80-2B (598 mg, 17% yield).

### Step 3a: (E)-tert-Butyl 3-(4-(1-(methoxyimino)-2-methylpropyl)thiazole-2-carbonyl)-1H-indole-1-carboxylate (80-3A)

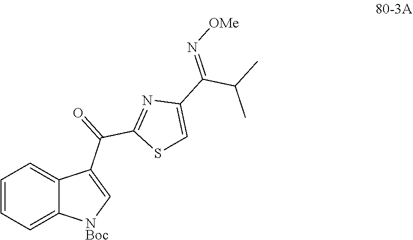 [SMILES: CO/N=C(/c1csc(C(=O)c2cn(C(=O)OC(C)(C)C)c3ccccc23)n1)C(C)C]

This compound was synthesized according to the protocol described in Example 127 from compound 80-2A (600 mg, 1.4 mmol) to give title compound 80-3A (310 mg, 52% yield).

### Step 3b: (Z)-tert-Butyl 3-(4-(1-(methoxyimino)-2-methylpropyl)thiazole-2-carbonyl)-1H-indole-1-carboxylate (80-3B)

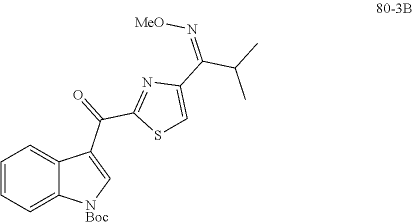 [SMILES: CO/N=C(\c1csc(C(=O)c2cn(C(=O)OC(C)(C)C)c3ccccc23)n1)C(C)C]

This compound was synthesized according to the protocol described in Example 127 from compound 80-2B (598 mg, 1.4 mmol) to give title compound 80-3B (301 mg, 50% yield).

### Step 4a: (E)-(1H-indol-3-yl)(4-(1-(methoxyimino)-2-methylpropyl)thiazol-2-yl) methanone (PTC17341-22-A, ARI-069)

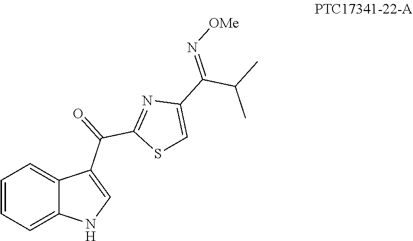 [SMILES: CO/N=C(/c1csc(C(=O)c2c[nH]c3ccccc23)n1)C(C)C]

This compound was synthesized according to the protocol described in Example 71 from compound 80-3A (300 mg, 0.7 mmol) to give the title compound ARI-069 (PTC17341-22-A) in the form of a yellow solid (130 mg, 57% yield). ¹H-NMR (400 MHz, DMSO-d6): δ 12.36 (bs, 1H), 9.09 (s, 1H), 8.30˜8.34 (m, 1H), 8.21 (s, 1H), 7.55˜7.60 (m, 1H), 7.26˜7.30 (m, 2H), 3.95 (s, 3H), 3.65˜3.69 (m, 1H), 1.24˜1.32 (m, 6H). LC-MS: m/z 328.3 [M+H]⁺.

### Step 4b: (Z)-(1H-indol-3-yl)(4-(1-(methoxyimino)-2-methylpropyl)thiazol-2-yl) methanone (PTC17341-22-B)

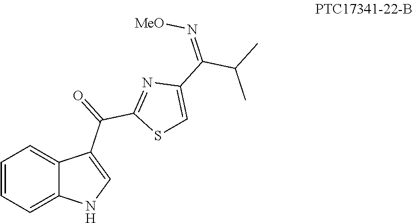 [SMILES: CO/N=C(\c1csc(C(=O)c2c[nH]c3ccccc23)n1)C(C)C]

This compound was synthesized according to the protocol described in Example 71 from compound 80-3B (300 mg, 0.7 mmol) to give the title compound ARI-070 (PTC17341-22-B) in the form of a yellow solid (172 mg, 75% yield). ¹H-NMR (400 MHz, DMSO-d6): δ 12.32 (bs, 1H), 9.02 (s, 1H), 8.74 (s, 1H), 8.31˜8.33 (m, 1H), 7.56˜7.59 (m, 1H), 7.28˜7.31 (m, 2H), 3.95 (s, 3H), 3.50˜3.55 (m, 1H), 1.24˜1.26 (d, J=6.8 Hz, 6H). LC-MS: m/z 326.3 [M−H]⁻.

### Example 79: Preparation of methyl 2-(1H-indole-2-carbonyl)thiazole-4-carboxylate (ARI-076)

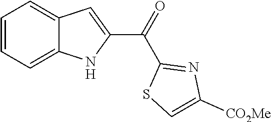 [SMILES: COC(=O)c1csc(C(=O)c2cc3ccccc3[nH]2)n1]

Prepared from indole 2-carboxylic acid by the method described in Example 130 to obtain 2-(1-(tert-butoxycarbonyl)-1H-indole-2-carbonyl) thiazole-4-carboxylic acid. 2-(1-(tert-butoxycarbonyl)-1H-indole-2-carbonyl) thiazole-4-carboxylic acid was then transformed to methyl 2-(1H-indole-2-carbonyl)thiazole-4-carboxylate (ARI-076) according to the method described in Example 65 except that iodomethane instead of iodoethane was used.

### Example 80: Preparation of 2-(5-methoxy-1H-indole-3-carbonyl)-N-methylthiazole-4-carboxamide (ARI-078)

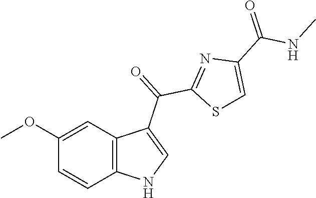

Prepared according to the method for preparing the key intermediate 2-(1H-indole-3-carbonyl)thiazole-4-carboxylic acid using 5-methoxy-1H-indole-3-carboxylic acid to obtain 2-(1-(tert-butoxycarbonyl)-5-methoxy-1H-indole-3-carbonyl)thiazole-4-carboxylic acid which was transformed to the final product using the HATU and TFA methods. See Example 27: Preparation of N-(2-hydroxyethyl)-2-(1H-indole-3-carbonyl)thiazole-4-carboxamide (ART-036).

### Example 81: Preparation of 2-(1H-indole-2-carbonyl)-N-methylthiazole-4-carboxamide (ARI-079)

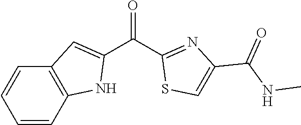 [SMILES: CNC(=O)c1csc(C(=O)c2cc3ccccc3[nH]2)n1]

Prepared according to the method for preparing the key intermediate 2-(1H-indole-3-carbonyl)thiazole-4-carboxylic acid using 1H-indole-2-carboxylic acid to obtain 2-(1-(tert-butoxycarbonyl)-1H-indole-2-carbonyl)thiazole-4-carboxylic acid which was transformed to the methylamide using conditions described in Example 24.

### Example 82: Preparation of 6-(1H-indole-3-carbonyl)pyrazine-2-carbonitrile (PTC17341-46, ARI-085)

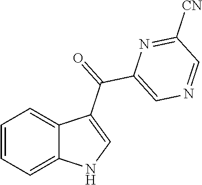 [SMILES: N#Cc1cncc(C(=O)c2c[nH]c3ccccc23)n1]

ARI-085 was synthesized according to the scheme of FIG. 27 and by the following method:

### Step 1: tert-Butyl 3-(6-bromopyrazine-2-carbonyl)-1H-indole-1-carboxylate (83-1)

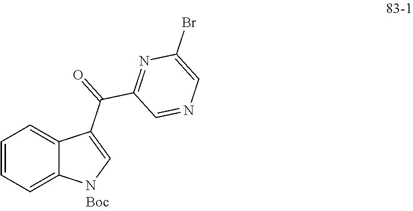 [SMILES: O=C(c1cncc(Br)n1)c1c[nH]c2ccccc12]

A solution of compound 1-1 (2.00 g, 6.6 mmol) and 2,6-dibromopyrazine (5.50 g, 23 mmol) in THF (100 mL) was cooled to −78° C., and n-BuLi (1.6 M solution in hexane, 8.4 mL, 13.4 mmol) was added dropwise at −78° C. over 10 min. The mixture was stirred for 0.5 h at this temperature, then allowed to warm to 0° C. and quenched with aqueous 10% NH₄Cl (100 mL) and EtOAc (100 mL). The organic phase was collected and washed with water (100 mL×2), saturated aqueous NaHCO₃ (100 mL×2), and brine (100 mL×1), dried (Na₂SO₄), filtered and concentrated to dryness. The residue was purified by silica gel chromatography (EtOAc/Hexane=1:5) and afforded compound 83-1 (2.10 g, 79% yield).

### Step 2: 6-(1H-indole-3-carbonyl)pyrazine-2-carbonitrile (PTC17341-46, ARI-085)

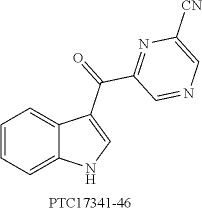 [SMILES: N#Cc1cncc(C(=O)c2c[nH]c3ccccc23)n1]

This compound was synthesized according to the protocol described in Example 118 from compound 83-1 (1.00 g, 2.5 mmol) to give title compound PTC17341-46 (ARI-085) in the form of a yellow solid (101 mg, 16% yield). ¹H-NMR (400 MHz, DMSO-d6): δ 12.30 (bs, 1H), 9.40˜9.44 (m, 2H), 8.59 (s, 1H), 8.32˜8.35 (m, 1H), 7.56˜7.60 (m, 1H), 7.29˜7.32 (m, 2H). LC-MS: m/z 248.8 [M+H]⁺.

### Example 83: Synthesis of methyl 6-(1H-indole-3-carbonyl)pyrimidine-4-carboxylate (PTC17341-35) (ARI-086)

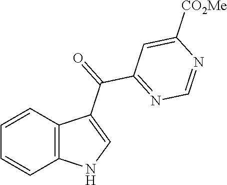 [SMILES: COC(=O)c1cc(C(=O)c2c[nH]c3ccccc23)ncn1]

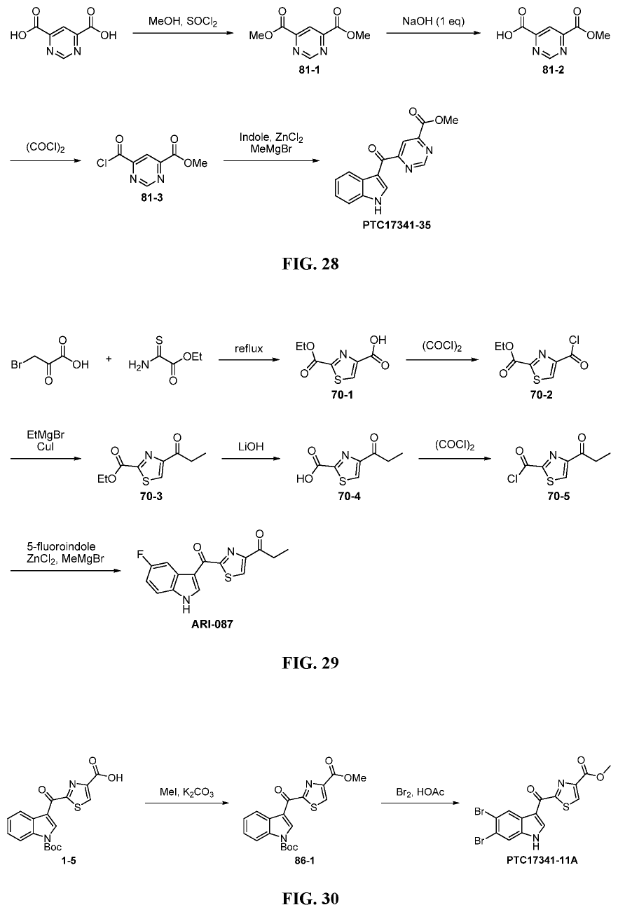

ARI-086 was synthesized according to the scheme of FIG. 28 and by the following method:

### Step 1: Dimethyl pyrimidine-4,6-dicarboxylate (81-1)

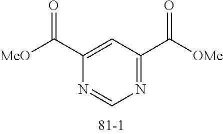 [SMILES: COC(=O)c1cc(C(=O)OC)ncn1]

SOCl₂ (4.76 g, 4 mmol) was added to a solution of pyrimidine-4,6-dicarboxylic acid (3.40 g, 2 mmol) in MeOH (250 mL) at 0° C. The mixture was heated under reflux and stirred for 5 h. After cooling to room temperature, the mixture was concentrated in vacuo. The residue was diluted with saturated aqueous NaHCO₃ (100 mL), and extracted with EtOAc (100 mL×3). The combined organic phases were washed with brine (100 mL×2), dried, concentrated to dryness. The residue was purified by silica gel chromatography (EtOAc/Hexane=1:5) and afforded compound 81-1 (3.10 g, 79% yield).

### Step 2: 6-(Methoxycarbonyl)pyrimidine-4-carboxylic acid (81-2)

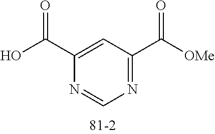 [SMILES: COC(=O)c1cc(C(=O)O)ncn1]

Sodium hydroxide (632 mg, 15.8 mmol) was added to a solution of compound 81-1 (3.10 g, 15.8 mmol) in MeOH (60 mL) and H₂O (6 mL) at 0° C. The resulting mixture was stirred for 2 h at room temperature, then acidified with 1M HCl aqueous to pH of 3. The mixture was concentrated to dryness. The residue was azeotroped two times with THF (50 mL portions) to afford crude compound 81-2 (3.30 g, ˜100% yield), which was used for next step without further purification.

### Step 3: Methyl 6-(chlorocarbonyl)pyrimidine-4-carboxylate (81-3)

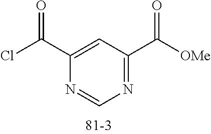 [SMILES: COC(=O)c1cc(C(=O)Cl)ncn1]

This compound was synthesized according to the protocol described in Example 84 from compound 81-2 (3.00 g, 16.5 mmol) to give title compound 81-3 (3.25 g, ˜100% yield), which was used for next step without further purification.

### Step 4: Methyl 6-(1H-indole-3-carbonyl)pyrimidine-4-carboxylate (PTC17341-35, ARI-086)

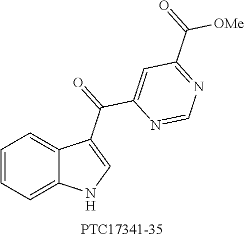 [SMILES: COC(=O)c1cc(C(=O)c2c[nH]c3ccccc23)ncn1]

This compound was synthesized according to the protocol described in Example 84 from compound 81-3 (3.25 g, 16.5 mmol) to give title compound PTC17341-35 (ARI-086) in the form of a yellow solid (275 mg, 6% yield). ¹H-NMR (400 MHz, DMSO-d6): δ 12.34 (bs, 1H), 9.58˜9.59 (d, J=1.6 Hz, 1H), 8.80 (s, 1H), 8.41 (s, 1H), 8.33˜8.35 (m, 1H), 7.55˜7.58 (m, 1H), 7.28˜7.32 (m, 2H), 3.97 (s, 3H). LC-MS: m/z 280.2 [M+H]⁺.

### Example 84: Preparation of 1-(2-(5-fluoro-1H-indole-3-carbonyl)thiazol-4-yl)propan-1-one (ARI-087)

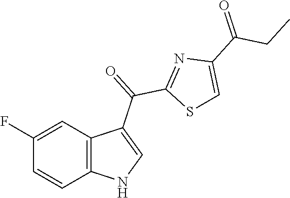 [SMILES: CCC(=O)c1csc(C(=O)c2c[nH]c3ccc(F)cc23)n1]

ARI-087 was synthesized according to the scheme of FIG. 29 and by the following method:

### Step 1: 2-(Ethoxycarbonyl)thiazole-4-carboxylic acid (70-1)

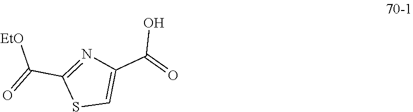 [SMILES: CCOC(=O)c1nc(C(=O)O)cs1]

Ethyl thiooxamate (1.0 Kg, 7.52 mol) was added portion-wise to a solution of 2-bromopyruvic acid (1.38 Kg, 8.27 mol) in THF (4 L) over 20 min while the reaction was cooled with water bath. The reaction mixture was stirred for 12 h at room temperature. The reaction mixture was filtered to remove solid. The filtrate was concentrated to dryness to afford crude compound 70-1 (1.8 kg). The crude 70-1 was triturated with EtOAc/hexane/H₂O (1:3:2, 6 L), filtered, and the solid was further triturated with EtOAc/hexane (1:8, 3 L), filtered, and the solid was dissolved in DCM (6 L), dried over anhydrous Na₂SO₄, concentrated to afford compound 70-1 (617 g, 41% yield based on ethyl thiooxamate) as light yellow solid. ¹H NMR (400 MHz, CDCl₃): δ 8.79 (s, 1H), 4.38˜4.46 (q, J=7.2 Hz, 2H), 1.3˜1.38 (t, J=7.2 Hz, 3H).

### Step 2: Ethyl 4-(chlorocarbonyl)thiazole-2-carboxylate (70-2)

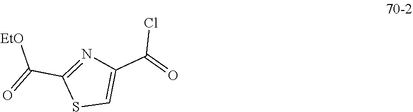 [SMILES: CCOC(=O)c1nc(C(=O)Cl)cs1]

Oxalyl chloride (63.1 g, 0.497 mol) was added dropwise to a suspension of compound 70-2 (50.0 g, 0.248 mol) in DCM (500 mL) at room temperature over 0.5 h. The reaction mixture was stirred for 4 h, then concentrated. The residue was azeotroped two times with DCM (500 mL portions) to afford crude compound 70-2 (55.1 g, ˜100% yield), which was used for next step without further purification.

### Step 3: Ethyl 4-propionylthiazole-2-carboxylate (70-3)

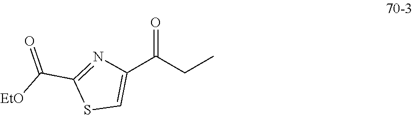

A mixture of compound 70-2 (55.1 g, 0.248 mol) and copper(I) iodide (9.5 g, 50 mmol) was stirred and cooled to −60° C. under N₂. EtMgBr (2M in THF, 150 mL) was added dropwise at −60˜−45° C. over 1 h. The mixture was stirred for 2 h at this temperature, and then quenched with saturated NH₄Cl aqueous (500 mL). The mixture was warmed to room temperature, then extracted with EtOAc (500 mL×3). The combined organic phases were washed with brine (500 mL×2), dried, concentrated to dryness. The residue was purified by silica gel column chromatography (hexane/EtOAc=20:1) to give compound 70-3 (23.7 g, 45% yield) as yellow solid. ¹H NMR (400 MHz, DMSO-d6): δ 8.83 (s, 1H), 4.39˜4.46 (q, J=7.2 Hz, 2H), 3.07˜3.14 (q, J=7.2 Hz, 2H), 1.33˜1.38 (t, J=7.2 Hz, 3H), 1.07˜1.11 (t, J=7.2 Hz, 3H).

### Step 4: 4-Propionylthiazole-2-carboxylic acid (70-4)

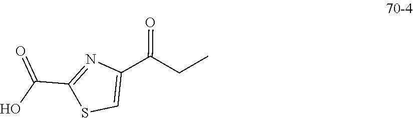

Lithium hydroxide monohydrate (3.8 g, 90 mmol) was added to a solution of compound 70-3 (6.4 g, 30 mmol) in THF (60 mL) and H₂O (6 mL) at 0° C. The resulting mixture was stirred for 2 h at room temperature, then acidified with 1M HCl aqueous to pH of 3. The mixture was concentrated to dryness. The residue was azeotroped two times with THF (50 mL portions) to afford crude compound 70-4 (10.9 g, ˜100% yield), which was used for next step without further purification.

### Step 5: 4-Propionylthiazole-2-carbonyl chloride (70-5)

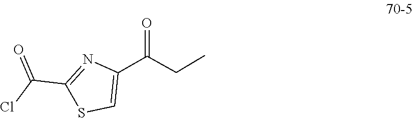

Oxalyl chloride (961 mg, 7.6 mol) was added dropwise to a suspension of compound 70-4 (700 mg, 3.8 mmol) in DCM (20 mL) at room temperature. The reaction mixture was stirred for 4 h, then concentrated. The residue was azeotroped two times with DCM (20 mL portions) to afford crude compound 70-5 (750 mg, ˜100% yield), which was used for next step without further purification.

### Step 6: 1-(2-(5-Fluoro-1H-indole-3-carbonyl)thiazol-4-yl)propan-1-one (ARI-087)

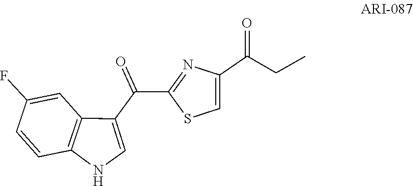 [SMILES: CCC(=O)c1csc(C(=O)c2c[nH]c3ccc(F)cc23)n1]

MeMgBr (3M in THF, 1.5 mL, 4.4 mmol) was added dropwise to a mixture of 7-fluoroindole (500 mg, 3.7 mmol) and anhydrous zinc chloride (1.5 g, 11 mmol) in DCM (20 mL) at 0° C. under N₂. The mixture was stirred for 1 h at this temperature, and then a solution of compound 70-5 (750 mg, 3.7 mmol) in THF (20 mL) was added at 0° C. The reaction mixture was allowed to warm to room temperature and stirred overnight. The mixture was quenched with saturated aqueous NH₄Cl (100 mL), stirred for 20 min, filtered. The solid was collected, washed with water (30 mL×3) , EtOAc (30 mL×3) and MeOH (30 mL×3), dried to afford ARI-087 (620 mg, 55% yield from compound 70-3) as yellow solid. ¹H-NMR (400 MHz, DMSO-d6): δ 12.42 (bs, 1H), 9.20 (s, 1H), 8.86 (s, 1H), 7.97˜8.00 (m, 1H), 7.60˜7.63 (m, 1H), 7.16˜7.19 (m, 1H), 3.22˜3.26 (q, J=7.2 Hz, 2H), 1.13˜1.18 (t, J=7.2 Hz, 3H). LC-MS: m/z 302.7 [M+H]⁺.

### Example 85: Preparation of 5-(1H-indole-3-carbonyl)pyrazine-2-carbonitrile (ARI-093)

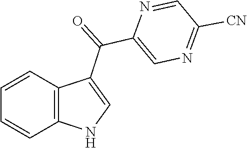 [SMILES: N#Cc1cnc(C(=O)c2c[nH]c3ccccc23)cn1]

Prepared from 2,5-dibromopyrazine according to the method described in Example 82.

### Example 86: Preparation of methyl 5-(1H-indole-3-carbonyl)pyrazine-2-carboxylate (ARI-095)

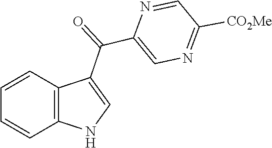 [SMILES: COC(=O)c1cnc(C(=O)c2c[nH]c3ccccc23)cn1]

Prepared according to the method described in Example 83, except that 3,6-carboxymethylpyrazine was used as the staring material.

### Example 87: Preparation of 1-(2-(5,6-dibromo-1H-indole-3-carbonyl)thiazol-4-yl)propan-1-one (ARI-097)

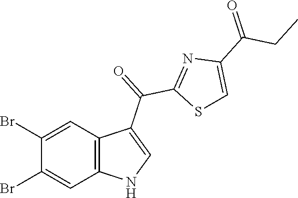 [SMILES: CCC(=O)c1csc(C(=O)c2c[nH]c3cc(Br)c(Br)cc23)n1]

Starting with methyl 2-(5,6-dibromo-1H-indole-3-carbonyl) thiazole-4-carboxylate (PTC17341-11A) (prepared as shown below and as shown in the scheme of FIG. 30) according to the method described in Example 75 except that ethylmagnesium bromide was used instead of methylmagnesium bromide.

### Step 1: Methyl 2-(1-(tert-butoxycarbonyl)-1H-indole-3-carbonyl)thiazole-4-carboxylate (86-1)

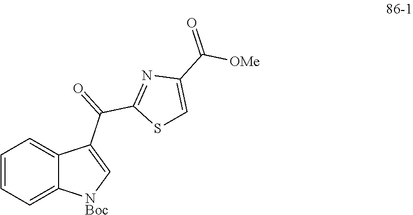 [SMILES: COC(=O)c1csc(C(=O)c2cn(C(=O)OC(C)(C)C)c3ccccc23)n1]

This compound was synthesized according to the protocol described in Example 65 from compound 1-5 (10.00 g, 27 mmol) with MeI to give title compound 86-1 in the form of a yellow solid (9.8 g, 94% yield).

### Step 2: Methyl 2-(5,6-dibromo-1H-indole-3-carbonyl)thiazole-4-carboxylate (PTC17341-11A)

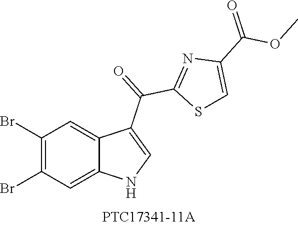 [SMILES: COC(=O)c1csc(C(=O)c2c[nH]c3cc(Br)c(Br)cc23)n1]

Compound 86-1 (3.0 g, 7.8 mmol) was dissolved in HOAc (25 mL), then bromine (5.0 g, 31 mmol) was added at room temperature. The mixture was stirred at 50° C. for 72 h. After cooled to room temperature, the mixture was filtered, and the solid was collected, washed by HOAc (10 mL×2) to afford crude PTC17341-11A. The crude was recrystallized with DMF/H2O (2:1, 50 mL) to give compound PTC17341-11A (2.3 g, 67% yield). ¹H-NMR (400 MHz, DMSO-d6): δ 12.49 (bs, 1H), 9.07 (s, 1H), 8.90 (s, 1H), 8.57 (s, 1H), 8.00 (s, 1H), 3.93 (s, 3H). LC-MS: m/z 422.6 [M+H]⁺.

### Example 88: Preparation of methyl 2-(7-fluoro-1H-indole-3-carbonyl)thiazole-4-carboxylate (ARI-101)

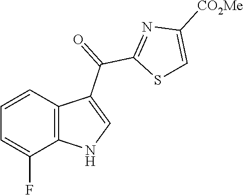 [SMILES: COC(=O)c1csc(C(=O)c2c[nH]c3c(F)cccc23)n1]

Starting from 7-fluoroindole-3-carboxylic acid, ARI-101 was prepared as described in Example 130 to obtain 2-(1-(tert-butoxycarbonyl)-7-fluoro-1H-indole-3-carbonyl)thiazole-4-carboxylate, which was then transformed to ARI-101 in the presence of K₂CO₃, MeI, and TFA by a method described in Example 65.

### Example 89: Preparation of methyl 2-(7-fluoro-1H-indole-3-carbonyl)thiazole-4-carboxylate (ARI-102)

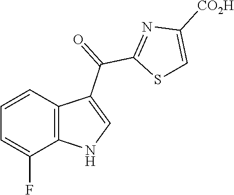 [SMILES: O=C(O)c1csc(C(=O)c2c[nH]c3c(F)cccc23)n1]

Prepared according to the method for preparing the key intermediate 2-(1H-indole-3-carbonyl)thiazole-4-carboxylic acid using 7-fluoro-1H-indole-3-carboxylic acid.

### Example 90: Preparation of 2-(5-chloro-1H-indole-2-carbonyl)-N-methylthiazole-4-carboxamide (ARI-103)

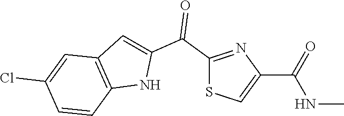 [SMILES: CNC(=O)c1csc(C(=O)c2cc3cc(Cl)ccc3[nH]2)n1]

Prepared according to the method for preparing the key intermediate 2-(1H-indole-3-carbonyl)thiazole-4-carboxylic acid using 5-chloro-1H-indole-2-carboxylic acid to obtain 2-(1-(tert-butoxycarbonyl)-5-chloro-1H-indole-2-carbonyl)thiazole-4-carboxylic acid which was transformed to the final product using conditions described in Example 24 or the HATU and TFA method described in Example 68.

### Example 91: Preparation of 2-(7-fluoro-1H-indole-3-carbonyl)thiazole-4-carbonitrile (ARI-104)

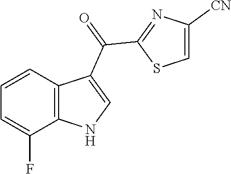 [SMILES: N#Cc1csc(C(=O)c2c[nH]c3c(F)cccc23)n1]

Prepared according to the method for preparing the key intermediate 2-(1H-indole-3-carbonyl)thiazole-4-carboxylic acid using 7-fluoro-1H-indole-3-carboxylic acid to obtain 2-(1-(tert-butoxycarbonyl)-7-fluoro-1H-indole-3-carbonyl)thiazole-4-carboxylic acid which was transformed to the final product using the method described in Example 57.

### Example 92: Preparation of 2-(5-fluoro-1H-indole-2-carbonyl)thiazole-4-carboxylic acid (ARI-105)

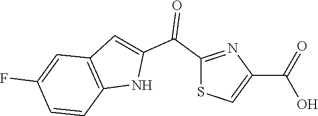 [SMILES: O=C(O)c1csc(C(=O)c2cc3cc(F)ccc3[nH]2)n1]

Prepared according to the method for preparing the key intermediate 2-(1H-indole-3-carbonyl)thiazole-4-carboxylic acid described in Example 130 using 5-fluoro-1H-indole-2-carboxylic acid.

### Example 93: Preparation of 2-(5-chloro-1H-indole-2-carbonyl)thiazole-4-carboxylic acid (ARI-106)

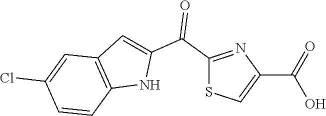 [SMILES: O=C(O)c1csc(C(=O)c2cc3cc(Cl)ccc3[nH]2)n1]

Prepared according to the method for preparing the key intermediate 2-(1H-indole-3-carbonyl)thiazole-4-carboxylic acid (Example 130) using 5-chloro-1H-indole-2-carboxylic acid.

### Example 94: Preparation of 2-(5-fluoro-1H-indole-2-carbonyl)thiazole-4-carbonitrile (ARI-107)

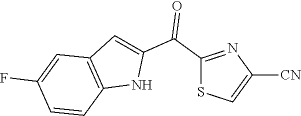 [SMILES: N#Cc1csc(C(=O)c2cc3cc(F)ccc3[nH]2)n1]

Prepared according to the method for preparing the key intermediate 2-(1H-indole-3-carbonyl)thiazole-4-carboxylic acid using 5-fluoro-1H-indole-2-carboxylic acid to obtain 2-(1-(tert-butoxycarbonyl)-7-fluoro-1H-indole-3-carbonyl)thiazole-4-carboxylic acid which was transformed to the final product using the method described in Example 57.

### Example 95: Preparation of 2-(5-fluoro-1H-indole-2-carbonyl)-N-methylthiazole-4-carboxamide (ARI-108)

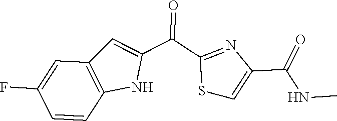 [SMILES: CNC(=O)c1csc(C(=O)c2cc3cc(F)ccc3[nH]2)n1]

Prepared according to the method for preparing the key intermediate 2-(1H-indole-3-carbonyl)thiazole-4-carboxylic acid using 5-fluoro-1H-indole-2-carboxylic acid to obtain 2-(1-(tert-butoxycarbonyl)-7-fluoro-1H-indole-3-carbonyl)thiazole-4-carboxylic acid which was transformed to the final product using methods described in Examples 24 or the HATU and TFA method described in Example 68.

### Example 96: Preparation of methyl 2-(6-cyano-1H-indole-3-carbonyl)thiazole-4-carboxylate (ARI-111)

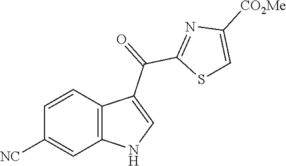 [SMILES: COC(=O)c1csc(C(=O)c2c[nH]c3cc(C#N)ccc23)n1]

Prepared according to the method described in Example 118 except that 6-bromoindole-3-carboxylic acid instead of 5-bromoindole 3-carboxylic acid was used.

### Example 97: Preparation of 2-(5-fluoro-1H-indole-3-carbonyl)thiazole-4-carbonitrile (ARI-112)

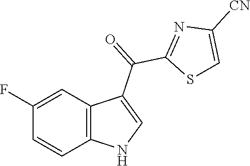 [SMILES: N#Cc1csc(C(=O)c2c[nH]c3ccc(F)cc23)n1]

Prepared according to the method for preparing the key intermediate 2-(1H-indole-3-carbonyl)thiazole-4-carboxylic acid using 5-fluoro-1H-indole-2-carboxylic acid to obtain 2-(1-(tert-butoxycarbonyl)-7-fluoro-1H-indole-3-carbonyl)thiazole-4-carboxylic acid which was transformed to the final 4-cyanothiazole product by the previously described trifluoroacetic anhydride (TFAA)-mediated dehydration of the primary amide (see method described in Example 57).

### Example 98: Preparation of 2-(5-chloro-1H-indole-2-carbonyl)thiazole-4-carbonitrile (ARI-113)

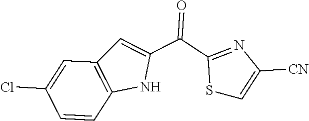 [SMILES: N#Cc1csc(C(=O)c2cc3cc(Cl)ccc3[nH]2)n1]

Prepared according to the method for preparing the key intermediate 2-(1H-indole-3-carbonyl)thiazole-4-carboxylic acid using 5-chloro-1H-indole-2-carboxylic acid to obtain 2-(1-(tert-butoxycarbonyl)-5-chloro-1H-indole-2-carbonyl)thiazole-4-carboxylic acid which was transformed to the final 4-cyanothiazole product by the previously described trifluoroacetic anhydride (TFAA)-mediated dehydration of the primary amide (see method described in Example 57).

### Example 99: Preparation of (7-fluoro-1H-indol-3-yl)(4-(5-methyl-1,2,4-oxadiazol-3-yl)thiazol-2-yl)methanone (ARI-114)

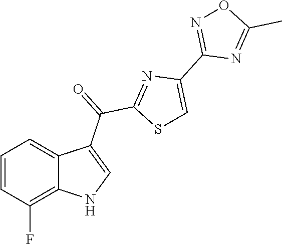 [SMILES: Cc1nc(-c2csc(C(=O)c3c[nH]c4c(F)cccc34)n2)no1]

Prepared from 2-(1-(tert-butoxycarbonyl)-7-fluoro-1H-indole-2-carbonyl)thiazole-4-carboxylic acid (itself prepared from 7-fluoroindole-3-carboxylic acid by the methods described in Example 130) by the method described in Example 59.

### Example 100: Preparation of (4-(5-amino-1,3,4-oxadiazol-2-yl)thiazol-2-yl)(7-fluoro-1H-indol-3-yl)methanone (ARI-118)

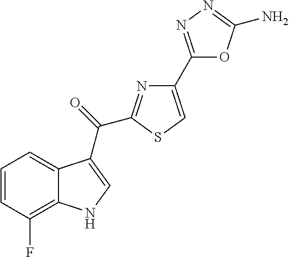 [SMILES: Nc1nnc(-c2csc(C(=O)c3c[nH]c4c(F)cccc34)n2)o1]

Prepared according to the method described in Example 131 except that 7-fluoroindole was used instead of 5,6-difluoroindole.

### Example 101: Preparation of (5-fluoro-1H-indol-3-yl)(4-(5-methyl-1,2,4-oxadiazol-3-yl)thiazol-2-yl)methanone (ARI-119)

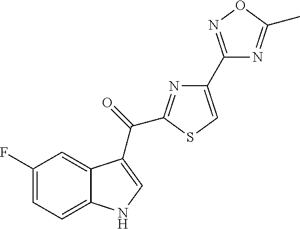 [SMILES: Cc1nc(-c2csc(C(=O)c3c[nH]c4ccc(F)cc34)n2)no1]

Prepared from 2-(1H-indole-5-fluoro-3-carbonyl)thiazole-4-carbonitrile according to the method described in Example 21.

### Example 102: Preparation of (7-fluoro-1H-indol-3-yl)(4-(3-methyl-1,2,4-oxadiazol-5-yl)thiazol-2-yl)methanone (PTC17341-95, ARI-123)

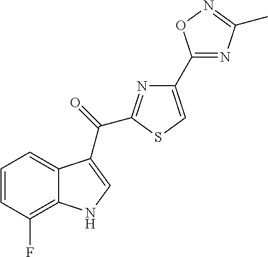 [SMILES: Cc1noc(-c2csc(C(=O)c3c[nH]c4c(F)cccc34)n2)n1]

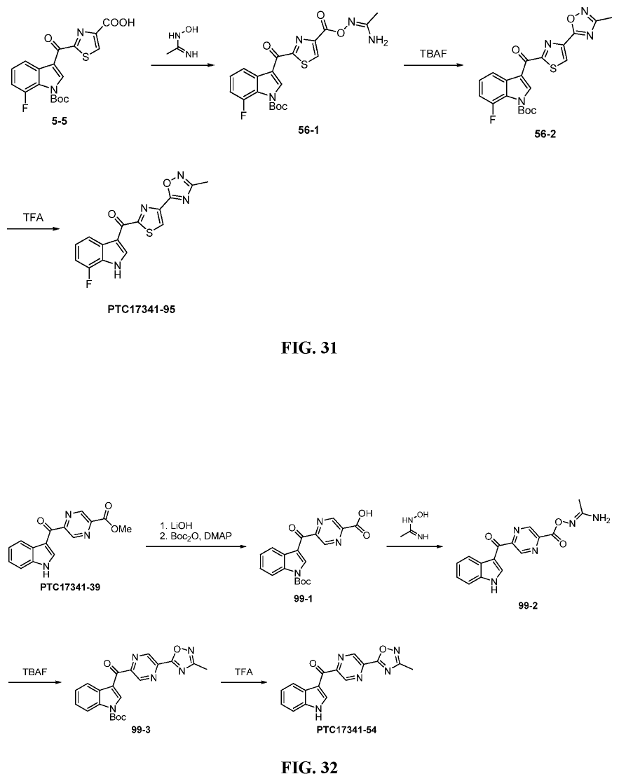

ARI-123 was synthesized according to the scheme of FIG. 31 and by the following method:

### Step 1: tert-Butyl 3-(4-((1-aminoethylideneaminooxy)carbonyl)thiazole-2-carbonyl)-7-fluoro-1H-indole-1-carboxylate (56-1)

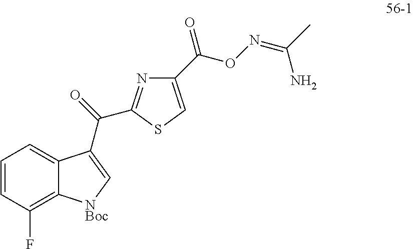 [SMILES: C/C(N)=N/OC(=O)c1csc(C(=O)c2cn(C(=O)OC(C)(C)C)c3c(F)cccc23)n1]

Oxalyl chloride (500 mg, 4 mmol) was added to a suspension of compound 5-5 (780 mg, 2 mmol) in DCM (20 mL) at 0° C. The mixture was allowed to warm to room temperature and stirred 5 h. The mixture was concentrated to dryness. The residue was dissolved in DCM (5 mL) and added dropwise to a suspension of N-hydroxyacetimidamide (220 mg, 3 mmol) and TEA (410 mg, 4 mmol) in DCM (20 mL) at 0° C. over 10 min. The resulting mixture was allowed to warm to room temperature and stirred 1 h. The mixture was concentrated to dryness. And the residue was purified by silica gel chromatography (EtOAc/Hexane/DCM=2:1:1) and afforded compound 56-1 (450 mg, 50% yield).

### Step 2: tert-Butyl 7-fluoro-3-(4-(3-methyl-1,2,4-oxadiazol-5-yl)thiazole-2-carbonyl)-1H-indole-1-carboxylate (56-2)

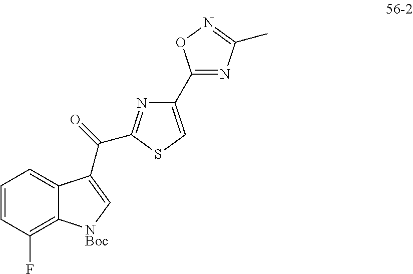 [SMILES: Cc1noc(-c2csc(C(=O)c3cn(C(=O)OC(C)(C)C)c4c(F)cccc34)n2)n1]

A solution of compound 56-1 (450 mg, 1 mmol) and TBAF (780 mg, 3 mmol) in THF (20 mL) was heated under reflux for 4 h. The mixture was cooled to room temperature, Boc₂O (430 mg, 2 mmol) and 4-dimethylaminopyridine (DMAP) (10 mg, cat.) were added to. The mixture was stirred for 2 h at room temperature, then concentrated to dryness. The residue was purified by silica gel chromatography (EtOAc/Hexane/DCM=1:2:1) and afforded compound 56-2 (150 mg, 35% yield).

### Step 3: (7-fluoro-1H-indol-3-yl)(4-(3-methyl-1,2,4-oxadiazol-5-yl) thiazol-2-yl)methanone (PTC17341-95, ARI-123)

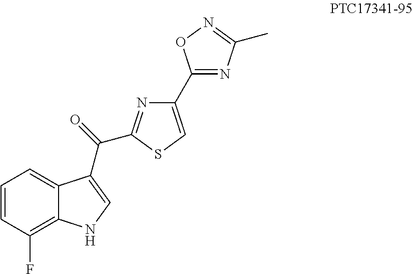 [SMILES: Cc1noc(-c2csc(C(=O)c3c[nH]c4c(F)cccc34)n2)n1]

This compound was synthesized according to the protocol described in Example 71 from compound 56-2 (150 mg, 0.35 mmol) to give title compound PTC17341-95 (ARI-123) in the form of a yellow solid (85% yield). ¹H-NMIR (400 MHz, DMSO-d6): δ 13.01 (bs, 1H), 9.12˜9.15 (d, J=8.0 Hz, 1H), 8.12˜8.15 (d, J=8.0 Hz, 1H), 7.27˜7.33 (m, 1H), 7.17˜7.23 (m, 1H), 2.51 (s, 3H). LC-MS: m/z 327.2 [M−H]⁻.

### Example 103: Preparation of methyl 2-(7-cyano-1H-indole-3-carbonyl) thiazole-4-carboxylate (ARI-124)

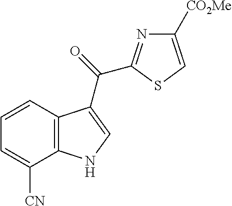 [SMILES: COC(=O)c1csc(C(=O)c2c[nH]c3c(C#N)cccc23)n1]

Prepared according to the method described in Example 118 except that 7-bromoindole-3-carboxylic acids was used.

### Example 104: Preparation of 4-(1H-indole-3-carbonyl)pyrimidine-2-carbonitrile (ARI-125)

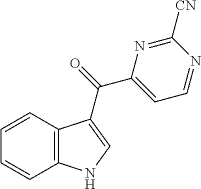 [SMILES: N#Cc1nccc(C(=O)c2c[nH]c3ccccc23)n1]

Prepared from 2,4-dibromopyrimidine as described in Example 82.

### Example 105: Preparation of (5-fluoro-1H-indol-2-yl)(4-(3-methyl-1,2,4-oxadiazol-3-yl)thiazol-2-yl)methanone (ARI-126)

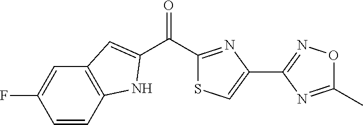 [SMILES: Cc1nc(-c2csc(C(=O)c3cc4cc(F)ccc4[nH]3)n2)no1]

Prepared from 2-(1H-indole-2-carbonyl)thiazole-4-carbonitrile according to the method described in Examples 21.

### Example 106: Preparation of (1H-indol-3-yl)(5-(3-methyl-1,2,4-oxadiazol-5-yl)pyrazin-2-yl)methanone (PTC17341-54, ARI-127)

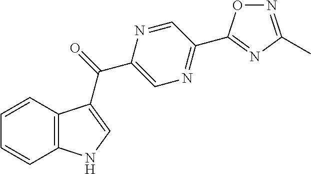 [SMILES: Cc1noc(-c2cnc(C(=O)c3c[nH]c4ccccc34)cn2)n1]

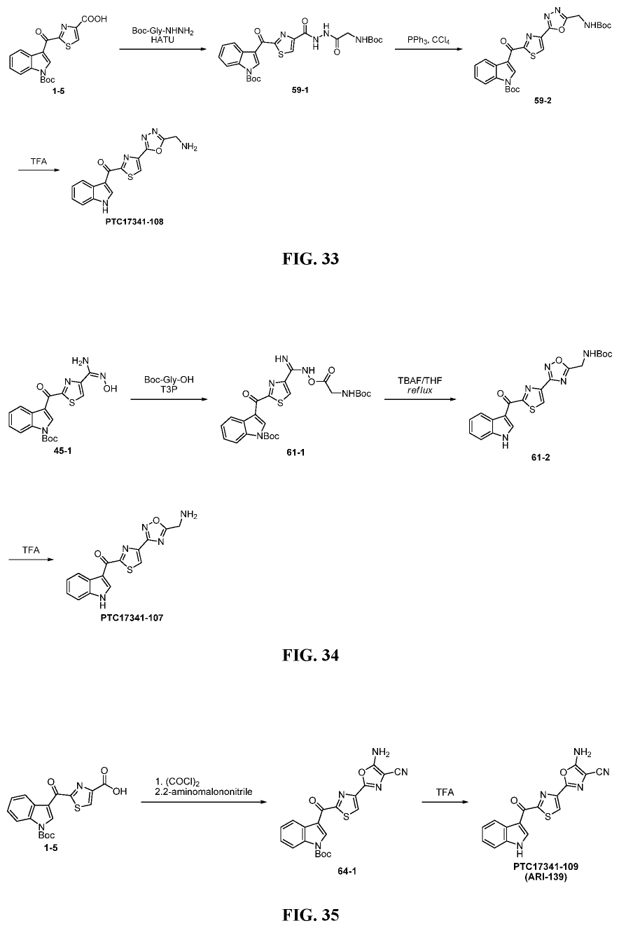

ARI-127 was synthesized according to the scheme of FIG. 32 and by the following method:

### Step 1: 5-(1-(tert-Butoxycarbonyl)-1H-indole-3-carbonyl)pyrazine-2-carboxylic acid (99-1)

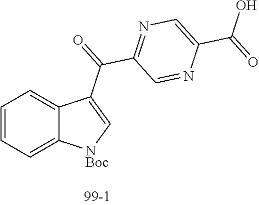 [SMILES: CC(C)(C)OC(=O)n1cc(C(=O)c2cnc(C(=O)O)cn2)c2ccccc21]

Lithium hydroxide monohydrate (380 mg, 9 mmol) was added to a solution of compound PTC17341-39 (840 mg, 3 mmol) in THF (10 mL) and H₂O (10 mL) at 0° C. The resulting mixture was stirred for 2 h at room temperature, then acidified with 1M HCl aqueous to pH of 3. The mixture was concentrated to dryness. The residue was azeotroped two times with THF (50 mL portions), then dissolved in DMF (10 mL). DMAP (730 mg, 6 mmol) and Boc₂O (1.3 g, 6 mmol) were added to. The resulting mixture was stirred overnight. The mixture was diluted with water (50 mL), acidified with 1M HCl aqueous to pH of 3, extracted with EtOAc (50 mL×3). The combined organic phases were washed with water (50 mL×2), and brine (50 mL×1), dried (Na₂SO₄), filtered and concentrated to dryness. The residue was triturated with EtOAc/hexane (1:5, 50 mL), filtered and dried to afford compound 99-1 (810 mg, 73% yield).

### Step 2: tert-Butyl 3-(5-((1-aminoethylideneaminooxy)carbonyl)pyrazine-2-carbonyl)-1H-indole-1-carboxylate (99-2)

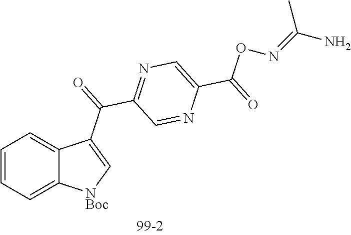 [SMILES: C/C(N)=N\OC(=O)c1cnc(C(=O)c2cn(C(=O)OC(C)(C)C)c3ccccc23)cn1]

This compound was synthesized according to the protocol described in Example 102 step 1 from compound 99-1 (800 mg, 2.2 mmol) to give title compound 99-2 (1.10 g, ˜100% yield).

### Steps 3/4: (1H-Indol-3-yl)(5-(3-methyl-1,2,4-oxadiazol-5-yl)pyrazin-2-yl) methanone (PTC17341-54, ARI-127)

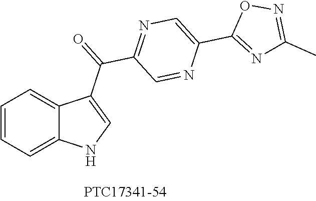 [SMILES: Cc1noc(-c2cnc(C(=O)c3c[nH]c4ccccc34)cn2)n1]

This compound was synthesized according to the protocol described in Example 102 step 2 and 3 from compound 99-2 (1.10 g, 2.2 mmol) to give title compound PTC17341-54 (ARI-127) in the form of a yellow solid (80 mg, 12% yield for two steps). ¹H-NMR (400 MHz, DMSO-d6): δ 12.35 (bs, 1H), 8.73˜8.72 (d, J=3.2 Hz, 1H), 8.37˜8.40 (d, m, 1H), 7.56˜7.60 (m, 1H), 7.29˜7.33 (m, 2H), 2.53 (s, 3H). LC-MS: m/z 303.6 [M−H]⁻.

### Example 107: Preparation of 2-(5-chloro-2-methyl-1H-indole-3-carbonyl)thiazole-4-carboxylic acid (ARI-128)

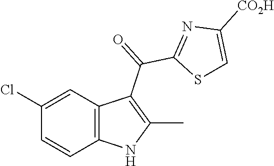 [SMILES: Cc1[nH]c2ccc(Cl)cc2c1C(=O)c1nc(C(=O)O)cs1]

Starting with 2-methyl-indole-3-carboxylic acid, prepared as described in Example 130.

### Example 108: Preparation of (4-(5-amino-1,3,4-oxadiazol-2-yl)thiazol-2-yl)(5-fluoro-1H-indol-2-yl)methanone (ARI-129)

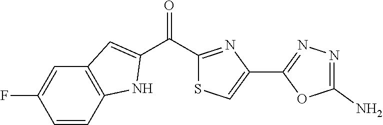 [SMILES: Nc1nnc(-c2csc(C(=O)c3cc4cc(F)ccc4[nH]3)n2)o1]

Prepared from 2-(1H-5-fluoro-indole-2-carbonyl)thiazole-4-carboxylic acid according to the method described in Example 131.

### Example 109: Preparation of (4-(5-amino-1,3,4-oxadiazol-2-yl)thiazol-2-yl)(5-fluoro-1H-indol-2-yl)methanone (ARI-131)

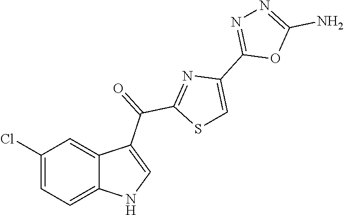 [SMILES: Nc1nnc(-c2csc(C(=O)c3c[nH]c4ccc(Cl)cc34)n2)o1]

Prepared from 2-(1H-5-chloro-indole-3-carbonyl)thiazole-4-carboxylic acid according to the method described in Example 131.

### Example 110: Preparation of 2-(5-fluoro-2-methyl-1H-indole-3-carbonyl)thiazole-4-carboxylic acid (ARI-130)

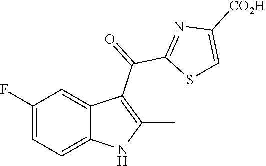 [SMILES: Cc1[nH]c2ccc(F)cc2c1C(=O)c1nc(C(=O)O)cs1]

Prepared from 2-methyl-5-fluoroindole-3-carboxylic acid according to the method described in Example 130.

### Example 111: Preparation of (5-fluoro-1H-indol-3-yl)(4-(3-methyl-1,2,4-oxadiazol-5-yl)thiazol-2-yl)methanone (ARI-132)

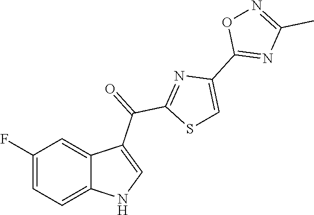 [SMILES: Cc1noc(-c2csc(C(=O)c3c[nH]c4ccc(F)cc34)n2)n1]

Prepared from 2-(1H-5-fluoro-indole-3-carbonyl)thiazole-4-carboxylic acid according to the method described in Example 22.

### Example 112: Preparation of (4-(5-(aminomethyl)-1,2,4-oxadiazol-3-yl)thiazol-2-yl)(5-chloro-1H-indol-3-yl)methanone (ARI-133)

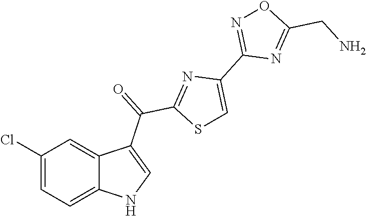 [SMILES: NCc1nc(-c2csc(C(=O)c3c[nH]c4ccc(Cl)cc34)n2)no1]

Starting from 2-(1H-indole-5-chloro-3-carbonyl)thiazole-4-hydroxyimidate (prepared as described in Example 21), this compound was prepared as described in Example 115.

### Example 113: Preparation of (4-(5-amino-1,3,4-oxadiazol-2-yl)thiazol-2-yl)(5-chloro-2-methyl-1H-indol-3-yl)methanone (ARI-134)

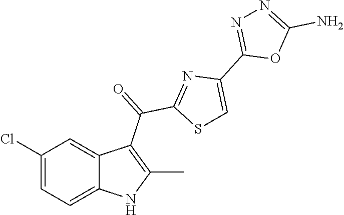 [SMILES: Cc1[nH]c2ccc(Cl)cc2c1C(=O)c1nc(-c2nnc(N)o2)cs1]

Prepared from 2-(1H-indole-2-methyl-5-chloro-3-carbonyl)thiazole-4-carboxylate according to the method described in Example 131.

### Example 114. Preparation of (4-(5-(aminomethyl)-1,3,4-oxadiazol-2-yl)thiazol-2-yl) (1H-indol-3-yl) methanone (PTC17341-108, ARI-137)

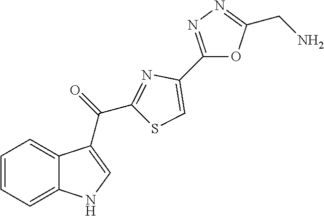 [SMILES: NCc1nnc(-c2csc(C(=O)c3c[nH]c4ccccc34)n2)o1]

ARI-137 was synthesized according to the scheme of FIG. 33 and by the following method:

### Step 1: tert-Butyl 3-(4-(2-(2-(tert-butoxycarbonylamino)acetyl)hydrazine carbonyl) thiazole-2-carbonyl)-1H-indole-1-carboxylate (59-1)

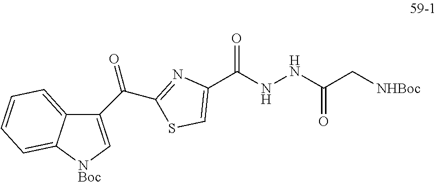 [SMILES: CC(C)(C)OC(=O)NCC(=O)NNC(=O)c1csc(C(=O)c2cn(C(=O)OC(C)(C)C)c3ccccc23)n1]

HATU (664 mg, 17. mmol) and DIPEA (520 mg, 4 mmol) were added to a solution of compound 1-5 (500 mg, 1.3 mmol) and Boc-glycine hydrazide (305 mg, 1.6 mmol) in DMF (10 mL) at room temperature. The mixture was stirred overnight, then quenched with H₂O (50 mL). The mixture was stirred for 0.5 h, then filtered to collect the solid. The solid was washed with water (10 mL×3) and EtOAc (10 mL×3), dried to afford 59-1 (700 mg, ˜100% yield) as off-white solid.

### Step 2: tert-Butyl 3-(4-(5-((tert-butoxycarbonylamino)methyl)-1,3,4-oxadiazol-2-yl) thiazole-2-carbonyl)-1H-indole-1-carboxylate (59-2)

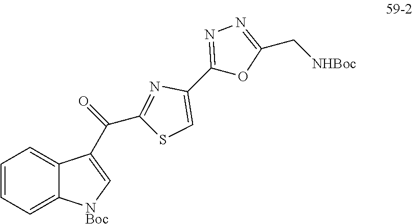 [SMILES: CC(C)(C)OC(=O)NCc1nnc(-c2csc(C(=O)c3cn(C(=O)OC(C)(C)C)c4ccccc34)n2)o1]

Triphenylphosphine (470 mg, 1.8 mmol) and TEA (209 mg, 2.1 mmol) were added to a solution of compound 59-1 (700 mg, 1.3 mmol) in ACN (20 mL) at room temperature. The mixture was stirred for 20 min, then was added CCl₄ (320 mg, 2.1 mmol). The mixture was heated to 50° C. and stirred for 5 h. The mixture was cooled to room temperature, then concentrated. The residue was purified by silica gel chromatography (EtOAc/Hexane/DCM=1:1:1) and afforded compound 59-2 (290 mg, 42% yield).

### Step 3: (4-(5-(Aminomethyl)-1,3,4-oxadiazol-2-yl)thiazol-2-yl)(1H-indol-3-yl) methanone (PTC17341-108, ARI-137)

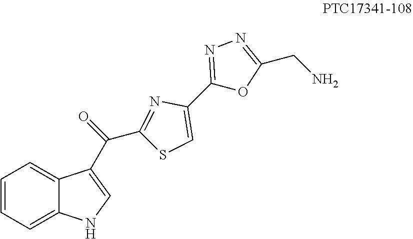 [SMILES: NCc1nnc(-c2csc(C(=O)c3c[nH]c4ccccc34)n2)o1]

This compound was synthesized according to the protocol described in Example 71 from compound 59-2 (290 mg, 0.55 mmol) to give title compound PTC17341-108 (ARI-137) in the form of a yellow solid (80% yield). ¹H-NMR (400 MHz, DMSO-d6): δ 12.42 (bs, 1H), 9.14 (s, 1H), 8.90 (s, 1H), 8.32˜8.35 (m, 1H), 7.60˜7.63 (m, 1H), 7.29˜7.34 (m, 2H), 4.04 (s, 2H), 1.99 (s, 2H). LC-MS: m/z 326.4 [M+H]⁺.

### Example 115: Preparation of (4-(5-(Aminomethyl)-1,2,4-oxadiazol-3-yl)thiazol-2-yl) (1H-indol-3-yl) methanone (PTC17341-107, ARI-138)

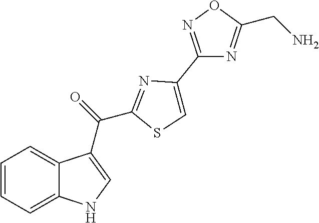 [SMILES: NCc1nc(-c2csc(C(=O)c3c[nH]c4ccccc34)n2)no1]

ARI-138 was synthesized according to the scheme of FIG. 34 and by the following method:

### Step 1: tert-Butyl 3-(4-(N-(2-(tert-butoxycarbonylamino)acetoxy)carbamimidoyl) thiazole-2-carbonyl)-1H-indole-1-carboxylate (61-1)

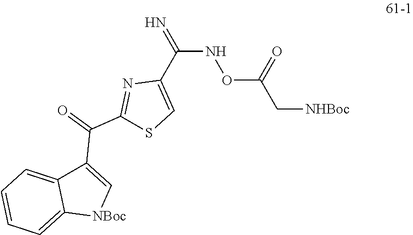 [SMILES: CC(C)(C)OC(=O)NCC(=O)ONC(=N)c1csc(C(=O)c2cn(C(=O)OC(C)(C)C)c3ccccc23)n1]

T3P (50% solution in EtOAc, 1.5 g, 5 mmol) and TEA (606 mg, 6 mmol) were added to a solution of compound 45-1 (770 mg, 2 mmol) and Boc-glycine (350 mg, 2 mmol) in EtOAc (150 mL) at room temperature. The mixture was heated under reflux for 8 h. The mixture was cooled to room temperature, washed with brine (100 mL×3), dried, concentrated to afford compound 61-1 (˜1 g), which was used for next step without further purification.

### Step 2: tert-Butyl (3-(2-(1H-indole-3-carbonyl)thiazol-4-yl)-1,2,4-oxadiazol-5-yl) methylcarbamate (61-2)

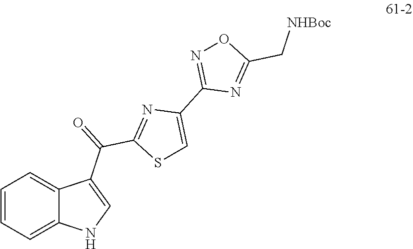 [SMILES: CC(C)(C)OC(=O)NCc1nc(-c2csc(C(=O)c3c[nH]c4ccccc34)n2)no1]

A solution of crude compound 61-1 (˜1 g) and TBAF (1.05 g, 4 mmol) in THF (50 mL) was heated under reflux for 4 h. The mixture was cooled to room temperature, then concentrated to dryness. The residue was purified by silica gel chromatography (EtOAc/Hexane/DCM=1:2:1) and afforded compound 61-2 (220 mg, 26% yield from compound 45-1).

### Step 3: (4-(5-(Aminomethyl)-1,2,4-oxadiazol-3-yl)thiazol-2-yl)(1H-indol-3-yl) methanone (PTC17341-107, ARI-138)

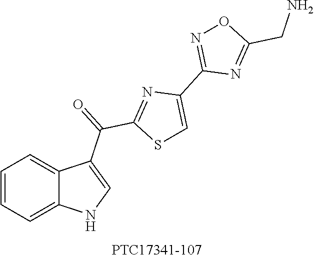 [SMILES: NCc1nc(-c2csc(C(=O)c3c[nH]c4ccccc34)n2)no1]

This compound was synthesized according to the protocol described in Example 71 from compound 61-2 (220 mg, 0.52 mmol) to give title compound PTC17341-107 (ARI-138) in the form of a yellow solid (70% yield). ¹H-NMR (400 MHz, DMSO-d6): δ 12.39 (bs, 1H), 9.15 (s, 1H), 8.80 (s, 1H), 8.32˜8.35 (m, 1H), 7.60˜7.62 (d, J=5.6 Hz, 1H), 7.30˜7.32 (m, 2H), 4.08 (s, 2H), 2.23 (s, 2H). LC-MS: m/z 326.4 [M+H]⁺.

### Example 116: Preparation of 2-(2-(1H-indole-3-carbonyl)thiazol-4-yl)-5-aminooxazole-4-carbonitrile (PTC17341-109, ARI-139)

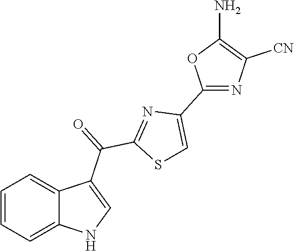 [SMILES: N#Cc1nc(-c2csc(C(=O)c3c[nH]c4ccccc34)n2)oc1N]

ARI-139 was synthesized according to the scheme of FIG. 35 and by the following method:

### Step 1: tert-butyl 3-(4-(5-amino-4-cyanooxazol-2-yl)thiazole-2-carbonyl)-1H-indole-1-carboxylate (64-1)

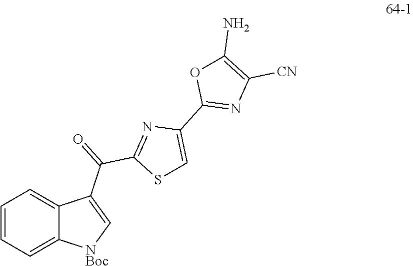 [SMILES: CC(C)(C)OC(=O)n1cc(C(=O)c2nc(-c3nc(C#N)c(N)o3)cs2)c2ccccc21]

Oxalyl chloride (890 mg, 5.3 mmol) was added to a suspension of compound 1-5 (1.3 g, 3.5 mmol) in DCM (20 mL) at 0° C. The mixture was allowed to warm to room temperature and stirred 5 h. The mixture was concentrated to dryness. The residue was dissolved in N-methyl-2-pyrrolidone (NMP) (5 mL) and 2-aminomalononitrile 4-methylbenzenesulfonate (1.15 g, 4.5 mmol) was added to at room temperature. The resulting mixture was stirred for 1 h. The mixture was diluted with H₂O (20 mL), extracted with EtOAc/THF (1:1, 30 mL×3). The combined organic phases were washed with brine (50 mL×2), dried, concentrated to dryness. The residue was purified by silica gel chromatography (EtOAc/Hexane/DCM=1:1:1) and afforded compound 64-1 (440 mg, 29% yield).

### Step 2: 2-(2-(1H-indole-3-carbonyl)thiazol-4-yl)-5-aminooxazole-4-carbonitrile (PTC17341-109, ARI-139)

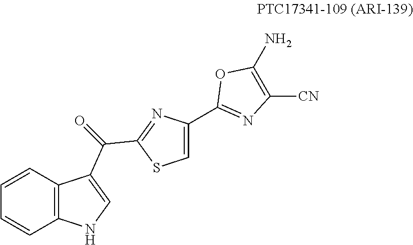 [SMILES: N#Cc1nc(-c2csc(C(=O)c3c[nH]c4ccccc34)n2)oc1N]

Compound 64-1 (440 mg, 1 mmol) was dissolved in THF (5 mL) and MeOH (5 mL), KHCO₃ (1.0 g) and Na₂CO₃ (1.0 g) were added. The mixture was stirred overnight at room temperature. The mixture was diluted with H₂O (20 mL), extracted with EtOAc/THF (1:1, 30 mL×3). The combined organic phases were washed with brine (50 mL×2), dried, concentrated to dryness. The residue was triturated with EtOAc (20 mL) and MeOH (20 mL) to give title compound PTC17341-109 (ARI-139) in the form of a yellow solid (190 mg, 57% yield). ¹H-NMR (400 MHz, DMSO-d6): δ 12.38 (bs, 1H), 9.08˜9.10 (d, J=2.8 Hz, 1H), 8.48 (s, 1H), 8.30˜9.34 (m, 1H), 8.17 (s, 1H), 7.58˜7.62 (d, J=6.4 Hz, 1H), 7.29˜7.33 (m, 2H). LC-MS: m/z 326.4 [M+H]⁺.

### Example 117: Preparation of 1-(2-(7-fluoro-1H-indole-3-carbonyl)thiazol-4-yl)propan-1-one (ARI-140)

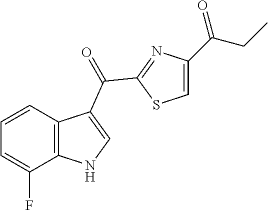 [SMILES: CCC(=O)c1csc(C(=O)c2c[nH]c3c(F)cccc23)n1]

Starting with 7-fluoroindole and using the procedure described in Example 84—Step 6, the title compound was prepared.

### Example 118: Preparation of methyl 2-(5-cyano-1H-indole-3-carbonyl) thiazole-4-carboxylate (PTC17341-60) (ARI-141)

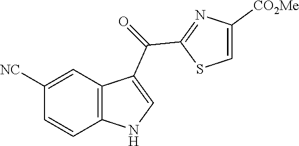 [SMILES: COC(=O)c1csc(C(=O)c2c[nH]c3ccc(C#N)cc23)n1]

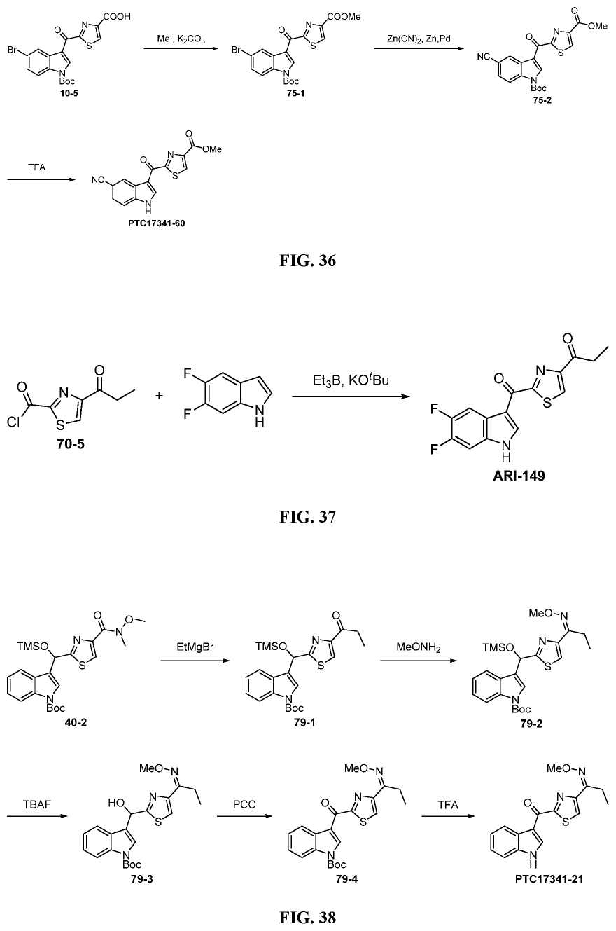

ARI-141 was synthesized according to the scheme of FIG. 36 and by the following method:

### Step 1: Methyl 2-(5-bromo-1-(tert-butoxycarbonyl)-1H-indole-3-carbonyl) thiazole-4-carboxylate (75-1)

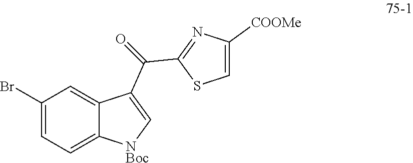 [SMILES: COC(=O)c1csc(C(=O)c2cn(C(=O)OC(C)(C)C)c3ccc(Br)cc23)n1]

This compound was synthesized according to the protocol described in Example 65 from compound 10-5 (1.0 g, 2.2 mmol) to give title compound 75-1 in the form of an off-white solid (0.93 g, 91% yield).

### Step 2: Methyl 2-(1-(tert-butoxycarbonyl)-5-cyano-1H-indole-3-carbonyl) thiazole-4-carboxylate (75-2)

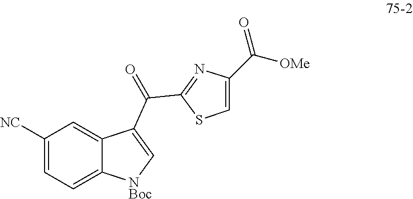 [SMILES: COC(=O)c1csc(C(=O)c2cn(C(=O)OC(C)(C)C)c3ccc(C#N)cc23)n1]

A mixture of compound 75-1 (800 mg, 1.7 mmol), Zn(CN)₂ (600 mg, 5.2 mmol), actived Zn (28 mg, 0.4 mmol) and fresh prepared Pd(PPh₃)₄ (0.5 g) in dry DMF (30 mL) was stirred at 120° C. under a nitrogen atmosphere overnight. The reaction mixture was cooled to room temperature and quenched with H₂O (50 mL), then extracted with EtOAc/THF (1:1, 50 mL×3). The combined organic phases were washed with brine (50 mL×3), dried over anhydrous Na₂SO₄, filtered and concentrated in vacuo to afford crude. The crude was triturated with EtOAc (20 mL), filtered, dried and afford 75-2 (250 mg, 35% yield).

### Step 3: Methyl 2-(5-cyano-1H-indole-3-carbonyl)thiazole-4-carboxylate (PTC17341-60, ARI-141)

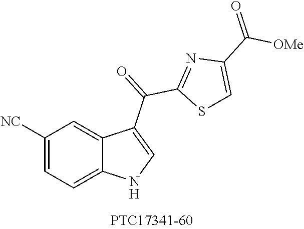 [SMILES: COC(=O)c1csc(C(=O)c2c[nH]c3ccc(C#N)cc23)n1]

This compound was synthesized according to the protocol described in Example 71 from compound 75-2 (250 mg, 0.6 mmol) to give title compound in the form of a yellow solid (65% yield). ¹H-NMR (400 MHz, DMSO-d6): δ 12.78 (bs, 1H), 9.22 (s, 1H), 8.95 (s, 1H), 8.66 (s, 1H), 7.78-7.82 (d, J=8.4 Hz, 1H), 7.68˜7.72 (d, J=8.4 Hz, 1H), 3.93 (s, 3H). LC-MS: m/z 310.1 [M−H]⁺.

### Example 119: Preparation of (4-(5-amino-1,3,4-oxadiazol-2-yl)thiazol-2-yl)(5-fluoro-1H-indol-3-yl)methanone (ARI-148)

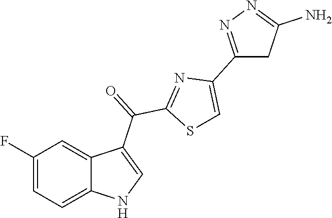 [SMILES: NC1=NN=C(c2csc(C(=O)c3c[nH]c4ccc(F)cc34)n2)C1]

Starting with 5-fluoroindole and using the procedure described in Example 131 the title compound was prepared.

### Example 120: Preparation of 1-(2-(4-fluoro-1H-indole-3-carbonyl)thiazol-4-yl)propan-1-one (ARI-142)

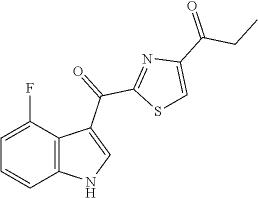 [SMILES: CCC(=O)c1csc(C(=O)c2c[nH]c3cccc(F)c23)n1]

Starting with 4-fluoroindole and using the procedure described in Example 84—Step 6, the title compound was prepared.

### Example 121: Preparation of 5-amino-2-(2-(7-fluoro-1H-indole-3-carbonyl)thiazol-4-yl)oxazole-4-carbonitrile (ARI-144)

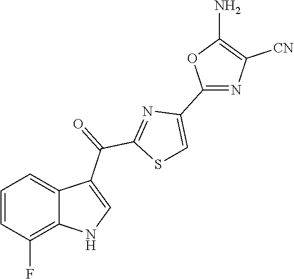 [SMILES: N#Cc1nc(-c2csc(C(=O)c3c[nH]c4c(F)cccc34)n2)oc1N]

Starting with 2-(1H-indole-7-fluoro-3-carbonyl)thiazole-4-carboxylic acid and using the method described in Example 116 the title compound was prepared.

### Example 122: Preparation of (4-(5-(aminomethyl)-1,3,4-oxadiazol-2-yl)thiazol-2-yl)(7-fluoro-1H-indol-3-yl)methanone (ARI-145)

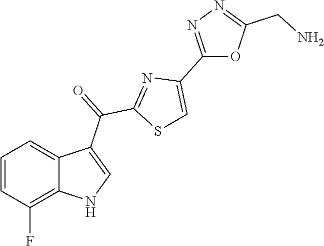 [SMILES: NCc1nnc(-c2csc(C(=O)c3c[nH]c4c(F)cccc34)n2)o1]

Starting with 2-(1H-indole-7-fluoro-3-carbonyl)thiazole-4-carboxylate and using the procedure outlined in Example 114, the title compound was prepared.

### Example 123: Preparation of (4-(5-(aminomethyl)-1,2,4-oxadiazol-3-yl)thiazol-2-yl)(7-fluoro-1H-indol-3-yl)methanone (ARI-146)

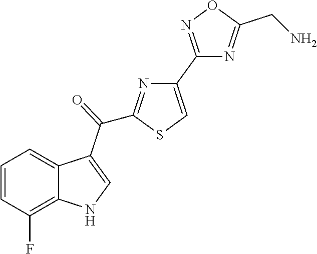 [SMILES: NCc1nc(-c2csc(C(=O)c3c[nH]c4c(F)cccc34)n2)no1]

Starting with 2-(1H-indole-7-fluoro-3-carbonyl)thiazole-4-hydroxyimidate and using the procedure described in Example 115 the title compound was prepared.

### Example 124: Preparation of 5-amino-2-(2-(5-fluoro-1H-indole-3-carbonyl)thiazol-4-yl)oxazole-4-carbonitrile (ARI-147)

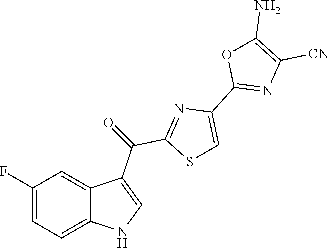 [SMILES: N#Cc1nc(-c2csc(C(=O)c3c[nH]c4ccc(F)cc34)n2)oc1N]

Starting with 2-(1H-indole-5-fluoro-3-carbonyl)thiazole-4-carboxylic acid and the procedure described in Example 116 the title compound was prepared.

### Example 125: Preparation of 1-(2-(5,6-difluoro-1H-indole-3-carbonyl)thiazol-4-yl)propan-1-one (ARI-149)

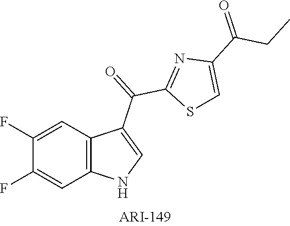 [SMILES: CCC(=O)c1csc(C(=O)c2c[nH]c3cc(F)c(F)cc23)n1]

ARI-149 was synthesized according to the scheme of FIG. 37 and by the following method. Potassium tert-butoxide (1.76 g, 16 mmol) was added to a solution of 6,7-difluoroindole (2.0 g, 13 mmol) in THF (100 mL) at 0° C. under N₂. The mixture was stirred for 1 h at this temperature, and then a solution of compound 70-5 (2.6 g, 13 mmol) in THF (20 mL) was added to it at 0° C. The reaction mixture was allowed to warm to room temperature and stirred overnight. The mixture was quenched with saturated NH4C1 aqueous (100 mL), stirred for 20 min, and filtered. The solid was collected, washed with water (30 mL×3) , EtOAc (30 mL×3) and MeOH (30 mL×3), dried to afford ARI-149 (459 mg, 11% yield) as yellow solid. ¹H-NMIR (400 MHz, DMSO-d6): δ 12.43 (bs, 1H), 9.19 (s, 1H), 8.87 (s, 1H), 8.13˜8.19 (m, 1H), 7.64˜7.70 (m, 1H), 3.21˜3.28 (q, J=7.2 Hz, 2H), 1.13˜1.18 (t, J=7.2 Hz, 3H). LC-MS: m/z 319.4 [M−H]⁻.

### Example 126: Preparation of 1-(2-(1H-indole-3-carbonyl)thiazol-4-yl)-2-methylpropan-1-one (ARI-048)

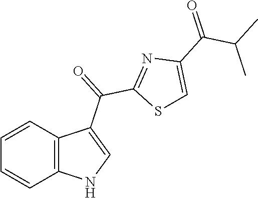 [SMILES: CC(C)C(=O)c1csc(C(=O)c2c[nH]c3ccccc23)n1]

Starting with 2-(1H-indole-3-carbonyl)thiazole-4-carboxylic acid and isopropylmagnesium bromide instead of methylmagnesium bromide using the procedure described in Example 75 the title compound was prepared.

### Example 127: Preparation of (1H-indol-3-yl)(4-(1-(methoxyimino) propyl)thiazol-2-yl) methanone (PTC17341-21, ARI-054)

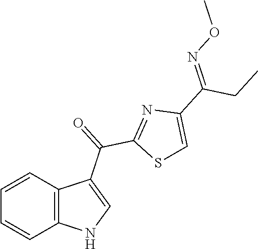 [SMILES: CC/C(=N\OC)c1csc(C(=O)c2c[nH]c3ccccc23)n1]

ARI-054 was synthesized according to the scheme of FIG. 38 and by the following method:

### Step 1: tert-Butyl 3-((4-propionylthiazol-2-yl)(trimethylsilyloxy)methyl)-1H-indole-1-carboxylate (79-1)

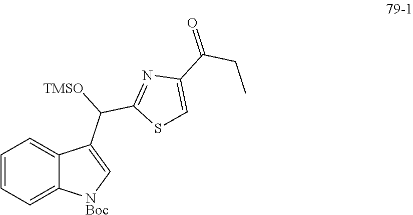

EtMgBr (2M in Et₂O, 40 mL, 80 mmol) was added portionwise to a solution of compound 40-2 (13.0 g, 26.6 mmol) in THF (200 mL) at 0° C. over 30 min. The resulting mixture was stirred for 0.5 h, then quenched with saturated aqueous NH₄Cl (500 mL), extracted with EtOAc (250 mL×3). The combined organic phases were washed with brine (500 mL×2), dried, concentrated to afford 79-1 (12.8 g, ˜100% yield) as an oil, which was used for next step without further purification.

### Step 2: tert-Butyl 3-((4-(1-(methoxyimino)propyl)thiazol-2-yl)(trimethylsilyloxy) methyl)-1H-indole-1-carboxylate (79-2)

NaOAc (722 mg, 8.8 mmol) and methoxylamine hydrochloride (355 mg, 4.2 mmol) were added to a solution of compound 79-1 (1.0 g, 2.2 mmol) in EtOH (5 mL) and H₂O (15 mL) at room temperature. The mixture was heated to 70° C. and stirred for 2 h. After cooled to room temperature, the mixture was concentrated in vacuo. The residue was purified by silica gel column chromatography (hexane/EtOAc=5:1) to give compound 79-2 (340 mg, 32% yield).

### Step 3: tert-Butyl 3-(hydroxy(4-(1-(methoxyimino)propyl)thiazol-2-yl)methyl)-1H-indole-1-carboxylate (79-3)

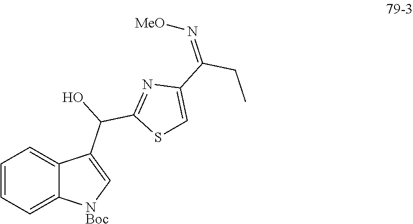 [SMILES: CC/C(=N/OC)c1csc(C(O)c2cn(C(=O)OC(C)(C)C)c3ccccc23)n1]

Compound 79-2 (340 mg, 0.7 mmol) was dissolved in THF (20 mL), TBAF (200 mg, 0.77 mmol) was added. The mixture was stirred for 2 h at room temperature, then quenched with water (50 mL), extracted with EtOAc (50 mL×3). The combined organic phases were washed with brine (100 mL×2), dried, concentrated to dryness. The residue was purified by silica gel chromatography (EtOAc/Hexane=1:3) and afforded compound 79-3 (210 mg, 72% yield).

### Step 4: tert-Butyl 3-(4-(1-(methoxyimino)propyl)thiazole-2-carbonyl)-1H-indole-1-carboxylate (79-4)

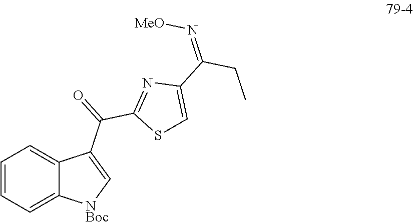 [SMILES: CC/C(=N/OC)c1csc(C(=O)c2cn(C(=O)OC(C)(C)C)c3ccccc23)n1]

This compound was synthesized according to the protocol described in Example 75 from compound 79-3 (470 mg, 1.1 mmol) to give title compound 79-4 in the form of a yellow solid (268 mg, 57% yield).

### Step 5: 1H-Indol-3-yl)(4-(1-(methoxyimino)propyl)thiazol-2-yl)methanone (PTC17341-21, ARI-054)

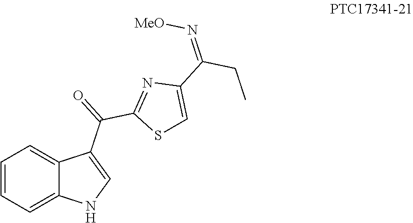 [SMILES: CC/C(=N/OC)c1csc(C(=O)c2c[nH]c3ccccc23)n1]

This compound was synthesized according to the protocol described in Example 71 from compound 79-4 (268 mg, 0.65 mmol) to give title compound PTC17341-21 (ARI-054) in the form of a yellow solid (95 mg, 47% yield). ¹H-NMR (400 MHz, DMSO-d6): δ 12.32 (bs, 1H), 9.07 (s, 1H), 8.31˜8.34 (m, 2H), 7.56˜7.59 (m, 1H), 7.27˜7.31 (m, 1H), 3.97 (s, 3H), 2.86˜2.94 (q, J=7.6 Hz, 2H), 1.14˜1.25 (m, 3H). LC-MS: m/z 314.3 [M+H]⁺.

### Example 128: Preparation of 1-(2-(6-fluoro-1H-indole-3-carbonyl)thiazol-4-yl)propan-1-one (ARI-143)

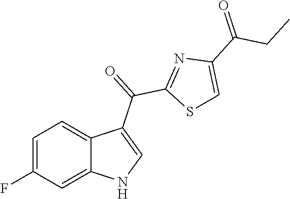 [SMILES: CCC(=O)c1csc(C(=O)c2c[nH]c3cc(F)ccc23)n1]

Starting with 6-fluoroindole and using the method described in Example 84—Step 6, the title compound was prepared.

### Example 129: Preparation of 1-(2-(5,7-difluoro-1H-indole-3-carbonyl)thiazol-4-yl)propan-1-one (ARI-150)

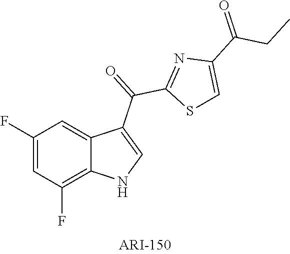 [SMILES: CCC(=O)c1csc(C(=O)c2c[nH]c3c(F)cc(F)cc23)n1]

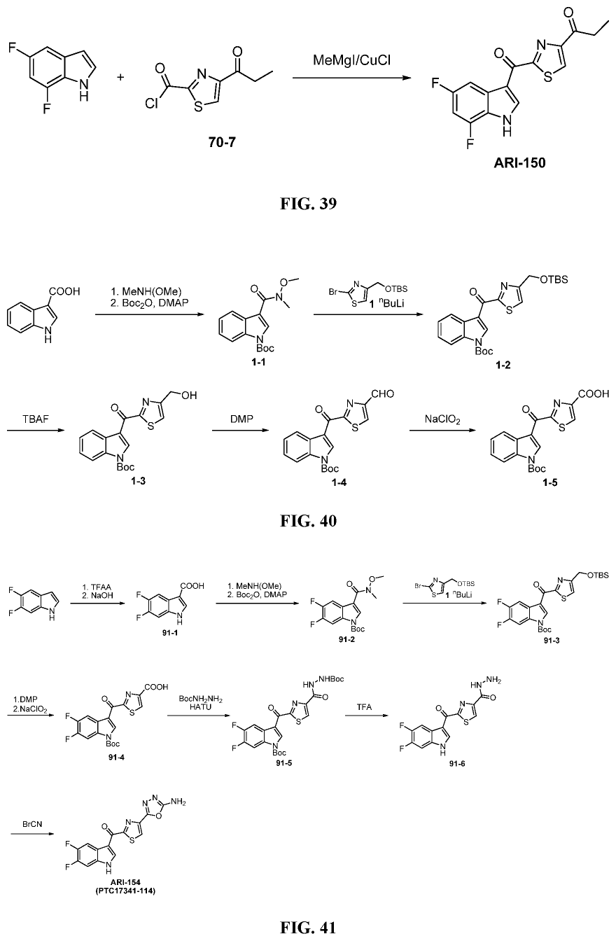

ARI-150 was synthesized according to the scheme of FIG. 39 and the protocol described in Example 84 from compound 70-7 (2.05 g, 10 mmol) and 5,7-difluoroindole to give title compound ARI-150 in the form of a yellow solid (1.51 g, 48% yield). ¹-NMR (400 MHz, DMSO-d6): δ 13.11 (bs, 1H), 9.19 (s, 1H), 8.88 (s, 1H), 7.83-7.87 (m, 1H), 7.24˜7.31 (m, 1H), 3.23˜3.28 (q, J=7.2 Hz, 2H), 1.13˜1.18 (t, J=7.2 Hz, 3H). LC-MS: m/z 318.9 [M−H]⁻

### Example 130: Preparation of 2-(1-(tert-butoxycarbonyl)-1H-indole-3-carbonyl) thiazole-4-carboxylic acid (1-5)

This compound was synthesized according to the scheme of FIG. 40 and by the following method:

### Step 1: tert-Butyl 3-(methoxy(methyl)carbamoyl)-1H-indole-1-carboxylate (1-1)

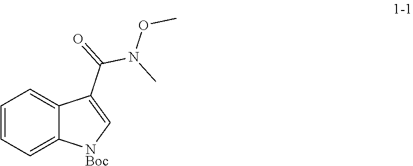 [SMILES: CON(C)C(=O)c1cn(C(=O)OC(C)(C)C)c2ccccc12]

Oxalyl chloride (473.3 g, 3.73 mol) was added dropwise to a suspension of indol-3-carboxylic acid (400.0 g, 2.48 mol) in DCM (4 L) at 0° C. over 1 h. The mixture was allowed to warm to room temperature and stirred overnight. The mixture was concentrated to dryness to afford 1H-indole-3-carbonyl chloride (446.0 g).

The above 1H-indole-3-carbonyl chloride (446.0 g) was added portion-wise to a suspension of N,O-dimethylhydroxylamine hydrochloride (266.0 g, 2.73 mol) and TEA (551.1 g, 5.46 mol) in DCM (5 L) at room temperature over 1 h. The mixture was stirred overnight, then quenched with water (2 L). The organic phase was collected and washed with water (2 L×2), saturated aqueous NaHCO₃ (2 L×2), and brine (2 L×1), dried (Na₂SO₄), filtered and concentrated to dryness. The residue and DMAP (15.1 g, 0.124 mol) was dissolved in DMF (1 L) and DCM (4 L), cooled to 0° C. Boc₂O (540.64 g, 2.48) and DMAP (15.1 g, 0.124 mol) were added dropwise to over 1 h. The resulting mixture was allowed to warm to room temperature and stirred overnight. The mixture was quenched with water (2 L). The organic phase was separated and washed with water (2 L×2), saturated aqueous NaHCO₃ (2 L×2), and brine (2 L×1), dried (Na₂SO₄), filtered and concentrated to dryness. The residue was triturated with EtOAc/hexane (1:5, 1 L), filtered and dried to afford compound 1-1 (557.9 g, 75% yield) as off-white solid. ¹H-NMR (400 MHz, DMSO-d6): δ 9.40 (s, 1H), 8.72 (s, 1H), 8.36˜8.38 (m 1H), 8.15˜8.18 (d, J=8.0 Hz, 1H), 7.40-7.50 (m, 2H), 3.80 (s, 3H), 3.40 (s, 3H), 1.69 (s, 9H).

### Step 2: tert-Butyl 3-(4-((tert-butyldimethylsilyloxy)methyl)thiazole-2-carbonyl)-1H-indole-1-carboxylate (1-2)

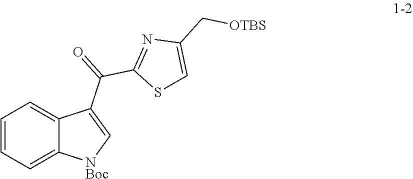 [SMILES: CC(C)(C)OC(=O)n1cc(C(=O)c2nc(CO[Si](C)(C)C(C)(C)C)cs2)c2ccccc21]

A solution of 2-bromo-4-((tert-butyldimethylsilyloxy)methyl)thiazole (135.0 g, 0.44 mol) in THF (1.5 L) was cooled to −78° C., and n-BuLi (1.6 M solution in hexane, 385 mL, 0.62 mol) was added dropwise at −78° C. over 1 h. The mixture was stirred for 0.5 h at this temperature, then a solution of compound 1-1 (120.0 g, 0.4 mol) in THF (500 mL) was added dropwise over 1 h. The mixture was stirred at −78° for 1 h then allowed to warm to 0° C. and quenched with aqueous 10% NH₄Cl (1 L). The organic phase was collected and washed with water (1 L×2), saturated aqueous NaHCO₃ (1 L×2), and brine (1 L×1), dried (Na₂SO₄), filtered and concentrated to dryness. The residue was triturated with EtOAc/hexane (1:5, 500 mL), filtered and dried to afford compound 1-2 (132.0 g, 70% yield) as off-white solid.

### Step 3: tert-Butyl 3-(4-(hydroxymethyl)thiazole-2-carbonyl)-1H-indole-1-carboxylate (1-3)

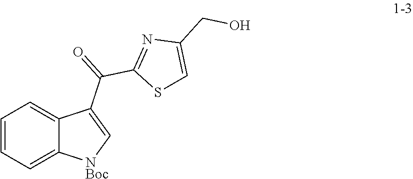 [SMILES: CC(C)(C)OC(=O)n1cc(C(=O)c2nc(CO)cs2)c2ccccc21]

A solution of compound 1-2 (91.0 g, 0.19 mol) in THF (500 mL) and pyridine (50 mL) was cooled to 0° C., and HF-pyridine (30%, 50 mL) was added dropwise over 10 min. The mixture was stirred for 0.5 h at this temperature, then allowed to warm to room temperature and stirred overnight. The mixture was quenched with aqueous 10% NH₄Cl (1 L) and EtOAc (500 mL). The organic phase was collected and washed with water (500 mL×2), saturated aqueous NaHCO₃ (500 mL×2), and brine (500 mL×1), dried (Na₂SO₄), filtered and concentrated to dryness. The residue was triturated with EtOAc/hexane (1:5, 100 mL), filtered and dried to afford compound 1-3 (49.6 g, 73% yield) as off-white solid.

### Step 4: tert-Butyl 3-(4-formylthiazole-2-carbonyl)-1H-indole-1-carboxylate (1-4)

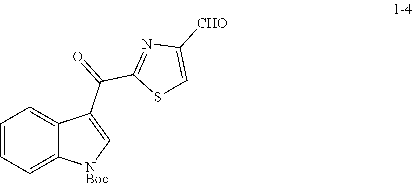 [SMILES: CC(C)(C)OC(=O)n1cc(C(=O)c2nc(C=O)cs2)c2ccccc21]

Dess-Martin periodinane (DMP, 26.1 g, 61 mmol) was added to a solution of compound 1-3 (20.0 g, 56 mmol) in DCM (350 mL) at 0° C. The mixture was stirred for 0.5 h at this temperature, then allowed to warm to room temperature and stirred overnight. The mixture was diluted with aqueous H₂O (500 mL) and DCM (500 mL), then filtered. The cake was washed with DCM (200 mL×3). The filtrate and washing were separated and the organic phase was collected, washed with aqueous 5% KHSO₄ (500 mL×3), saturated aqueous NaHCO₃ (500 mL×3), and brine (500 mL×1), dried (Na₂SO₄), filtered and concentrated to dryness. The residue was triturated with EtOAc/hexane (1:2, 50 mL), filtered and dried to afford compound 1-4 (20.2 g, 93% yield) as off-white solid. ¹H-NMR (400 MHz, DMSO-d6): δ 10.06 (s, 1H), 9.52 (s, 1H), 9.12 (s, 1H), 8.35˜8.40 (d, J=7.6 Hz, 1H), 8.14˜8.17 (d, J=8.0 Hz, 1H), 7.40˜7.50 (m, 2H), 1.71 (s, 9H). LC-MS: m/z: 357.4 [M+H]⁺

### Example 131: Preparation of (4-(5-amino-1,3,4-oxadiazol-2-yl)thiazol-2-yl)(5,6-difluoro-1H-indol-3-yl)methanone (ARI-154)

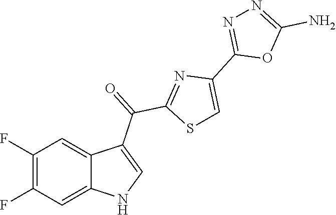 [SMILES: Nc1nnc(-c2csc(C(=O)c3c[nH]c4cc(F)c(F)cc34)n2)o1]

ARI-154 was synthesized according to the scheme of FIG. 41 and by the following method:

### Step 1: 5,6-Difluoro-1H-indole-3-carboxylic acide (91-1)

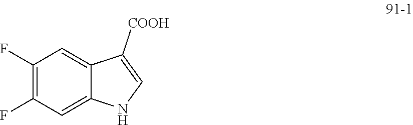 [SMILES: O=C(O)c1c[nH]c2cc(F)c(F)cc12]

Trifluoroacetic anhydride (38 mL, 56.0 g, 0.27 mol) was added dropwise to a solution of 5,6-difluoro-1H-indole (0.22 mol) in DMF (300 mL) over 0.5 h at 0° C. The reaction mixture was allowed to warm to room temperature and stirred overnight. The mixture was quenched with water (1 L), many solid began to form, the mixture was stirred for 0.5 h, then filtered. The solid was collected, washed with water (200 mL×3), then added to aqueous sodium hydroxide (20%, 150 mL, 0.75 mol) and heated under reflux for 8 h. The reaction mixture was cooled and acidified with aqueous 3N HCl to pH of 3. Many solid began to form. The solid was collected by filter, washed with water (200 mL×3), dried to give title compound 91-1 (15.53 g, 59% yield).

### Steps 2/3/4: 2-(1-(tert-butoxycarbonyl)-5,6-difluoro-1H-indole-3-carbonyl) thiazole-4-carboxylic acid (91-4)

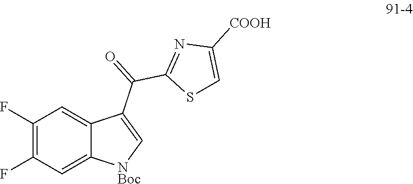 [SMILES: CC(C)(C)OC(=O)n1cc(C(=O)c2nc(C(=O)O)cs2)c2cc(F)c(F)cc21]

This compound was synthesized according to the protocol described in Example 130 from compound 91-1 (8.80 g, 44 mmol) to give title compound 91-4 in 36% yield.

### Step 5: tert-Butyl 3-(4-(2-(tert-butoxycarbonyl)hydrazinecarbonyl)thiazole-2-carbonyl)-5,6-difluoro-1H-indole-1-carboxylate (91-5)

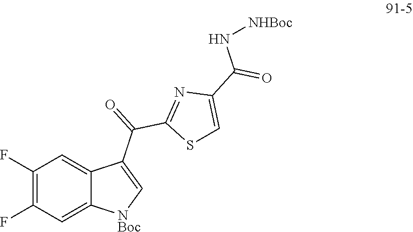 [SMILES: CC(C)(C)OC(=O)NNC(=O)c1csc(C(=O)c2cn(C(=O)OC(C)(C)C)c3cc(F)c(F)cc23)n1]

HATU (3.60 g, 95 mmol) and DIPEA (2.80 g, 22 mmol) were added to a suspension of compound 91-4 (3.00 g, 7.3 mmol) and Boc-hydrazine (1.50 g, 11 mmol) in DMF (20 mL) at 0° C. The mixture was allowed to warm to room temperature and stirred for 5 h. The mixture was diluted with H₂O (100 mL), extracted with EtOAc (100 mL×3). The combined organic phases were washed with brine (100 mL×2), dried, concentrated to dryness. The residue was purified by silica gel chromatography (EtOAc/Hexane=1:2 to 1:1) and afforded compound 91-5 (1.61 g, 42% yield).

### Step 6: 2-(5,6-difluoro-1H-indole-3-carbonyl)thiazole-4-carbohydrazide (91-6)

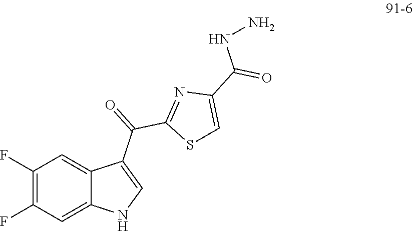 [SMILES: NNC(=O)c1csc(C(=O)c2c[nH]c3cc(F)c(F)cc23)n1]

A solution of compound 91-6 (1.60 g, 3 mmol) in DCM (50 mL) and TFA (50 mL) was stirred at room temperature for 3 h. The mixture concentrated to dryness. The residue was suspended in EtOAc (20 mL), alkalified by saturated aqueous NaHCO₃ to pH of 7˜8. The mixture was filtered to collect the solid. The solid was washed with water (10 mL×3) and EtOAc (10 mL×3), dried to afford 91-6 (0.92 g, 95% yield).

### Step 7: (4-(5-amino-1,3,4-oxadiazol-2-yl)thiazol-2-yl)(5,6-difluoro-1H-indol-3-yl) methanone (ARI-154)

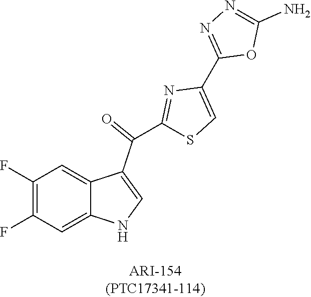 [SMILES: Nc1nnc(-c2csc(C(=O)c3c[nH]c4cc(F)c(F)cc34)n2)o1]

BrCN (0.50 g, 4.5 mmol) was added to a suspension of compound 91-6 (0.90 g, 2.8 mmol) in EtOH (250 mL) at room temperature. The mixture was heated to 65° C. and stirred for 20 h. after cooled to room temperature, the mixture was filtered to collect the solid. The solid was washed with EtOH (10 mL×3), dried to afford ARI-154 (520 mg, 52% yield) as yellow solid. 1H-NMR (400 MHz, DMSO-d6): δ12.55 (bs, 1H), 9.11 (s, 1H), 8.58 (s, 1H), 8.13˜8.16 (m, 1H), 7.67˜7.70 (m, 1H), 7.44 (s, 2H). LC-MS: m/z 346.0 [M−H]⁻.

### Example 132: Preparation of methyl 2-(5-methoxy-1H-indole-3-carbonyl)thiazole-4-carboxylate (ARI-080)

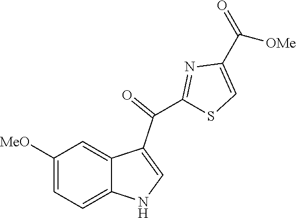 [SMILES: COC(=O)c1csc(C(=O)c2c[nH]c3ccc(OC)cc23)n1]

Starting with 2-(5-methoxy-1H-indole-3-carbonyl)thiazole-4-carboxylate and the method described in Example 65, the title compound was prepared.

### Example 133: Preparation of (4-(5-amino-1,3,4-oxadiazol-2-yl)thiazol-2-yl)(5-fluoro-2-methyl-1H-indol-3-yl)methanone (ARI-135)

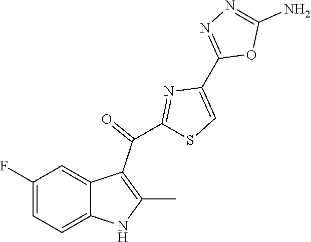 [SMILES: Cc1[nH]c2ccc(F)cc2c1C(=O)c1nc(-c2nnc(N)o2)cs1]

Starting with 5-fluoro-2-methyl-1H-indole and the method described in Example 131, the title compound was prepared.

### Example 134: Preparation of (2-(1H-indole-3-carbonyl)thiazol-4-yl)(3-hydroxyazetidin-1-yl)methanone (ARI-045)

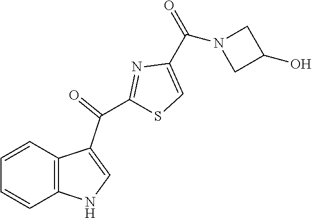 [SMILES: O=C(c1nc(C(=O)N2CC(O)C2)cs1)c1c[nH]c2ccccc12]

Using azetidine-3-ol instead of ethylamine and the method described in Example 24, the title compound was prepared.

### Example 135: Dibromo Indole Compounds

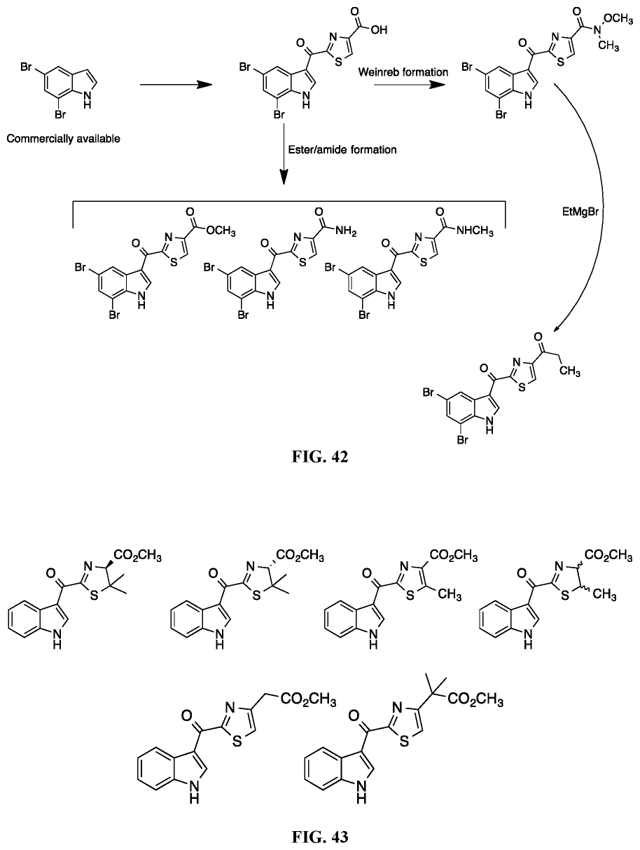

5,7-dibromo indole 3-carboxylic acid may be prepared according to Katner et al., “An Improved Synthesis of Indole-3-Carboxylic Acids,” Organic Preparations and Procedures Vol. 2, Iss. 4, 1970, incorporated herein by reference in its entirety. FIG. 42 shows a scheme of synthesizing dibromo indole compounds.

### Example 136: Modification of the Thiazole and Ester Fragments to Potentially Slow Down Ester Hydrolysis

FIG. 43 shows exemplary indole compounds where thiazole and ester fragments are modified to potentially slow ester hydrolysis. The compounds can be synthesized using cysteine derivatives, such as L and D penicillamine and 2-amino-3-sulfanyl butanoic acid, which are commercially available.

### Example 137: Synthesis of ARI-1073 and ARI-024

FIG. 44 describes a route of synthesis for ARI-1073 and ARI-024.

### Example 138: Synthesis of ARI-068, ARI-092, and ARI-094

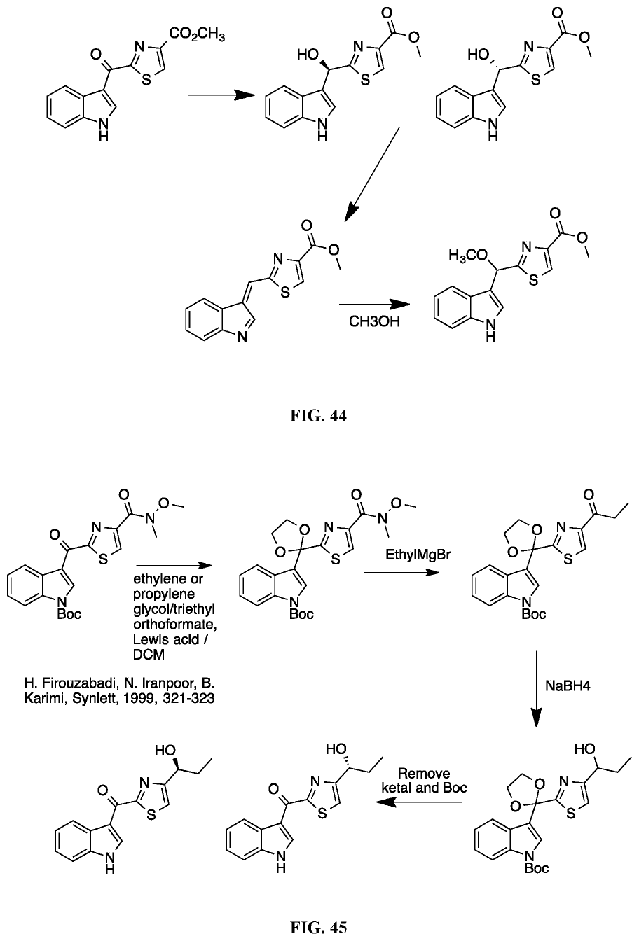

FIG. 45 illustrates a synthesis route for ARI-068, ARI-092, and ARI-094.

### Example 139: Synthesis of ARI-1029 and ARI-1030

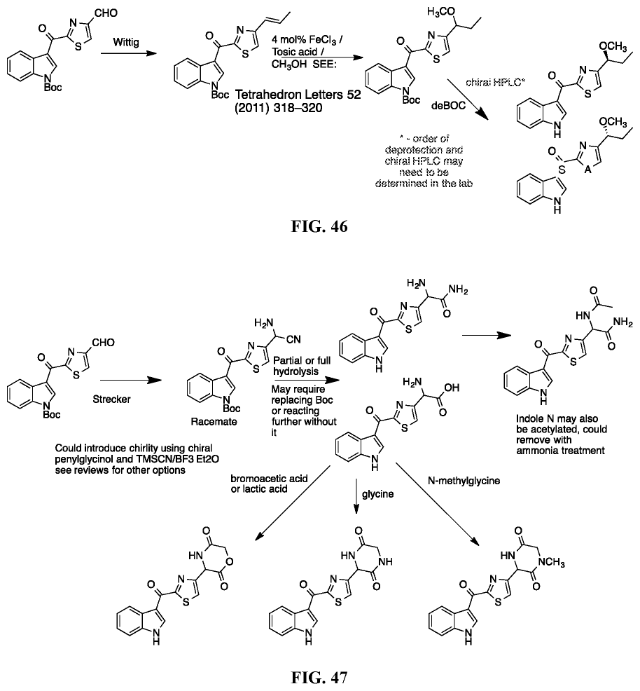

FIG. 46 illustrates a synthesis route for ARI-1029 and ARI-1030.

### Example 140: Synthesis of Amino Amides and Cyclic Versions of Indole Compounds

FIG. 47 illustrates a synthesis route for amino amides and cyclic versions of indole compounds.

### Example 141: Synthesis of Oxime Compounds with Hindered Ketones

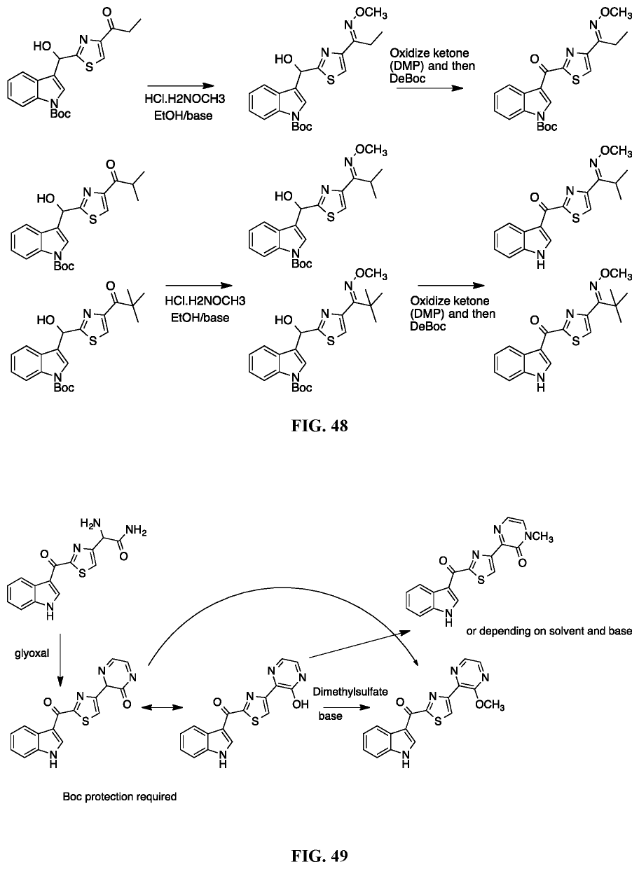

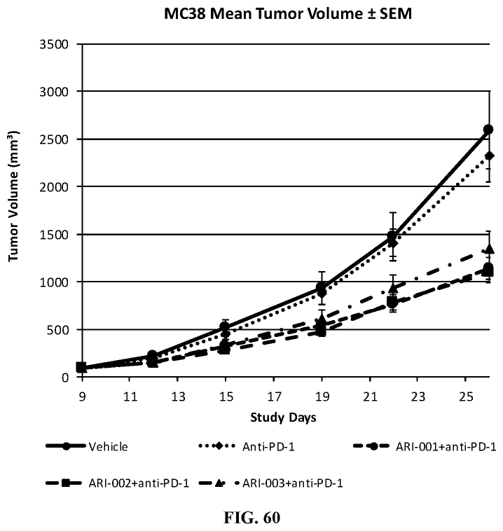

FIG. 48 illustrates a synthesis route for oxime compounds with hindered ketones. Additional routes to hindered ketones are shown in FIG. 59.

### Example 142: Synthesis of Pyrazine Compounds

FIG. 49 illustrates a synthesis route for pyrazine compounds.

### Example 143: Properties of Compounds with Thiazole and Indole Replacements

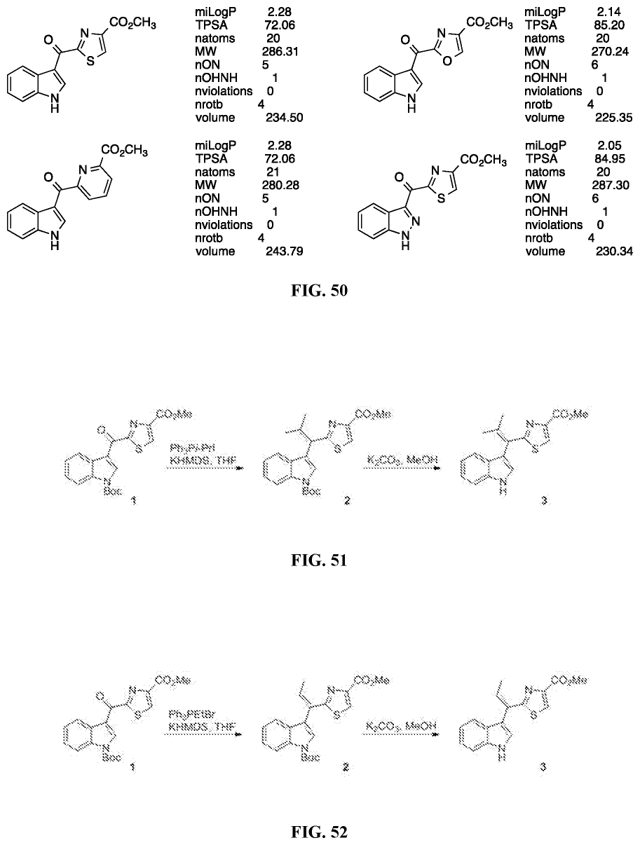

FIG. 50 compares the properties of compounds with thiazole and indole replacements.

### Example 144: Synthesis of ARI-020

FIG. 51 illustrates a synthesis scheme of ARI-020 (corresponding to product 3 in the synthesis scheme). According to this scheme, the yield of product 2 from 300 mg starting material 1 was 224 mg (70%). 1H NMR and MS results were consistent. Additionally, the yield of product 3 from 224 mg of starting material 2 was 45 mg (27%). ARI-020 was isolated as a lyophilized white solid with an HPLC purity >99%. The structure was confirmed by 1H NMR and MS.

### Example 145: Synthesis of ARI-018

FIG. 52 illustrates a synthesis scheme of ARI-018 (corresponding to product 3 in the synthesis scheme). According to this scheme, the yield of product 2 (a mixture of E/Z isomers) from 300 mg starting material 1 was 230 mg (74%).

### Example 146: Synthesis of ARI-019

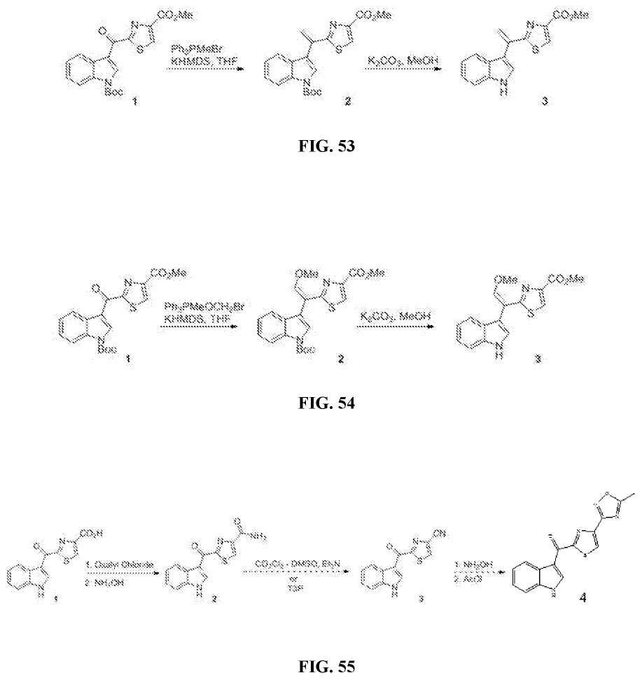

FIG. 53 illustrates a synthesis scheme of ARI-019 (corresponding to product 3 in the synthesis scheme). According to this scheme, the yield of product 2 from starting material 1 was 36%; and the yield of product 3 from starting material 2 was 22%. The synthesized ARI-019 was isolated in 90% HPLC purity after 2 columns.

### Example 147: Synthesis of ARI-017

FIG. 54 illustrates a synthesis scheme of ARI-017 (corresponding to product 3 in the synthesis scheme). According to this scheme, the yield of product 2 (E/Z isomers after column) from 300 mg starting material 1 was 277 mg (86%).

### Example 148: Synthesis of ARI-030

FIG. 55 illustrates a synthesis scheme for the preparation of ARI-030 (corresponding to product 4 in the synthesis scheme).

### Example 149: Synthesis of an Aldehyde Intermediate

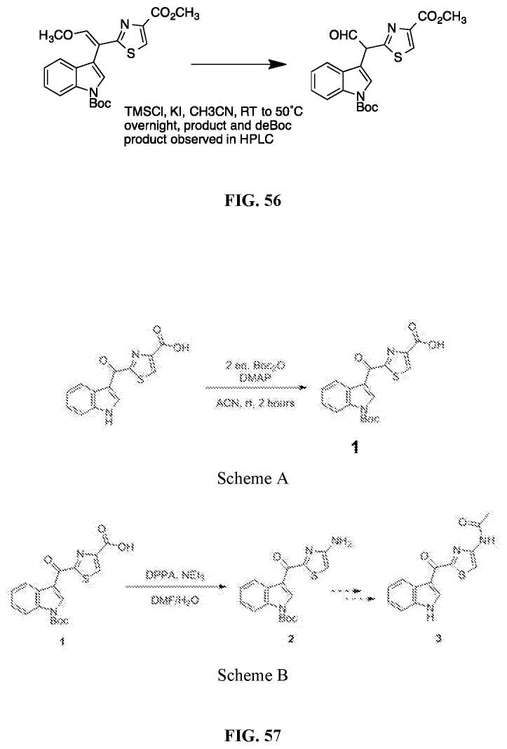

FIG. 56 shows a synthesis scheme of an aldehyde intermediate.

### Example 150: Synthesis of ARI-021

FIG. 57 illustrates a synthesis scheme for the preparation of ARI-021 (corresponding to product 3 in the synthesis Scheme B). Scheme A shows Boc protection of the starting carboxylic acid, with a yield of 81% (product 1). Scheme B shows the subsequent Curtius reaction on product 1, with a yield of product 2 from starting material 1 (0.266 g) of 113 mg (48%). 1H NMR and MS results were consistent with the proposed structure.

### Example 151: Synthesis of ARI-1057

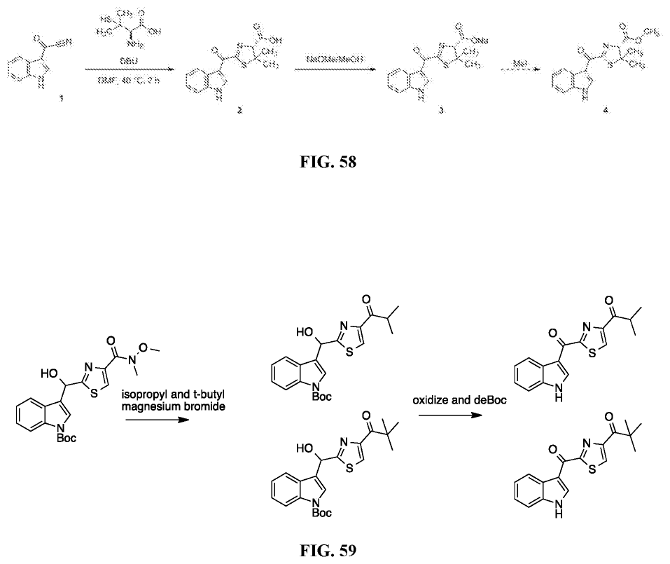

FIG. 58 illustrates a synthesis scheme of ARI-1057 (corresponding to product 4 in the synthesis scheme).

### Example 152: In Vivo Anti-Tumor Activity of ARI-001, ARI-002, ARI-003, or ARI-143 in Combination with an Anti-PD-1 Antibody

In this example, the in vivo anti-tumor efficacy of ARI-001, ARI-002, ARI-003, and ARI-143 and their combinations with an anti-PD-1 antibody was evaluated using a panel of twelve subcutaneous syngeneic mouse tumor models.

### Materials and Methods

### Subcutaneous Syngeneic Mouse Tumor Models

Eleven subcutaneous syngeneic mouse tumor models were generated by innoculating female BALB/C or C57BL/6 mice with cancer cells at their right lower or right front flank followed by randomization as detailed in Table 4 below.

TABLE 4

| | | | | | Age at Tumor | |
|---|---|---|---|---|---|---|
| Mouse | Cell | Cancer | Cell | Inoculation | Innoculation | Randomization |
| Strain | line | Type | Number | site | (weeks) | on Day |
| C57BL/6 | MC38 | Colon | 1 × 10⁶ | right lower flank | 7-9 | 9 |
| BALB/C | EMT-6 | Breast | 5 × 10⁵ | right lower flank | 7-9 | 6 |
| C57BL/6 | Pan02 | Pancreatic | 3 × 10⁶ | right front flank | 6-8 | 4 |
| BALB/C | CT26 | Colon | 5 × 10⁵ | right lower flank | 6-8 | 8 |
| BALB/C | A20 | Lymphoma | 5 × 10⁵ | right lower flank | 7-9 | 12 |
| C57BL/6 | LL/2 | Lung | 3 × 10⁵ | right lower flank | 7-9 | 16 |
| C57BL/6 | RM-1 | Prostate | 1 × 10⁶ | right lower flank | 6-8 | 10 |
| BALB/C | Renca | kidney | 1 × 10⁶ | right lower flank | 6-8 | 10 |
| C57BL/6 | Hepa1-6 | liver | 5 × 10⁶ | right front flank | 7-9 | 5 |
| C57BL/6 | B16F10 | Melanoma | 2 × 10⁵ | right lower flank | 7-9 | 9 |
| BALB/C | H22 | Liver | 1 × 10⁶ | right front flank | 6-8 | 5 |

### Formulation of Anti-PD-1 Antibody, ARI-001, ARI-002, and ARI-003

A solution of a rat monoclonal anti-mouse PD-1 antibody (isotype IgG_{2a,} κ) at a concentration of 6.61 mg/ml was obtained from BioXcell (InVivoMAb anti-mouse PD-1 (CD279), Clone RMP1-14, Cat #BE0146)) and store at 4° C. The antibody solution was diluted with PBS to obtain a 1 mg/ml dosing solution.

ARI-001, ARI-002, and ARI-003 powder was stored at −20° C. The powder of each compound was dissolved in DMSO to obtain dosing solutions at 106.7, 80, and 53.33 mg/ml for administration to mice at 160, 120, and 80 mg/kg, respectively. ARI-143 was similarly prepared.

### Study Design and Randomization

Twelve studies using the twelve subcutaneous syngeneic mouse tumor models were performed. In each study, 80 mice were enrolled and randomly allocated to eight different study groups, with 10 mice in each study group. The mean tumor size at randomization was approximately 80-120 mm³ (around 100 mm³). Randomization was performed based on “Matched distribution” randomization method (StudyDirector™ software, version 3.1.399.19). Table 5 shows the study design and the actual dosing frequency and number of doses. All the drugs and vehicle controls were injected to the mice intraperitoneally.

TABLE 5

| | | | | |
|---|---|---|---|---|
| Group | | | Dose Vol. | Dose Freq. & |
| No. | Treatment | Dose | (ml/kg) | Numbers |

Study 1 - MC38

| | | | | |
|---|---|---|---|---|
| 1 | Vehicle (PBS) | 0 | 10 | BIW × 5 doses |
| 3 | Anti-PD-1 | 10 | 10 | BIW × 5 doses |
| 6 | ARI-001 | 160 | 1.5 | QD × 17 doses |
| 7 | Anti-PD-1 | 10 | 10 | BIW × 5 doses |
| | ARI-001 | 160 | 1.5 | QD × 17 doses |
| 8 | ARI-002 | 160 | 1.5 | QD × 17 doses |
| 9 | Anti-PD-1 | 10 | 10 | BIW × 5 doses |
| | ARI-002 | 160 | 1.5 | QD × 17doses |
| 10 | ARI-003 | 160 | 1.5 | QD × 17 doses |
| 11 | Anti-PD-1 | 10 | 10 | BIW × 5 doses |
| | ARI-003 | 160 | 1.5 | QD × 17 doses |

Study 2 - EMT-6

| | | | | |
|---|---|---|---|---|
| 1 | Vehicle (PBS) | 0 | 10 | BIW × 6 doses |
| 3 | Anti-PD-1 | 10 | 10 | BIW × 6 doses |
| 6 | ARI-001 | 160/ | 1.5 | QD × 15 doses/ |
| | | 120 | | QD × 6 doses |
| 7 | Anti-PD-1 | 10 | 10 | BIW × 6 doses |
| | ARI-001 | 160/ | 1.5 | QD × 15 doses/ |
| | | 120 | | QD × 6 doses |
| 8 | ARI-002 | 160 | 1.5 | QD × 21 doses |
| 9 | Anti-PD-1 | 10 | 10 | BIW × 6 doses |
| | ARI-002 | 160 | 1.5 | QD × 21 doses |
| 10 | ARI-003 | 160 | 1.5 | QD × 21 doses |
| 11 | Anti-PD-1 | 10 | 10 | BIW × 6 doses |
| | ARI-003 | 160 | 1.5 | QD × 21 doses |

Study 3 - Pan02

| | | | | |
|---|---|---|---|---|
| 1 | Vehicle (PBS) | 0 | 10 | BIW × 8 doses |
| 3 | Anti-PD-1 | 10 | 10 | BIW × 8 doses |
| 6 | ARI-001 | 160 | 1.5 | QD × 28 doses |
| 7 | Anti-PD-1 | 10 | 10 | BIW × 8 doses |
| | ARI-001 | 160 | 1.5 | QD × 28 doses |
| 8 | ARI-002 | 160 | 1.5 | QD × 28 doses |
| 9 | Anti-PD-1 | 10 | 10 | BIW × 8 doses |
| | ARI-002 | 160 | 1.5 | QD × 28 doses |
| 10 | ARI-003 | 160 | 1.5 | QD × 28 doses |
| 11 | Anti-PD-1 | 10 | 10 | BIW × 8 doses |
| | ARI-003 | 160 | 1.5 | QD × 28 doses |

Study 4 - CT-26

| | | | | |
|---|---|---|---|---|
| 1 | Vehicle (PBS) | 0 | 10 | BIW × 5 doses |
| 3 | Anti-PD-1 | 10 | 10 | BIW × 5 doses |
| 6 | ARI-001 | 160 | 1.5 | QD × 15 doses |
| 7 | Anti-PD-1 | 10 | 10 | BIW × 5 doses |
| | ARI-001 | 160 | 1.5 | QD × 15 doses |
| 8 | ARI-002 | 160 | 1.5 | QD × 15 doses |
| 9 | Anti-PD-1 | 10 | 10 | BIW × 5 doses |
| | ARI-002 | 160 | 1.5 | QD × 15 doses |
| 10 | ARI-003 | 160 | 1.5 | QD × 15 doses |
| 11 | Anti-PD-1 | 10 | 10 | BIW × 5 doses |
| | ARI-003 | 160 | 1.5 | QD × 15 doses |

Study 5 - A20

| | | | | |
|---|---|---|---|---|
| 1 | Vehicle (PBS) | 0 | 10 | BIW × 4 doses |
| 3 | Anti-PD-1 | 10 | 10 | BIW × 4 doses |
| 6 | ARI-001 | 160 | 1.5 | QD × 14 doses |
| 7 | Anti-PD-1 | 10 | 10 | BIW × 4 doses |
| | ARI-001 | 160 | 1.5 | QD × 14 doses |
| 8 | ARI-002 | 160 | 1.5 | QD × 14 doses |
| 9 | Anti-PD-1 | 10 | 10 | BIW × 4 doses |
| | ARI-002 | 160 | 1.5 | QD × 14 doses |
| 10 | ARI-003 | 160 | 1.5 | QD × 14 doses |
| 11 | Anti-PD-1 | 10 | 10 | BIW × 4 doses |
| | ARI-003 | 160 | 1.5 | QD × 14 doses |

Study 6 - LL/2

| | | | | |
|---|---|---|---|---|
| 1 | Vehicle (PBS) | 0 | 10 | BIW × 5 doses |
| 3 | Anti-PD-1 | 10 | 10 | BIW × 5 doses |
| 6 | ARI-001 | 160/ | 1.5 | QD × 13 doses/ |
| | | 120/ | | QD × 2 doses/ |
| | | 80 | | QD × 1 dose |
| 7 | Anti-PD-1 | 10 | 10 | BIW × 5 doses |
| | ARI-001 | 160/ | 1.5 | QD × 13 doses/ |
| | | 120/ | | QD × 2 doses/ |
| | | 80 | | QD × 1 dose |
| 8 | ARI-002 | 160/ | 1.5 | QD × 13 doses/ |
| | | 120/ | | QD × 2 doses/ |
| | | 80 | | QD × 1 doses |
| 9 | Anti-PD-1 | 10 | 10 | BIW × 5 doses |
| | ARI-002 | 160/ | 1.5 | QD × 13 doses/ |
| | | 120/ | | QD × 2 doses/ |
| | | 80 | | QD × 1 doses |
| 10 | ARI-003 | 160/ | 1.5 | QD × 13 doses/ |
| | | 120/ | | QD × 2 doses/ |
| | | 80 | | QD × 1 dose |
| 11 | Anti-PD-1 | 10 | 10 | BIW × 5 doses |
| | ARI-003 | 160/ | 1.5 | QD × 13 doses/ |
| | | 120/ | | QD × 2 doses/ |
| | | 80 | | QD × 1 dose |

Study 7 - RM-1

| | | | | |
|---|---|---|---|---|
| 1 | Vehicle (PBS) | 0 | 10 | BIW × 3 doses |
| 3 | Anti-PD-1 | 10 | 10 | BIW × 3 doses |
| 6 | ARI-001 | 160 | 1.5 | QD × 10 doses |
| 7 | Anti-PD-1 | 10 | 10 | BIW × 3 doses |
| | ARI-001 | 160 | 1.5 | QD × 10 doses |
| 8 | ARI-002 | 160 | 1.5 | QD × 10 doses |
| 9 | Anti-PD-1 | 10 | 10 | BIW × 3 doses |
| | ARI-002 | 160 | 1.5 | QD × 10 doses |
| 10 | ARI-003 | 160 | 1.5 | QD × 10 doses |
| 11 | Anti-PD-1 | 10 | 10 | BIW × 3 doses |
| | ARI-003 | 160 | 1.5 | QD × 10 doses |

Study 8 - Renca

| | | | | |
|---|---|---|---|---|
| 1 | Vehicle (PBS) | 0 | 10 | BIW × 6 doses |
| 3 | Anti-PD-1 | 10 | 10 | BIW × 6 doses |
| 6 | ARI-001 | 160/ | 1.5 | QD × 5 doses/ |
| | | 120/ | | QD × 3 doses/ |
| | | 80 | | QD × 10 doses |
| 7 | Anti-PD-1 | 10 | 10 | BIW × 6 doses |
| | ARI-001 | 160/ | 1.5 | QD × 5 doses/ |
| | | 120/ | | QD × 3 doses/ |
| | | 80 | | QD × 10 doses |
| 8 | ARI-002 | 160/ | 1.5 | QD × 5 doses/ |
| | | 120/ | | QD × 3 doses/ |
| | | 80 | | QD × 10 doses |
| 9 | Anti-PD-1 | 10 | 10 | BIW × 6 doses |
| | ARI-002 | 160/ | 1.5 | QD × 5 doses/ |
| | | 120/ | | QD × 3 doses/ |
| | | 80 | | QD × 10 doses |
| 10 | ARI-003 | 160/ | 1.5 | QD × 5 doses/ |
| | | 120/ | | QD × 3 doses/ |
| | | 80 | | QD × 10 doses |
| 11 | Anti-PD-1 | 10 | 10 | BIW × 6 doses |
| | ARI-003 | 160/ | 1.5 | QD × 5 doses/ |
| | | 120/ | | QD × 3 doses/ |
| | | 80 | | QD × 10 doses |

Study 9 - Hepa1-6

| | | | | |
|---|---|---|---|---|
| 1 | Vehicle (PBS) | 0 | 10 | BIW × 6 doses |
| 3 | Anti-PD-1 | 10 | 10 | BIW × 6 doses |
| 6 | ARI-001 | 160 | 1.5 | QD × 21 doses |
| 7 | Anti-PD-1 | 10 | 10 | BIW × 6 doses |
| | ARI-001 | 160 | 1.5 | QD × 21 doses |
| 8 | ARI-002 | 160 | 1.5 | QD × 21 doses |
| 9 | Anti-PD-1 | 10 | 10 | BIW × 6 doses |
| | ARI-002 | 160 | 1.5 | QD × 21 doses |
| 10 | ARI-003 | 160 | 1.5 | QD × 21 doses |
| 11 | Anti-PD-1 | 10 | 10 | BIW × 6 doses |
| | ARI-003 | 160 | 1.5 | QD × 21 doses |

Study 10 - B16F10

| | | | | |
|---|---|---|---|---|
| 1 | Vehicle (PBS) | 0 | 10 | BIW × 3 doses |
| 3 | Anti-PD-1 | 10 | 10 | BIW × 3 doses |
| 6 | ARI-001 | 160 | 1.5 | QD × 13 doses |
| 7 | Anti-PD-1 | 10 | 10 | BIW × 4 doses |
| | ARI-001 | 160 | 1.5 | QD × 15 doses |
| 8 | ARI-002 | 160 | 1.5 | QD × 13 doses |
| 9 | Anti-PD-1 | 10 | 10 | BIW × 4 doses |
| | ARI-002 | 160 | 1.5 | QD × 15 doses |
| 10 | ARI-003 | 160 | 1.5 | QD × 12 doses |
| 11 | Anti-PD-1 | 10 | 10 | BIW × 4 doses |
| | ARI-003 | 160 | 1.5 | QD × 12 doses |

Study 11-H22

| | | | | |
|---|---|---|---|---|
| 1 | Vehicle (PBS) | 0 | 10 | BIW × 4 doses |
| 3 | Anti-PD-1 | 10 | 10 | BIW × 4 doses |
| 6 | ARI-001 | 160 | 1.5 | QD × 13 doses |
| 7 | Anti-PD-1 | 10 | 10 | BIW × 4 doses |
| | ARI-001 | 160 | 1.5 | QD × 13 doses |
| 8 | ARI-002 | 160 | 1.5 | QD × 13 doses |
| 9 | Anti-PD-1 | 10 | 10 | BIW × 4 doses |
| | ARI-002 | 160 | 1.5 | QD × 13 doses |
| 10 | ARI-003 | 160 | 1.5 | QD × 13 doses |
| 11 | Anti-PD-1 | 10 | 10 | BIW × 4 doses |
| | ARI-003 | 160 | 1.5 | QD × 13 doses |

STUDY 12 - EMT-6

| | | | | |
|---|---|---|---|---|
| 1 | Vehicle (DMS) | 0 | 1.5 | QD × 28 doses |
| 2 | Anti-PD-1 | 10 | 10 | BIW × 8 doses |
| 3 | ARI-143 | 40 | 1.5 | QD × 28 doses |

### Observation and Data Collection

After tumor cell inoculation, the animals were checked daily for morbidity and mortality. At the time of routine monitoring, the animals were checked for any effects of tumor growth and treatments on behavior such as mobility, food and water consumption, body weight gain/loss (body weights were measured twice or three times per week after randomization), and any other abnormalities. Mortality and observed clinical signs were recorded for individual animals. Tumor volumes were measured twice or three times weekly after randomization in two dimensions using a caliper, and the volume was expressed in mm³ using the formula:

V=(L×W×W)/2,

where V is tumor volume, L is tumor length (the longest tumor dimension) and W is tumor width (the longest tumor dimension perpendicular to L).

Dosing as well as tumor and body weight measurement were conducted in a Laminar Flow Cabinet.

### Termination

The study was terminated when the mean tumor volume (MTV) in the vehicle group reached a value of 2,000 mm³. Tumor growth inhibition (TGI) is an indication of antitumor activity, and expressed as:

TGI(%)=100×(1−T/C),

where T and C are the MTV (or weight) of the treated and control groups, respectively, on a given day. Statistical analysis of the difference in MTV among the groups was conducted using the data collected on the day when the MTV of the vehicle group reached the humane endpoints, so that TGI could be derived for all or most mice enrolled in the study.

Individual dosing holidays were implemented in the EMT-6, LL/2, B16BL6, Pan02 and Renca models.

### Statistical Analysis

Statistical analysis of differences in MTV among the groups was conducted by Independent-Samples T Test using the data collected. All data were analyzed with SPSS (Statistical Product and Service Solutions) version 18.0 (IBM, Armonk, N.Y., U.S.). P-values were rounded to three decimal places, with the exception that raw P-values less than 0.001 were stated as P<0.001. All tests were two-sided. P<0.05 was considered to be statistically significant.

### Results

### Tumor Growth Inhibition

FIG. 60 is a graph showing the mean tumor volume on different study days in the study groups as indicated according to Study 1 (MC38). The TGI data are summarized in Table 6. The study end day was Day 26.

TABLE 6
TGI in MC38 Model

| Group | Treatment | Tumor size^{a} | TGI(%) | P value^{b} | P value^{c} | P value^{d} |
|---|---|---|---|---|---|---|
| 1 | Vehicle (PBS) | 2592.31 ± 408.27 | — | — | — | — |
| 3 | Anti-PD-1 | 2324.55 ± 273.09 | 10 | 0.592 | | |
| 6 | ARI-001 | 1624.33 ± 148.56 | 37 | 0.047 | | |
| 7 | Anti-PD-1 + | 1139.95 ± 113.76 | 56 | 0.006 | 0.001 | 0.018 |
| | ARI-001 | | | | | |
| 8 | ARI-002 | 1419.94 ± 141.48 | 45 | 0.020 | | |
| 9 | Anti-PD-1 + | 1096.83 ± 102.93 | 58 | 0.005 | 0.001 | 0.083 |
| | ARI-002 | | | | | |
| 10 | ARI-003 | 1691.11 ± 123.57 | 35 | 0.059 | | |
| 11 | Anti-PD-1 + | 1351.75 ± 180.49 | 48 | 0.016 | 0.008 | 0.138 |
| | ARI-003 | | | | | |

^{a}Mean ± SEM;
^{b}vs. group-1;
^{c}vs. group-3;
^{d}vs. ARI-001, 002 or 003.
P values indicating statistical significance are shown in boldface.
P < 0.05 indicates statistic significance.

The data of FIG. 60 and Table 6 show that in the MC38 syngeneic colon cancer model, the anti-tumor activity of the anti-PD-1 antibody was not significantly different from that of the vehicle (PBS). The data show that anti-PD-1 antibody, when combined with any one of ARI-001, ARI-002, and ARI-003, had a statistically significant increase in anti-tumor potency compared to the antibody alone or the vehicle. The data also show that ARI-001 had a statistically significant increase in anti-tumor potency when combined with anti-PD-1 antibody, compared to being used alone.

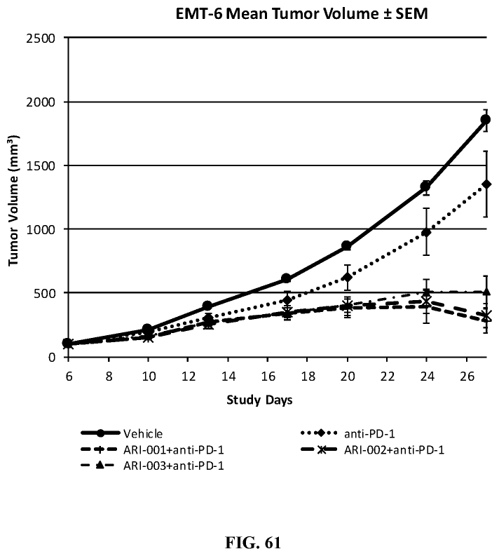

FIG. 61 is a graph showing the mean tumor volume on different study days in the study groups as indicated according to Study 2 (EMT-6). The TGI data are summarized in Table 7. The study end day was Day 27.

TABLE 7
TGI in EMT-6 Model

| Group | Treatment | Tumor size^{a} | TGI(%) | P value^{b} | P value^{c} | P value^{d} |
|---|---|---|---|---|---|---|
| 1 | Vehicle (PBS) | 1853.38 ± 85.42 | — | — | — | — |
| 3 | Anti-PD-1 | 1349.94 ± 257.90 | 27 | 0.094 | — | — |
| 6 | ARI-001 | 871.63 ± 142.67 | 53 | <0.001 | — | — |
| 7 | Anti-PD-1 + | 280.80 ± 91.59 | 85 | <0.001 | 0.003 | 0.014 |
| | ARI-001 | | | | | |
| 8 | ARI-002 | 842.08 ± 135.38 | 55 | <0.001 | — | — |
| 9 | Anti-PD-1 + | 321.80 ± 92.37 | 83 | <0.001 | 0.004 | 0.006 |
| | ARI-002 | | | | | |
| 11 | Anti-PD-1 + | 508.81 ± 126.72 | 73 | <0.001 | 0.013 | — |
| | ARI-003 | | | | | |

^{a}Mean ± SEM;
^{b}vs. group-1;
^{c}vs. group-3;
^{d}vs. ARI-001, 002 or 003.
P values indicating statistical significance are shown in boldface.

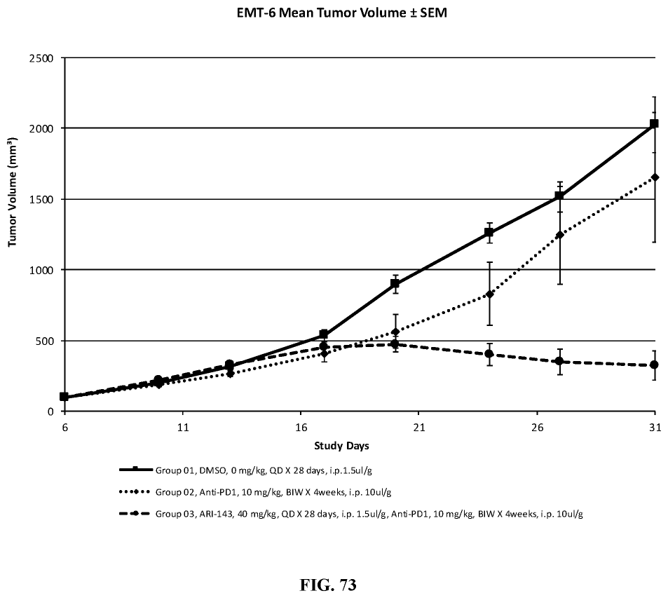

The data of FIG. 61 and Table 7 show that in the EMT-6 syngeneic breast cancer model, the anti-tumor activity of the anti-PD-1 antibody was not significantly different from that of the vehicle (PBS). The data show that anti-PD-1 antibody, when combined with any one of ARI-001, ARI-002, and ARI-003, had a statistically significant increase in anti-tumor potency compared to the antibody alone or the vehicle. The data also show that ARI-001 and ARI-002 had a statistically significant increase in anti-tumor potency when combined with anti-PD-1 antibody, compared to being used alone. Similar results were obtained when ARI-143 was used in lieu of ARI-001 and ARI-002 (FIG. 73).

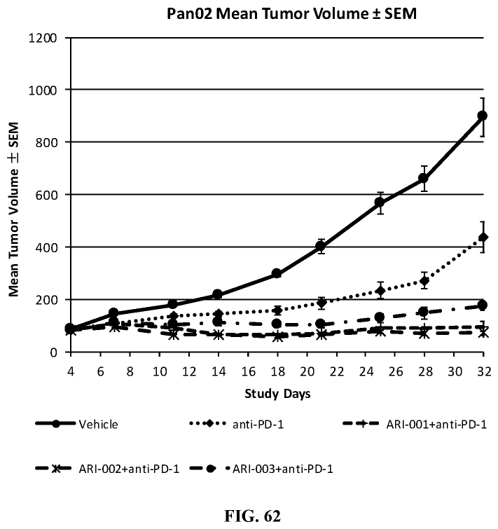

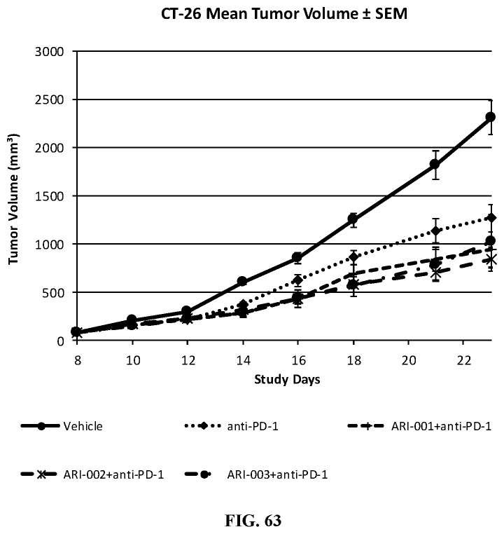

FIG. 62 is a graph showing the mean tumor volume on different study days in the study groups as indicated according to Study 3 (Pan02). The TGI data are summarized in Table 8. The study end day was Day 32.

TABLE 8
TGI in Pan02 Model

| Group | Treatment | Tumor size^{a} | TGI(%) | P value^{b} | P value^{c} | P value^{d} |
|---|---|---|---|---|---|---|
| 1 | Vehicle (PBS) | 896.94 ± 73.08 | — | — | — | — |
| 3 | Anti-PD-1 | 438.55 ± 59.07 | 51 | <0.001 | — | — |
| 6 | ARI-001 | 357.19 ± 232.32 | 60 | 0.033 | — | — |
| 7 | Anti-PD-1 + | 96.15 ± 18.17 | 89 | <0.001 | <0.001 | 0.295 |
| | ARI-001 | | | | | |
| 8 | ARI-002 | 308.02 ± 177.67 | 66 | 0.005 | — | — |
| 9 | Anti-PD-1 + | 75.87 ± 12.95 | 92 | <0.001 | <0.001 | 0.228 |
| | ARI-002 | | | | | |
| 10 | ARI-003 | 340.16 ± 49.76 | 62 | <0.001 | — | — |
| 11 | Anti-PD-1 + | 175.08 ± 18.65 | 80 | <0.001 | 0.001 | 0.006 |
| | ARI-003 | | | | | |

^{a}Mean ± SEM;
^{b}vs. group-1;
^{c}vs. group-3;
^{d}vs. ARI-001, 002 or 003.
P values indicating statistical significance are shown in boldface.
P values indicating statistical significance are shown in boldface.

The data of FIG. 62 and Table 8 show that in the Pan02 syngeneic pancreatic cancer model, the anti-tumor activity of the anti-PD-1 antibody was significantly better than that of the vehicle (PBS). The data show that anti-PD-1 antibody, when combined with any one of ARI-001, ARI-002, and ARI-003, had a statistically significant increase in anti-tumor potency compared to the antibody alone. The data also show that ARI-003 had a statistically significant increase in anti-tumor potency when combined with anti-PD-1 antibody, compared to being used alone.

FIG. 63 is a graph showing the mean tumor volume on different study days in the study groups as indicated according to Study 4 (CT-26). The TGI data are summarized in Table 9. The study end day was Day 23.

TABLE 9
TGI in CT-26 Model

| Group | Treatment | Tumor size^{a} | TGI(%) | P value^{b} | P value^{c} | P value^{d} |
|---|---|---|---|---|---|---|
| 1 | Vehicle (PBS) | 2308.06 ± 175.95 | — | — | — | — |
| 3 | Anti-PD-1 | 1268.52 ± 141.55 | 45 | <0.001 | — | — |
| 6 | ARI-001 | 1015.32 ± 114.50 | 56 | <0.001 | — | — |
| 7 | Anti-PD-1 + | 940.18 ± 179.74 | 59 | <0.001 | 0.168 | 0.728 |
| | ARI-001 | | | | | |
| 8 | ARI-002 | 1112.29 ± 124.36 | 51 | <0.001 | — | — |
| 9 | Anti-PD-1 + | 841.91 ± 121.51 | 64 | <0.001 | 0.035 | 0.139 |
| | ARI-002 | | | | | |
| 10 | ARI-003 | 836.51 ± 110.61 | 64 | <0.001 | — | — |
| 11 | Anti-PD-1 + | 1026.05 ± 277.54 | 56 | 0.001 | 0.447 | 0.534 |
| | ARI-003 | | | | | |

^{a}Mean ± SEM;
^{b}vs. group-1;
^{c}vs. group-3;
^{d}vs. ARI-001, 002 or 003.
P values indicating statistical significance are shown in boldface.
P values indicating statistical significance are shown in boldface.

The data of FIG. 63 and Table 9 show that in the CT-26 syngeneic colon cancer model, the anti-tumor activity of the anti-PD-1 antibody was significantly better than that of the vehicle (PBS). The data show that anti-PD-1 antibody, when combined with ARI-002, had a statistically significant increase in anti-tumor potency compared to the antibody alone.

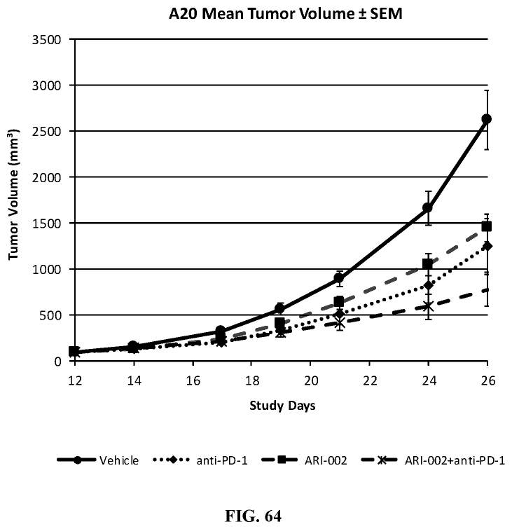

FIG. 64 is a graph showing the mean tumor volume on different study days in the study groups as indicated according to Study 5 (A20). The TGI data are summarized in Table 10. The study end day was Day 26.

TABLE 10
TGI in A20 Model

| Group | Treatment | Tumor size^{a} | TGI(%) | P value^{b} | P value^{c} | P value^{d} |
|---|---|---|---|---|---|---|
| 1 | Vehicle (PBS) | 2616.67 ± 320.98 | — | — | — | — |
| 3 | Anti-PD-1 | 1244.90 ± 302.87 | 52 | 0.006 | — | — |
| 6 | ARI-001 | 1190.08 ± 205.09 | 55 | 0.001 | — | — |
| 7 | Anti-PD-1 + | 811.44 ± 184.03 | 69 | <0.001 | 0.237 | 0.186 |
| | ARI-001 | | | | | |
| 8 | ARI-002 | 1446.75 ± 149.90 | 45 | 0.006 | — | — |
| 9 | Anti-PD-1 + | 773.87 ± 183.76 | 70 | <0.001 | 0.204 | 0.011 |
| | ARI-002 | | | | | |
| 10 | ARI-003 | 1206.83 ± 121.06 | 54 | 0.002 | — | — |
| 11 | Anti-PD-1 + | 1044.26 ± 199.72 | 60 | 0.001 | 0.587 | 0.497 |
| | ARI-003 | | | | | |

^{a}Mean ± SEM;
^{b}vs. group-1;
^{c}vs. group-3;
^{d}vs. ARI-001, 002 or 003.
P values indicating statistical significance are shown in boldface.
P values indicating statistical significance are shown in boldface.

The data of FIG. 64 and Table 10 show that in the A20 syngeneic lymphoma tumor model, the anti-tumor activity of the anti-PD-1 antibody was significantly better than that of the vehicle (PBS). The data show that ARI-002 had a statistically significant increase in anti-tumor potency when combined with anti-PD-1 antibody, compared to being used alone.

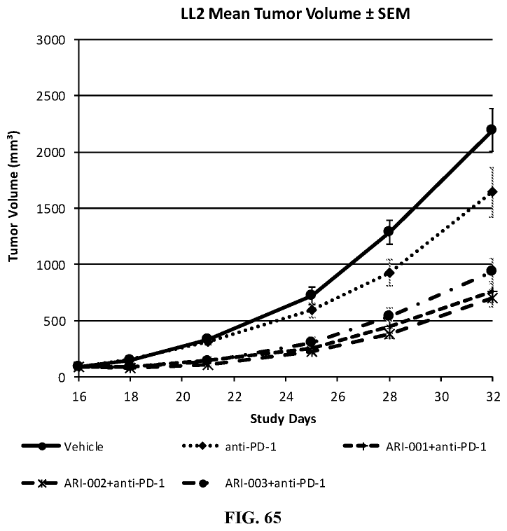

FIG. 65 is a graph showing the mean tumor volume on different study days in the study groups as indicated according to Study 6 (LL/2). The TGI data are summarized in Table 11. The study end day was Day 32.

TABLE 11
TGI in LL/2 Model

| Group | Treatment | Tumor size^{a} | TGI(%) | P value^{b} | P value^{c} | P value^{d} |
|---|---|---|---|---|---|---|
| 1 | Vehicle (PBS) | 2194.05 ± 190.66 | — | — | — | |
| 3 | Anti-PD-1 | 1643.42 ± 218.52 | 25 | 0.074 | — | — |
| 6 | ARI-001 | 881.1 ± 93.59 | 60 | <0.001 | — | — |
| 7 | Anti-PD-1 + | 756.03 ± 89.48 | 66 | <0.001 | 0.003 | 0.350 |
| | ARI-001 | | | | | |
| 8 | ARI-002 | 837.75 ± 117.58 | 62 | <0.001 | — | — |
| 9 | Anti-PD-1 + | 698.38 ± 77.63 | 68 | <0.001 | 0.003 | 0.356 |
| | ARI-002 | | | | | |
| 10 | ARI-003 | 839.79 ± 99.43 | 62 | <0.001 | — | — |
| 11 | Anti-PD-1 + | 940.1 ± 113.72 | 57 | <0.001 | 0.011 | 0.52 |
| | ARI-003 | | | | | |

^{a}Mean ± SEM;
^{b}vs. group-1;
^{c}vs. group-3;
^{d}vs. ARI-001, 002 or 003.
P values indicating statistical significance are shown in boldface.
P values indicating statistical significance are shown in boldface.

The data of FIG. 65 and Table 11 show that in the LL/2 syngeneic lung cancer model, the anti-tumor activity of the anti-PD-1 antibody was not significantly different from that of the vehicle (PBS). The data show that anti-PD-1 antibody, when combined with any one of ARI-001, ARI-002, and ARI-003, had a statistically significant increase in anti-tumor potency compared to the antibody alone or the vehicle.

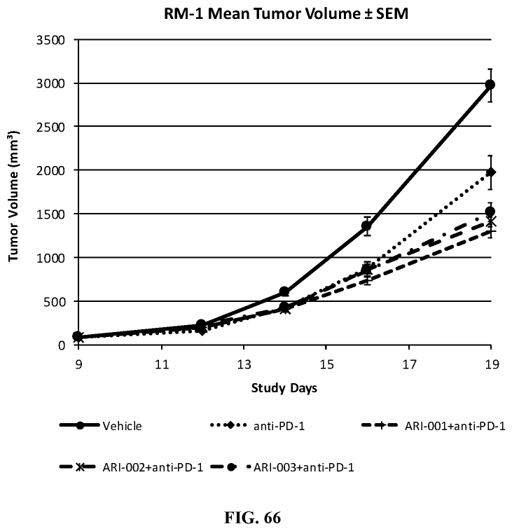

FIG. 66 is a graph showing the mean tumor volume on different study days in the study groups as indicated according to Study 7 (RM-1). The TGI data are summarized in Table 12. The study end day was Day 19.

TABLE 12
TGI in RM-1 Model

| Group | Treatment | Tumor size^{a} | TGI(%) | P value^{b} | P value^{c} | P value^{d} |
|---|---|---|---|---|---|---|
| 1 | Vehicle (PBS) | 2972.90 ± 186.53 | — | — | — | — |
| 3 | Anti-PD-1 | 1977.35 ± 195.05 | 33 | 0.002 | — | — |
| 6 | ARI-001 | 1732.55 ± 162.92 | 41 | <0.001 | — | — |
| 7 | Anti-PD-1 + | 1300.20 ± 78.09 | 56 | <0.001 | 0.005 | 0.028 |
| | ARI-001 | | | | | |
| 8 | ARI-002 | 1744.11 ± 144.25 | 41 | <0.001 | — | — |
| 9 | Anti-PD-1 + | 1409.05 ± 55.75 | 52 | <0.001 | 0.018 | 0.055 |
| | ARI-002 | | | | | |
| 10 | ARI-003 | 1703.05 ± 39.79 | 43 | <0.001 | — | — |
| 11 | Anti-PD-1 + | 1518.47 ± 108.12 | 49 | <0.001 | 0.054 | 0.136 |
| | ARI-003 | | | | | |

^{a}Mean ± SEM;
^{b}vs. group-1;
^{c}vs. group-3;
^{d}vs. ARI-001, 002 or 003.
P values indicating statistical significance are shown in boldface.
P values indicating statistical significance are shown in boldface.

The data of FIG. 66 and Table 12 show that in the RM-1 syngeneic prostate cancer model, the anti-tumor activity of the anti-PD-1 antibody was significantly better than that of the vehicle (PBS). The data show that anti-PD-1 antibody, when combined with ARI-001 or ARI-002, had a statistically significant increase in anti-tumor potency compared to the antibody alone. The data also show that ARI-001 had a statistically significant increase in anti-tumor potency when combined with anti-PD-1 antibody, compared to being used alone.

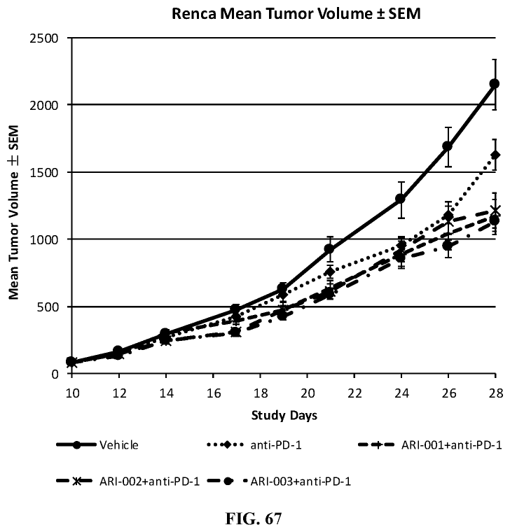

FIG. 67 is a graph showing the mean tumor volume on different study days in the study groups as indicated according to Study 8 (Renca). The TGI data are summarized in Table 13. The study end day was Day 28.

TABLE 13
TGI in Renca Model

| Group | Treatment | Tumor size^{a} | TGI(%) | P value^{b} | P value^{c} | P value^{d} |
|---|---|---|---|---|---|---|
| 1 | Vehicle (PBS) | 2147.63 ± 189.05 | — | — | — | — |
| 3 | Anti-PD-1 | 1628.04 ± 114.57 | 24 | 0.033 | — | — |
| 6 | ARI-001 | 1367.52 ± 118.53 | 36 | 0.003 | — | — |
| 7 | Anti-PD-1 + | 1176.68 ± 114.81 | 45 | <0.001 | 0.012 | 0.264 |
| | ARI-001 | | | | | |
| 8 | ARI-002 | 1101.99 ± 92.71 | 49 | <0.001 | — | — |
| 9 | Anti-PD-1 + | 1211.34 ± 128.71 | 44 | 0.001 | 0.027 | 0.500 |
| | ARI-002 | | | | | |
| 10 | ARI-003 | 1240.04 ± 90.09 | 42 | 0.001 | — | — |
| 11 | Anti-PD-1 + | 1132.31 ± 98.50 | 47 | <0.001 | 0.004 | 0.432 |
| | ARI-003 | | | | | |

^{a}Mean ± SEM;
^{b}vs. group-1;
^{c}vs. group-3;
^{d}vs. ARI-001, 002 or 003.
P values indicating statistical significance are shown in boldface.
P values indicating statistical significance are shown in boldface.

The data of FIG. 67 and Table 13 show that in the Renca syngeneic kidney cancer model, the anti-tumor activity of the anti-PD-1 antibody was significantly better than that of the vehicle (PBS). The data show that anti-PD-1 antibody, when combined with any one of ARI-001, ARI-002, and ARI-003, had a statistically significant increase in anti-tumor potency compared to the antibody alone.

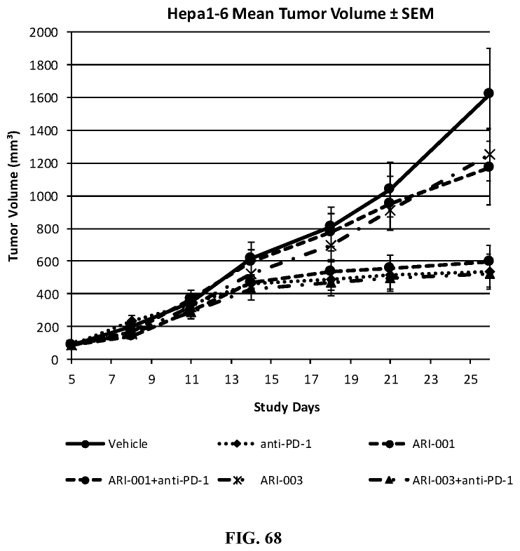

FIG. 68 is a graph showing the mean tumor volume on different study days in the study groups as indicated according to Study 9 (Hepal-6). The TGI data are summarized in Table 14. The study end day was Day 26.

TABLE 14
TGI in Hepa1-6 Model

| Group | Treatment | Tumor size^{a} | TGI(%) | P value^{b} | P value^{c} | P value^{d} |
|---|---|---|---|---|---|---|
| 1 | Vehicle (PBS) | 1616.92 ± 285.91 | — | — | — | — |
| 3 | Anti-PD-1 | 537.24 ± 108.36 | 67 | 0.004 | — | — |
| 6 | ARI-001 | 1174.25 ± 229.74 | 27 | 0.251 | — | — |
| 7 | Anti-PD-1 + | 599.17 ± 96.72 | 63 | 0.006 | 0.678 | 0.042 |
| | ARI-001 | | | | | |
| 8 | ARI-002 | 1018.26 ± 232.72 | 37 | 0.122 | — | — |
| 9 | Anti-PD-1 + | 550.17 ± 74.42 | 66 | 0.005 | 0.923 | 0.082 |
| | ARI-002 | | | | | |
| 10 | ARI-003 | 1251.31 ± 158.72 | 23 | 0.278 | — | — |
| 11 | Anti-PD-1 + | 525.57 ± 82.83 | 68 | 0.004 | 0.933 | 0.001 |
| | ARI-003 | | | | | |

^{a}Mean ± SEM;
^{b}vs. group-1;
^{c}vs. group-3;
^{d}vs. ARI-001, 002 or 003.
P values indicating statistical significance are shown in boldface.
P values indicating statistical significance are shown in boldface.

The data of FIG. 68 and Table 14 show that in the Hepal-6 syngeneic liver cancer model, the anti-tumor activity of the anti-PD-1 antibody was significantly better than that of the vehicle (PBS). The data show that ARI-001 and ARI-003 had a statistically significant increase in anti-tumor potency when combined with anti-PD-1 antibody, compared to being used alone.

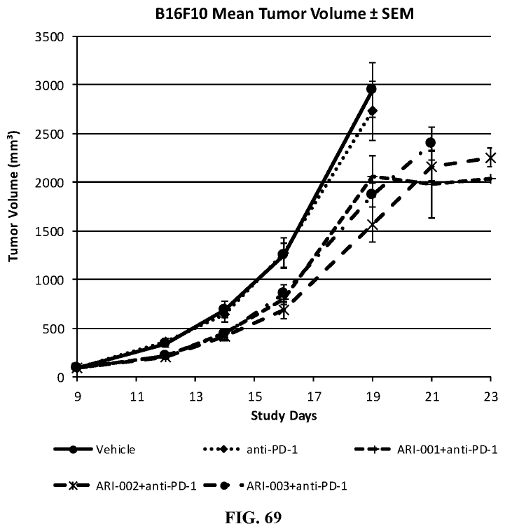

FIG. 69 is a graph showing the mean tumor volume on different study days in the study groups as indicated according to Study 10 (B16F10). The TGI data are summarized in Table 15. The study end day was Day 19.

TABLE 15
TGI in B16F10 Model

| Group | Treatment | Tumor size^{a} | TGI(%) | P value^{b} | P value^{c} | P value^{d} |
|---|---|---|---|---|---|---|
| 1 | Vehicle (PBS) | 2947.57 ± 283.83 | — | — | — | — |
| 3 | Anti-PD-1 | 2732.70 ± 302.03 | 7 | 0.613 | — | — |
| 6 | ARI-001 | 2089.97 ± 201.33 | 29 | 0.023 | — | — |
| 7 | Anti-PD-1 + | 2063.5 ± 211.49 | 30 | 0.022 | 0.086 | 0.929 |
| | ARI-001 | | | | | |
| 8 | ARI-002 | 1994.41 ± 180.33 | 32 | 0.010 | — | — |
| 9 | Anti-PD-1 + | 1566.00 ± 178.12 | 47 | 0.001 | 0.004 | 0.108 |
| | ARI-002 | | | | | |
| 10 | ARI-003 | 2234.18 ± 248.14 | 24 | 0.081 | — | — |
| 11 | Anti-PD-1 + | 1868.24 ± 120.67 | 37 | 0.002 | 0.016 | 0.176 |
| | ARI-003 | | | | | |

^{a}Mean ± SEM;
^{b}vs. group-1;
^{c}vs. group-3;
^{d}vs. ARI-001, 002 or 003.
P values indicating statistical significance are shown in boldface.
P values indicating statistical significance are shown in boldface.

The data of FIG. 69 and Table 15 show that in the B16F10 syngeneic melanoma model, the anti-tumor activity of the anti-PD-1 antibody was not significantly different from that of the vehicle (PBS). The data show that anti-PD-1 antibody, when combined with any one of ARI-002 or ARI-003, had a statistically significant increase in anti-tumor potency compared to the antibody alone or the vehicle.

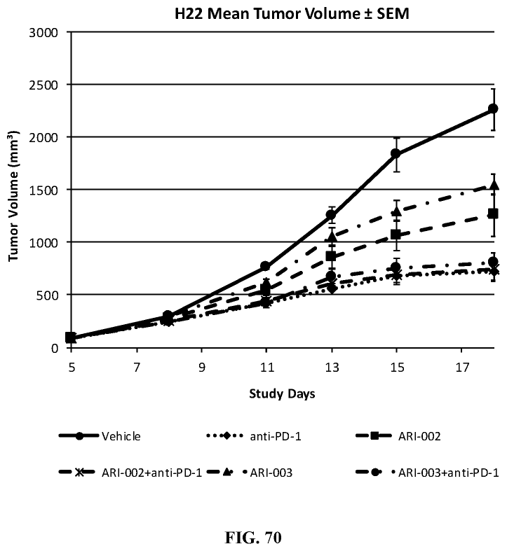

FIG. 70 is a graph showing the mean tumor volume on different study days in the study groups as indicated according to Study 11 (H22). The TGI data are summarized in Table 16. The study end day was Day 18.

TABLE 16
TGI in H22 Model

| Group | Treatment | Tumor size^{a} | TGI(%) | P value^{b} | P value^{c} | P value^{d} |
|---|---|---|---|---|---|---|
| 1 | Vehicle (PBS) | 2257.74 ± 197.15 | — | — | — | — |
| 3 | Anti-PD-1 | 719.6 ± 93.64 | 68 | <0.001 | — | — |
| 6 | ARI-001 | 1139.95 ± 207.21 | 50 | 0.001 | — | — |
| 7 | Anti-PD-1 + | 664.55 ± 56.61 | 71 | <0.001 | 0.621 | 0.050 |
| | ARI-001 | | | | | |
| 8 | ARI-002 | 1261.07 ± 203.55 | 44 | 0.002 | — | — |
| 9 | Anti-PD-1 + | 744.01 ± 106.50 | 67 | <0.001 | 0.865 | 0.042 |
| | ARI-002 | | | | | |
| 10 | ARI-003 | 1547.54 ± 100.51 | 31 | 0.005 | — | — |
| 11 | Anti-PD-1 + | 808.55 ± 91.06 | 64 | <0.001 | 0.505 | <0.001 |
| | ARI-003 | | | | | |

^{a}Mean ± SEM;
^{b}vs. group-1;
^{c}vs. group-3;
^{d}vs. ARI-001, 002 or 003.
P values indicating statistical significance are shown in boldface.
P values indicating statistical significance are shown in boldface.

The data of FIG. 70 and Table 16 show that in the H22 syngeneic liver cancer model, the anti-tumor activity of the anti-PD-1 antibody was significantly better than that of the vehicle (PBS). The data also show that ARI-002 and ARI-003 had a statistically significant increase in anti-tumor potency when combined with anti-PD-1 antibody, compared to being used alone.

### Example 153: In Vivo Anti-Tumor Activity of ARI-001 in Combination with Anti-CTLA-4 Antibody

In this example, the in vivo anti-tumor efficacy of ARI-001, an antibody against mouse CTLA-4 (mCTLA-4), and a combination of the two agents was evaluated using the subcutaneous syngeneic CT26.WT (aka CT-26) colon carcinoma mouse model.

### Materials and Methods

### Subcutaneous Syngeneic CT26. WT Colon Carcinoma Mouse Tumor Model

CT26.WT cells were obtained from the ATCC and grown in RPMI 1640 medium supplemented with 10% FBS at 37° C. in a 5% CO₂ atmosphere. Female Envigo BALB/c mice (BALB/cANNHsd) of 5-6 weeks old were implanted subcutaneously in the right axilla (high) on Day 0 with 5.0×10⁵ cells/mouse in 200 μl using a 27-gauge needle and syringe.

### Formulation of ARI-001 and the Anti-mCTLA-4 Antibody

ARI-001 powder was dissolved in 100% DMSO to obtain a 106.67 mg/ml dosing formulation with a pH of 7.6. The dosing solution was stored at room temperature and protected from light. The anti-mCTLA-4 9D9 was obtained from BioXcell. It was stored at 4° C. and protected from light; the 1.0 mg/mL formulation in PBS had a pH of 7.2.

### Study Design and Randomization

All mice were sorted into study groups based on caliper estimation of tumor burden. Each study group had ten mice. The mice were distributed to ensure that the mean tumor burden for all groups was within 10% of the overall mean tumor burden for the study population. Treatment began on Day 7 at an overall mean tumor burden of 89 mm³ (range of group means, 87-91 mm³). All mice were dosed according to individual body weight on the day of treatment (0.2 mL/20 g for anti-mCTLA-4 and vehicle (PBS), 30 μL/20 g for ARI-001 and vehicle (DMSO)). All agents were given to the mice intraperitoneally. Table 17 shows the details of the study design.

TABLE 17

| | | | Dose (mg/kg/ |
|---|---|---|---|
| Group | Treatment | Schedule | injection) |
| 1 | Vehicle | QD × 18 (Days 7-24) + | 30 μL/20 g + |
| | (DMSO) + | (Q3D × 2, 3 off) × 2 | 200 μL/20 g |
| | Vehicle (PBS) | (Days 7, 10, 14, 17) | |
| 2 | Vehicle | QD × 35 (Days 7-41) + | 30 μL/20 g + |
| | (DMSO) + | (Q3D × 2, 3 off) × 2 | 10 mg/kg/inj. |
| | 9D9 | then (Q3D × 3, 3 off) × | |
| | | 2, D 25 (Days 7, 10, 14, | |
| | | 17, 19, 22, 25, 29, 32, | |
| | | 35) | |
| 3 | ARI-001 + | QD × 41 (Days 7-47) + | 160 mg/kg/inj. + |
| | Vehicle (PBS) | (Q3D × 2, 3 off) × 2 | 200 μL/20 g |
| | | then (Q3D × 3, 3 off) × | |
| | | 2, D 25 (Days 7, 10, 14, | |
| | | 17, 19, 22, 25, 29, 32, | |
| | | 35) | |
| 4 | ARI-001 + | QD × 41 (Days 7-47) + | 160 mg/kg/inj. + |
| | 9D9 | (Q3D × 2, 3 off) × 2 | 10 mg/kg/inj. |
| | | then (Q3D × 3, 3 off) × | |
| | | 2, D 25 (Days 7, 10, 14, | |
| | | 17,19, 22, 25, 29, 32, 35) | |

### Measurement and Endpoints

Tumor measurements were recorded three times weekly. Tumor burden (mm³) was estimated from caliper measurements by the formula for the volume of a prolate ellipsoid assuming unit density as:

Tumor burden(mm³)=(L×W²)/2,

where L and W are the respective orthogonal tumor length and width measurements (mm).

The primary endpoints used to evaluate efficacy at the group level were: tumor growth delay, increased time to progression (% ITP), median ΔT/ΔC, complete and partial tumor response, and the number of tumor-free survivors at the end of the study. Tumor growth delay for this experiment was evaluated at 750 mm³. Time to progression study for this experiment was evaluated at 2,000 mm³. This is the IACUC required tumor burden limit, and the time to euthanasia can be thought of as a surrogate for lifespan.

In this experiment, median ΔT/ΔC was evaluated when the median control tumor burden reached 1,000 mm³ (Day 17). A mouse was considered a putative responder if it met at least one of the following criteria: complete regression of the tumor or being at least two standard deviations larger than the median time to progression of the control group. The following are the definitions of the efficacy parameters mentioned above: ΔC and ΔT are individual mouse endpoints that are calculated for each mouse as follows:

ΔT=Tt−T0 and ΔC=Ct−C0,

where Tt and T0 are the tumor burdens of a treated mouse at time t or at the initiation of dosing, respectively. ΔC reflects similar calculations for the control mice.

Median ΔT/ΔC is a group endpoint. It is calculated for each day of treatment as:

$$Median \frac{Δ T}{Δ C} = ( \frac{{Δ T}_{med}}{{Δ C}_{med}} ) * 100 = ( \frac{median ( {T}_{t} - {T}_{0} )}{median ( {C}_{t} - {C}_{0} )} ) * 100$$

The results are presented as %. When the median ΔT/ΔC is negative (the median treated tumor burden is regressing), the median ΔT/ΔC is not reported and the Median % Regression is reported instead.

% Regression is a group endpoint. It indicates the percentage reduction in the Median tumor volume from baseline. It is calculated as:

$$\% Regression = - ( \frac{{Δ T}_{med}}{{T}_{0} med} ) * 100$$

Time to Evaluation Size (TES)—TES is an individual mouse endpoint and it is expressed in days from tumor implant. It is the time it takes the tumor burden to reach a specified value, and it can be calculated from any method of evaluating tumor burden (caliper measurements, BLI, anatomical imaging, etc.). It is calculated by log-linear interpolation between the two closest data points that bracket the chosen tumor burden.

$${D}_{ES} = {D}_{h} - \frac{( ( log {V}_{h} - log ES ) * ( {D}_{h} - {D}_{l} ) )}{log {V}_{h} - log {V}_{i}}$$

where:

D_{ES}=TESᵢ—the day evaluation size is reached

Dₕ—the day of the first measurement greater than the ES was reached

Dₗ—the day of the last measurement before the ES was reached

Vₕ—The tumor volume on day Dₕ

Vₗ—the tumor volume on Dₗ

ES—the evaluation size

Time to Progression (TP)—Time to progression is a surrogate for lifespan, time on study, or lifespan. It is used for studies that involve IACUC mandated euthanasia of animals for excessive tumor burdens (even if the animals otherwise appear normal). The mandated tumor burden limit is tumor model dependent. TP data is analyzed by Kaplan Meier methods just as traditional life span data. The Time to Progression for an individual animal is the number of days between initiation of treatment and the death or required euthanasia of that animal. (The day of first treatment is the day of first treatment in the study as a whole and is not specific to the group in question.) When euthanasia is prompted for excessive tumor burden (typically >2000 mm³, but model dependent), the day of euthanasia is calculated from a log-linear interpolation between the adjacent data points on either side of the tumor burden limit, not from the actual day of euthanasia. This puts all animals on the same footing, and removes the impact of possibly delayed euthanasia (which may occur for sampling, or weekends and holidays). Animals euthanized for scheduled sampling or other causes unrelated to disease progression or therapy are excluded from this calculation. The median Time to Progression for a group is used to calculate the % Increase in Time to Progression (% ITP).

% Increase in Time to Progression (% ITP)—% ITP is a group endpoint. It is calculated as:

$$\% ITP = \{ \frac{[ ( median Treated TP ) - ( median Control TP ) ]}{median Control TP} \} * 100$$

Tumor doubling time (Td)—Td is an individual and group parameter, typically expressed as the median Td of the group. It is measured in days. Td can be calculated from any type of volumetric data (caliper measurements, BLI signals, etc). For QC purposes it is calculated for the exponential portion of the tumor growth curve. Data points during any lag phase and in the Gompertzian advanced stage are not included. Typical tumor burden limits are between 100 and 1000 mm³, but actual selection is data driven. Td is calculated for each mouse from a least squares best fit of a log/linear plot of tumor burden vs day as:

Td=log 2/slope.

On rare occasions the median Td is used as a potential indicator of efficacy. As such it is calculated as the median for every group, over a specified range of days thought to reflect a period of response to therapy.

Tumor growth delay (TGD, or T-C)—TGD is a group endpoint. Tumor growth delay is expressed in units of days and is calculated from the median times it takes the mice in a group to reach a specified tumor burden (time to evaluation size, TES). It can be calculated as:

TGD=median TES_{treated}−median TES_{control}

### Tumor Regressions

Complete Regression (CR)—An animal is credited a complete regression if its tumor burden is reduced to an immeasurable volume at any point after the first treatment. The convention is to record any tumor measurement less than 5 mm as a “0.” The CR must be maintained for at least 2 consecutive measurements. This is in keeping with the convention of the NCI and reflects the inherent and unacceptably high mechanical error in such measurements in addition to the uncertain biology of what is measured at those small sizes. (Individual efficacy parameter)

Partial regression—An animal is credited with a partial regression if its tumor burden decreases to less than half of the tumor burden at first treatment. The PR must be maintained for at least 2 consecutive measurements for caliper driven studies. PRs are tabulated exclusive of CRs, thus an animal that achieves a CR is not also counted as a PR. (Individual efficacy parameter)

Late regressions—In some studies (e.g., immuno-oncology studies), tumors may initially progress during treatment, followed by a period of regression. In that case Late CRs and Late PRs may be recorded. These are defined as described above with the exception that they are measured from the initial apex of the tumor growth curve.

Tumor-free Survivor (TFS)—A TFS is any animal that (1) survives until termination of the study, and (2) has no reliably measurable evidence of disease at study termination. Mice that are tumor-free at some point during the study but are then euthanized for sampling or other purposes prior to the end of the study are not considered TFS. They are excluded from calculation of the % TFS. TFS status does not imply “cure.”

### Assessment of Side Effects

All animals were observed for clinical signs at least once daily. Animals were weighed on each day of treatment. Individual body weights were recorded three times weekly. Animals with tumors more than 2,000 mm³ were euthanized, as were those found in obvious distress or in a moribund condition.

Treatment-related weight loss in excess of 20% is generally considered unacceptably toxic. In this example, a dosage level is described as tolerated if treatment-related weight loss (during and two weeks after treatment) is <20% and mortality during this period in the absence of potentially lethal tumor burdens is ≤10%.

### Statistics

The data were analyzed by the application of a one-way analysis of variance (ANOVA), with post-hoc analysis by the method of Shapiro-Wilk. In cases where the data did not pass testing for either normality or equal variance, a Kruskal-Wallis ANOVA by ranks was performed with post-hoc analysis by the method of Tukey/Dunn. The following statistical comparisons were performed:

1. Time to Progression, 2,000 mm³

2. ΔTs and ΔCs between groups on Day 17.

### Results

The mean estimated tumor burden for all groups in the experiment on the first day of treatment was 89 mm³ and all the groups in the experiment were well-matched (range of group means, 87-91 mm³). All animals weighed at least 15.4 g at the initiation of therapy. Mean group body weights at first treatment were also well-matched (range of group means, 16.4-17.9 g). In the Control Group (Group 1), the median time to 750 mm³ was 14.9 days, and the median tumor volume doubling time was 2.3 days. There were no spontaneous regressions in the Control Group. The following table is a comparison of mean body weight (MBW) change during treatment and key efficacy parameters (increase in median time to progression (MTP), complete response (CR), incidence of putative responders) among the study groups.

TABLE 18

| | | | Tumor | Day 17 | | | Incidence |
|---|---|---|---|---|---|---|---|
| | | MBW | Growth | Median | Increase in | | of putative |
| Group | Treatment | Change | Delay | ΔT/ΔC | MTP | CR | responders |
| 1 | Vehicle (DMSO) + | +12.1% | — | — | — | 0 | — |
| | Vehicle (PBS) | | | | | | |
| 2 | Vehicle (DMSO) + | +11.8% | 8.8 days | 22% | 77% | 0 | 60% |
| | 9D9 | | | (p < 0.05^{a}) | (p > 0.05^{a}) | | |
| 3 | ARI-001 + Vehicle | −8.3% | 2.9 days | 44% | 40% | 0 | 70% |
| | (PBS) | | | (p < 0.05^{a}) | (p < 0.05^{a}) | | |
| 4 | ARI-001 + 9D9 | −5.8% | 8.6 days | 33% | 183% | 10% | 60% |
| | | | | (p < 0.05^{a}) | (p < 0.05^{a}) | | |

^{a}p values were calculated relative to Group 1.

The data above demonstrate that the efficacy of the combination therapy with ARI-001 and the anti-mCTLA-4 antibody 9D9 was dramatically improved with a 183% increase in time to progression and 10% complete response rate as compared to the monotherapy with ARI-001 or the anti-mCTLA-4 antibody.

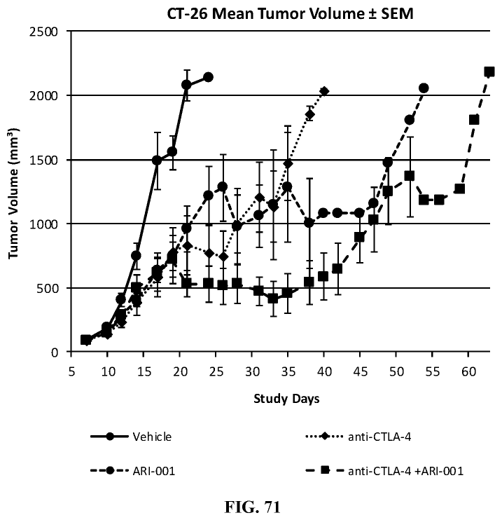

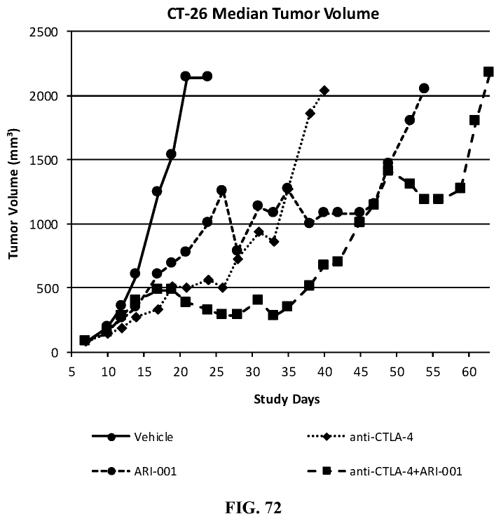

FIG. 71 is a graph showing the mean tumor volume on different days post tumor implant in the four study groups. FIG. 72 is a graph showing the median tumor volume on different days post tumor implant in the four study groups. These data demonstrate that the combination therapy with ARI-001 and the anti-mCTLA-4 antibody in Group 4 inhibited tumor growth more effectively than the anti-mCTLA-4 antibody monotherapy in Group 2 or the ARI-001 monotherapy in Group 3.

Taken together, this example shows that the combination therapy of ARI-001 with anti-CTLA-4 antibody was more effective than either agent alone in the tested mouse tumor model.

### Example 154: B16F10 Study in AhR Knockout Mice

To further validate the superior efficacy of the combination therapy described herein, the effect of ARI-001 and anti-PD-1 antibody was studied in B16F10 syngeneic mouse model as described in Example 153 above, except that the mice used were homozygous for a null mutation of AhR. The data showed that the tumor inhibitory effect of the ARI-001/anti-PD-1 combination was abrogated in the AhR^{-/-} mice. This result confirms that the superior anti-tumor efficacy observed with the ARI-001/anti-PD-1 combination was AhR-dependent.

Unless otherwise defined herein, scientific and technical terms used in connection with the present invention shall have the meanings that are commonly understood by those of ordinary skill in the art. Exemplary methods and materials are described below, although methods and materials similar or equivalent to those described herein can also be used in the practice or testing of the present invention. In case of conflict, the present specification, including definitions, will control. Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

1. A method of treating cancer in a patient, comprising administering to the patient (1) a therapeutically effective amount of a compound of formula 2, or an enantiomer, diastereomer, or pharmaceutically acceptable salt thereof, and (2) a therapeutically effective amount of an inhibitor of an immune checkpoint protein, 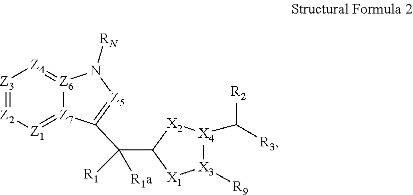
wherein:
X₁ is N (nitrogen), O (oxygen), S (sulfur), or C (carbon); X₂ is N (nitrogen), O (oxygen) S (sulfur), or C (carbon); X₃ is N (nitrogen), O (oxygen), S (sulfur) or C (carbon); and X₄ is N (nitrogen) O (oxygen), S (sulfur), or C (carbon), such that at least one of X₁, X₂, X₃ and X₄ is N, each of X₁, X₂, X₃ and X₄ is optionally selected to form a heteroaromatic, wherein the bond between X₁ and the adjacent carbon, between X₂ and the adjacent carbon, between X₁ and X₄, between X₂ and X₃, and between X₃ and X₄ can be a single bond or a double bond and the valence of X₁, X₂, X₃ and X₄ is completed with H or C₁-C₆ alkyl (i.e., the ring can be aromatic, partially saturated, or saturated);
Z₁ is N or CR₄, Z₂ is N or CR₅, Z₃ is N or CR₆, Z₄ is N or CR₇, Z₅ is N or CR₈, Z₆ is N or C, Z₇ is N or C, wherein no more than two of Z₁, Z₂, Z₃, Z₄, Z₅, Z₆, and Z₇ are N;
R₂ and R₃ are together selected from the group consisting of ═O, ═S, or ═NRₐ (Rₐ is H, C₁-C₆ alkyl, C₁-C₆ acyl, or —OR, R is H, C₁-C₆ alkyl, or C₁-C₆ acyl), or
R₂ and R₃ are each independently selected from the group consisting of —NRₐR_{b} (Rₐ and R_{b} are each independently H, C₁-C₆ alkyl, or C₁-C₆ acyl), hydrogen, deuterium, halo, amino, hydroxy, cyano, formyl, furyl, nitro, alkyl, haloalkyl, alkenyl, haloalkenyl, alkynyl, haloalkynyl, C₁-C₆ acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₀ (n=0 to 2, R₁₀ is directly connected to S), wherein R₁₀ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;
R₄, R₅, R₆, R₇, R₈, and R₉ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;
R_{N} is H, CN, C₁-C₆ alkyl, —OH, —(CO)—OR, or —OR, wherein R is H, C₁-C₆ alkyl, or C₁-C₆ acyl; and
R₁ and R₁ₐ are taken together to form ═NR_{b}, wherein R_{b} is H, C₁-C₆ alkyl, hydroxy, C₁-C₆ alkoxy (—O-alkyl), C₁-C₆ acyloxy, amino, or C₁-C₆ acyl, R₂ preferably can be ═O, R₃ preferably can be —OR, wherein R is H or C₁-C₆ alkyl; or
R₁ and R₁ₐ are taken together to form ═CR_{b}R_{c}, wherein R_{b} and R_{c} are each independently H, C₁-C₆ alkyl, alkoxy (—O-alkyl), thioalkoxy (—S-alkyl), cyano (—CN), or amino, R₂ preferably can be ═O, R₃ preferably can be —OR, wherein R is H or C₁-C₆ alkyl; or
R₁ and R₁ₐ are taken together to form ═O, ═NORₐ, or ═S, R₂ and R₃ preferably can be each independently —OR or —NRₐR_{b}, wherein R, Rₐ, and R_{b} are each independently H, C₁-C₆ alkyl, or C₁-C₆ acyl; or
R₁ and R₁ₐ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₂ (n=0 to 2, R₁₂ is directly connected to S), wherein R₁₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio; and optionally, adjacent R groups, together, can form a six- to twelve-membered ring.

2. A method of treating cancer in a patient, comprising administering to the patient (1) a therapeutically effective amount of a compound of formula 2a, or an enantiomer, diastereomer, or pharmaceutically acceptable salt thereof, and (2) a therapeutically effective amount of an inhibitor of an immune checkpoint protein, 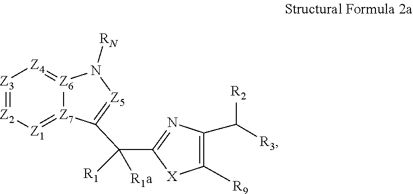
wherein:
X is either O (oxygen) or S (sulfur);
Z₁ is N or CR₄, Z₂ is N or CR₅, Z₃ is N or CR₆, Z₄ is N or CR₇, Z₅ is N or CR₈, Z₆ is N or C, Z₇ is N or C, wherein no more than two of Z₁, Z₂, Z₃, Z₄, Z₅, Z₆, and Z₇ are N;
R₂ and R₃ are together selected from the group consisting of ═O, ═S, or ═NRₐ (Rₐ is H, C₁-C₆ alkyl, C₁-C₆ acyl, or —OR, R is H, C₁-C₆ alkyl, or C₁-C₆ acyl), or
R₂ and R₃ are each independently selected from the group consisting of —NRₐR_{b} (Rₐ and R_{b} are each independently H, C₁-C₆ alkyl, or C₁-C₆ acyl), hydrogen, deuterium, halo, amino, hydroxy, cyano, formyl, furyl, nitro, alkyl, haloalkyl, alkenyl, haloalkenyl, alkynyl, haloalkynyl, C₁-C₆ acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₀ (n=0 to 2, R₁₀ is directly connected to S), wherein R₁₀ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;
R₄, R₅, R₆, R₇, R₈, and R₉ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;
R_{N} is H, CN, C₁-C₆ alkyl, —OH, —(CO)—OR, or —OR, wherein R is H, C₁-C₆ alkyl, or C₁-C₆ acyl; and
R₁ and R₁ₐ are taken together to form ═NR_{b}, wherein R_{b} is H, C₁-C₆ alkyl, hydroxy, C₁-C₆ alkoxy (—O-alkyl), C₁-C₆ acyloxy, amino, or C₁-C₆ acyl, R₂ preferably can be ═O, R₃ preferably can be —OR, wherein R is H or C₁-C₆ alkyl, or
R₁ and R₁ₐ are taken together to form ═CR_{b}R_{c}, wherein R_{b} and R_{c} are each independently H, C₁-C₆ alkyl, alkoxy (—O-alkyl), thioalkoxy (—S-alkyl), cyano (—CN), or amino, R₂ preferably can be ═O, R₃ preferably can be —OR, wherein R is H or C₁-C₆ alkyl, or
R₁ and R₁ₐ are taken together to form ═O, ═NORₐ, or ═S, R₂ and R₃ preferably can be each independently —OR or —NRₐR_{b}, wherein R, Rₐ, and R_{b} are each independently H, C₁-C₆ alkyl, or C₁-C₆ acyl, or
R₁ and R₁ₐ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₂ (n=0 to 2, R₁₂ is directly connected to S), wherein R₁₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio; and
optionally, adjacent R groups, together, can form a six- to twelve-membered ring.

3. A method of treating cancer in a patient, comprising administering to the patient (1) a therapeutically effective amount of a compound of formula 3, or an enantiomer, diastereomer, or pharmaceutically acceptable salt thereof, and (2) a therapeutically effective amount of an inhibitor of an immune checkpoint protein, 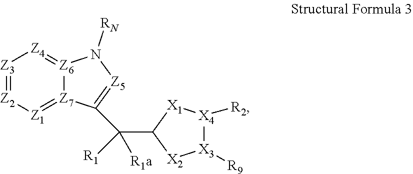
wherein:
X₁ is N (nitrogen), O (oxygen), S (sulfur), or C (carbon); X₂ is N (nitrogen), O (oxygen) S (sulfur), or C (carbon); X₃ is N (nitrogen), O (oxygen), S (sulfur) or C (carbon); and X₄ is N (nitrogen) O (oxygen), S (sulfur), or C (carbon), such that at least one of X₁, X₂, X₃ and X₄ is N, each of X₁, X₂, X₃ and X₄ is optionally selected to form a heteroaromatic, wherein the bond between X₁ and the adjacent carbon, between X₂ and the adjacent carbon, between X₁ and X₄, between X₂ and X₃, and between X₃ and X₄ can be a single bond or a double bond and the valence of X₁, X₂, X₃ and X₄ is completed with H or C₁-C₆ alkyl (i.e., the ring can be aromatic, partially saturated, or saturated);
Z₁ is N or CR₄, Z₂ is N or CR₅, Z₃ is N or CR₆, Z₄ is N or CR₇, Z₅ is N or CR₈, Z₆ is N or C, Z₇ is N or C, wherein no more than two of Z₁, Z₂, Z₃, Z₄, Z₅, Z₆, and Z₇ are N;
R₁ and R₁ₐ are taken together to form ═NR_{b}, wherein R_{b} is H, C₁-C₆ alkyl, hydroxy, C₁-C₆ alkoxy (—O-alkyl), C₁-C₆ acyloxy, amino, or C₁-C₆ acyl; or
R₁ and R₁ₐ are taken together to form ═CR_{b}R_{c}, wherein R_{b} and R_{c} are each independently H, C₁-C₆ alkyl, alkoxy (—O-alkyl), thioalkoxy (—S-alkyl), cyano (—CN), or amino; or
R₁ and R₁ₐ are taken together to form ═O, ═NORₐ, or ═S, wherein Rₐ is H, C₁-C₆ alkyl, or C₁-C₆ acyl, or
R₁ and R₁ₐ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₂ (n=0 to 2, R₁₂ is directly connected to S), wherein R₁₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio; and
R₂ and R₉ are each independently selected from the group consisting of hydrogen, aryl, heteroaryl, cycloalkyl, heterocycloalkyl, alkyl, —NR₂ₐC(O)OR_{2b}, —NR₂ₐC(O)R_{2b}, —(C₀-C₆ alkyl)-CONHSO₂R₂ₐ, —(C₀-C₆ alkyl)-CONHSO₂NR₂ₐR_{2b}, —(C₀-C₆ alkyl)-SO₂NHCOR₂ₐ, —(C₀-C₆ alkyl)-SO₂NHR₂ₐ, —(C₀-C₆ alkyl)-CONR₂ₐOR_{2b}, 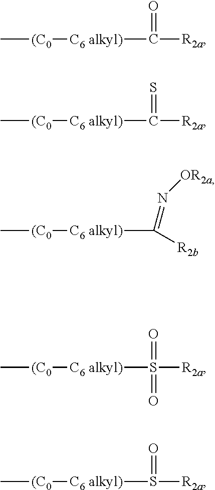
deuterium, halo, amino, hydroxy, cyano, formyl, nitro, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₀ (n=0 to 2, R₁₀ is directly connected to S), wherein R₁₀ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio, wherein R₂ₐ and R_{2b} are each independently H, C₁-C₆ alkyl, alkoxy (—O-alkyl), hydroxy, thioalkoxy (—S-alkyl), cyano (—CN), or amino;
R₄, R₅, R₆, R₇, and R₈ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;
R_{N} is H, CN, C₁-C₆ alkyl, —OH, —(CO)—OR, or —OR, wherein R is H, C₁-C₆ alkyl, or C₁-C₆ acyl; and
optionally, adjacent R groups, together, can form a six- to twelve-membered ring.

4. A method of treating cancer in a patient, comprising administering to the patient (1) a therapeutically effective amount of a compound of formula 3c, or an enantiomer, diastereomer, or pharmaceutically acceptable salt thereof, and (2) a therapeutically effective amount of an inhibitor of an immune checkpoint protein, 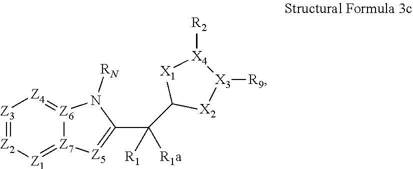
wherein:
X₁ is N (nitrogen), O (oxygen), S (sulfur), or C (carbon); X₂ is N (nitrogen), O (oxygen) S (sulfur), or C (carbon); X₃ is N (nitrogen), O (oxygen), S (sulfur) or C (carbon); and X₄ is N (nitrogen) O (oxygen), S (sulfur), or C (carbon), such that at least one of X₁, X₂, X₃ and X₄ is N, each of X₁, X₂, X₃ and X₄ is optionally selected to form a heteroaromatic, wherein the bond between X₁ and the adjacent carbon, between X₂ and the adjacent carbon, between X₁ and X₄, between X₂ and X₃, and between X₃ and X₄ can be a single bond or a double bond and the valence of X₁, X₂, X₃ and X₄ is completed with H or C₁-C₆ alkyl (i.e., the ring can be aromatic, partially saturated, or saturated);
Z₁ is N or CR₄, Z₂ is N or CR₅, Z₃ is N or CR₆, Z₄ is N or CR₇, Z₅ is N or CR₈, Z₆ is N or C, Z₇ is N or C, wherein no more than two of Z₁, Z₂, Z₃, Z₄, Z₅, Z₆, and Z₇ are N;
R₁ and R₁ₐ are taken together to form ═NR_{b}, wherein R_{b} is H, C₁-C₆ alkyl, hydroxy, C₁-C₆ alkoxy (—O-alkyl), C₁-C₆ acyloxy, amino, or C₁-C₆ acyl, or
R₁ and R₁ₐ are taken together to form ═CR_{b}R_{c}, wherein R_{b} and R_{c} are each independently H, C₁-C₆ alkyl, alkoxy (—O-alkyl), thioalkoxy (—S-alkyl), cyano (—CN), or amino, or
R₁ and R₁ₐ are taken together to form ═O, ═NORₐ, or ═S, wherein Rₐ is H, C₁-C₆ alkyl, or C₁-C₆ acyl, or
R₁ and R₁ₐ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₂ (n=0 to 2, R₁₂ is directly connected to S), wherein R₁₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio; and
R₂ and R₉ are each independently selected from the group consisting of hydrogen, aryl, heteroaryl, cycloalkyl, heterocycloalkyl, alkyl, —NR₂ₐC(O)OR_{2b}, —NR₂ₐC(O)R_{2b}, —(C₀-C₆ alkyl)-CONHSO₂R₂ₐ, —(C₀-C₆ alkyl)-CONHSO₂NR₂ₐR_{2b}, —(C₀-C₆ alkyl)-SO₂NHCOR₂ₐ, —(C₀-C₆ alkyl)-SO₂NHR₂ₐ, —(C₀-C₆ alkyl)-CONR₂ₐOR_{2b}, 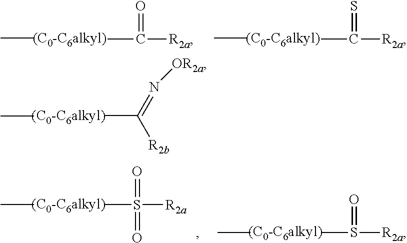
deuterium, halo, amino, hydroxy, cyano, formyl, nitro, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₀ (n=0 to 2, R₁₀ is directly connected to S), wherein R₁₀ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio, wherein R₂ₐ and R_{2b} are each independently H, C₁-C₆ alkyl, alkoxy (—O-alkyl), hydroxy, thioalkoxy (—S-alkyl), cyano (—CN), or amino;
R₄, R₅, R₆, R₇, and R₈ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;
R_{N} is H, CN, C₁-C₆ alkyl, —OH, —(CO)—OR, or —OR, wherein R is H, C₁-C₆ alkyl, or C₁-C₆ acyl; and
optionally, adjacent R groups, together, can form a six- to twelve-membered ring.

5. A method of treating cancer in a patient, comprising administering to the patient (1) a therapeutically effective amount of a compound of formula 3a, or an enantiomer, diastereomer, or pharmaceutically acceptable salt thereof, and (2) a therapeutically effective amount of an inhibitor of an immune checkpoint protein, 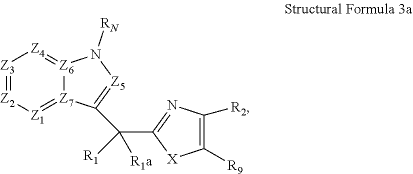
wherein:
X is either O (oxygen) or S (sulfur);
Z₁ is N or CR₄, Z₂ is N or CR₅, Z₃ is N or CR₆, Z₄ is N or CR₇, Z₅ is N or CR₈, Z₆ is N or C, Z₇ is N or C, wherein no more than two of Z₁, Z₂, Z₃, Z₄, Z₅, Z₆, and Z₇ are N;
R₁ and R₁ₐ are taken together to form ═NR_{b}, wherein R_{b} is H, C₁-C₆ alkyl, hydroxy, C₁-C₆ alkoxy (—O-alkyl), C₁-C₆ acyloxy, amino, or C₁-C₆ acyl, or
R₁ and R₁ₐ are taken together to form ═CR_{b}R_{c}, wherein R_{b} and R_{c} are each independently H, C₁-C₆ alkyl, alkoxy (—O-alkyl), thioalkoxy (—S-alkyl), cyano (—CN), or amino, or
R₁ and R₁ₐ are taken together to form ═O, ═NORₐ, or ═S, wherein Rₐ is H, C₁-C₆ alkyl, or C₁-C₆ acyl, or
R₁ and R₁ₐ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₂ (n=0 to 2, R₁₂ is directly connected to S), wherein R₁₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio; and
R₂ and R₉ are each independently selected from the group consisting of hydrogen, aryl, heteroaryl, cycloalkyl, heterocycloalkyl, alkyl, —NR₂ₐC(O)OR_{2b}, —NR₂ₐC(O)R_{2b}, —(C₀-C₆ alkyl)-CONHSO₂R₂ₐ, —(C₀-C₆ alkyl)-CONHSO₂NR₂ₐR_{2b}, —(C₀-C₆ alkyl)-SO₂NHCOR₂ₐ, —(C₀-C₆ alkyl)-SO₂NHR₂ₐ, —(C₀-C₆ alkyl)-CONR₂ₐOR_{2b}, 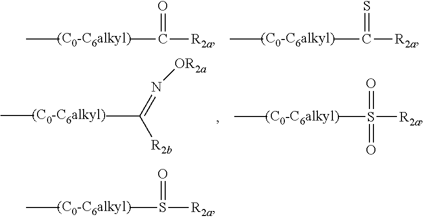
deuterium, halo, amino, hydroxy, cyano, formyl, nitro, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₀ (n=0 to 2, R₁₀ is directly connected to S), wherein R₁₀ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio, wherein R₂ₐ and R_{2b} are each independently H, C₁-C₆ alkyl, alkoxy (—O-alkyl), hydroxy, thioalkoxy (—S-alkyl), cyano (—CN), or amino;
R₄, R₅, R₆, R₇, and R₈ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;
R_{N} is H, CN, C₁-C₆ alkyl, —OH, —(CO)—OR, or —OR, wherein R is H, C₁-C₆ alkyl, or C₁-C₆ acyl; and
optionally, adjacent R groups, together, can form a six- to twelve-membered ring.

6. A method of treating cancer in a patient, comprising administering to the patient (1) a therapeutically effective amount of a compound of formula 3b, or an enantiomer, diastereomer, or pharmaceutically acceptable salt thereof, and (2) a therapeutically effective amount of an inhibitor of an immune checkpoint protein, 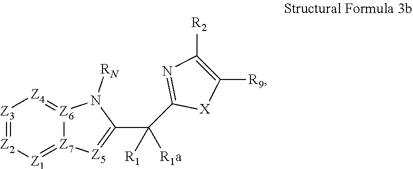
wherein:
X is either O (oxygen) or S (sulfur);
Z₁ is N or CR₄, Z₂ is N or CR₅, Z₃ is N or CR₆, Z₄ is N or CR₇, Z₅ is N or CR₈, Z₆ is N or C, Z₇ is N or C, wherein no more than two of Z₁, Z₂, Z₃, Z₄, Z₅, Z₆, and Z₇ are N;
R₁ and R₁ₐ are taken together to form ═NR_{b}, wherein R_{b} is H, C₁-C₆ alkyl, hydroxy, C₁-C₆ alkoxy (—O-alkyl), C₁-C₆ acyloxy, amino, or C₁-C₆ acyl; or
R₁ and R₁ₐ are taken together to form ═CR_{b}R_{c}, wherein R_{b} and R_{c} are each independently H, C₁-C₆ alkyl, alkoxy (—O-alkyl), thioalkoxy (—S-alkyl), cyano (—CN), or amino, or
R₁ and R₁ₐ are taken together to form ═O, ═NORₐ, or ═S, wherein Rₐ is H, C₁-C₆ alkyl, or C₁-C₆ acyl, or
R₁ and R₁ₐ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₂ (n=0 to 2, R₁₂ is directly connected to S), wherein R₁₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio; and
R₂ and R₉ are each independently selected from the group consisting of hydrogen, aryl, heteroaryl, cycloalkyl, heterocycloalkyl, alkyl, —NR₂ₐC(O)OR_{2b}, —NR₂ₐC(O)R_{2b}, —(C₀-C₆ alkyl)-CONHSO₂R₂ₐ, —(C₀-C₆ alkyl)-CONHSO₂NR₂ₐR_{2b}, —(C₀-C₆ alkyl)-SO₂NHCOR₂ₐ, —(C₀-C₆ alkyl)-SO₂NHR₂ₐ, —(C₀-C₆ alkyl)-CONR₂ₐOR_{2b}, 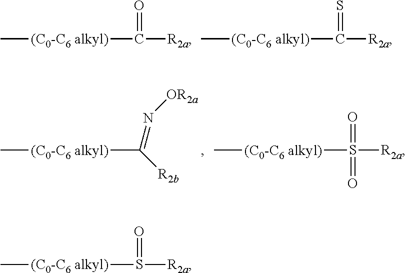
deuterium, halo, amino, hydroxy, cyano, formyl, nitro, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₀ (n=0 to 2, R₁₀ is directly connected to S), wherein R₁₀ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio, wherein R₂ₐ and R_{2b} are each independently H, C₁-C₆ alkyl, alkoxy (—O-alkyl), hydroxy, thioalkoxy (—S-alkyl), cyano (—CN), or amino;
R₄, R₅, R₆, R₇, and R₈ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;
R_{N} is H, CN, C₁-C₆ alkyl, —OH, —(CO)—OR, or —OR, wherein R is H, C₁-C₆ alkyl, or C₁-C₆ acyl; and
optionally, adjacent R groups, together, can form a six- to twelve-membered ring.

7. A method of treating cancer in a patient, comprising administering to the patient (1) a therapeutically effective amount of a compound of formula 4, or an enantiomer, diastereomer, or pharmaceutically acceptable salt thereof, and (2) a therapeutically effective amount of an inhibitor of an immune checkpoint protein, 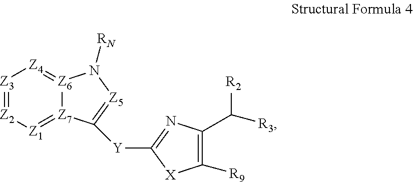
wherein:
X is O (oxygen) or S (sulfur);
Y is a bond, O (oxygen), S (sulfur), or 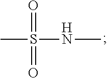
Z₁ is N or CR₄, Z₂ is N or CR₅, Z₃ is N or CR₆, Z₄ is N or CR₇, Z₅ is N or CR₈, Z₆ is N or C, Z₇ is N or C, wherein no more than two of Z₁, Z₂, Z₃, Z₄, Z₅, Z₆, and Z₇ are N;
R₄, R₅, R₆, R₇, R₈, and R₉ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₀ (n=0 to 2, R₁₀ is directly connected to S), wherein R₁₀ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;
R_{N} is H, CN, C₁-C₆ alkyl, —OH, —(CO)—OR, or —OR, wherein R is H, C₁-C₆ alkyl, or C₁-C₆ acyl;
R₂ and R₃ are together selected from the group consisting of ═O, ═S, or ═NRₐ (Rₐ is H, C₁-C₆ alkyl, C₁-C₆ acyl, or —OR, R is H, C₁-C₆ alkyl, or C₁-C₆ acyl), or
R₂ and R₃ are each independently selected from the group consisting of —NRₐR_{b} (Rₐ and R_{b} are each independently H, C₁-C₆ alkyl, or C₁-C₆ acyl), hydrogen, deuterium, halo, amino, hydroxy, cyano, formyl, furyl, nitro, alkyl, haloalkyl, alkenyl, haloalkenyl, alkynyl, haloalkynyl, C₁-C₆ acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio; and
optionally, adjacent R groups, together, can form a six- to twelve-membered ring.

8. A method of treating cancer in a patient, comprising administering to the patient (1) a therapeutically effective amount of a compound of formula 5, or an enantiomer, diastereomer, or pharmaceutically acceptable salt thereof, and (2) a therapeutically effective amount of an inhibitor of an immune checkpoint protein, 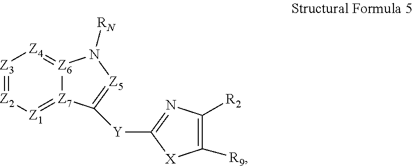
wherein:
X is O (oxygen) or S (sulfur);
Y is a bond, O (oxygen), S (sulfur), or 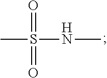
Z₁ is N or CR₄, Z₂ is N or CR₅, Z₃ is N or CR₆, Z₄ is N or CR₇, Z₅ is N or CR₈, Z₆ is N or C, Z₇ is N or C, wherein no more than two of Z₁, Z₂, Z₃, Z₄, Z₅, Z₆, and Z₇ are N;
R₂ and R₉ are each independently selected from the group consisting of hydrogen, aryl, heteroaryl, cycloalkyl, heterocycloalkyl, alkyl, —NR₂ₐC(O)OR_{2b}, —NR₂ₐC(O)R_{2b}, —(C₀-C₆ alkyl)-CONHSO₂R₂ₐ, —(C₀-C₆ alkyl)-CONHSO₂NR₂ₐR_{2b}, —(C₀-C₆ alkyl)-SO₂NHCOR₂ₐ, —(C₀-C₆ alkyl)-SO₂NHR₂ₐ, —(C₀-C₆ alkyl)-CONR₂ₐOR_{2b}, 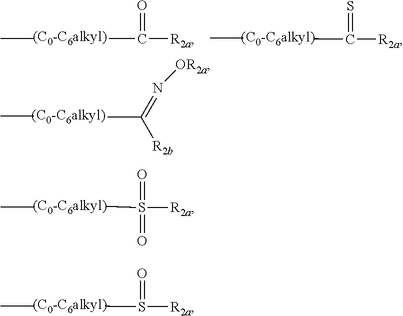
deuterium, halo, amino, hydroxy, cyano, formyl, nitro, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₀ (n=0 to 2, R₁₀ is directly connected to S), wherein R₁₀ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio, wherein R₂ₐ and R_{2b} are each independently H, C₁-C₆ alkyl, alkoxy (—O-alkyl), hydroxy, thioalkoxy (—S-alkyl), cyano (—CN), or amino;
R₄, R₅, R₆, R₇, and R₈ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;
R_{N} is H, CN, C₁-C₆ alkyl, —OH, —(CO)—OR, or —OR, wherein R is H, C₁-C₆ alkyl, or C₁-C₆ acyl; and
optionally, adjacent R groups, together, can form a six- to twelve-membered ring.

9. A method of treating cancer in a patient, comprising administering to the patient (1) a therapeutically effective amount of a compound of formula 6, or an enantiomer, diastereomer, or pharmaceutically acceptable salt thereof, and (2) a therapeutically effective amount of an inhibitor of an immune checkpoint protein, 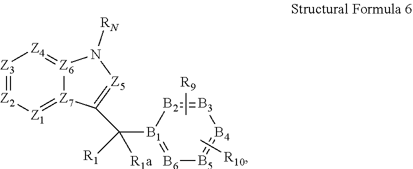
wherein:
R₁ and R₁ₐ are taken together to form ═NR_{b}, wherein R_{b} is H, C₁-C₆ alkyl, hydroxy, C₁-C₆ alkoxy (—O-alkyl), C₁-C₆ acyloxy, amino, or C₁-C₆ acyl, or
R₁ and R₁ₐ are taken together to form ═CR_{b}R_{c}, wherein R_{b} and R_{c} are each independently H, C₁-C₆ alkyl, alkoxy (—O-alkyl), thioalkoxy (—S-alkyl), cyano (—CN), or amino, or
R₁ and R₁ₐ are taken together to form ═O, ═NORₐ, or ═S, wherein Rₐ is H, C₁-C₆ alkyl, or C₁-C₆ acyl, or
R₁ and R₁ₐ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₄ (n=0 to 2, R₁₄ is directly connected to S), wherein R₁₄ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;
Z₁ is N or CR₄, Z₂ is N or CR₅, Z₃ is N or CR₆, Z₄ is N or CR₇, Z₅ is N or CR₈, Z₆ is N or C, Z₇ is N or C, wherein no more than two of Z₁, Z₂, Z₃, Z₄, Z₅, Z₆, and Z₇ are N;
R₄, R₅, R₆, R₇, and R₈ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;
R_{N} is H, CN, C₁-C₆ alkyl, —OH, —(CO)—OR, or —OR, wherein R is H, C₁-C₆ alkyl, or C₁-C₆ acyl;
B₁, B₂, B₃, B₄, B₅, and B₆ are each independently C or N;
R₉ and R₁₀, the number of which, together, complete the valence of each of B₁, B₂, B₃, B₄, B₅, and B₆, are each independently selected from the group consisting of hydrogen, aryl, heteroaryl, cycloalkyl, heterocycloalkyl, alkyl, —NR₂ₐC(O)OR_{2b}, —NR₂ₐC(O)R_{2b}, —(C₀-C₆ alkyl)-CONHSO₂R₂ₐ, —(C₀-C₆ alkyl)-CONHSO₂NR₂ₐR_{2b}, —(C₀-C₆ alkyl)-SO₂NHCOR₂ₐ, —(C₀-C₆ alkyl)-SO₂NHR₂ₐ, —(C₀-C₆ alkyl)-CONR₂ₐOR_{2b}, 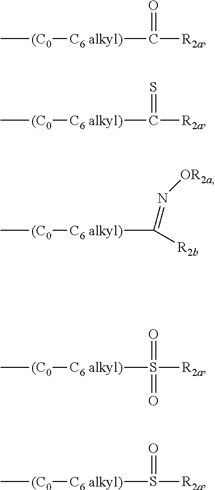
deuterium, halo, amino, hydroxy, cyano, formyl, nitro, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₂ (n=0 to 2, R₁₂ is directly connected to S), wherein R₁₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and 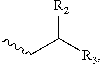
wherein R₂ₐ and R_{2b} are each independently H, C₁-C₆ alkyl, alkoxy (—O-alkyl), hydroxy, thioalkoxy (—S-alkyl), cyano (—CN), or amino;
wherein R₂ and R₃ are together selected from the group consisting of ═O, ═S, or ═NRₐ (Rₐ is H, C₁-C₆ alkyl, C₁-C₆ acyl, or —OR, R is H, C₁-C₆ alkyl, or C₁-C₆ acyl), or
R₂ and R₃ are each independently selected from the group consisting of —NRₐR_{b} (Rₐ and R_{b} are each independently H, C₁-C₆ alkyl, or C₁-C₆ acyl), hydrogen, deuterium, halo, amino, hydroxy, cyano, formyl, furyl, nitro, alkyl, haloalkyl, alkenyl, haloalkenyl, alkynyl, haloalkynyl, C₁-C₆ acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₃ (n=0 to 2, R₁₃ is directly connected to S), wherein R₁₃ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio; and
optionally, adjacent R groups, together, can form a six- to twelve-membered ring.

10. A method of treating cancer in a patient, comprising administering to the patient (1) a therapeutically effective amount of a compound of formula 7, or an enantiomer, diastereomer, or pharmaceutically acceptable salt thereof, and (2) a therapeutically effective amount of an inhibitor of an immune checkpoint protein, 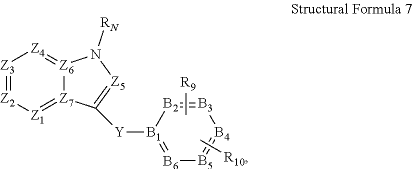
wherein:
Y is a bond, O (oxygen), S (sulfur), or 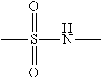
Z₁ is N or CR₄, Z₂ is N or CR₅, Z₃ is N or CR₆, Z₄ is N or CR₇, Z₅ is N or CR₈, Z₆ is N or C, Z₇ is N or C, wherein no more than two of Z₁, Z₂, Z₃, Z₄, Z₅, Z₆, and Z₇ are N;
R₄, R₅, R₆, R₇, and R₈ are each independently selected from the group consisting of hydrogen, deuterium, halo, amino, hydroxy, cyano, formyl, furyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₁ (n=0 to 2, R₁₁ is directly connected to S), wherein R₁₁ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio;
R_{N} is H, CN, C₁-C₆ alkyl, —OH, —(CO)—OR, or —OR, wherein R is H, C₁-C₆ alkyl, or C₁-C₆ acyl;
B₁, B₂, B₃, B₄, B₅, and B₆ are each independently C or N;
R₉ and R₁₀, the number of which, together, complete the valence of each of B₁, B₂, B₃, B₄, B₅, and B₆, are each independently selected from the group consisting of hydrogen, aryl, heteroaryl, cycloalkyl, heterocycloalkyl, alkyl, —NR₂ₐC(O)OR_{2b}, —NR₂ₐC(O)R_{2b}, —(C₀-C₆ alkyl)-CONHSO₂R₂ₐ, —(C₀-C₆ alkyl)-CONHSO₂NR₂ₐR_{2b}, —(C₀-C₆ alkyl)-SO₂NHCOR₂ₐ, —(C₀-C₆ alkyl)-SO₂NHR₂ₐ, —(C₀-C₆ alkyl)-CONR₂ₐOR_{2b}, 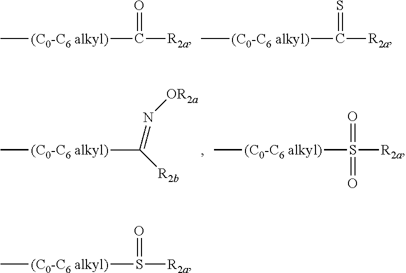
deuterium, halo, amino, hydroxy, cyano, formyl, nitro, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₂ (n=0 to 2, R₁₂ is directly connected to S), wherein R₁₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and 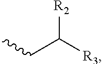
wherein R₂ₐ and R_{2b} are each independently H, C₁-C₆ alkyl, alkoxy (—O-alkyl), hydroxy, thioalkoxy (—S-alkyl), cyano (—CN), or amino;
wherein R₂ and R₃ are together selected from the group consisting of ═O, ═S, or ═NRₐ (Rₐ is H, C₁-C₆ alkyl, C₁-C₆ acyl, or —OR, R is H, C₁-C₆ alkyl, or C₁-C₆ acyl), or
R₂ and R₃ are each independently selected from the group consisting of —NRₐR_{b} (Rₐ and R_{b} are each independently H, C₁-C₆ alkyl, or C₁-C₆ acyl), hydrogen, deuterium, halo, amino, hydroxy, cyano, formyl, furyl, nitro, alkyl, haloalkyl, alkenyl, haloalkenyl, alkynyl, haloalkynyl, C₁-C₆ acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₙR₁₃ (n=0 to 2, R₁₃ is directly connected to S), wherein R₁₃ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, and halothiocarbonylthio; and
optionally, adjacent R groups, together, can form a six- to twelve-membered ring.

11. The method of any one of claims 1-10, wherein each of R₄, R₅, R₆, and R₇ is hydrogen.

12. The method of any one of claims 1-10, wherein at least one of R₄, R₅, R₆, and R₇ is F, Cl or Br and the others of R₄, R₅, R₆, and R₇ are hydrogen.

13. The method of any one of claims 1-10, wherein at least two of R₄, R₅, R₆, and R₇, independently, are F, Cl or Br and the others of R₄, R₅, R₆, and R₇ are hydrogen.

14. The method of claim 12 or 13, wherein the F, Cl or Br is at the indole ring carbon 5, 6, or 7.

15. The method of any one of claims 1, 2, and 7, wherein R₂ is hydroxyl and R₃ is alkyl, aryl, nitro, or cyano.

16. The method of any one of claims 1, 2, and 7, wherein R₂ is amino and R₃ is alkyl, aryl, nitro, or cyano.

17. The method of claim 16, wherein the amino is unsubstituted.

18. The method of any one of claims 1, 2, 7, and 15-17, wherein R₉ is hydrogen.

19. The method of any one of claims 3-6 and 8, wherein R₂ is acyl, cyano, hydroxyl-substituted C1-C6 alkyl, amino-substituted C1-C6 alkyl, aryl, or heteroaryl.

20. The method of claim 19, wherein the aryl is substituted aryl.

21. The method of claim 20, wherein the aryl is substituted with halo, amino, hydroxyl, or C1-C6 alkyl.

22. The method of claim 21, wherein the amino is unsubstituted amino.

23. The method of claim 19, wherein the heteroaryl is substituted heteroaryl.

24. The method of claim 23, wherein the heteroary is substituted with halo, amino, hydroxyl, or C1-C6 alkyl.

25. The method of claim 24, wherein the amino is unsubstituted amino.

26. The method of any one of claims 3-6, 8, and 19-25, wherein R₉ is hydrogen.

27. A method of treating cancer in a patient, comprising administering to the patient (1) a therapeutically effective amount of any one of the compounds in Table 1 and Table 2, or an enantiomer, diastereomer, or pharmaceutically acceptable salt thereof, and (2) a therapeutically effective amount of an inhibitor of an immune checkpoint protein.

28. The method of claim 27, wherein the compound is selected from the group consisting of ARI-001, ARI-002, ARI-003, ARI-017, ARI-018, ARI-019, and ARI-020, and an enantiomer, diastereomer, or pharmaceutically acceptable salt thereof.

29. A method of treating cancer in a patient, comprising administering to the patient (1) a therapeutically effective amount of a compound of formula 8, or an enantiomer, diastereomer, or pharmaceutically acceptable salt thereof, and (2) a therapeutically effective amount of an inhibitor of an immune checkpoint protein, 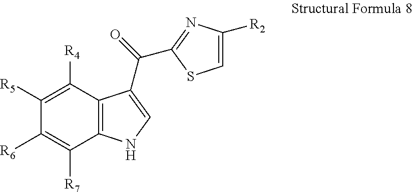
wherein R₂ is selected from the group consisting of substituted alkyl, heteroaryl, and 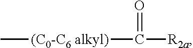
wherein R₂ₐ is H, C1-C6 alkyl, alkoxy (—O-alkyl), hydroxy, thioalkoxy (—S-alkyl), cyano (—CN), or amino; and
R₄, R₅, R₆, and R₇, are each, independently, selected from the group consisting of hydrogen and halo.

30. The method of claim 29, wherein R₂ is substituted alkyl.

31. The method of claim 30, wherein the substituted alkyl is a C1-C6 alkyl substituted with one or more hydroxyl, amino, nitro, or cyano.

32. The method of claim 29, wherein R₂ is heteroaryl.

33. The method of claim 32, wherein the heteroaryl is oxadiazolyl or thiadiazolyl, optionally substituted with one or more hydroxyl, amino, nitro, cyano, C1-C6 alkyl, or C1-C6 alkyl amino.

34. The method of claim 29, wherein R₂ is —C(O)—R₂ₐ, and wherein R₂ₐ is C1-C6 alkyl.

35. The method of any one of claims 29-34, wherein at least one of R₄, R₅, R₆, and R₇ is F, Cl or Br and the others of R₄, R₅, R₆, and R₇ are hydrogen.

36. The method of any one of claims 29-34, wherein at least two of R₄, R₅, R₆, and R₇ are F, Cl or Br and the others of R₄, R₅, R₆, R₇ are hydrogen.

37. The method of any one of claims 29-34, wherein R₅ is F and R₄, R₆, and R₇ are hydrogen.

38. The method of any one of claims 29-34, wherein R₆ is F and R₄, R₅, and R₇ are hydrogen.

39. The method of any one of claims 29-34, wherein R₇ is F and R₄, R₅, and R₆ are hydrogen.

40. The method of any one of claims 29-34, wherein R₅ is Cl and R₄, R₆, and R₇ are hydrogen.

41. The method of any one of claims 29-34, wherein R₆ is Cl and R₄, R₅, and R₇ are hydrogen.

42. The method of any one of claims 29-34, wherein R₇ is Cl and R₄, R₅, and R₆ are hydrogen.

43. The method of any one of claims 29-34, wherein R₅ and R₆ are F and R₄ and R₇ are hydrogen.

44. The method of any one of claims 29-34, wherein R₅ and R₇ are F and R₄ and R₆ are hydrogen.

45. The method of any one of claims 29-34, wherein R₆ and R₇ are F and R₄ and R₅ are hydrogen.

46. The method of any one of claims 29-34, wherein R₅ and R₆ are Cl and R₄ and R₇ are hydrogen.

47. The method of any one of claims 29-34, wherein R₅ and R₇ are Cl and R₄ and R₆ are hydrogen.

48. The method of any one of claims 29-34, wherein R₆ and R₇ are Cl and R₄ and R₅ are hydrogen.

49. The method of any one of claims 29-34, wherein each of R₄, R₅, R₆ and R₇ is hydrogen.

50. The method of claim 29, wherein the compound is selected from any one of the compounds in the following table, or an enantiomer, diastereomer, or pharmaceutically acceptable salt thereof:

| ARI-# | Structural Formula |
|---|---|
| 031 | 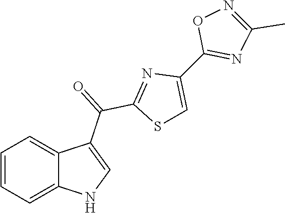 [SMILES: Cc1noc(-c2csc(C(=O)c3c[nH]c4ccccc34)n2)n1] |
| 060 | 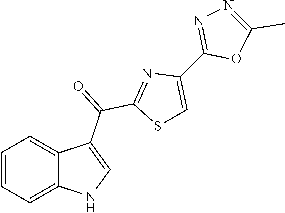 [SMILES: Cc1nnc(-c2csc(C(=O)c3c[nH]c4ccccc34)n2)o1] |
| 083 | 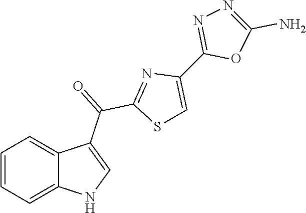 [SMILES: Nc1nnc(-c2csc(C(=O)c3c[nH]c4ccccc34)n2)o1] |
| 087 | 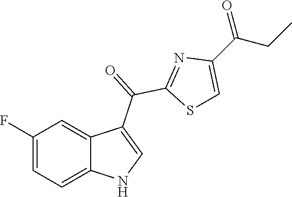 [SMILES: CCC(=O)c1csc(C(=O)c2c[nH]c3ccc(F)cc23)n1] |
| 090 | 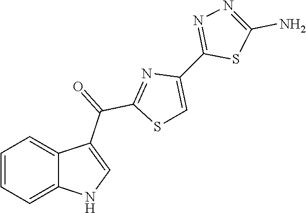 [SMILES: Nc1nnc(-c2csc(C(=O)c3c[nH]c4ccccc34)n2)s1] |
| 118 | 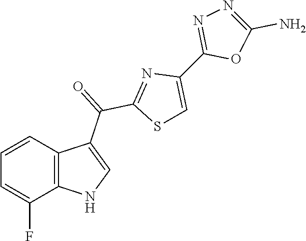 [SMILES: Nc1nnc(-c2csc(C(=O)c3c[nH]c4c(F)cccc34)n2)o1] |
| 120 | 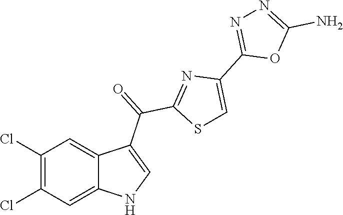 [SMILES: Nc1nnc(-c2csc(C(=O)c3c[nH]c4cc(Cl)c(Cl)cc34)n2)o1] |
| 140 | 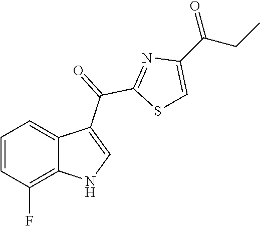 [SMILES: CCC(=O)c1csc(C(=O)c2c[nH]c3c(F)cccc23)n1] |
| 143 | 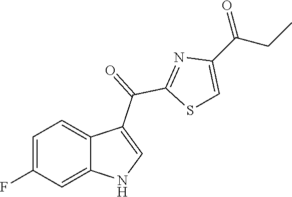 [SMILES: CCC(=O)c1csc(C(=O)c2c[nH]c3cc(F)ccc23)n1] |
| 145 | 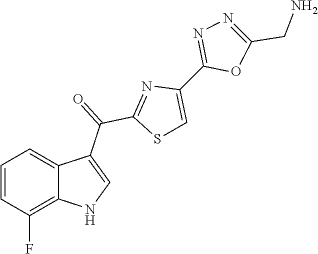 [SMILES: NCc1nnc(-c2csc(C(=O)c3c[nH]c4c(F)cccc34)n2)o1] |
| 146 | 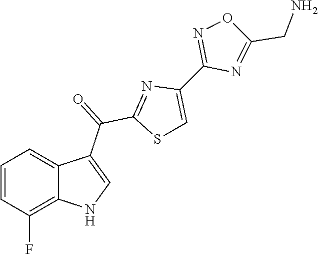 [SMILES: NCc1nc(-c2csc(C(=O)c3c[nH]c4c(F)cccc34)n2)no1] |
| 148 | 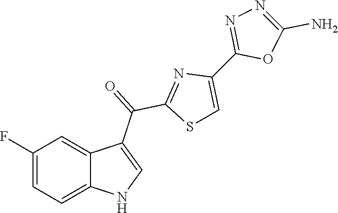 [SMILES: Nc1nnc(-c2csc(C(=O)c3c[nH]c4ccc(F)cc34)n2)o1] |
| 149 | 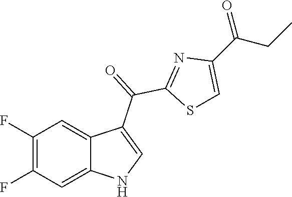 [SMILES: CCC(=O)c1csc(C(=O)c2c[nH]c3cc(F)c(F)cc23)n1] |
| 150 | 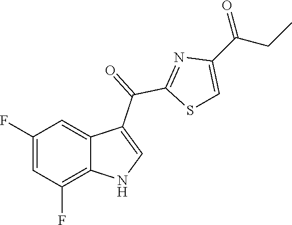 [SMILES: CCC(=O)c1csc(C(=O)c2c[nH]c3c(F)cc(F)cc23)n1] |

51. A method of treating cancer in a patient, comprising administering to the patient (1) a therapeutically effective amount of a compound of Formula I, and (2) a therapeutically effective amount of an inhibitor of an immune checkpoint protein, 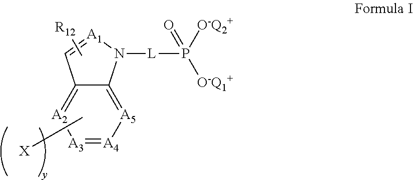
wherein:
R₁₂ is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio,
each of A₁, A₂, A₃, A₄, and A₅, independently, is CR₂ or N;
L is —(CR₂R₃—O)ₙ— or a bond;
R₂ is H or C1-C6 alkyl;
R₃ is H or C1-C6 alkyl;
or, together, R₂ and R₃ form a C3-C8 cycloalkyl;
n is 0, 1, 2, 3, 4, 5, or 6;
y is 0, 1, 2, 3, or 4;
each X, independently, is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio; and
Q₁⁺ and Q₂⁺ are each, independently, a monocation, or together are a dication or one of Q₁⁺ or Q₂⁺ is C1-C6 alkyl, benzyl, allyl or —(CR₂R₃—O)—R₂₃, and R₂₃ is H, or C1-C6 alkyl.

52. The method claim 51, wherein the compound is of Formula II, 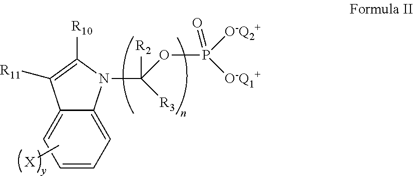
wherein:
R₁₀ is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio;
R₁₁ is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio, wherein one of R₁₀ and R₁₁ is H or C1-C6 alkyl;
R₂ is H or C1-C6 alkyl;
R₃ is H or C1-C6 alkyl;
or, together, R₂ and R₃ form a C3-C8 cycloalkyl;
y is 0, 1, 2, 3, or 4;
each X, independently, is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio;
Q₁⁺ and Q₂⁺ are each, independently, a monocation, or together are a dication or one of Q₁⁺ or Q₂⁺ is C1-C6 alkyl, benzyl, allyl or —(CR₂R₃—O)—R₂₃, and R₂₃ is H, or C1-C6 alkyl; and
n is 0, 1, 2, 3, 4, 5, or 6.

53. The method claim 51, wherein the compound is of Formula III, 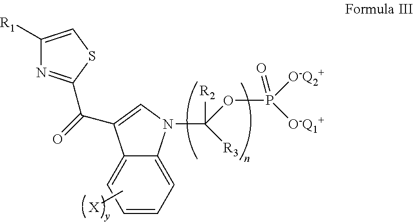
wherein:
R₁ is —C(═O)—R₄, cyano, an oxadiazole, or a thiadiazole;
R₂ and R₃ are each, independently, hydrogen, or C₁-C₆ alkyl; and
R₄ is selected from the group consisting of —NRₐR_{b} (Rₐ and R_{b} are each independently H, C₁-C₆ alkyl, or C₁-C₆ acyl), hydrogen, deuterium, halo, amino, hydroxy, cyano, formyl, furyl, nitro, alkyl, haloalkyl, alkenyl, haloalkenyl, alkynyl, haloalkynyl, unsubstituted or substituted C₁-C₆ acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₘR₂₂ (m=0 to 2, R₂₂ is directly connected to S), wherein R₂₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carbonyloxy, halocarbonyloxy, carbonylthio, halocarbonylthio, thiocarbonyloxy, halothiocarbonyloxy, thiocarbonylthio, and halothiocarbonylthio;
y is 0, 1, 2, 3, or 4;
each X, independently, is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio;
Q₁⁺ and Q₂⁺ is each, independently, a monocation, or together are a dication or one of Q₁⁺ or Q₂⁺ is C1-C6 alkyl, benzyl, allyl or —(CR₂R₃—O)—R₂₃, and R₂₃ is H or C1-C6 alkyl, and the other of Q₁⁺ or Q₂⁺ can be a monocation; and
n is 0, 1, 2, 3, 4, 5, or 6.

54. The method claim 51, wherein the compound is of Formula IV, 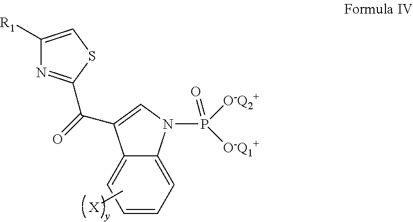
wherein:
R₁ is —C(═O)—R₄, cyano, an oxadiazole, or a thiadiazole; and
R₄ is selected from the group consisting of —NRₐR_{b} (Rₐ and R_{b} are each independently H, C₁-C₆ alkyl, or C₁-C₆ acyl), hydrogen, deuterium, halo, amino, hydroxy, cyano, formyl, furyl, nitro, alkyl, haloalkyl, alkenyl, haloalkenyl, alkynyl, haloalkynyl, unsubstituted or substituted C₁-C₆ acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₘR₂₂ (m=0 to 2, R₂₂ is directly connected to S), wherein R₂₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carbonyloxy, halocarbonyloxy, carbonylthio, halocarbonylthio, thiocarbonyloxy, halothiocarbonyloxy, thiocarbonylthio, and halothiocarbonylthio;
y is 0, 1, 2, 3, or 4;
each X, independently, is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio; and
Q₁⁺ and Q₂⁺ is each, independently, a monocation, or together are a dication or one of Q₁⁺ or Q₂⁺ is C1-C6 alkyl, benzyl, allyl or —(CR₂R₃—O)—R₂₃, and R₂₃ is H or C1-C6 alkyl, and the other of Q₁⁺ or Q₂⁺ can be a monocation.

55. The method claim 51, wherein the compound is of Formula V, 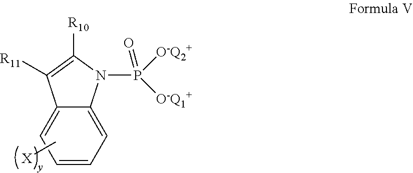
wherein:
R₁ is —C(═O)—R₄, cyano, an oxadiazole, or a thiadiazole;
R₂ and R₃ are each independently hydrogen, or C₁-C₆ alkyl; and
R₄ is selected from the group consisting of —NRₐR_{b} (Rₐ and R_{b} are each independently H, C₁-C₆ alkyl, or C₁-C₆ acyl), hydrogen, deuterium, halo, amino, hydroxy, cyano, formyl, furyl, nitro, alkyl, haloalkyl, alkenyl, haloalkenyl, alkynyl, haloalkynyl, unsubstituted or substituted C₁-C₆ acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₘR₂₂ (m=0 to 2, R₂₂ is directly connected to S), wherein R₂₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carbonyloxy, halocarbonyloxy, carbonylthio, halocarbonylthio, thiocarbonyloxy, halothiocarbonyloxy, thiocarbonylthio, and halothiocarbonylthio;
y is 0, 1, 2, 3, or 4;
each X, independently, is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio; and
Q₁⁺ and Q₂⁺ is each, independently, a monocation, or together are a dication or one of Q₁⁺ or Q₂⁺ is C1-C6 alkyl, benzyl, allyl or —(CR₂R₃—O)—R₂₃, and R₂₃ is H or C1-C6 alkyl, and the other of Q₁⁺ or Q₂⁺ can be a monocation.

56. The method of any one of claims 51-55, wherein Q₁⁺ and Q₂⁺ are each, independently, an alkali metal.

57. The method of any one of claims 51-55, wherein Q₁⁺ and Q₂⁺ are each, independently, selected from the group consisting of ammonium and alkyl ammonium.

58. The method of any one of claims 51-55, wherein Q₁⁺ and Q₂⁺ together are selected from the group consisting of an alkaline earth metal salt.

59. The method of any one of claims 51-55, wherein Q₁⁺ and Q₂⁺ are each independently selected from the group consisting of zinc, calcium and magnesium.

60. The method of any one of claims 51-55, wherein Q₁⁺ and Q₂⁺ are each independently lithium, sodium, or potassium, y is 0, 1 or 2, and X is F, Cl, or Br.

61. The method of any one of claim 53 or 54, wherein R₁ is —C(═O)—R₄, and R₄ is C₁-C₆ alkyl or C₁-C₆ alkoxy.

62. The method of any one of claim 53 or 54, wherein R₁ is an oxadiazole or a thiadiazole, wherein the oxadiazole, or the thiadiazole is optionally substituted by amino, alkyl amino, amino alkyl, alkoxy, alkyl or haloalkyl.

63. The method of any one of claims 51-53, wherein n is 0 or 1.

64. The method of claim 52, wherein the compound is selected from the group consisting of:

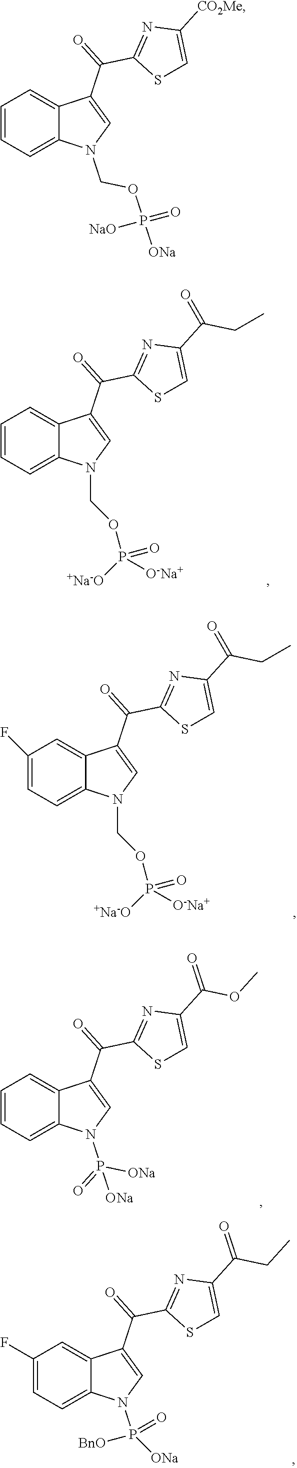

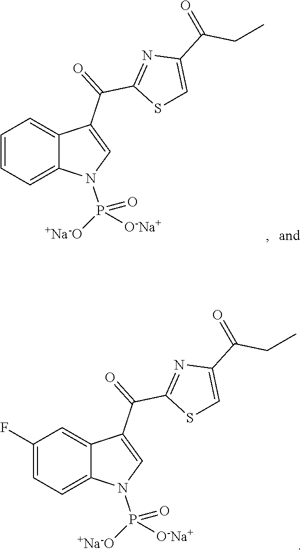

65. The method of any one of claim 53 or 54, wherein R1 is an unsubstituted or substituted oxadiazole.

66. The method of claim 65, wherein the substituted oxadiazole is a C1-C6 alkyl oxadiazole, haloalkyl oxadiazole, halo oxadiazole, amino oxadiazole, alkyl amino oxadiazole, amino alkyl oxadiazole, or hydroxy oxadiazole.

67. The method of claim 66, wherein n is 0.

68. The method of claim 67, wherein Q₁⁺ and Q₂⁺ are each lithium, sodium, or potassium.

69. The method of claim 66, wherein the indole is a fluorinated indole.

70. The method of claim 52, wherein the compound is selected from the group consisting of:

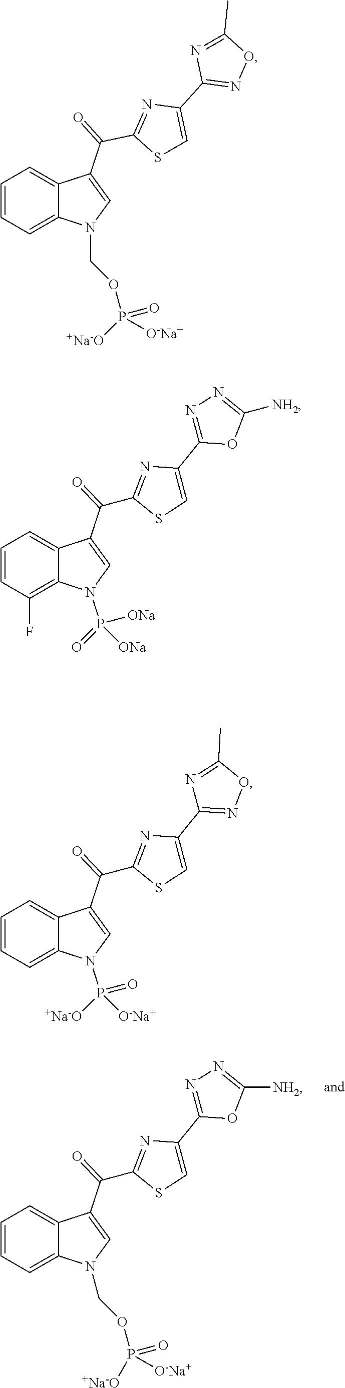

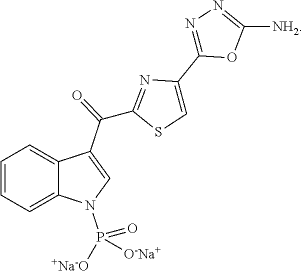

71. A method of treating cancer in a patient, comprising administering to the patient (1) a therapeutically effective amount of a compound of Formula VI, and (2) a therapeutically effective amount of an inhibitor of an immune checkpoint protein, 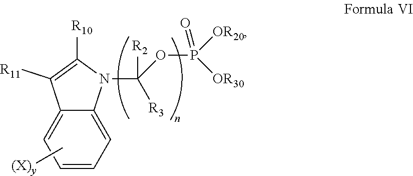
wherein:
R₁₀ is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio;
R₁₁ is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio, wherein one of R₁₀ and R₁₁ is H or C1-C6 alkyl;
R₂ is H or C1-C6 alkyl;
R₃ is H or C1-C6 alkyl;
or, together, R₂ and R₃ form a C3-C8 cycloalkyl;
y is 0, 1, 2, 3, or 4;
each X, independently, is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio;
R₂₀ and R₃₀ each, independently, is C1-C6 alkyl or benzyl, or one of R₂₀ or R₃₀ is H, C1-C6 alkyl, allyl, or benzyl and the other of R₂₀ or R₃₀ is a cation; and
n is 0, 1, 2, 3, 4, 5, or 6.

72. The method of claim 51 or 71, wherein the compound is any one of the compounds in Table 3.

73. An indole compound for use in treating cancer in a method of any one of claims 1-72.

74. An inhibitor of an immune checkpoint protein for use in treating cancer in a method of any one of claims 1-72.

75. Use of an indole compound for the manufacture of a medicament for treating cancer in a method of any one of claims 1-72.

76. Use of an inhibitor of an immune checkpoint protein for the manufacture of a medicament for treating cancer in a method of any one of claims 1-72.

77. The method of any one of claims 1-72, compound of claim 73, inhibitor of claim 74, or use of claim 75 or 76, wherein the immune checkpoint protein is PD-1, PD-L1, PD-L2, or CTLA-4.

78. The method, compound, inhibitor, or use of claim 77, wherein the inhibitor of the immune checkpoint protein is an anti-PD-1 antibody or an anti-CTLA-4 antibody.

79. The method, compound, inhibitor, or use of any one of the proceeding claims, wherein the cancer is refractory to anti-PD-1 antibody treatment.

80. The method, compound, inhibitor, or use of any one of the preceding claims, wherein the cancer is a lymphoma.

81. The method, compound, inhibitor, or use of any one of the preceding claims, wherein the cancer is a solid tumor.

82. The method, compound, inhibitor, or use of any one of the proceeding claims, wherein the cancer is selected from the group consisting of diffuse large B-cell lymphoma, marginal zone lymphoma, chronic lymphocytic leukemia, small lymphocytic lymphoma, prolymphocytic leukemia, acute lymphocytic leukemia, Waldenström's Macroglobulinemia, follicular lymphoma, mantle cell lymphoma, Hodgkin lymphoma, non-Hodgkin lymphoma, multiple myeloma, prostate cancer, ovarian cancer, fallopian tube cancer, cervical cancer, breast cancer, lung cancer, skin cancer, colon cancer, colorectal cancer, stomach cancer, pancreatic cancer, liver cancer, kidney cancer, bladder cancer, soft tissue cancer, glioma, and head and neck cancer.

83. The method, compound, inhibitor, or use of claim 82, wherein the cancer is selected from the group consisting of colon cancer, breast cancer, pancreatic cancer, lung cancer, prostate cancer, kidney cancer, and melanoma.
